(19) United States
(12) Patent Application Publication
(10) Pub. No.: US 2026/0337644 A1
(43) Pub. Date: Oct. 29, 2026

(54) PROCESS FOR THE PRODUCTION OF (1R,2S)-2,6-DIMETHYL-1-INDANAMINE

(71) Applicant: BAYER AKTIENGESELLSCHAFT, Leverkusen (DE)

(72) Inventors: Florian ERVER, Wiesbaden (DE); Alba HERNANDEZ MARTIN, Düsseldorf (DE); Mark James FORD, Oberreifenberg (DE); Dirk BROHM, Mettmann (DE); Arne GERLACH, Odenthal (DE)

(21) Appl. No.: 19/104,730

(22) PCT Filed: Aug. 17, 2023
(86) PCT No.: PCT/EP2023/072654

§ 371 Date: Feb. 19, 2025

(30) Foreign Application Priority Data
Aug. 23, 2022 (EP) 22191617.4

(51) Int. Cl. C12P 13/02; C07C 209/68; C12N 9/20; C12P 13/00

(52) U.S. Cl. CPC C12P 13/02; C07C 209/68; C12N 9/20; C12P 13/001; C12Y 301/01003; C07C 2523/44

(57) ABSTRACT

There is described a process for preparing virtually enantiopure (1R,2S)-2,6-dimethyl-1-indanamine, characterized by reaction of a mixture of the four stereoisomers of 2,6-dimethyl-1-indanamine with an acylation or carboxylation agent in the presence of a protein having the activity of a lipase.

The present invention relates to a process for preparing virtually enantiopure (1R,2S)-2,6-dimethyl-1-indanamine, which is a valuable building block for the synthesis of the herbicidal active ingredient indaziflam. Specifically, the present invention relates to a process for preparing virtually enantiopure (1R,2S)-2,6-dimethyl-1-indanamine by enzyme-catalysed, stereoselective acylation of racemic 2,6-dimethyl-1-indanamine.

From the prior art, the only processes for preparing virtually enantiopure (1R,2S)-2,6-dimethyl-1-indanamine that are known to date are those which, owing to the use of costly reaction components and catalysts, are suitable only for the laboratory scale, but not for industrial use. For instance, WO 2004/69814 A1 discloses a process characterized by the following five reaction steps:
1. Preparation of a mixture of the four stereoisomers of 2,6-dimethyl-1-aminoindane by palladium-catalysed reduction of the corresponding oxime.
2. Column chromatography separation of said four stereoisomers into the cis and trans isomers.
3. Reaction of the trans isomer mixture with methyl 2-methoxyacetate in the presence of the enzyme Novozym 435® to form the corresponding acetylated (1R,2S)-2,6-dimethyl-1-indanamine.
4. Isolation of the acetylated (1R,2S)-2,6-dimethyl-1-indanamine.
5. Acidic hydrolysis of the acetylated (1R,2S)-2,6-dimethyl-1-indanamine to form the free (1R,2S)-2,6-dimethyl-1-indanamine.

Tetrahedron 2007, 63 (29), 6755-6763 describes a process for preparing (1R,2S)-2,6-dimethyl-1-indanamine, characterized by the following two reaction steps:
1. Racemic 1,6-dimethylindan-1-one is reduced by means of a chiral ruthenium catalyst both diastereoselectively (96:4 d.r.) and enantioselectively (98:2 e.r.) to form the corresponding (1S,2S)-2,6-dimethylindan-1-ol in a yield of 80%.
2. The reaction of the (1S,2S)-2,6-dimethylindan-1-ol thus obtained with diphenylphosphoryl azide and subsequent reduction with lithium aluminium hydride results in (1R,2S)-2,6-dimethyl-1-indanamine at a yield of 76%.

The disadvantages of this process are considered to be not only the use of costly reagents, but also the long reaction time of eight days in total.

It is an object of the present invention to provide a process for preparing virtually enantiopure (1R,2S)-2,6-dimethyl-1-indanamine that overcomes the disadvantages known from the prior art.

We found a process for preparing virtually enantiopure (1R,2S)-2,6-dimethyl-1-indanamine, characterized by reaction of a mixture of the four stereoisomers of 2,6-dimethyl-1-indanamine with an acylation or carboxylation agent in the presence of an enzyme.

The present invention provides a process for preparing virtually enantiopure (1R,2S)-2,6-dimethyl-1-indanamine, characterized
1. in that, in a first step, a mixture of the four stereoisomers of 2,6-dimethyl-1-indanamine (I) is reacted with an acylation or carboxylation agent R—C(═O)R¹ in the presence of a protein having the activity of a lipase to form the corresponding amide or carbamate (II) and a mixture (III) of the unreacted stereoisomers of 2,6-dimethyl-1-indanamine:

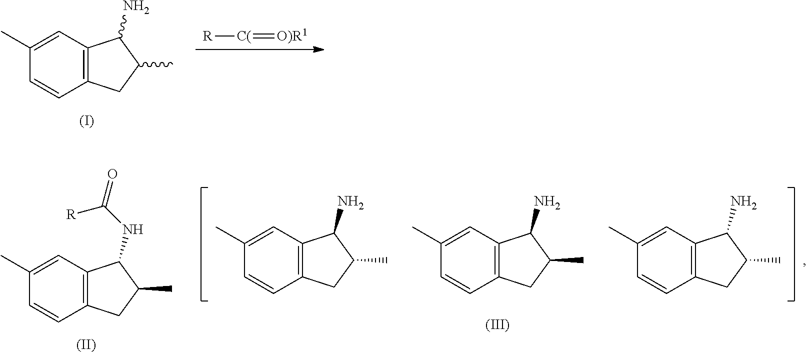

wherein the protein is encoded by an amino acid sequence selected from the group consisting of
I. proteins having at least 80%, preferably 85%, more preferably 90%, even more preferably 95%, even more preferably 96%, even more preferably 97%, particularly preferably 98%, most preferably 99% identity to the amino acid sequence shown under SEQ ID No. 1,
II. proteins having at least 80%, preferably 85%, more preferably 90%, even more preferably 95%, even more preferably 96%, even more preferably 97%, particularly preferably 98%, most preferably 99% identity to the amino acid sequence shown under SEQ ID No. 1 apart from the fact that the amino acid sequence having at least 80%, preferably 85%, more preferably 90%, even more preferably 95%, even more preferably 96%, even more preferably 97%, particularly preferably 98%, most preferably 99% identity to the amino acid sequence shown under SEQ ID No. 1 has a modification selected from the group consisting of
   i. the amino acid at position 186 is different from L;
   ii. the amino acid at position 280 is different from L;
   iii. the amino acid at position 312 is different from P;
   iv. the amino acid at position 3 is different from M;
   v. the amino acid at position 29 is different from N;
   vi. the amino acid at position 17 is different from L;
   vii. the amino acid at position 4 is different from S;
   viii. the amino acid at position 18 is different from V;
   ix. the amino acid at position 202 is different from A;
   x. the amino acid at position 301 is different from D;
   xi. the amino acid at position 309 is different from P;
   xii. the amino acid at position 31 is different from Q;
   xiii. the amino acid at position 111 is different from Q;
   xiv. the amino acid at position 85 is different from W;
   xv. the amino acid at position 8 is different from K;
   xvi. the amino acid at position 79 is different from E;
   xvii. the amino acid at position 40 is different from K;
2. in that, in a second step, the amide or carbamate (II) is separated from the remaining 2,6-dimethyl-1-indanamines (III) and secondary components by crystallization, and
3. in that, in a third step, the amide or carbamate (II) is converted to the virtually enantiopure (1R,2S)-2,6-dimethyl-1-indanamine (IV) using an acid or a base,

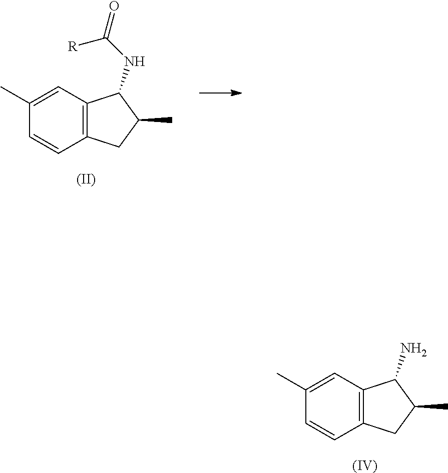

in which R means a radical from the group consisting of CH₂OCH₃, CH₂OCH₂CH₃, CH₃, OCH₃, OCH₂CH₃, OCH(CH₃)₂, OCH₂CH₂CH₂CH₃, OCH₂CHCH₂ and OCH₂(C₆H₅),
and
in which R¹ means a radical from the group consisting of OCH₃, OCH₂CH₃, OCH(CH₃)₂, OCH₂CH₂CH₂CH₃, OCH₂CHCH₂ and OCH₂(C₆H₅).

The mixture of the four stereoisomers of 2,6-dimethyl-1-indanamine (I) required as starting material for the process according to the invention is known and can, for example be prepared as described in WO 2004/69814.

Preferably, R means a radical from the group consisting of OCH₃ and OCH₂CH₃,
and
R¹ means a radical from the group consisting of OCH₃ and OCH₂CH₃.

In the process according to the invention, proteins having the activity of a lipolytic enzyme or a lipase are used.

SEQ ID No. 1 is the amino acid sequence of a lipolytic wild-type protein. The wild-type lipase is derived from a non-cultured bacterium from an environmental sample which can be derived from GenPept (PDB) under the accession number QRD81023 (version ORD81023.1). In case of doubt between the amino acid sequence shown in SEQ ID No. 1 and the sequence shown in the above database entry, SEQ ID No. 1 has precedence.

Further described are proteins having the activity of a lipolytic enzyme or a lipase, wherein the amino acid sequences of these proteins are variants of a known protein having the activity of a lipolytic enzyme or a lipase. In particular, the amino acid sequences of proteins having the activity of a lipase that are described herein are variants of the amino acid sequence described by the amino acids shown in SEQ ID No. 1, wherein, in the amino acid sequence shown under SEQ ID No. 1, at least the amino acid at position 186, the amino acid at position 280, the amino acid at position 312, the amino acid at position 3, the amino acid at position 29, the amino acid at position 17, the amino acid at position 4, the amino acid at position 18, the amino acid at position 202, the amino acid at position 301, the amino acid at position 309, the amino acid at position 31, the amino acid at position 111, the amino acid at position 85, the amino acid at position 8, the amino acid at position 79 or the amino acid at position 40 is different from the amino acid present at the corresponding amino acid position in the sequence shown under SEQ ID No. 1.

The term “variant”, as used herein, means an entity different from an entity known according to the prior art. With regard to nucleic acid molecules and proteins, variants are understood to mean a nucleic acid sequence or an amino acid sequence which deviates from correspondingly known sequences, but which encode a protein having the same function or catalysing the same reaction, for example the function of encoding a protein having the activity of a lipase. “Deviation” of nucleic acid molecule sequences and amino acid sequences from known nucleic acid sequences and protein sequences means that the sequences comprise substitutions (replacements) and/or deletions and/or insertions of nucleotides or amino acids compared to the corresponding known nucleic acid sequences or amino acid sequences. Use is usually made of proteins having the activity of a lipase, wherein the proteins are encoded by an amino acid sequence having at least 80%, preferably 85%, more preferably 90%, even more preferably 95%, even more preferably 96%, even more preferably 97%, particularly preferably 98%, most preferably 99% identity to the amino acid sequence shown under SEQ ID No. 1.

Use is likewise made of proteins having the activity of a lipase, wherein the proteins are encoded by an amino acid sequence having at least 80%, preferably 85%, more preferably 90%, even more preferably 95%, even more preferably 96%, even more preferably 97%, particularly preferably 98%, most preferably 99% identity to the amino acid sequence shown under SEQ ID No. 1 apart from the fact that the amino acid sequence having at least 80%, preferably 85%, more preferably 90%, even more preferably 95%, even more preferably 96%, even more preferably 97%, particularly preferably 98%, most preferably 99% identity to the amino acid sequence shown under SEQ ID No. 1 has a modification selected from the group consisting of
i. the amino acid at position 186 is different from L, preferably the amino acid at position 186 is F, W, Y, E, D, Q, T, H, P, C, K, S, N, I or V, more preferably the amino acid at position 186 is F, W, Y, E, D or K, particularly preferably the amino acid at position 186 is W or Y, most preferably the amino acid at position 186 is Y;
ii. the amino acid at position 280 is different from L, preferably the amino acid at position 280 is E, S, K, D or A, more preferably the amino acid at position 280 is A;
iii. the amino acid at position 312 is different from P, preferably the amino acid at position 312 is N, F, D, Q or K, more preferably the amino acid at position 312 is N;
iv. the amino acid at position 3 is different from M, preferably the amino acid at position 3 is L, Q or C, more preferably the amino acid at position 3 is Q;
v. the amino acid at position 29 is different from N, preferably the amino acid at position 29 is H, W or Y, more preferably the amino acid at position 29 is H or W, most preferably the amino acid at position 29 is H;
vi. the amino acid at position 17 is different from L, preferably the amino acid at position 17 is P or T, more preferably the amino acid at position 17 is P;
vii. the amino acid at position 4 is different from S, preferably the amino acid at position 4 is P or L, more preferably the amino acid at position 4 is P;
viii. the amino acid at position 18 is different from V, preferably the amino acid at position 18 is A, T, C or S, more preferably the amino acid at position 18 is A or C;
ix. the amino acid at position 202 is different from A, preferably the amino acid at position 202 is Q or N, more preferably the amino acid at position 202 is N;
x. the amino acid at position 301 is different from D, preferably the amino acid at position 301 is A;
xi. the amino acid at position 309 is different from P, preferably the amino acid at position 309 is C;
xii. the amino acid at position 31 is different from Q, preferably the amino acid at position 31 is W;
xiii. the amino acid at position 111 is different from Q, preferably the amino acid at position 111 is E;
xiv. the amino acid at position 85 is different from W, preferably the amino acid at position 85 is H;
xv. the amino acid at position 8 is different from K, preferably the amino acid at position 8 is E;
xvi. the amino acid at position 79 is different from K, preferably the amino acid at position 79 is S, I or W, more preferably the amino acid at position 79 is S;
xvii. the amino acid at position 40 is different from K, preferably the amino acid at position 40 is M.

The meaning of the amino acid abbreviations A, C, D, E, F, G, H, I, K, L, M, N, P, Q, R, S, TV, W, Y can be found herein in what follows in Table 2 in the section titled “Description of the sequences”.

A further embodiment according to the invention concerns proteins having the activity of a lipase, wherein the proteins are selected from the group consisting of
a) proteins comprising the amino acid sequence shown in SEQ ID No. 1 apart from the fact that the amino acid at position 186 is different from L;
b) proteins having an amino acid sequence having at least 80%, preferably 85%, more preferably 90%, even more preferably 95%, even more preferably 96%, even more preferably 97%, particularly preferably 98%, most preferably 99% identity to the amino acid sequence shown under a) provided that the amino acid at position 186 is different from L.

Preferably, the amino acid in the proteins according to a) or b) at position 186 is F, W, Y, E, D, Q, T, H, P, C, K, S, N, I or v. More preferably, the amino acid at position 186 is F, W, Y, E, D or K. Particularly preferably, the amino acid at position 186 is W or Y. Most preferably, the amino acid at position 186 is Y.

An “amino acid corresponding to position X” in a first amino acid sequence (e.g. position 3 in SEQ ID No. 1) means herein that an amino acid of a second amino acid sequence, compared with the first amino acid sequence, appears at position x of the first amino acid sequence in a pairwise sequence alignment of the first amino acid sequence with the second amino acid sequence if the numbering of the amino acids of the second amino acid sequence deviates from the amino acid numbering of the first amino acid sequence.

In connection with the present invention, the term “identity” in relation to sequence identity or identical sequences is to be understood to mean the number of identical amino acids or nucleotides that, over the entire sequence length, a first nucleic acid or amino acid sequence has in common with another (second) nucleic acid or amino acid sequence, expressed in percent.

“Sequence identity” can be determined by alignment of two amino acid or two nucleotide sequences using global or local alignment algorithms, as contained for example in known software such as GAP or BESTFIT or the Emboss program “Needle”. This software uses the global alignment algorithm by Needleman and Wunsch for aligning two sequences over their entire length, for maximizing the number of matches and for minimizing the number of gaps. In general, the default parameters are used, with a Gap Creation Penalty=10 and a Gap Extension Penalty=0.5 (for both nucleotide alignment and protein alignment). For nucleotides, the default scoring matrix used is DNAFULL, and for proteins, the default scoring matrix is Blosum62 (Henikoff & Henikoff, 1992, PNAS 89, 10915-10919). Sequence alignments and scores for the percentage sequence identity can, for example, be determined using software such as EMBOSS, available on the EBI website (ebi.ac.uk/Tools/emboss/). Alternatively, sequence similarity or identity can be determined by searching in databases (e.g. EMBL, GenBank) using commonly known algorithms and output formats such as FASTA, BLAST, etc., but hits should preferably be retrieved and aligned pairwise in order to lastly determine sequence identity.

If sequences to be compared with one another differ in length, identity should be determined by determining the identity in percent of the number of amino acids or nucleotides that the shorter sequence has in common with the longer sequence. Preferably, identity is determined with the aid of the known and publicly available computer program ClustalW (Thompson et al., Nucleic Acids Research 22 (1994), 4673-4680). ClustalW is publicly available from Julie Thompson (Thompson@EMBL-Heidelberg.DE) and Toby Gibson (Gibson@EMBL-Heidelberg.DE), European Molecular Biology Laboratory, Meyerhofstraße 1, D 69117 Heidelberg, Germany. ClustalW can also be downloaded from various websites, such as from IGBMC (Institut de Génétique et de Biologie Moléculaire et Cellulaire, B.P.163, 67404 Illkirch Cedex, France; ftp://ftp-igbmc.u-strasbg.fr/pub/) and from EBI (ftp://ftp.ebi.ac.uk/pub/software/) and from the mirror websites of the EBI (European Bioinformatics Institute, Wellcome Trust Genome Campus, Hinxton, Cambridge CB10 1SD, UK).

Preferably, version 1.8 of the computer program ClustalW is used for determining the identity between proteins described in the context of the present invention and other proteins. Here, the parameters must be set as follows: KTUPLE=1, TOPDIAG=5, WINDOW=5, PAIRGAP=3, GAPOPEN=10, GAPEXTEND=0.05, GAPDIST=8, MAXDIV=40, MATRIX=GONNET, ENDGAPS (OFF), NOPGAP, NOHGAP.

Preferably, version 1.8 of the computer program ClustalW is used for determining the identity between, for example, the nucleotide sequence of the nucleic acid molecules described in connection with the present invention and the nucleotide sequence of other nucleic acid molecules. Here, the parameters must be set as follows:
KTUPLE=2, TOPDIAGS=4, PAIRGAP-5, DNAMATRIX: IUB, GAPOPEN=10, GAPEXT=5, MAXDIV=40, TRANSITIONS: unweighted.

“Identity” also means that there is a functional and/or structural equivalence between the nucleic acid molecules in question or the proteins encoded thereby. Functional equivalence means that the nucleic acid molecule sequences or the amino acid sequences encode a protein having the activity of a lipase. The nucleic acid molecules which are homologous to the above-described molecules and are derivatives of these molecules are generally variants of these molecules which are modifications which have the same biological function or catalyse the same reaction, i.e. encode a protein having the activity of a lipase. They can be either naturally occurring variants, for example sequences from other species, or mutations, wherein said mutations can have occurred naturally or were introduced by targeted mutagenesis. Furthermore, the variants can be synthetically produced sequences. The allelic variants can be naturally occurring variants or synthetically produced variants, or variants produced by recombinant DNA techniques. However, what is crucial for the present invention is that these variants encode proteins having lipase activity and include the presently described amino acid substitutions (replacements), deletions or insertions regarding the proteins according to the invention.

A particular type of derivatives are, for example, nucleic acid molecules which differ from the nucleic acid molecules described in the context of the present invention through the degeneracy of the genetic code.

According to the NC-IUBMB (Nomenclature Committee of the International Union of Biochemistry and Molecular Biology), lipases belong to the class of the hydrolases (EC 3). Hydrolase is a class of enzymes which usually act as biochemical catalysts and use water to break a chemical bond, which typically leads to splitting of a larger molecule into smaller molecules. The group of the hydrolases comprises enzymes which act on ester bonds (EC 3.1), such as carboxylic ester hydrolases (EC 3.1.1) and, as a subgroup, lipases (EC 3.1.1.3). Lipases have been identified from plants, mammals and microorganisms, for example Pseudomonas, Vibrio, Acinetobacter, Burkholderia, Chromobacterium, cutinase from Fusarium solani (FSC), Candida antarctica A (CalA), Rhizopus oryzae (ROL), Thermomyces lanuginosus (TLL), Rhizomucor miehei (RML), Aspergillus Niger, Fusarium heterosporum, Fusarium oxysporum or Fusarium culmorum.

If a protein has the activity of a lipase, it can be detected using methods known and described according to the prior art. It is not crucial what method is used for detecting whether a protein according to the invention has the activity of a lipase. Preferably, a description is given of the method in the “Example” section in conjunction with the present invention.

Lipase variant proteins according to the invention can have further amino acid modifications (amino acid substitutions, deletions or insertions) compared to the amino acid sequence described herein above in relation to the amino acid sequence shown under SEQ ID No. 1.

The lipase variants described herein above under point a) in relation to the amino acid sequences shown under SEQ ID No. 1 can have additionally at least one, two, three, four, five, six or seven further amino acid substitutions at positions 79, 202, 280, 301, 3, 11, 17, 40 or 111. In other words, the protein according to the invention having the activity of a lipase is selected from the group consisting of a) proteins which comprise the amino acid sequence shown in SEQ ID No. 1 apart from that fact that the amino acid at position 186 is different from L and that they have at least one, two, three, four, five, six, seven or more further amino acid substitutions selected from the group consisting of (i) the amino acid at position 79 is different from E; (ii) the amino acid at position 202 is different from A; (iii) the amino acid at position 280 is different from L; (iv) the amino acid at position 301 is different from D; (v) the amino acid at position 3 is different from M; (vi) the amino acid at position 11 is different from C; (vii) the amino acid at position 17 is different from L; (viii) the amino acid at position 40 is different from K; (ix) the amino acid at position 111 is different from Q; and b) proteins having an amino acid sequence having at least 80%, preferably 85%, more preferably 90%, even more preferably 95%, even more preferably 96%, even more preferably 97%, particularly preferably 98%, most preferably 99% identity to the amino acid sequence shown directly above under a) provided that the amino acid at position 186 is different from L and having at least one further amino acid substitution selected from groups (i) to (ix) mentioned directly above. Preferably, the amino acid at position 79 is S, W or I, more preferably S; preferably, the amino acid at position 202 is N; preferably, the amino acid at position 280 is A; preferably, the amino acid at position 301 is A; preferably, the amino acid at position 3 is Q; preferably, the amino acid at position 11 is A; preferably, the amino acid at position 17 is P; preferably, the amino acid at position 40 is M; preferably, the amino acid at position 111 is E.

Furthermore, apart from the further amino acid modifications, lipase variant proteins according to the invention can have additional amino acid substitutions compared to the amino acid sequences described herein above in relation to the amino acid sequence shown under SEQ ID No. 1. These additional amino acid substitutions concern positions of the amino acid sequence that are different from position(s) 79, 202, 280, 301, 3, 11, 17, 40 or 111 relating to the further amino acid modifications. The lipase variants described herein above under point a) in relation to the amino acid sequences shown under SEQ ID No. 1 can have at least one, two, three, four, five, six, seven or more additional amino acid substitutions at positions 4, 8, 18, 29, 31, 42, 84, 85, 192, 217, 309 or 312. The amino acid at position 4 is different from S, preferably the amino acid at this position is P. The amino acid at position 8 is different from K, preferably the amino acid at this position is E. The amino acid at position 18 is different from V, preferably the amino acid at this position is C. The amino acid at position 29 is different from N, preferably the amino acid at this position is W or H. The amino acid at position 31 is different from Q, preferably the amino acid at this position is W. The amino acid at position 42 is different from L, preferably the amino acid at this position is D. The amino acid at position 84 is different from N, preferably the amino acid at this position is T. The amino acid at position 85 is different from W, preferably the amino acid at this position is H. The amino acid at position 192 is different from F, preferably the amino acid at this position is A or v. The amino acid at position 217 is different from Q, preferably the amino acid at this position is M. The amino acid at position 309 is different from P, preferably the amino acid at this position is C. The amino acid at position 312 is different from P, preferably the amino acid at this position is N.

Therefore, a further embodiment according to the invention concerns proteins according to the invention having further amino acid modifications; preferably, these embodiments are proteins having the activity of a lipase, wherein the proteins are selected from the group consisting of:
proteins having the amino acid sequence shown in SEQ ID No. 1 with the exception that
   the amino acid at position 186 is different from L and the amino acid at position 79 is different from E, wherein it is preferred that the amino acid at position is 186 is preferably W or Y, more preferably Y, and that the amino acid at position 79 is preferably S, W or I, more preferably S;
   the amino acid at position 186 is different from L and the amino acid at position 202 is different from A, wherein it is preferred that the amino acid at position is 186 is preferably W or Y and that the amino acid at position 202 is preferably N;
   the amino acid at position 186 is different from L and the amino acid at position 280 is different from L, wherein it is preferred that the amino acid at position is 186 is preferably W or Y and that the amino acid at position 280 is preferably A;
the amino acid at position 186 is different from L and the amino acid at position 301 is different from D, wherein it is preferred that the amino acid at position is 186 is preferably W or Y and that the amino acid at position 301 is preferably A;
   the amino acid at position 186 is different from L and the amino acid at position 3 is different from M, wherein it is preferred that the amino acid at position is 186 is preferably W or Y and that the amino acid at position 3 is preferably Q;
   the amino acid at position 186 is different from L and the amino acid at position 11 is different from C, wherein it is preferred that the amino acid at position is 186 is preferably W or Y and that the amino acid at position 11 is preferably A;
   the amino acid at position 186 is different from L and the amino acid at position 17 is different from L, wherein it is preferred that the amino acid at position is 186 is preferably W or Y and that the amino acid at position 17 is preferably P;
   the amino acid at position 186 is different from L and the amino acid at position 40 is different from K, wherein it is preferred that the amino acid at position is 186 is preferably W or Y and that the amino acid at position 40 is preferably M;
the amino acid at position 186 is different from L and the amino acid at position 111 is different from Q, wherein it is preferred that the amino acid at position is 186 is preferably W or Y and that the amino acid at position 111 is preferably E;
   proteins having an amino acid sequence at least 80%, preferably 85%, more preferably 90%, even more preferably 95%, even more preferably 96%, even more preferably 97%, particularly preferably 98%, most preferably 99% identical to the amino acid sequence shown under a) provided that the amino acid at position 186 is different from L, wherein it is preferred that the amino acid at position 186 is preferably W or Y, and wherein the proteins have at least one further amino acid substitution selected from the group shown directly above under the symbols listed.

Preferably, the lipase variants described herein above under point a) in relation to the amino acid sequences shown under SEQ ID No. 1 can have at least two further amino acid substitutions at positions 79, 202, 280, 301, 3, 11, 17, 40 or 111. Preferably, the amino acid at position 79 is S, W or I, more preferably S; preferably, the amino acid at position 202 is N; preferably, the amino acid at position 280 is A; preferably, the amino acid at position 301 is A; preferably, the amino acid at position 3 is Q; preferably, the amino acid at position 11 is A; preferably, the amino acid at position 17 is P; preferably, the amino acid at position 40 is M; preferably, the amino acid at position 111 is E.

Therefore, a further embodiment according to the invention concerns proteins according to the invention having further amino acid modifications; preferably, these embodiments are proteins having the activity of a lipase, wherein the proteins are selected from the group consisting of proteins having the amino acid sequence shown in SEQ ID No. 1 with the exception that
the amino acid at position 186 is different from L and the amino acid at position 79 is different from E and the amino acid at position 202 is different from A, wherein it is preferred that the amino acid at position 186 is preferably W or Y and that the amino acid at position 79 is preferably S, W or I, more preferably S, and that the amino acid at position 202 is preferably N;
the amino acid at position 186 is different from L and the amino acid at position 79 is different from E and the amino acid at position 280 is different from L, wherein it is preferred that the amino acid at position 186 is preferably W or Y and that the amino acid at position 79 is preferably S, W or I, more preferably S, and that the amino acid at position 280 is preferably A;
the amino acid at position 186 is different from L and the amino acid at position 79 is different from E and the amino acid at position 301 is different from D, wherein it is preferred that the amino acid at position 186 is preferably W or Y and that the amino acid at position 79 is preferably S, W or I, more preferably S, and that the amino acid at position 301 is preferably A;
the amino acid at position 186 is different from L and the amino acid at position 79 is different from E and the amino acid at position 3 is different from M, wherein it is preferred that the amino acid at position 186 is preferably W or Y and that the amino acid at position 79 is preferably S, W or I, more preferably S, and that the amino acid at position 3 is preferably Q;
the amino acid at position 186 is different from L and the amino acid at position 79 is different from E and the amino acid at position 11 is different from C, wherein it is preferred that the amino acid at position 186 is preferably W or Y and that the amino acid at position 79 is preferably S, W or I, more preferably S, and that the amino acid at position 11 is preferably A;
the amino acid at position 186 is different from L and the amino acid at position 79 is different from E and the amino acid at position 17 is different from L, wherein it is preferred that the amino acid at position 186 is preferably W or Y and that the amino acid at position 79 is preferably S, W or I, more preferably S, and that the amino acid at position 17 is preferably P;
the amino acid at position 186 is different from L and the amino acid at position 79 is different from E and the amino acid at position 40 is different from K, wherein it is preferred that the amino acid at position 186 is preferably W or Y and that the amino acid at position 79 is preferably S, W or I, more preferably S, and that the amino acid at position 40 is preferably M;
the amino acid at position 186 is different from L and the amino acid at position 79 is different from E and the amino acid at position 111 is different from Q, wherein it is preferred that the amino acid at position 186 is preferably W or Y and that the amino acid at position 79 is preferably S, W or I, more preferably S, and that the amino acid at position 111 is preferably E;
the amino acid at position 186 is different from L and the amino acid at position 202 is different from A and the amino acid at position 280 is different from L, wherein it is preferred that the amino acid at position 186 is preferably W or Y and that the amino acid at position 202 is preferably N and that the amino acid at position 280 is preferably A;
the amino acid at position 186 is different from L and the amino acid at position 202 is different from A and the amino acid at position 301 is different from D, wherein it is preferred that the amino acid at position 186 is preferably W or Y and that the amino acid at position 202 is preferably N and that the amino acid at position 301 is preferably A;
the amino acid at position 186 is different from L and the amino acid at position 202 is different from A and the amino acid at position 3 is different from M, wherein it is preferred that the amino acid at position 186 is preferably W or Y and that the amino acid at position 202 is preferably N and that the amino acid at position 3 is preferably Q;
the amino acid at position 186 is different from L and the amino acid at position 202 is different from A and the amino acid at position 11 is different from C, wherein it is preferred that the amino acid at position 186 is preferably W or Y and that the amino acid at position 202 is preferably N and that the amino acid at position 11 is preferably A;
the amino acid at position 186 is different from L and the amino acid at position 202 is different from A and the amino acid at position 17 is different from L, wherein it is preferred that the amino acid at position 186 is preferably W or Y and that the amino acid at position 202 is preferably N and that the amino acid at position 17 is preferably P;
the amino acid at position 186 is different from L and the amino acid at position 202 is different from A and the amino acid at position 40 is different from K, wherein it is preferred that the amino acid at position 186 is preferably W or Y and that the amino acid at position 202 is preferably N and that the amino acid at position 40 is preferably M;
the amino acid at position 186 is different from L and the amino acid at position 202 is different from A and the amino acid at position 111 is different from Q, wherein it is preferred that the amino acid at position 186 is preferably W or Y and that the amino acid at position 202 is preferably N and that the amino acid at position 111 is preferably E;
the amino acid at position 186 is different from L and the amino acid at position 280 is different from L and the amino acid at position 301 is different from D, wherein it is preferred that the amino acid at position 186 is preferably W or Y and that the amino acid at position 280 is preferably A and that the amino acid at position 301 is preferably A;
the amino acid at position 186 is different from L and the amino acid at position 280 is different from L and the amino acid at position 3 is different from M, wherein it is preferred that the amino acid at position 186 is preferably W or Y and that the amino acid at position 280 is preferably A and that the amino acid at position 3 is preferably Q;
the amino acid at position 186 is different from L and the amino acid at position 280 is different from L and the amino acid at position 11 is different from C, wherein it is preferred that the amino acid at position 186 is preferably W or Y and that the amino acid at position 280 is preferably A and that the amino acid at position 11 is preferably A;
the amino acid at position 186 is different from L and the amino acid at position 280 is different from L and the amino acid at position 17 is different from L, wherein it is preferred that the amino acid at position 186 is preferably W or Y and that the amino acid at position 280 is preferably A and that the amino acid at position 17 is preferably P;
the amino acid at position 186 is different from L and the amino acid at position 280 is different from L and the amino acid at position 40 is different from K, wherein it is preferred that the amino acid at position 186 is preferably W or Y and that the amino acid at position 280 is preferably A and that the amino acid at position 40 is preferably M;
the amino acid at position 186 is different from L and the amino acid at position 280 is different from L and the amino acid at position 111 is different from Q, wherein it is preferred that the amino acid at position 186 is preferably W or Y and that the amino acid at position 280 is preferably A and that the amino acid at position 111 is preferably E;
the amino acid at position 186 is different from L and the amino acid at position 301 is different from D and the amino acid at position 3 is different from M, wherein it is preferred that the amino acid at position 186 is preferably W or Y and that the amino acid at position 301 is preferably A and that the amino acid at position 3 is preferably Q;
the amino acid at position 186 is different from L and the amino acid at position 301 is different from D and the amino acid at position 11 is different from C, wherein it is preferred that the amino acid at position 186 is preferably W or Y and that the amino acid at position 301 is preferably A and that the amino acid at position 11 is preferably A;
the amino acid at position 186 is different from L and the amino acid at position 301 is different from D and the amino acid at position 17 is different from L, wherein it is preferred that the amino acid at position 186 is preferably W or Y and that the amino acid at position 301 is preferably A and that the amino acid at position 17 is preferably P;
the amino acid at position 186 is different from L and the amino acid at position 301 is different from D and the amino acid at position 40 is different from K, wherein it is preferred that the amino acid at position 186 is preferably W or Y and that the amino acid at position 301 is preferably A and that the amino acid at position 40 is preferably M;
the amino acid at position 186 is different from L and the amino acid at position 301 is different from D and the amino acid at position 111 is different from Q, wherein it is preferred that the amino acid at position 186 is preferably W or Y and that the amino acid at position 301 is preferably A and that the amino acid at position 111 is preferably E;
the amino acid at position 186 is different from L and the amino acid at position 3 is different from M and the amino acid at position 11 is different from C, wherein it is preferred that the amino acid at position 186 is preferably W or Y and that the amino acid at position 3 is preferably Q and that the amino acid at position 11 is preferably A;
the amino acid at position 186 is different from L and the amino acid at position 3 is different from M and the amino acid at position 17 is different from L, wherein it is preferred that the amino acid at position 186 is preferably W or Y and that the amino acid at position 3 is preferably Q and that the amino acid at position 17 is preferably P;
the amino acid at position 186 is different from L and the amino acid at position 3 is different from M and the amino acid at position 40 is different from K, wherein it is preferred that the amino acid at position 186 is preferably W or Y and that the amino acid at position 3 is preferably Q and that the amino acid at position 40 is preferably M;
the amino acid at position 186 is different from L and the amino acid at position 3 is different from M and the amino acid at position 111 is different from Q, wherein it is preferred that the amino acid at position 186 is preferably W or Y and that the amino acid at position 3 is preferably Q and that the amino acid at position 111 is preferably E;
the amino acid at position 186 is different from L and the amino acid at position 11 is different from C and the amino acid at position 17 is different from L, wherein it is preferred that the amino acid at position 186 is preferably W or Y and that the amino acid at position 11 is preferably A and that the amino acid at position 17 is preferably P;
the amino acid at position 186 is different from L and the amino acid at position 11 is different from C and the amino acid at position 40 is different from K, wherein it is preferred that the amino acid at position 186 is preferably W or Y and that the amino acid at position 11 is preferably A and that the amino acid at position 40 is preferably M;
the amino acid at position 186 is different from L and the amino acid at position 11 is different from C and the amino acid at position 111 is different from Q, wherein it is preferred that the amino acid at position 186 is preferably W or Y and that the amino acid at position 11 is preferably A and that the amino acid at position 111 is preferably E;
the amino acid at position 186 is different from L and the amino acid at position 17 is different from L and the amino acid at position 40 is different from K, wherein it is preferred that the amino acid at position 186 is preferably W or Y and that the amino acid at position 17 is preferably P and that the amino acid at position 40 is preferably M;
the amino acid at position 186 is different from L and the amino acid at position 17 is different from L and the amino acid at position 111 is different from Q, wherein it is preferred that the amino acid at position 186 is preferably W or Y and that the amino acid at position 17 is preferably P and that the amino acid at position 111 is preferably E;
the amino acid at position 186 is different from L and the amino acid at position 40 is different from K and the amino acid at position 111 is different from Q, wherein it is preferred that the amino acid at position 186 is preferably W or Y and that the amino acid at position 40 is preferably M and that the amino acid at position 111 is preferably E;
proteins having an amino acid sequence at least 80%, preferably 85%, more preferably 90%, even more preferably 95%, even more preferably 96%, even more preferably 97%, particularly preferably 98%, most preferably 99% identical to the amino acid sequence shown under a) provided that the amino acid at position 186 is different from L, wherein it is preferred that the amino acid at position 186 is preferably W or Y, and wherein the proteins have at least two further amino acid substitutions selected from the group shown directly above under the symbols listed.

Preferably, the lipase variants described herein above under point a) in relation to the amino acid sequences shown under SEQ ID No. 1 can have at least three further amino acid substitutions at positions 79, 202, 280, 301, 3, 11, 17, 40 or 111. Preferably, the amino acid at position 79 is S, W or I, more preferably S; preferably, the amino acid at position 202 is N; preferably, the amino acid at position 280 is A; preferably, the amino acid at position 301 is A; preferably, the amino acid at position 3 is Q; preferably, the amino acid at position 11 is A; preferably, the amino acid at position 17 is P; preferably, the amino acid at position 40 is M; preferably, the amino acid at position 111 is E.

Therefore, a further embodiment according to the invention concerns proteins according to the invention having further amino acid modifications; preferably, these embodiments are proteins having the activity of a lipase, wherein the proteins are selected from the group consisting of proteins having the amino acid sequence shown in SEQ ID No. 1 with the exception that
the amino acid at position 186 is different from L and the amino acid at position 79 is different from E and the amino acid at position 202 is different from A and the amino acid at position 280 is different from L, wherein it is preferred that the amino acid at position 186 is preferably W or Y and that the amino acid at position 79 is preferably S, W or I, more preferably S, and that the amino acid at position 202 is preferably N and that the amino acid at position 280 is preferably A;
the amino acid at position 186 is different from L and the amino acid at position 79 is different from E and the amino acid at position 202 is different from A and the amino acid at position 301 is different from D, wherein it is preferred that the amino acid at position 186 is preferably W or Y and that the amino acid at position 79 is preferably S, W or I, more preferably S, and that the amino acid at position 202 is preferably N and that the amino acid at position 301 is preferably A;
the amino acid at position 186 is different from L and the amino acid at position 79 is different from E and the amino acid at position 202 is different from A and the amino acid at position 3 is different from M, wherein it is preferred that the amino acid at position 186 is preferably W or Y and that the amino acid at position 79 is preferably S, W or I, more preferably S, and that the amino acid at position 202 is preferably N and that the amino acid at position 3 is preferably Q;
the amino acid at position 186 is different from L and the amino acid at position 79 is different from E and the amino acid at position 202 is different from A and the amino acid at position 11 is different from C, wherein it is preferred that the amino acid at position 186 is preferably W or Y and that the amino acid at position 79 is preferably S, W or I, more preferably S, and that the amino acid at position 202 is preferably N and that the amino acid at position 11 is preferably A;
the amino acid at position 186 is different from L and the amino acid at position 79 is different from E and the amino acid at position 202 is different from A and the amino acid at position 17 is different from L, wherein it is preferred that the amino acid at position 186 is preferably W or Y and that the amino acid at position 79 is preferably S, W or I, more preferably S, and that the amino acid at position 202 is preferably N and that the amino acid at position 17 is preferably P;
the amino acid at position 186 is different from L and the amino acid at position 79 is different from E and the amino acid at position 202 is different from A and the amino acid at position 40 is different from K, wherein it is preferred that the amino acid at position 186 is preferably W or Y and that the amino acid at position 79 is preferably S, W or I, more preferably S, and that the amino acid at position 202 is preferably N and that the amino acid at position 40 is preferably M;
the amino acid at position 186 is different from L and the amino acid at position 79 is different from E and the amino acid at position 202 is different from A and the amino acid at position 111 is different from Q, wherein it is preferred that the amino acid at position 186 is preferably W or Y and that the amino acid at position 79 is preferably S, W or I, more preferably S, and that the amino acid at position 202 is preferably N and that the amino acid at position 111 is preferably E;
the amino acid at position 186 is different from L and the amino acid at position 79 is different from E and the amino acid at position 280 is different from L and the amino acid at position 301 is different from D, wherein it is preferred that the amino acid at position 186 is preferably W or Y and that the amino acid at position 79 is preferably S, W or I, more preferably S, and that the amino acid at position 280 is preferably A and that the amino acid at position 301 is preferably A;
the amino acid at position 186 is different from L and the amino acid at position 79 is different from E and the amino acid at position 280 is different from L and the amino acid at position 3 is different from M, wherein it is preferred that the amino acid at position 186 is preferably W or Y and that the amino acid at position 79 is preferably S, W or I, more preferably S, and that the amino acid at position 280 is preferably A and that the amino acid at position 3 is preferably Q;
the amino acid at position 186 is different from L and the amino acid at position 79 is different from E and the amino acid at position 280 is different from L and the amino acid at position 11 is different from C, wherein it is preferred that the amino acid at position 186 is preferably W or Y and that the amino acid at position 79 is preferably S, W or I, more preferably S, and that the amino acid at position 280 is preferably A and that the amino acid at position 11 is preferably A;
the amino acid at position 186 is different from L and the amino acid at position 79 is different from E and the amino acid at position 280 is different from L and the amino acid at position 17 is different from L, wherein it is preferred that the amino acid at position 186 is preferably W or Y and that the amino acid at position 79 is preferably S, W or I, more preferably S, and that the amino acid at position 280 is preferably A and that the amino acid at position 17 is preferably P;
the amino acid at position 186 is different from L and the amino acid at position 79 is different from E and the amino acid at position 280 is different from L and the amino acid at position 40 is different from K, wherein it is preferred that the amino acid at position 186 is preferably W or Y and that the amino acid at position 79 is preferably S, W or I, more preferably S, and that the amino acid at position 280 is preferably A and that the amino acid at position 40 is preferably M;
the amino acid at position 186 is different from L and the amino acid at position 79 is different from E and the amino acid at position 280 is different from L and the amino acid at position 111 is different from Q, wherein it is preferred that the amino acid at position 186 is preferably W or Y and that the amino acid at position 79 is preferably S, W or I, more preferably S, and that the amino acid at position 280 is preferably A and that the amino acid at position 111 is preferably E;
the amino acid at position 186 is different from L and the amino acid at position 79 is different from E and the amino acid at position 301 is different from D and the amino acid at position 3 is different from M, wherein it is preferred that the amino acid at position 186 is preferably W or Y and that the amino acid at position 79 is preferably S, W or I, more preferably S, and that the amino acid at position 301 is preferably A and that the amino acid at position 3 is preferably Q;
the amino acid at position 186 is different from L and the amino acid at position 79 is different from E and the amino acid at position 301 is different from D and the amino acid at position 11 is different from C, wherein it is preferred that the amino acid at position 186 is preferably W or Y and that the amino acid at position 79 is preferably S, W or I, more preferably S, and that the amino acid at position 301 is preferably A and that the amino acid at position 11 is preferably A;
the amino acid at position 186 is different from L and the amino acid at position 79 is different from E and the amino acid at position 301 is different from D and the amino acid at position 17 is different from L, wherein it is preferred that the amino acid at position 186 is preferably W or Y and that the amino acid at position 79 is preferably S, W or I, more preferably S, and that the amino acid at position 301 is preferably A and that the amino acid at position 17 is preferably P;
the amino acid at position 186 is different from L and the amino acid at position 79 is different from E and the amino acid at position 301 is different from D and the amino acid at position 40 is different from K, wherein it is preferred that the amino acid at position 186 is preferably W or Y, more preferably Y, and that the amino acid at position 79 is preferably S, W or I, more preferably S, and that the amino acid at position 301 is preferably A and that the amino acid at position 40 is preferably M, wherein it is particularly preferred that the amino acid at position 186 is Y and that the amino acid at position 79 is S and that the amino acid at position 301 is A and that the amino acid at position 40 is M;
the amino acid at position 186 is different from L and the amino acid at position 79 is different from E and the amino acid at position 301 is different from D and the amino acid at position 111 is different from Q, wherein it is preferred that the amino acid at position 186 is preferably W or Y and that the amino acid at position 79 is preferably S, W or I, more preferably S, and that the amino acid at position 301 is preferably A and that the amino acid at position 111 is preferably E;
the amino acid at position 186 is different from L and the amino acid at position 79 is different from E and the amino acid at position 3 is different from M and the amino acid at position 11 is different from C, wherein it is preferred that the amino acid at position 186 is preferably W or Y and that the amino acid at position 79 is preferably S, W or I, more preferably S, and that the amino acid at position 3 is preferably Q and that the amino acid at position 11 is preferably A;
the amino acid at position 186 is different from L and the amino acid at position 79 is different from E and the amino acid at position 3 is different from M and the amino acid at position 17 is different from L, wherein it is preferred that the amino acid at position 186 is preferably W or Y and that the amino acid at position 79 is preferably S, W or I, more preferably S, and that the amino acid at position 3 is preferably Q and that the amino acid at position 17 is preferably P;
the amino acid at position 186 is different from L and the amino acid at position 79 is different from E and the amino acid at position 3 is different from M and the amino acid at position 40 is different from K, wherein it is preferred that the amino acid at position 186 is preferably W or Y and that the amino acid at position 79 is preferably S, W or I, more preferably S, and that the amino acid at position 3 is preferably Q and that the amino acid at position 40 is preferably M;
the amino acid at position 186 is different from L and the amino acid at position 79 is different from E and the amino acid at position 3 is different from M and the amino acid at position 111 is different from Q, wherein it is preferred that the amino acid at position 186 is preferably W or Y and that the amino acid at position 79 is preferably S, W or I, more preferably S, and that the amino acid at position 3 is preferably Q and that the amino acid at position 111 is preferably E;
the amino acid at position 186 is different from L and the amino acid at position 79 is different from E and the amino acid at position 11 is different from C and the amino acid at position 17 is different from L, wherein it is preferred that the amino acid at position 186 is preferably W or Y and that the amino acid at position 79 is preferably S, W or I, more preferably S, and that the amino acid at position 11 is preferably A and that the amino acid at position 17 is preferably P;
the amino acid at position 186 is different from L and the amino acid at position 79 is different from E and the amino acid at position 11 is different from C and the amino acid at position 40 is different from K, wherein it is preferred that the amino acid at position 186 is preferably W or Y and that the amino acid at position 79 is preferably S, W or I, more preferably S, and that the amino acid at position 11 is preferably A and that the amino acid at position 40 is preferably M;
the amino acid at position 186 is different from L and the amino acid at position 79 is different from E and the amino acid at position 11 is different from C and the amino acid at position 111 is different from Q, wherein it is preferred that the amino acid at position 186 is preferably W or Y and that the amino acid at position 79 is preferably S, W or I, more preferably S, and that the amino acid at position 11 is preferably A and that the amino acid at position 111 is preferably E;
the amino acid at position 186 is different from L and the amino acid at position 79 is different from E and the amino acid at position 17 is different from L and the amino acid at position 40 is different from K, wherein it is preferred that the amino acid at position 186 is preferably W or Y and that the amino acid at position 79 is preferably S, W or I, more preferably S, and that the amino acid at position 17 is preferably P and that the amino acid at position 40 is preferably M;
the amino acid at position 186 is different from L and the amino acid at position 79 is different from E and the amino acid at position 17 is different from L and the amino acid at position 111 is different from Q, wherein it is preferred that the amino acid at position 186 is preferably W or Y and that the amino acid at position 79 is preferably S, W or I, more preferably S, and that the amino acid at position 17 is preferably P and that the amino acid at position 111 is preferably E;
the amino acid at position 186 is different from L and the amino acid at position 79 is different from E and the amino acid at position 40 is different from K and the amino acid at position 111 is different from Q, wherein it is preferred that the amino acid at position 186 is preferably W or Y and that the amino acid at position 79 is preferably S, W or I, more preferably S, and that the amino acid at position 40 is preferably M and that the amino acid at position 111 is preferably E;
the amino acid at position 186 is different from L and the amino acid at position 202 is different from A and the amino acid at position 280 is different from L and the amino acid at position 301 is different from D, wherein it is preferred that the amino acid at position 186 is preferably W or Y and that the amino acid at position 202 is preferably N and that the amino acid at position 280 is preferably A and that the amino acid at position 301 is preferably A;
the amino acid at position 186 is different from L and the amino acid at position 202 is different from A and the amino acid at position 280 is different from L and the amino acid at position 3 is different from M, wherein it is preferred that the amino acid at position 186 is preferably W or Y and that the amino acid at position 202 is preferably N and that the amino acid at position 280 is preferably A and that the amino acid at position 3 is preferably Q;
the amino acid at position 186 is different from L and the amino acid at position 202 is different from A and the amino acid at position 280 is different from L and the amino acid at position 11 is different from C, wherein it is preferred that the amino acid at position 186 is preferably W or Y and that the amino acid at position 202 is preferably N and that the amino acid at position 280 is preferably A and that the amino acid at position 11 is preferably A;
the amino acid at position 186 is different from L and the amino acid at position 202 is different from A and the amino acid at position 280 is different from L and the amino acid at position 17 is different from L, wherein it is preferred that the amino acid at position 186 is preferably W or Y and that the amino acid at position 202 is preferably N and that the amino acid at position 280 is preferably A and that the amino acid at position 17 is preferably P;
the amino acid at position 186 is different from L and the amino acid at position 202 is different from A and the amino acid at position 280 is different from L and the amino acid at position 40 is different from K, wherein it is preferred that the amino acid at position 186 is preferably W or Y and that the amino acid at position 202 is preferably N and that the amino acid at position 280 is preferably A and that the amino acid at position 40 is preferably M;
the amino acid at position 186 is different from L and the amino acid at position 202 is different from A and the amino acid at position 280 is different from L and the amino acid at position 111 is different from Q, wherein it is preferred that the amino acid at position 186 is preferably W or Y and that the amino acid at position 202 is preferably N and that the amino acid at position 280 is preferably A and that the amino acid at position 111 is preferably E;
the amino acid at position 186 is different from L and the amino acid at position 202 is different from A and the amino acid at position 301 is different from D and the amino acid at position 3 is different from M, wherein it is preferred that the amino acid at position 186 is preferably W or Y and that the amino acid at position 202 is preferably N and that the amino acid at position 301 is preferably A and that the amino acid at position 3 is preferably Q;
the amino acid at position 186 is different from L and the amino acid at position 202 is different from A and the amino acid at position 301 is different from D and the amino acid at position 11 is different from C, wherein it is preferred that the amino acid at position 186 is preferably W or Y and that the amino acid at position 202 is preferably N and that the amino acid at position 301 is preferably A and that the amino acid at position 11 is preferably A;
the amino acid at position 186 is different from L and the amino acid at position 202 is different from A and the amino acid at position 301 is different from D and the amino acid at position 17 is different from L, wherein it is preferred that the amino acid at position 186 is preferably W or Y and that the amino acid at position 202 is preferably N and that the amino acid at position 301 is preferably A and that the amino acid at position 17 is preferably P;
the amino acid at position 186 is different from L and the amino acid at position 202 is different from A and the amino acid at position 301 is different from D and the amino acid at position 40 is different from K, wherein it is preferred that the amino acid at position 186 is preferably W or Y and that the amino acid at position 202 is preferably N and that the amino acid at position 301 is preferably A and that the amino acid at position 40 is preferably M;
the amino acid at position 186 is different from L and the amino acid at position 202 is different from A and the amino acid at position 301 is different from D and the amino acid at position 111 is different from Q, wherein it is preferred that the amino acid at position 186 is preferably W or Y and that the amino acid at position 202 is preferably N and that the amino acid at position 301 is preferably A and that the amino acid at position 111 is preferably E;
the amino acid at position 186 is different from L and the amino acid at position 202 is different from A and the amino acid at position 3 is different from M and the amino acid at position 11 is different from C, wherein it is preferred that the amino acid at position 186 is preferably W or Y and that the amino acid at position 202 is preferably N and that the amino acid at position 3 is preferably Q and that the amino acid at position 11 is preferably A;
the amino acid at position 186 is different from L and the amino acid at position 202 is different from A and the amino acid at position 3 is different from M and the amino acid at position 17 is different from L, wherein it is preferred that the amino acid at position 186 is preferably W or Y and that the amino acid at position 202 is preferably N and that the amino acid at position 3 is preferably Q and that the amino acid at position 17 is preferably P;
the amino acid at position 186 is different from L and the amino acid at position 202 is different from A and the amino acid at position 3 is different from M and the amino acid at position 40 is different from K, wherein it is preferred that the amino acid at position 186 is preferably W or Y and that the amino acid at position 202 is preferably N and that the amino acid at position 3 is preferably Q and that the amino acid at position 40 is preferably M;
the amino acid at position 186 is different from L and the amino acid at position 202 is different from A and the amino acid at position 3 is different from M and the amino acid at position 111 is different from Q, wherein it is preferred that the amino acid at position 186 is preferably W or Y and that the amino acid at position 202 is preferably N and that the amino acid at position 3 is preferably Q and that the amino acid at position 111 is preferably E;
the amino acid at position 186 is different from L and the amino acid at position 202 is different from A and the amino acid at position 11 is different from C and the amino acid at position 17 is different from L, wherein it is preferred that the amino acid at position 186 is preferably W or Y and that the amino acid at position 202 is preferably N and that the amino acid at position 11 is preferably A and that the amino acid at position 17 is preferably P;
the amino acid at position 186 is different from L and the amino acid at position 202 is different from A and the amino acid at position 11 is different from C and the amino acid at position 40 is different from K, wherein it is preferred that the amino acid at position 186 is preferably W or Y and that the amino acid at position 202 is preferably N and that the amino acid at position 11 is preferably A and that the amino acid at position 40 is preferably M;
the amino acid at position 186 is different from L and the amino acid at position 202 is different from A and the amino acid at position 11 is different from C and the amino acid at position 111 is different from Q, wherein it is preferred that the amino acid at position 186 is preferably W or Y and that the amino acid at position 202 is preferably N and that the amino acid at position 11 is preferably A and that the amino acid at position 111 is preferably E;
the amino acid at position 186 is different from L and the amino acid at position 202 is different from A and the amino acid at position 17 is different from L and the amino acid at position 40 is different from K, wherein it is preferred that the amino acid at position 186 is preferably W or Y and that the amino acid at position 202 is preferably N and that the amino acid at position 17 is preferably P and that the amino acid at position 40 is preferably M;
the amino acid at position 186 is different from L and the amino acid at position 202 is different from A and the amino acid at position 17 is different from L and the amino acid at position 111 is different from Q, wherein it is preferred that the amino acid at position 186 is preferably W or Y and that the amino acid at position 202 is preferably N and that the amino acid at position 17 is preferably P and that the amino acid at position 111 is preferably E;
the amino acid at position 186 is different from L and the amino acid at position 202 is different from A and the amino acid at position 40 is different from K and the amino acid at position 111 is different from Q, wherein it is preferred that the amino acid at position 186 is preferably W or Y and that the amino acid at position 202 is preferably N and that the amino acid at position 40 is preferably M and that the amino acid at position 111 is preferably E;
the amino acid at position 186 is different from L and the amino acid at position 280 is different from L and the amino acid at position 301 is different from D and the amino acid at position 3 is different from M, wherein it is preferred that the amino acid at position 186 is preferably W or Y and that the amino acid at position 280 is preferably A and that the amino acid at position 301 is preferably A and that the amino acid at position 3 is preferably Q;
the amino acid at position 186 is different from L and the amino acid at position 280 is different from L and the amino acid at position 301 is different from D and the amino acid at position 11 is different from C, wherein it is preferred that the amino acid at position 186 is preferably W or Y and that the amino acid at position 280 is preferably A and that the amino acid at position 301 is preferably A and that the amino acid at position 11 is preferably A;
the amino acid at position 186 is different from L and the amino acid at position 280 is different from L and the amino acid at position 301 is different from D and the amino acid at position 17 is different from L, wherein it is preferred that the amino acid at position 186 is preferably W or Y and that the amino acid at position 280 is preferably A and that the amino acid at position 301 is preferably A and that the amino acid at position 17 is preferably P;
the amino acid at position 186 is different from L and the amino acid at position 280 is different from L and the amino acid at position 301 is different from D and the amino acid at position 40 is different from K, wherein it is preferred that the amino acid at position 186 is preferably W or Y and that the amino acid at position 280 is preferably A and that the amino acid at position 301 is preferably A and that the amino acid at position 40 is preferably M;
the amino acid at position 186 is different from L and the amino acid at position 280 is different from L and the amino acid at position 301 is different from D and the amino acid at position 111 is different from Q, wherein it is preferred that the amino acid at position 186 is preferably W or Y and that the amino acid at position 280 is preferably A and that the amino acid at position 301 is preferably A and that the amino acid at position 111 is preferably E;
the amino acid at position 186 is different from L and the amino acid at position 280 is different from L and the amino acid at position 3 is different from M and the amino acid at position 11 is different from C, wherein it is preferred that the amino acid at position 186 is preferably W or Y and that the amino acid at position 280 is preferably A and that the amino acid at position 3 is preferably Q and that the amino acid at position 11 is preferably A;
the amino acid at position 186 is different from L and the amino acid at position 280 is different from L and the amino acid at position 3 is different from M and the amino acid at position 17 is different from L, wherein it is preferred that the amino acid at position 186 is preferably W or Y and that the amino acid at position 280 is preferably A and that the amino acid at position 3 is preferably Q and that the amino acid at position 17 is preferably P;
the amino acid at position 186 is different from L and the amino acid at position 280 is different from L and the amino acid at position 3 is different from M and the amino acid at position 40 is different from K, wherein it is preferred that the amino acid at position 186 is preferably W or Y and that the amino acid at position 280 is preferably A and that the amino acid at position 3 is preferably Q and that the amino acid at position 40 is preferably M;
the amino acid at position 186 is different from L and the amino acid at position 280 is different from L and the amino acid at position 3 is different from M and the amino acid at position 111 is different from Q, wherein it is preferred that the amino acid at position 186 is preferably W or Y and that the amino acid at position 280 is preferably A and that the amino acid at position 3 is preferably Q and that the amino acid at position 111 is preferably E;
the amino acid at position 186 is different from L and the amino acid at position 280 is different from L and the amino acid at position 11 is different from C and the amino acid at position 17 is different from L, wherein it is preferred that the amino acid at position 186 is preferably W or Y and that the amino acid at position 280 is preferably A and that the amino acid at position 11 is preferably A and that the amino acid at position 17 is preferably P;
the amino acid at position 186 is different from L and the amino acid at position 280 is different from L and the amino acid at position 11 is different from C and the amino acid at position 40 is different from K, wherein it is preferred that the amino acid at position 186 is preferably W or Y and that the amino acid at position 280 is preferably A and that the amino acid at position 11 is preferably A and that the amino acid at position 40 is preferably M;
the amino acid at position 186 is different from L and the amino acid at position 280 is different from L and the amino acid at position 11 is different from C and the amino acid at position 111 is different from Q, wherein it is preferred that the amino acid at position 186 is preferably W or Y and that the amino acid at position 280 is preferably A and that the amino acid at position 11 is preferably A and that the amino acid at position 111 is preferably E;
the amino acid at position 186 is different from L and the amino acid at position 280 is different from L and the amino acid at position 17 is different from L and the amino acid at position 40 is different from K, wherein it is preferred that the amino acid at position 186 is preferably W or Y and that the amino acid at position 280 is preferably A and that the amino acid at position 17 is preferably P and that the amino acid at position 40 is preferably M;
the amino acid at position 186 is different from L and the amino acid at position 280 is different from L and the amino acid at position 17 is different from L and the amino acid at position 111 is different from Q, wherein it is preferred that the amino acid at position 186 is preferably W or Y and that the amino acid at position 280 is preferably A and that the amino acid at position 17 is preferably P and that the amino acid at position 111 is preferably E;
the amino acid at position 186 is different from L and the amino acid at position 280 is different from L and the amino acid at position 40 is different from K and the amino acid at position 111 is different from Q, wherein it is preferred that the amino acid at position 186 is preferably W or Y and that the amino acid at position 280 is preferably A and that the amino acid at position 40 is preferably M and that the amino acid at position 111 is preferably E;
the amino acid at position 186 is different from L and the amino acid at position 301 is different from D and the amino acid at position 3 is different from M and the amino acid at position 11 is different from C, wherein it is preferred that the amino acid at position 186 is preferably W or Y and that the amino acid at position 301 is preferably A and that the amino acid at position 3 is preferably Q and that the amino acid at position 11 is preferably A;
the amino acid at position 186 is different from L and the amino acid at position 301 is different from D and the amino acid at position 3 is different from M and the amino acid at position 17 is different from L, wherein it is preferred that the amino acid at position 186 is preferably W or Y and that the amino acid at position 301 is preferably A and that the amino acid at position 3 is preferably Q and that the amino acid at position 17 is preferably P;
the amino acid at position 186 is different from L and the amino acid at position 301 is different from D and the amino acid at position 3 is different from M and the amino acid at position 40 is different from K, wherein it is preferred that the amino acid at position 186 is preferably W or Y and that the amino acid at position 301 is preferably A and that the amino acid at position 3 is preferably Q and that the amino acid at position 40 is preferably M;
the amino acid at position 186 is different from L and the amino acid at position 301 is different from D and the amino acid at position 3 is different from M and the amino acid at position 111 is different from Q, wherein it is preferred that the amino acid at position 186 is preferably W or Y and that the amino acid at position 301 is preferably A and that the amino acid at position 3 is preferably Q and that the amino acid at position 111 is preferably E;
the amino acid at position 186 is different from L and the amino acid at position 301 is different from D and the amino acid at position 11 is different from C and the amino acid at position 17 is different from L, wherein it is preferred that the amino acid at position 186 is preferably W or Y and that the amino acid at position 301 is preferably A and that the amino acid at position 11 is preferably A and that the amino acid at position 17 is preferably P;
the amino acid at position 186 is different from L and the amino acid at position 301 is different from D and the amino acid at position 11 is different from C and the amino acid at position 40 is different from K, wherein it is preferred that the amino acid at position 186 is preferably W or Y and that the amino acid at position 301 is preferably A and that the amino acid at position 11 is preferably A and that the amino acid at position 40 is preferably M;
the amino acid at position 186 is different from L and the amino acid at position 301 is different from D and the amino acid at position 11 is different from C and the amino acid at position 111 is different from Q, wherein it is preferred that the amino acid at position 186 is preferably W or Y and that the amino acid at position 301 is preferably A and that the amino acid at position 11 is preferably A and that the amino acid at position 111 is preferably E;
the amino acid at position 186 is different from L and the amino acid at position 301 is different from D and the amino acid at position 17 is different from L and the amino acid at position 40 is different from K, wherein it is preferred that the amino acid at position 186 is preferably W or Y and that the amino acid at position 301 is preferably A and that the amino acid at position 17 is preferably P and that the amino acid at position 40 is preferably M;
the amino acid at position 186 is different from L and the amino acid at position 301 is different from D and the amino acid at position 17 is different from L and the amino acid at position 111 is different from Q, wherein it is preferred that the amino acid at position 186 is preferably W or

Y and that the amino acid at position 301 is preferably A and that the amino acid at position 17 is preferably P and that the amino acid at position 111 is preferably E;
the amino acid at position 186 is different from L and the amino acid at position 301 is different from D and the amino acid at position 40 is different from K and the amino acid at position 111 is different from Q, wherein it is preferred that the amino acid at position 186 is preferably W or Y and that the amino acid at position 301 is preferably A and that the amino acid at position 40 is preferably M and that the amino acid at position 111 is preferably E;
the amino acid at position 186 is different from L and the amino acid at position 3 is different from M and the amino acid at position 11 is different from C and the amino acid at position 17 is different from L, wherein it is preferred that the amino acid at position 186 is preferably W or Y and that the amino acid at position 3 is preferably Q and that the amino acid at position 11 is preferably A and that the amino acid at position 17 is preferably P;
the amino acid at position 186 is different from L and the amino acid at position 3 is different from M and the amino acid at position 11 is different from C and the amino acid at position 40 is different from K, wherein it is preferred that the amino acid at position 186 is preferably W or Y and that the amino acid at position 3 is preferably Q and that the amino acid at position 11 is preferably A and that the amino acid at position 40 is preferably M;
the amino acid at position 186 is different from L and the amino acid at position 3 is different from M and the amino acid at position 11 is different from C and the amino acid at position 111 is different from Q, wherein it is preferred that the amino acid at position 186 is preferably W or Y and that the amino acid at position 3 is preferably Q and that the amino acid at position 11 is preferably A and that the amino acid at position 111 is preferably E;
the amino acid at position 186 is different from L and the amino acid at position 3 is different from M and the amino acid at position 17 is different from L and the amino acid at position 40 is different from K, wherein it is preferred that the amino acid at position 186 is preferably W or Y and that the amino acid at position 3 is preferably Q and that the amino acid at position 17 is preferably P and that the amino acid at position 40 is preferably M;
the amino acid at position 186 is different from L and the amino acid at position 3 is different from M and the amino acid at position 17 is different from L and the amino acid at position 111 is different from Q, wherein it is preferred that the amino acid at position 186 is preferably W or Y and that the amino acid at position 3 is preferably Q and that the amino acid at position 17 is preferably P and that the amino acid at position 111 is preferably E;
the amino acid at position 186 is different from L and the amino acid at position 3 is different from M and the amino acid at position 40 is different from K and the amino acid at position 111 is different from Q, wherein it is preferred that the amino acid at position 186 is preferably W or Y and that the amino acid at position 3 is preferably Q and that the amino acid at position 40 is preferably M and that the amino acid at position 111 is preferably E;
the amino acid at position 186 is different from L and the amino acid at position 11 is different from C and the amino acid at position 17 is different from L and the amino acid at position 40 is different from K, wherein it is preferred that the amino acid at position 186 is preferably W or Y and that the amino acid at position 11 is preferably A and that the amino acid at position 17 is preferably P and that the amino acid at position 40 is preferably M;
the amino acid at position 186 is different from L and the amino acid at position 11 is different from C and the amino acid at position 17 is different from L and the amino acid at position 111 is different from Q, wherein it is preferred that the amino acid at position 186 is preferably W or Y and that the amino acid at position 11 is preferably A and that the amino acid at position 17 is preferably P and that the amino acid at position 111 is preferably E;
the amino acid at position 186 is different from L and the amino acid at position 11 is different from C and the amino acid at position 40 is different from K and the amino acid at position 111 is different from Q, wherein it is preferred that the amino acid at position 186 is preferably W or Y and that the amino acid at position 11 is preferably A and that the amino acid at position 40 is preferably M and that the amino acid at position 111 is preferably E;
the amino acid at position 186 is different from L and the amino acid at position 17 is different from L and the amino acid at position 40 is different from K and the amino acid at position 111 is different from Q, wherein it is preferred that the amino acid at position 186 is preferably W or Y and that the amino acid at position 17 is preferably P and that the amino acid at position 40 is preferably M and that the amino acid at position 111 is preferably E;
proteins having an amino acid sequence at least 80%, preferably 85%, more preferably 90%, even more preferably 95%, even more preferably 96%, even more preferably 97%, particularly preferably 98%, most preferably 99% identical to the amino acid sequence shown under a) provided that the amino acid at position 186 is different from L, wherein it is preferred that the amino acid at position 186 is preferably W or Y, more preferably Y, and wherein the proteins have at least three further amino acid substitutions selected from the group shown directly above under the symbols listed.

The lipase variants described herein above under point a) in relation to the amino acid sequences shown under SEQ ID No. 1 can have at least four further amino acid substitutions at positions 79, 202, 280, 301, 3, 11, 17, 40 or 111. Preferably, the amino acid at position 79 is S, W or I, more preferably S; preferably, the amino acid at position 202 is N; preferably, the amino acid at position 280 is A; preferably, the amino acid at position 301 is A; preferably, the amino acid at position 3 is Q; preferably, the amino acid at position 11 is A; preferably, the amino acid at position 17 is P; preferably, the amino acid at position 40 is M; preferably, the amino acid at position 111 is E.

The lipase variants described herein above under point a) in relation to the amino acid sequences shown under SEQ ID No. 1 can have at least five further amino acid substitutions at positions 79, 202, 280, 301, 3, 11, 17, 40 or 111. Preferably, the amino acid at position 79 is S, W or I, more preferably S; preferably, the amino acid at position 202 is N; preferably, the amino acid at position 280 is A; preferably, the amino acid at position 301 is A; preferably, the amino acid at position 3 is Q; preferably, the amino acid at position 11 is A; preferably, the amino acid at position 17 is P; preferably, the amino acid at position 40 is M; preferably, the amino acid at position 111 is E.

The lipase variants described herein above under point a) in relation to the amino acid sequences shown under SEQ ID No. 1 can have at least six further amino acid substitutions at positions 79, 202, 280, 301, 3, 11, 17, 40 or 111. Preferably, the amino acid at position 79 is S, W or I, more preferably S; preferably, the amino acid at position 202 is N; preferably, the amino acid at position 280 is A; preferably, the amino acid at position 301 is A; preferably, the amino acid at position 3 is Q; preferably, the amino acid at position 11 is A; preferably, the amino acid at position 17 is P; preferably, the amino acid at position 40 is M; preferably, the amino acid at position 111 is E.

The lipase variants described herein above under point a) in relation to the amino acid sequences shown under SEQ ID No. 1 can have at least seven further amino acid substitutions at positions 79, 202, 280, 301, 3, 11, 17, 40 or 111. Preferably, the amino acid at position 79 is S, W or I, more preferably S; preferably, the amino acid at position 202 is N; preferably, the amino acid at position 280 is A; preferably, the amino acid at position 301 is A; preferably, the amino acid at position 3 is Q; preferably, the amino acid at position 11 is A; preferably, the amino acid at position 17 is P; preferably, the amino acid at position 40 is M; preferably, the amino acid at position 111 is E.

Preferred embodiments according to the invention are proteins which encode lipases having the amino acid sequences shown under SEQ ID Nos. 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, 239, 241, 243, 245, 247, 249, 251, 253, 255, 257, 259, 261, 263, 265, 267, 269, 271, 273, 275, 277, 279, 281, 283, 285, 287, 289, 291, 293, 295, 297, 299, 301, 303, 305, 307, 309, 311, 313, 315, 317, 319, 321, 323, 325, 327, 329, 331, 333, 335, 337, 339, 341, 343, 345, 347, 349, 351, 353, 355, 357, 359, 361, 363, 365, 367, 369, 371, 373, 375, 377, 379, 381, 383, 385, 387, 389, 391, 393, 395, 397, 399, 401.

Particularly preferred embodiments according to the invention are proteins which encode lipases having the amino acid sequences shown under SEQ ID Nos. 233 and 399.

Further proteins having the activity of a lipolytic enzyme or a lipase have been tested. The amino acid sequences of these further proteins are variants of the amino acid sequence described by the amino acids in SEQ ID No. 1, wherein:
in the case of the amino acid sequence shown under SEQ ID No. 1, the two amino acids at positions 40 and 79 are different from the amino acids indicated at the corresponding amino acid positions in the sequence shown under SEQ ID No. 1. In a particular tested variant in relation to the amino acid sequence shown under SEQ ID No. 1, the amino acid at position 40 is M and the amino acid at position 79 is S;
in the case of the amino acid sequence shown under SEQ ID No. 1, the two amino acids at positions 40 and 186 are different from the amino acids indicated at the corresponding amino acid positions in the sequence shown under SEQ ID No. 1. In a particular tested variant in relation to the amino acid sequence shown under SEQ ID No. 1, the amino acid at position 40 is M and the amino acid at position 186 is Y;
in the case of the amino acid sequence shown under SEQ ID No. 1, the two amino acids at positions 40 and 301 are different from the amino acids indicated at the corresponding amino acid positions in the sequence shown under SEQ ID No. 1. In a particular tested variant in relation to the amino acid sequence shown under SEQ ID No. 1, the amino acid at position 40 is M and the amino acid at position 301 is A;
in the case of the amino acid sequence shown under SEQ ID No. 1, the two amino acids at positions 79 and 186 are different from the amino acids indicated at the corresponding amino acid positions in the sequence shown under SEQ ID No. 1. In a particular tested variant in relation to the amino acid sequence shown under SEQ ID No. 1, the amino acid at position 79 is S and the amino acid at position 186 is Y;
in the case of the amino acid sequence shown under SEQ ID No. 1, the two amino acids at positions 79 and 301 are different from the amino acids indicated at the corresponding amino acid positions in the sequence shown under SEQ ID No. 1. In a particular tested variant in relation to the amino acid sequence shown under SEQ ID No. 1, the amino acid at position 79 is S and the amino acid at position 301 is A;
in the case of the amino acid sequence shown under SEQ ID No. 1, the two amino acids at positions 186 and 301 are different from the amino acids indicated at the corresponding amino acid positions in the sequence shown under SEQ ID No. 1. In a particular tested variant in relation to the amino acid sequence shown under SEQ ID No. 1, the amino acid at position 186 is Y and the amino acid at position 301 is A.

Yet further proteins having the activity of a lipolytic enzyme or a lipase have been tested. The amino acid sequences of these further proteins are variants of the amino acid sequence described by the amino acids in SEQ ID No. 1, wherein:
in the case of the amino acid sequence shown under SEQ ID No. 1, the three amino acids at positions 40, 79 and 186 are different from the amino acids indicated at the corresponding amino acid positions in the sequence shown under SEQ ID No. 1. In a particular tested variant in relation to the amino acid sequence shown under SEQ ID No. 1, the amino acid at position 40 is M and the amino acid at position 79 is S and the amino acid at position 186 is Y;
in the case of the amino acid sequence shown under SEQ ID No. 1, the three amino acids at positions 40, 79 and 301 are different from the amino acids indicated at the corresponding amino acid positions in the sequence shown under SEQ ID No. 1. In a particular tested variant in relation to the amino acid sequence shown under SEQ ID No. 1, the amino acid at position 40 is M and the amino acid at position 79 is S and the amino acid at position 301 is A;
in the case of the amino acid sequence shown under SEQ ID No. 1, the three amino acids at positions 40, 186 and 301 are different from the amino acids indicated at the corresponding amino acid positions in the sequence shown under SEQ ID No. 1. In a particular tested variant in relation to the amino acid sequence shown under SEQ ID No. 1, the amino acid at position 40 is M and the amino acid at position 186 is Y and the amino acid at position 301 is A;
in the case of the amino acid sequence shown under SEQ ID No. 1, the three amino acids at positions 79, 186 and 301 are different from the amino acids indicated at the corresponding amino acid positions in the sequence shown under SEQ ID No. 1. In a particular tested variant in relation to the amino acid sequence shown under SEQ ID No. 1, the amino acid at position 79 is S and the amino acid at position 186 is Y and the amino acid at position 301 is A.

The lipases and lipase variants according to the invention have high selectivity and/or high specific activity in relation to the stereoselective acylation or carboxylation of 2,6-dimethyl-1-indanamine (DMAI) and are capable of producing enantiomerically enriched or virtually pure methyl[(1R,2S)-2,6-dimethyl-2,3-dihydro-1H-inden-1-yl]carbamate. Methyl [(1R,2S)-2,6-dimethyl-2,3-dihydro-1H-inden-1-yl]carbamate is an important intermediate for the synthesis of the compound indaziflam having herbicidal action.

What is meant herein by “enantiomerically enriched” is that one of two enantiomers in a composition is present in larger amounts than the other enantiomer, preferably one enantiomer in the composition is present to an extent of at least 60%, more preferably one enantiomer in the composition is present to an extent of at least 65%, even more preferably one enantiomer in the composition is present to an extent of at least 70%, even more preferably one enantiomer in the composition is present to an extent of at least 75%, even more preferably one enantiomer in the composition is present to an extent of at least 80%, particularly preferably one enantiomer in the composition is present to an extent of at least 85%, most preferably one enantiomer in the composition is present to an extent of at least 90% or very particularly preferably one enantiomer in the composition is present to an extent of at least 94%.

What is meant herein by “enantiomerically virtually pure” is that one of two enantiomers in a composition is present in amounts of at least 95.0%, preferably one of two enantiomers in a composition is present in amounts of at least 95.5%, more preferably one of two enantiomers in a composition is present in amounts of at least 96.0%, even more preferably one of two enantiomers in a composition is present in amounts of at least 96.5%, even more preferably one of two enantiomers in a composition is present in amounts of at least 97.0%, even more preferably one of two enantiomers in a composition is present in amounts of at least 98.0%, particularly preferably one of two enantiomers in a composition is present in amounts of at least 98.5%, most preferably one of two enantiomers in a composition is present in amounts of at least 99.0% or very particularly preferably one of two enantiomers in a composition is present in amounts of at least 99.5%.

A further embodiment according to the invention concerns nucleic acid molecules which encode a protein according to the invention.

Nucleic acid molecules according to the invention can be any kind of nucleic acid provided that the nucleic acid encodes a protein according to the invention. The nucleic acids can be ribonucleic acid molecules (e.g. RNA, mRNA) or deoxyribonucleic molecules (DNA, including genomic DNA, which may or may not include introns and coding DNA).

Of particular interest to the invention are nucleic acid molecules which encode proteins having the activity of a lipase, comprising the amino acid sequences shown under SEQ ID Nos. 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, 239, 241, 243, 245, 247, 249, 251, 253, 255, 257, 259, 261, 263, 265, 267, 269, 271, 273, 275, 277, 279, 281, 283, 285, 287, 289, 291, 293, 295, 297, 299, 301, 303, 305, 307, 309, 311, 313, 315, 317, 319, 321, 323, 325, 327, 329, 331, 333, 335, 337, 339, 341, 343, 345, 347, 349, 351, 353, 355, 357, 359, 361, 363, 365, 367, 369, 371, 373, 375, 377, 379, 381, 383, 385, 387, 389, 391, 393, 395, 397, 399, 401.

The invention further concerns nucleic acid molecules which encode a protein having the activity of a lipase, selected from the group consisting of
a) nucleic acid molecules comprising the nucleic acid sequences shown under SEQ ID Nos. 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 394, 396, 398, 400, 402;
b) nucleic acid molecules having at least 60%, preferably 70%, more preferably 80%, even more preferably 90%, even more preferably 95%, even more preferably 96%, particularly preferably 97%, most preferably 98% or very particularly preferably 99% identity to the nucleic acid sequences shown under a).

In connection with the present invention, “hybridize to” means hybridization under conventional hybridization conditions, preferably under stringent conditions, as described for example in Sambrook et al. (Molecular Cloning, A Laboratory Manual, 3rd edition (2001) Cold Spring Harbor Laboratory Press, Cold Spring Harbor, NY. ISBN: 0879695773) or in Ausubel et al. (Short Protocols in Molecular Biology, John Wiley & Sons; 5th edition (2002), ISBN: 0471250929). Particularly preferably, “hybridization” means hybridization under the following conditions:

### Hybridization Buffer:

2×SSC; 10×Denhardt's solution (Fikoll 400+PEG+BSA; 1:1:1 ratio); 0.1% SDS; 5 mM EDTA; 50 mM Na2HPO4; 250 μg/ml herring sperm DNA; 50 μg/ml tRNA;
or
25 M sodium phosphate buffer, pH 7.2; 1 mM EDTA; 7% SDS
Hybridization temperature: T=65° C. to 68° C.
Wash buffer: 0.1×SSC; 0.1% SDS
Wash temperature: T=65° C. to 68° C.

Nucleic acid molecules which hybridize to nucleic acid molecules encoding a protein having the activity of a lipase can originate from any organism; accordingly, they can originate from bacteria, fungi, animals, humans, plants or viruses.

Nucleic acid molecules which hybridize to nucleic acid molecules encoding a protein having the activity of a lipase preferably originate from microorganisms, more preferably from fungi or bacteria, most preferably from bacteria.

Nucleic acid molecules which hybridize to the aforementioned molecules can, for example, be isolated from genomic DNA or cDNA libraries. These nucleic acid molecules can be identified and isolated using the nucleic acid molecules described herein or they can be identified and isolated using parts of these molecules or the reverse complements of these molecules, for example by hybridization according to standard methods (see, for example, Sambrook et al., Molecular Cloning, A Laboratory Manual, 3rd edition (2001) Cold Spring Harbor Laboratory Press, Cold Spring Harbor, NY. ISBN: 0879695773; Ausubel et al., Short Protocols in Molecular Biology, John Wiley & Sons; 5th edition (2002), ISBN: 0471250929) or by amplification with the aid of PCR.

The fragments used as hybridization probes can also be synthetic fragments, or oligonucleotides produced using conventional synthesis techniques, the sequence of which is substantially identical to the nucleic acid molecule described in the context of the present invention. When genes which hybridize to the nucleic acid sequences described in connection with the present invention are identified and isolated, the sequence should be determined and the properties of the proteins encoded by said sequence should be analysed in order to determine whether they are proteins having the activity of a lipase. Methods for determining whether a protein has the activity of a protein having the activity of a lipase are known to those of ordinary skill in the art.

The molecules which hybridize to the nucleic acid molecules described in the context of the present invention include especially fragments, derivatives and allelic variants of the aforementioned nucleic acid molecules. In connection with the present invention, the term “derivative” means that the sequences of these molecules differ from the sequences of the above-described nucleic acid molecules in one or more positions and are identical to these sequences to a high degree. The differences in relation to the above-described nucleic acid molecules can, for example, be attributable to deletion, addition, substitution, insertion or recombination.

Preferred nucleic acid molecules according to the invention are the nucleic acid molecules shown under SEQ ID Nos. 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 394, 396, 398, 400, 402.

The meaning of the nucleotide abbreviations a, c, g, t and of the abbreviations for degenerate nucleotides r, y, s, w, k, m, b, d, h, v, n can be found herein in what follows in Table 1 in the section titled “Description of the sequences”. The amino acids encoded by degenerate nucleotides can be found herein in what follows in Table 3 in the section titled “Description of the sequences”.

Also disclosed are recombinant nucleic acid molecules comprising a nucleic acid molecule suitable for the process according to the invention.

The term “recombinant nucleic acid molecule” is to be understood to mean a nucleic acid molecule which contains not only the nucleic acid molecule suitable for the process according to the invention, but also further sequences which do not naturally occur in the combination in which they occur in the recombinant nucleic acids for the process according to the invention. The aforementioned additional sequences can be any sequence; preferably they are functional or regulatory sequences (promoters, termination signals, enhancers, ribosome binding sites (rbs), leader sequences which increase transcription, translation or RNA stability, subcellular targeting sequences, etc.), particularly preferably they are functional or regulatory sequences which are active in microorganisms, and very particularly preferably they are regulatory sequences which are active in fungi, in particular in yeasts, or in bacteria. Methods for producing the recombinant nucleic acid molecules suitable for the process according to the invention are known to those of ordinary skill in the art. These include genetic methods, such as binding of nucleic acid molecules by ligation, genetic recombination or de novo synthesis of nucleic acid molecules. These method are, for example, described in Sambrok et al. (Molecular Cloning, A Laboratory Manual, 3rd edition (2001) Cold Spring Harbour Laboratory Press, Cold Spring Harbour, NY. ISBN: 0879695773) or in Ausubel et al. (Short Protocols in Molecular Biology, John Wiley & Sons; 5th edition (2002), ISBN: 0471250929).

The recombinant nucleic acid molecule usable for the process according to the invention comprises a suitable nucleic acid molecule which is linked to regulatory sequences which initiate transcription in prokaryotic or eukaryotic cells.

Regulatory sequences which initiate transcription in a cell are also known as promoters. Information regarding regulatory sequences and plasmids are well known to those of ordinary skill in the art and are, for example, described in the Registry of Standard Biological Parts, supported by The International Genetically Engineered Machine (iGEM) Foundation (One Kendall Square, Suite B6104, Cambridge, MA 02139, USA) on the Internet (http://parts.igem.org/Catalog).

Regulatory sequences which initiate transcription in prokaryotic organisms, such as E. coli, and in eukaryotic organisms are extensively described in the literature; described in particular are those in relation to expression in yeasts, for example Saccharomyces cerevisiae. An overview of different systems for the expression of proteins in different host organisms can, for example, be found in Methods in Enzymology 153 (1987), 383-516 and in Bitter et al. (Methods in Enzymology 153 (1987), 516-544) or in Gomes et al. (2016, Advances in Animal and Veterinary Sciences, 4 (4), 346) and Baghban et al. (2018, Current Pharmaceutical Biotechnology, 19 (6)). Customary yeast promoters are pAOX1, pHIS4, pGAL, pScADH2 (Baghban et al., 2018, see above). Customary bacterial promoters are T5 promoter, T7 promoter, rhamnose-inducible promoter, arabinose-inducible promoter, PhoA promoter, artificial trc (trp-lac) promoter, as described by Marschall et al. (2017, Appl Microbiol Biotechnol 101, 501-512) and Tegel et al. (2011, FEBS Journal 278, 729-739).

A further embodiment of recombinant nucleic acid molecules usable for the process according to the invention are vectors of plasmids comprising the suitable nucleic acid molecules.

“Vectors” are well known in the field of molecular biology and are herein a nucleic acid sequence or a vehicle comprising a nucleic acid sequence that is used for transferring genetic material (DNA or RNA) into a target cell. Vectors can be plasmids, for example T-DNA or binary vectors for generation of transgenic plants, expression vectors for the expression of nucleic acid sequences in a host cell, shuttle vectors capable of multiplying in different hosts, or vectors can be virus particles or bacteriophages that have been modified to deliver foreign genetic material into a host.

“Plasmids” are well known in the field of molecular biology and are herein an autonomously self-replicating, commonly circular DNA molecule which, when present in a host cell, is separate from the chromosomal DNA.

Suitable nucleic acid molecules, recombinant nucleic acid molecules, vectors or plasmids can be used for producing proteins for the process according to the invention, for example by expression of the suitable nucleic acid molecule in host cells.

Also disclosed are hosts or host cells which comprise or express a nucleic acid molecule suitable for the process according to the invention or comprise suitable proteins having the activity of a lipase or comprise a recombinant nucleic acid molecule suitable for the process according to the invention or comprise a vector suitable for the process according to the invention or comprise a plasmid suitable for the process according to the invention.

Suitable nucleic acid molecules encoding a protein having the activity of a lipase can be expressed in host cells, for example for the multiplication thereof or for production of proteins having the activity of a lipase. For expression in host cells, suitable nucleic acid molecules can be present on vectors or plasmids or be stably integrated into the genome of a particular host cell. The suitable nucleic acid molecules can also be present in vectors which assist their introduction into host cells.

Also disclosed is a host or host cell that is suitable for the process according to the invention, comprising a nucleic acid molecule suitable for the process according to the invention or comprising a recombinant nucleic acid molecule suitable for the process according to the invention or comprising a vector suitable for the process according to the invention or comprising a plasmid suitable for the process according to the invention, each comprising a protein suitable for the process according to the invention. Also disclosed is a host or host cell that is suitable for the process according to the invention, comprising a nucleic acid molecule suitable for the process according to the invention or comprising a recombinant nucleic acid molecule suitable for the process according to the invention or comprising a vector suitable for the process according to the invention or comprising a plasmid suitable for the process according to the invention, each expressing a protein suitable for the process according to the invention, wherein the protein preferably has the activity of a lipase.

“Expression of a nucleic acid molecule” is to be understood herein to mean that, if the nucleic acid molecule is RNA or mRNA, the nucleic acid molecule is translated into a protein, preferably into a protein having the activity of a lipase or, if the nucleic acid molecule is DNA or cDNA, it is transcribed into mRNA (and processed in the case of genomic DNA containing introns), preferably into an mRNA encoding a protein having the activity of a lipase, and is subsequently translated into a protein, preferably translated into a protein having the activity of a lipase.

The transcription of a particular nucleic acid molecule in a host can be detected by methods known to those of ordinary skill in the art, for example by detection of specific transcripts (mRNA) of foreign nucleic acid molecules by Northern blot analysis or by RT-PCR.

Determination of whether hosts or host cells comprise a particular protein or comprise a protein derived from the expression of a nucleic acid molecule can be carried out by methods known to those of ordinary skill in the art, for example by immunological methods, such as Western blot analysis, ELISA (enzyme-linked immunosorbent assay) or RIA (radioimmunoassay). Those of ordinary skill in the art are familiar with methods for producing antibodies which react specifically with a particular protein, i.e. which bind specifically to a particular protein (see, for example, Lottspeich and Zorbas (editors), 1998, Bioanalytik, Spektrum akad, Verlag, Heidelberg, Berlin, ISBN 3-8274-0041-4). Some companies (Thermo Fisher Scientific, 168 Third Avenue, Waltham, MA USA 0245; GenScript, 60 Centennial Ave., Piscataway, NJ 08854, USA) provide a made-to-order service for the production of said antibodies.

Furthermore, those of ordinary skill in the art can check whether a host or a host cell comprises a protein suitable for the process according to the invention by detecting the (additional) activity of proteins having the activity of a lipase in a corresponding host cell. Preferably, the activity of proteins having additional activity of a lipase in a corresponding host cell is detected by comparing the activities of lipases in a host cell used for the process according to the invention with the corresponding activity of host cells which do not comprise a protein suitable for the process according to the invention. Checking whether a protein has the activity of a lipase can be carried out using methods known according to the prior art.

A host or host cell used for the process according to the invention can be produced by those of ordinary skill in the art with the aid of known methods for genetic modification or transformation of organisms.

Therefore, also disclosed are a host or host cell suitable for the process according to the invention, in particular a prokaryotic or eukaryotic host or host cell, genetically modified (or transformed) using a suitable nucleic acid molecule or using a suitable recombinant nucleic acid molecule or using a suitable vector or using a suitable plasmid. Preferably, the genetically modified (transformed) host or host cell used for the process according to the invention expresses a protein having the activity of a lipase; more preferably, the genetically modified (transformed) host or host cell expresses a protein suitable for the process according to the invention. “Genetically modified using a nucleic acid molecule” or “transformed using a nucleic acid molecule” is to be understood herein to mean that a nucleic acid molecule is or has been introduced into a host or into a host cell by technical and/or non-naturally occurring means, preferably by technical methods from the area of molecular biology, of biotechnology or of gene technology.

Descendants or progenies of hosts or host cells used for the process according to the invention are likewise disclosed; preferably said descendants or progenies comprise a nucleic acid molecule suitable for the process according to the invention or comprise a suitable recombinant nucleic acid molecule or comprise a suitable vector or comprise a suitable plasmid or comprise a suitable protein, more preferably said descendants or progenies comprise a suitable nucleic acid molecule or comprise a suitable recombinant nucleic acid molecule or comprise a suitable vector or comprise a suitable plasmid and they express in any case a protein having the activity of a lipase, even more preferably said descendants or progenies comprise a suitable nucleic acid molecule or comprise a suitable recombinant nucleic acid molecule or comprise a suitable vector or comprise a suitable plasmid and they express in any case a protein having the activity of a lipase which can be used in the process according to the invention.

The host or host cell for the process according to the invention can be a host or host cell from a prokaryotic organism or a eukaryotic organism. The host or the host cells can be bacteria or bacterial cells (e.g. E. coli, bacteria of the genus Bacillus, in particular Bacillus subtilis, Agrobacterium, in particular Agrobacterium tumefaciens or Agrobacterium rhizogenes, Pseudomonas, in particular Pseudomonas fluorescens, Streptomyces spp, Rhodococcus spp, in particular Rhodococcus rhodochrous, Vibrio natrigens, Corynebacterium, in particular Corynebacterium glutamicum) or fungi or fungal cells (e.g. Agaricus, in particular Agaricus bisporus, Aspergillus, Trichoderma or yeasts, in particular S. cerevisiae, Pichia ssp, such as P. pastoris), and also plants or plant cells or they can be animals or animal cells. Preferred host cells are cells of microorganisms. In the context of the present patent application, it is assumed that all bacteria and protists (e.g. fungi, in particular yeasts and algae), as defined for example in Schlegel “General Microbiology” (Georg Thieme Verlag (1985), 1-2), are included. With regard to microorganisms, the hosts or host cells used for the process according to the invention are preferably bacteria/bacterial cells or yeasts/yeast cells, more preferably bacteria/bacterial cells, even more preferably Bacillus species/Bacillus species cells or Escherichia coli/Escherichia coli cells, most preferably Escherichia coli/Escherichia coli cells. Alternatively, Pseudomonas, in particular Pseudomonas fluorescens, Streptomyces spp, Rhodococcus spp, in particular Rhodococcus rhodochrous, Vibrio spp, in particular Vibrio natrigens, Corynebacterium, in particular Corynebacterium glutamicum, or other hosts or host cells can be used for the process according to the invention.

Preferred hosts or host cells for the process according to the invention comprise a suitable nucleic acid molecule which is characterized in that the codons of the nucleic acid molecule have been altered in such a way that they are adapted to the usage frequency of the codons in the host or in a host cell.

### Description of the Sequences

Throughout the application, use is made of abbreviations for nucleotides and amino acids according to the following IUPAC codes:

TABLE 1

| IUPAC nucleotide | |
|---|---|
| code | Base |
| A | Adenine |
| C | Cytosine |
| G | Guanine |
| T (or U) | Thymine (or uracil) |
| R | A or G |
| Y | C or T |
| S | G or C |
| W | A or T |
| K | G or T |
| M | A or C |
| B | Cor Gor T |
| D | A or G or T |
| H | A or C or T |
| V | A or C or G |
| N | Any base |
| - | Gap |

To distinguish between amino acids and nucleotides, the abbreviated nucleotide codes which are capitalized in the table above are uncapitalized herein.

TABLE 2

| IUPAC | | |
|---|---|---|
| amino acid | Three- | |
| code | letter code | Amino acid |
| A | Ala | Alanine |
| C | Cys | Cysteine |
| D | Asp | Aspartic acid |
| E | Glu | Glutamic acid |
| F | Phe | Phenylalanine |
| G | Gly | Glycine |
| H | His | Histidine |
| I | Ile | Isoleucine |
| K | Lys | Lysine |
| L | Leu | Leucine |
| M | Met | Methionine |
| N | Asn | Asparagine |
| P | Pro | Proline |
| Q | Gln | Glutamine |
| R | Arg | Arginine |
| S | Ser | Serine |
| T | Thr | Threonine |
| V | Val | Valine |
| W | Trp | Tryptophan |
| Y | Tyr | Tyrosine |

The use of codons herein follows the so-called “general genetic code” according to the following table, with replacement of “t” by “u” in ribonucleic acid (RNA) sequences.

TABLE 3

| | | | | Codons due |
|---|---|---|---|---|
| | Three- | One- | DNA | to degenerate |
| Amino acid | letter code | letter code | codons | genetic code |
| Alanine | Ala | A | gca | gcn |
| Alanine | Ala | A | gcc | gcn |
| Alanine | Ala | A | gcg | gcn |
| Alanine | Ala | A | gct | gcn |
| Arginine | Arg | R | aga | mgn |
| Arginine | Arg | R | agg | mgn |
| Arginine | Arg | R | cga | mgn |
| Arginine | Arg | R | cgc | mgn |
| Arginine | Arg | R | cgg | mgn |
| Arginine | Arg | R | cgt | mgn |
| Asparagine | Asn | N | aac | aay |
| Asparagine | Asn | N | aat | aay |
| Aspartic acid | Asp | D | gac | gay |
| Aspartic acid | Asp | D | gat | gay |
| Cysteine | Cys | C | tgc | tgy |
| Cysteine | Cys | C | tgt | tgy |
| Glutamic acid | Glu | E | gaa | gar |
| Glutamic acid | Glu | E | gag | gar |
| Glutamine | Gln | Q | caa | car |
| Glutamine | Gln | Q | cag | car |
| Glycine | Gly | G | gga | ggn |
| Glycine | Gly | G | ggc | ggn |
| Glycine | Gly | G | ggg | ggn |
| Glycine | Gly | G | ggt | ggn |
| Histidine | His | H | cac | cay |
| Histidine | His | H | cat | cay |
| Isoleucine | Ile | I | ata | ath |
| Isoleucine | Ile | I | atc | ath |
| Isoleucine | Ile | I | att | ath |
| Leucine | Leu | L | cta | ytn |
| Leucine | Leu | L | ctc | ytn |
| Leucine | Leu | L | ctg | ytn |
| Leucine | Leu | L | ctt | ytn |
| Leucine | Leu | L | tta | ytn |
| Leucine | Leu | L | ttg | ytn |
| Lysine | Lys | K | aaa | aar |
| Lysine | Lys | K | aag | aar |
| Methionine | Met | M | atg | atg |
| Phenylalanine | Phe | F | ttc | tty |
| Phenylalanine | Phe | F | ttt | tty |
| Proline | Pro | P | cca | ccn |
| Proline | Pro | P | ccc | ccn |
| Proline | Pro | P | ccg | ccn |
| Proline | Pro | P | cct | ccn |
| Serine | Ser | S | agc | wsn |
| Serine | Ser | S | agt | wsn |
| Serine | Ser | S | tca | wsn |
| Serine | Ser | S | tcc | wsn |
| Serine | Ser | S | tcg | wsn |
| Serine | Ser | S | tct | wsn |
| Threonine | Thr | T | aca | acn |
| Threonine | Thr | T | acc | acn |
| Threonine | Thr | T | acg | acn |
| Threonine | Thr | T | act | acn |
| Tryptophan | Thr | W | tgg | tgg |
| Tyrosine | Tyr | Y | tac | tay |
| Tyrosine | Tyr | Y | tat | tay |
| Valine | Val | V | gta | gtn |
| Valine | Val | V | gt | gtn |
| Valine | Val | V | gtg | gtn |
| Valine | Val | V | gtt | gtn |
| Stop codons | Stop | Stop | taa | trr |
| Stop codons | Stop | Stop | tag | trr |
| Stop codons | Stop | Stop | tga | trr |

TABLE 4
The sequence listing linked to this application is filed in
electronic format and is hereby incorporated by
reference in this patent document in its entirety. ″PRT″ means
″protein″ and ″NUC″ means ″nucleic acid″.

| SEQ ID No. | Subject | Mutations/variations | Type |
|---|---|---|---|
| 1 | Wild type (WT) | - | PRT |
| 2 | Wild type (WT) | - | NUC |
| 3 | Lipase variant | L186F | PRT |
| 4 | Lipase variant | L186F | NUC |
| 5 | Lipase variant | L280E | PRT |
| 6 | Lipase variant | L280E | NUC |
| 7 | Lipase variant | L280S | PRT |
| 8 | Lipase variant | L280S | NUC |
| 9 | Lipase variant | L280K | PRT |
| 10 | Lipase variant | L280K | NUC |
| 11 | Lipase variant | P312N | PRT |
| 12 | Lipase variant | P312N | NUC |
| 13 | Lipase variant | P312F | PRT |
| 14 | Lipase variant | P312F | NUC |
| 15 | Lipase variant | L186W | PRT |
| 16 | Lipase variant | L186W | NUC |
| 17 | Lipase variant | M3L | PRT |
| 18 | Lipase variant | M3L | NUC |
| 19 | Lipase variant | N29Y | PRT |
| 20 | Lipase variant | N29Y | NUC |
| 21 | Lipase variant | L17P | PRT |
| 22 | Lipase variant | L17P | NUC |
| 23 | Lipase variant | S4P | PRT |
| 24 | Lipase variant | S4P | NUC |
| 25 | Lipase variant | L280D | PRT |
| 26 | Lipase variant | L280D | NUC |
| 27 | Lipase variant | V18A | PRT |
| 28 | Lipase variant | V18A | NUC |
| 29 | Lipase variant | M3Q | PRT |
| 30 | Lipase variant | M3Q | NUC |
| 31 | Lipase variant | P312D | PRT |
| 32 | Lipase variant | P312D | NUC |
| 33 | Lipase variant | N29W | PRT |
| 34 | Lipase variant | N29W | NUC |
| 35 | Lipase variant | L186Y | PRT |
| 36 | Lipase variant | L186Y | NUC |
| 37 | Lipase variant | V18T | PRT |
| 38 | Lipase variant | V18T | NUC |
| 39 | Lipase variant | L186E | PRT |
| 40 | Lipase variant | L186E | NUC |
| 41 | Lipase variant | A202Q | PRT |
| 42 | Lipase variant | A202Q | NUC |
| 43 | Lipase variant | D301A | PRT |
| 44 | Lipase variant | D301A | NUC |
| 45 | Lipase variant | P309C | PRT |
| 46 | Lipase variant | P309C | NUC |
| 47 | Lipase variant | L186D | PRT |
| 48 | Lipase variant | L186D | NUC |
| 49 | Lipase variant | Q31W | PRT |
| 50 | Lipase variant | Q31W | NUC |
| 51 | Lipase variant | A202N | PRT |
| 52 | Lipase variant | A202N | NUC |
| 53 | Lipase variant | Q111E | PRT |
| 54 | Lipase variant | Q111E | NUC |
| 55 | Lipase variant | L186Q | PRT |
| 56 | Lipase variant | L186Q | NUC |
| 57 | Lipase variant | L186T | PRT |
| 58 | Lipase variant | L186T | NUC |
| 59 | Lipase variant | M3C | PRT |
| 60 | Lipase variant | M3C | NUC |
| 61 | Lipase variant | W85H | PRT |
| 62 | Lipase variant | W85H | NUC |
| 63 | Lipase variant | N29H | PRT |
| 64 | Lipase variant | N29H | NUC |
| 65 | Lipase variant | K8E | PRT |
| 66 | Lipase variant | K8E | NUC |
| 67 | Lipase variant | L186H | PRT |
| 68 | Lipase variant | L186H | NUC |
| 69 | Lipase variant | E79I | PRT |
| 70 | Lipase variant | E79I | NUC |
| 71 | Lipase variant | E79W | PRT |
| 72 | Lipase variant | E79W | NUC |
| 73 | Lipase variant | L17T | PRT |
| 74 | Lipase variant | L17T | NUC |
| 75 | Lipase variant | V18C | PRT |
| 76 | Lipase variant | V18C | NUC |
| 77 | Lipase variant | L186P | PRT |
| 78 | Lipase variant | L186P | NUC |
| 79 | Lipase variant | P312Q | PRT |
| 80 | Lipase variant | P312Q | NUC |
| 81 | Lipase variant | K40M | PRT |
| 82 | Lipase variant | K40M | NUC |
| 83 | Lipase variant | P312K | PRT |
| 84 | Lipase variant | P312K | NUC |
| 85 | Lipase variant | L186C | PRT |
| 86 | Lipase variant | L186C | NUC |
| 87 | Lipase variant | L280A | PRT |
| 88 | Lipase variant | L280A | NUC |
| 89 | Lipase variant | S4L | PRT |
| 90 | Lipase variant | S4L | NUC |
| 91 | Lipase variant | V18S | PRT |
| 92 | Lipase variant | V18S | NUC |
| 93 | Lipase variant | L186K | PRT |
| 94 | Lipase variant | L186K | NUC |
| 95 | Lipase variant | L186S | PRT |
| 96 | Lipase variant | L186S | NUC |
| 97 | Lipase variant | E79T | PRT |
| 98 | Lipase variant | E79T | NUC |
| 99 | Lipase variant | L186N | PRT |
| 100 | Lipase variant | L186N | NUC |
| 101 | Lipase variant | L186I | PRT |
| 102 | Lipase variant | L186I | NUC |
| 103 | Lipase variant | L186V | PRT |
| 104 | Lipase variant | L186V | NUC |
| 105 | Lipase variant | E79S, L186Y | PRT |
| 106 | Lipase variant | E79S, L186Y | NUC |
| 107 | Lipase variant | C11A, L186Y, P312N | PRT |
| 108 | Lipase variant | C11A, L186Y, P312N | NUC |
| 109 | Lipase variant | L186Y, L280A, P312N | PRT |
| 110 | Lipase variant | L186Y, L280A, P312N | NUC |
| 111 | Lipase variant | L17P, L186Y, P312N | PRT |
| 112 | Lipase variant | L17P, L186Y, P312N | NUC |
| 113 | Lipase variant | L186W, L280A, P312N | PRT |
| 114 | Lipase variant | L186W, L280A, P312N | NUC |
| 115 | Lipase variant | N29H, L186W, L280A | PRT |
| 116 | Lipase variant | N29H, L186W, L280A | NUC |
| 117 | Lipase variant | L17P, L186Y, P309C | PRT |
| 118 | Lipase variant | L17P, L186Y, P309C | NUC |
| 119 | Lipase variant | S4P, L186W, A202N | PRT |
| 120 | Lipase variant | S4P, L186W, A202N | NUC |
| 121 | Lipase variant | L186W, A202N, P312N | PRT |
| 122 | Lipase variant | L186W, A202N, P312N | NUC |
| 123 | Lipase variant | E79S, L186W, P309C, P312N | PRT |
| 124 | Lipase variant | E79S, L186W, P309C, P312N | NUC |
| 125 | Lipase variant | L186Y, F192A, A202N, P312N | PRT |
| 126 | Lipase variant | L186Y, F192A, A202N, P312N | NUC |
| 127 | Lipase variant | K8E, C11A, L186W, P312N | PRT |
| 128 | Lipase variant | K8E, C11A, L186W, P312N | NUC |
| 129 | Lipase variant | E79S, L186Y, P309C, P312N | PRT |
| 130 | Lipase variant | E79S, L186Y, P309C, P312N | NUC |
| 131 | Lipase variant | E79S, N84T, L186W, P309C | PRT |
| 132 | Lipase variant | E79S, N84T, L186W, P309C | NUC |
| 133 | Lipase variant | E79S, L186W, F192A, P312N | PRT |
| 134 | Lipase variant | E79S, L186W, F192A, P312N | NUC |
| 135 | Lipase variant | N29H, N84T, L186Y, A202N | PRT |
| 136 | Lipase variant | N29H, N84T, L186Y, A202N | NUC |
| 137 | Lipase variant | V18C, K40M, N84T, L186W, P312N | PRT |
| 138 | Lipase variant | V18C, K40M, N84T, L186W, P312N | NUC |
| 139 | Lipase variant | S4P, N29H, L186Y, A202N, P312N | PRT |
| 140 | Lipase variant | S4P, N29H, L186Y, A202N, P312N | NUC |
| 141 | Lipase variant | V18A, L186Y, F192A, D301A, P312N | PRT |
| 142 | Lipase variant | V18A, L186Y, F192A, D301A, P312N | NUC |
| 143 | Lipase variant | N29H, Q111E, L186W, P309C, P312N | PRT |
| 144 | Lipase variant | N29H, Q111E, L186W, P309C, P312N | NUC |
| 145 | Lipase variant | M3Q, S4P, K8E, N84T, L186W, P312N | PRT |
| 146 | Lipase variant | M3Q, S4P, K8E, N84T, L186W, P312N | NUC |
| 147 | Lipase variant | E79S, L186Y, D301A | PRT |
| 148 | Lipase variant | E79S, L186Y, D301A | NUC |
| 149 | Lipase variant | L186Y, A202N, D301A | PRT |
| 150 | Lipase variant | L186Y, A202N, D301A | NUC |
| 151 | Lipase variant | L17P, L186W, A202N | PRT |
| 152 | Lipase variant | L17P, L186W, A202N | NUC |
| 153 | Lipase variant | M3Q, E79S, L186Y, P312N | PRT |
| 154 | Lipase variant | M3Q, E79S, L186Y, P312N | NUC |
| 155 | Lipase variant | K40M, E79S, L186Y, P312N | PRT |
| 156 | Lipase variant | K40M, E79S, L186Y, P312N | NUC |
| 157 | Lipase variant | K8E, L186Y, L280A, D301A | PRT |
| 158 | Lipase variant | K8E, L186Y, L280A, D301A | NUC |
| 159 | Lipase variant | L17P, E79S, L186Y, P309C | PRT |
| 160 | Lipase variant | L17P, E79S, L186Y, P309C | NUC |
| 161 | Lipase variant | L186Y, F192A, L280A, D301A | PRT |
| 162 | Lipase variant | L186Y, F192A, L280A, D301A | NUC |
| 163 | Lipase variant | L186Y, A202N, L280A, P312N | PRT |
| 164 | Lipase variant | L186Y, A202N, L280A, P312N | NUC |
| 165 | Lipase variant | K40M, L186W, L280A, P309C | PRT |
| 166 | Lipase variant | K40M, L186W, L280A, P309C | NUC |
| 167 | Lipase variant | C11A, N29W, E79S, L186Y | PRT |
| 168 | Lipase variant | C11A, N29W, E79S, L186Y | NUC |
| 169 | Lipase variant | N29W, L186Y, A202N, D301A | PRT |
| 170 | Lipase variant | N29W, L186Y, A202N, D301A | NUC |
| 17 | Lipase variant | L17P, N29H, E79S, L186Y | PRT |
| 172 | Lipase variant | L17P, N29H, E79S, L186Y | NUC |
| 173 | Lipase variant | K8E, C11A, L186Y, D301A | PRT |
| 174 | Lipase variant | K8E, C11A, L186Y, D301A | NUC |
| 175 | Lipase variant | S4P, E79S, L186W, A202N, P309C | PRT |
| 176 | Lipase variant | S4P, E79S, L186W, A202N, P309C | NUC |
| 177 | Lipase variant | Q31W, E79S, L186W, A202N, P309C | PRT |
| 178 | Lipase variant | Q31W, E79S, L186W, A202N, P309C | NUC |
| 179 | Lipase variant | E79S, L186W, L280A, P309C, P312N | PRT |
| 180 | Lipase variant | E79S, L186W, L280A, P309C, P312N | NUC |
| 181 | Lipase variant | Q31W, E79S, L186W, F192A, D301A | PRT |
| 182 | Lipase variant | Q31W, E79S, L186W, F192A, D301A | NUC |
| 183 | Lipase variant | Q31W, E79S, N84T, L186W, A202N | PRT |
| 184 | Lipase variant | Q31W, E79S, N84T, L186W, A202N | NUC |
| 185 | Lipase variant | E79S, N84T, L186Y, F192A, L280A | PRT |
| 186 | Lipase variant | E79S, N84T, L186Y, F192A, L280A | NUC |
| 187 | Lipase variant | E79S, N84T, L186W, F192A, L280A | PRT |
| 188 | Lipase variant | E79S, N84T, L186W, F192A, L280A | NUC |
| 189 | Lipase variant | S4P, L17P, N29W, L186Y, D301A | PRT |
| 190 | Lipase variant | S4P, L17P, N29W, L186Y, D301A | NUC |
| 191 | Lipase variant | N29H, E79S, L186W, F192A, A202N | PRT |
| 192 | Lipase variant | N29H, E79S, L186W, F192A, A202N | NUC |
| 193 | Lipase variant | E79S, W85H, L186Y, F192A, D301A | PRT |
| 194 | Lipase variant | E79S, W85H, L186Y, F192A, D301A | NUC |
| 195 | Lipase variant | L17P, N29H, E79S, L186Y, P309C | PRT |
| 196 | Lipase variant | L17P, N29H, E79S, L186Y, P309C | NUC |
| 197 | Lipase variant | N29H, Q31W, L186Y, L280A, D301A | PRT |
| 198 | Lipase variant | N29H, Q31W, L186Y, L280A, D301A | NUC |
| 199 | Lipase variant | S4P, C11A, W85H, L186Y, A202N | PRT |
| 200 | Lipase variant | S4P, C11A, W85H, L186Y, A202N | NUC |
| 201 | Lipase variant | S4P, E79S, W85H, L186Y, L280A | PRT |
| 202 | Lipase variant | S4P, E79S, W85H, L186Y, L280A | NUC |
| 203 | Lipase variant | K8E, K40M, E79S, N84T, L186Y, P312N | PRT |
| 204 | Lipase variant | K8E, K40M, E79S, N84T, L186Y, P312N | NUC |
| 205 | Lipase variant | Q31W, N84T, Q111E, L186Y, L280A, P312N | PRT |
| 206 | Lipase variant | Q31W, N84T, Q111E, L186Y, L280A, P312N | NUC |
| 207 | Lipase variant | Q31W, L186Y, F192A, L280A, D301A, P312N | PRT |
| 208 | Lipase variant | Q31W, L186Y, F192A, L280A, D301A, P312N | NUC |
| 209 | Lipase variant | S4P, K8E, N29W, E79S, Q111E, L186W | PRT |
| 210 | Lipase variant | S4P, K8E, N29W, E79S, Q111E, L186W | NUC |
| 211 | Lipase variant | L17P, V18A, L186W, L280A, P309C, P312N | PRT |
| 212 | Lipase variant | L17P, V18A, L186W, L280A, P309C, P312N | NUC |
| 213 | Lipase variant | E79S, N84T, Q111E, L186Y, P309C, P312N | PRT |
| 214 | Lipase variant | E79S, N84T, Q111E, L186Y, P309C, P312N | NUC |
| 215 | Lipase variant | N29H, L186Y, F192A, L280A, D301A, P312N | PRT |
| 216 | Lipase variant | N29H, L186Y, F192A, L280A, D301A, P312N | NUC |
| 217 | Lipase variant | K8E, C11A, N29H, Q31W, L186Y, D301A | PRT |
| 218 | Lipase variant | K8E, C11A, N29H, Q31W, L186Y, D301A | NUC |
| 219 | Lipase variant | S4P, N29H, Q31W, E79S, L280A, D301A | PRT |
| 220 | Lipase variant | S4P, N29H, Q31W, E79S, L280A, D301A | NUC |
| 221 | Lipase variant | K8E, N29H, Q31W, E79S, L186W, D301A | PRT |
| 222 | Lipase variant | K8E, N29H, Q31W, E79S, L186W, D301A | NUC |
| 223 | Lipase variant | K8E, L17P, V18A, Q111E, L186W, P309C | PRT |
| 224 | Lipase variant | K8E, L17P, V18A, Q111E, L186W, P309C | NUC |
| 225 | Lipase variant | C11A, N29W, E79S, N84T, W85H, L186Y, P309C | PRT |
| 226 | Lipase variant | C11A, N29W, E79S, N84T, W85H, L186Y, P309C | NUC |
| 227 | Lipase variant | N29H, N84T, L186Y, A202N, L280A, P309C, P312N | PRT |
| 228 | Lipase variant | N29H, N84T, L186Y, A202N, L280A, P309C, P312N | NUC |
| 229 | Lipase variant | N29H, Q31W, E79S, N84T, W85H, L186Y, F192A, L280A, P309C | PRT |
| 230 | Lipase variant | N29H, Q31W, E79S, N84T, W85H, L186Y, F192A, L280A, P309C | NUC |
| 231 | Lipase variant | L17P, E79S, L186W, A202N | PRT |
| 232 | Lipase variant | L17P, E79S, L186W, A202N | NUC |
| 233 | Lipase variant | K40M, E79S, L186Y, D301A | PRT |
| 234 | Lipase variant | K40M, E79S, L186Y, D301A | NUC |
| 235 | Lipase variant | E79S, L186W, A202N, L280A | PRT |
| 236 | Lipase variant | E79S, L186W, A202N, L280A | NUC |
| 237 | Lipase variant | L17P, Q111E, L186Y, D301A | PRT |
| 238 | Lipase variant | L17P, Q111E, L186Y, D301A | NUC |
| 239 | Lipase variant | L17P, E79S, L186Y, A202N | PRT |
| 240 | Lipase variant | L17P, E79S, L186Y, A202N | NUC |
| 241 | Lipase variant | M3Q, E79S, L186Y, D301A | PRT |
| 242 | Lipase variant | M3Q, E79S, L186Y, D301A | NUC |
| 243 | Lipase variant | E79S, L186W, L280A, D301A | PRT |
| 244 | Lipase variant | E79S, L186W, L280A, D301A | NUC |
| 245 | Lipase variant | L17P, E79S, Q111E, L186W, P309C | PRT |
| 246 | Lipase variant | L17P, E79S, Q111E, L186W, P309C | NUC |
| 247 | Lipase variant | L17P, V18C, E79S, L186W, L280A | PRT |
| 248 | Lipase variant | L17P, V18C, E79S, L186W, L280A | NUC |
| 249 | Lipase variant | N29H, K40M, E79S, L186W, D301A | PRT |
| 250 | Lipase variant | N29H, K40M, E79S, L186W, D301A | NUC |
| 251 | Lipase variant | N29H, E79S, L186W, A202N, L280A | PRT |
| 252 | Lipase variant | N29H, E79S, L186W, A202N, L280A | NUC |
| 253 | Lipase variant | N29H, E79S, Q111E, L186W, L280A | PRT |
| 254 | Lipase variant | N29H, E79S, Q111E, L186W, L280A | NUC |
| 255 | Lipase variant | N29H, E79S, L186W, A202N, D301A | PRT |
| 256 | Lipase variant | N29H, E79S, L186W, A202N, D301A | NUC |
| 257 | Lipase variant | C11A, L186Y, L280A, D301A, P309C | PRT |
| 258 | Lipase variant | C11A, L186Y, L280A, D301A, P309C | NUC |
| 259 | Lipase variant | K8E, E79S, L186Y, L280A, D301A | PRT |
| 260 | Lipase variant | K8E, E79S, L186Y, L280A, D301A | NUC |
| 261 | Lipase variant | L17P, N29W, Q111E, L186W, A202N | PRT |
| 262 | Lipase variant | L17P, N29W, Q111E, L186W, A202N | NUC |
| 263 | Lipase variant | L17P, L186Y, L280A, D301A, P309C | PRT |
| 264 | Lipase variant | L17P, L186Y, L280A, D301A, P309C | NUC |
| 265 | Lipase variant | Q111E, L186W, A202N, L280A, P312N | PRT |
| 266 | Lipase variant | Q111E, L186W, A202N, L280A, P312N | NUC |
| 267 | Lipase variant | N29H, E79S, Q111E, L186Y, D301A | PRT |
| 268 | Lipase variant | N29H, E79S, Q111E, L186Y, D301A | NUC |
| 269 | Lipase variant | L17P, N29W, E79S, L186Y, A202N, P312N | PRT |
| 270 | Lipase variant | L17P, N29W, E79S, L186Y, A202N, P312N | NUC |
| 271 | Lipase variant | L17P, E79S, N84T, L186Y, L280A, P309C | PRT |
| 272 | Lipase variant | L17P, E79S, N84T, L186Y, L280A, P309C | NUC |
| 273 | Lipase variant | L17P, N29H, E79S, W85H, L186W, L280A | PRT |
| 274 | Lipase variant | L17P, N29H, E79S, W85H, L186W, L280A | NUC |
| 275 | Lipase variant | L17P, E79S, L186Y, F192A, A202N, P309C | PRT |
| 276 | Lipase variant | L17P, E79S, L186Y, F192A, A202N, P309C | NUC |
| 277 | Lipase variant | V18A, K40M, E79S, L186W, F192A, D301A | PRT |
| 278 | Lipase variant | V18A, K40M, E79S, L186W, F192A, D301A | NUC |
| 279 | Lipase variant | L17P, N29H, Q31W, E79S, L186W, D301A | PRT |
| 280 | Lipase variant | L17P, N29H, Q31W, E79S, L186W, D301A | NUC |
| 281 | Lipase variant | K8E, K40M, Q111E, L186W, L280A, P309C | PRT |
| 282 | Lipase variant | K8E, K40M, Q111E, L186W, L280A, P309C | NUC |
| 283 | Lipase variant | M3Q, K8E, L17P, L186Y, D301A, P312N | PRT |
| 284 | Lipase variant | M3Q, K8E, L17P, L186Y, D301A, P312N | NUC |
| 285 | Lipase variant | L17P, N29W, L186W, A202N, L280A, P309C | PRT |
| 286 | Lipase variant | L17P, N29W, L186W, A202N, L280A, P309C | NUC |
| 287 | Lipase variant | K8E, C11A, L186W, A202N, L280A, P309C | PRT |
| 288 | Lipase variant | K8E, C11A, L186W, A202N, L280A, P309C | NUC |
| 289 | Lipase variant | S4P, N29W, E79S, L186W, A202N, L280A, P309C | PRT |
| 290 | Lipase variant | S4P, N29W, E79S, L186W, A202N, L280A, P309C | NUC |
| 291 | Lipase variant | N29W, E79S, Q111E, L186W, L280A,P309C, P312N | PRT |
| 292 | Lipase variant | N29W, E79S, Q111E, L186W, L280A, P309C, P312N | NUC |
| 293 | Lipase variant | K8E, C11A, K40M, E79S, W85H, L186W, P309C | PRT |
| 294 | Lipase variant | K8E, C11A, K40M, E79S, W85H, L186W, P309C | NUC |
| 295 | Lipase variant | M3Q, S4P, N29H, K40M, L186Y, A202N, P309C | PRT |
| 296 | Lipase variant | M3Q, S4P, N29H, K40M, L186Y, A202N, P309C | NUC |
| 297 | Lipase variant | V18A, K40M, N84T, Q111E, L186Y, L280A, P309C, P312N | PRT |
| 298 | Lipase variant | V18A, K40M, N84T, Q111E, L186Y, L280A, P309C, P312N | NUC |
| 299 | Lipase variant | S4P, C11A, V18A, K40M, N84T, L186Y, D301A, P312N | PRT |
| 300 | Lipase variant | S4P, C11A, V18A, K40M, N84T, L186Y, D301A, P312N | NUC |
| 301 | Lipase variant | S4P, K8E, C11A, L186Y, F192A, A202N, L280A, P309C | PRT |
| 302 | Lipase variant | S4P, K8E, C11A, L186Y, F192A, A202N, L280A, P309C | NUC |
| 303 | Lipase variant | E79S, Q111E, L186W, L280A, D301A | PRT |
| 304 | Lipase variant | E79S, Q111E, L186W, L280A, D301A | NUC |
| 305 | Lipase variant | M3Q, L17P, K40M, E79S, L186Y | PRT |
| 306 | Lipase variant | M3Q, L17P, K40M, E79S, L186Y | NUC |
| 307 | Lipase variant | L17P, K40M, Q111E, L186W, L280A | PRT |
| 308 | Lipase variant | L17P, K40M, Q111E, L186W, L280A | NUC |
| 309 | Lipase variant | C11A, E79S, L186Y, A202N, D301A | PRT |
| 310 | Lipase variant | C11A, E79S, L186Y, A202N, D301A | NUC |
| 311 | Lipase variant | L17P, K40M, E79S, Q111E, L186Y | PRT |
| 312 | Lipase variant | L17P, K40M, E79S, Q111E, L186Y | NUC |
| 313 | Lipase variant | C11A, L17P, K40M, E79S, L186Y | PRT |
| 314 | Lipase variant | C11A, L17P, K40M, E79S, L186Y | NUC |
| 315 | Lipase variant | K40M, Q111E, L186Y, L280A, D301A | PRT |
| 316 | Lipase variant | K40M, Q111E, L186Y, L280A, D301A | NUC |
| 317 | Lipase variant | E79S, N84T, L186W, A202N, L280A, D301A | PRT |
| 318 | Lipase variant | E79S, N84T, L186W, A202N, L280A, D301A | NUC |
| 319 | Lipase variant | M3Q, L17P, N29H, E79S, L186Y, D301A | PRT |
| 320 | Lipase variant | M3Q, L17P, N29H, E79S, L186Y, D301A | NUC |
| 32 | Lipase variant | L17P, Q111E, L186Y, A202N, L280A, P312N | PRT |
| 322 | Lipase variant | L17P, Q111E, L186Y, A202N, L280A, P312N | NUC |
| 323 | Lipase variant | M3Q, C11A, L186Y, A202N, L280A, P312N | PRT |
| 324 | Lipase variant | M3Q, C11A, L186Y, A202N, L280A, P312N | NUC |
| 325 | Lipase variant | L17P, E79S, Q111E, L186Y, F192A, D301A | PRT |
| 326 | Lipase variant | L17P, E79S, Q111E, L186Y, F192A, D301A | NUC |
| 327 | Lipase variant | C11A, E79S, W85H, Q111E, L186Y, L280A, P312N | PRT |
| 328 | Lipase variant | C11A, E79S, W85H, Q111E, L186Y, L280A, P312N | NUC |
| 329 | Lipase variant | L17P, K40M, E79S, N84T, L186Y, A202N, P309C | PRT |
| 330 | Lipase variant | L17P, K40M, E79S, N84T, L186Y, A202N, P309C | NUC |
| 331 | Lipase variant | C11A, Q31W, E79S, L186Y, L280A, D301A, P309C | PRT |
| 332 | Lipase variant | C11A, Q31W, E79S, L186Y, L280A, D301A, P309C | NUC |
| 333 | Lipase variant | K8E, K40M, E79S, L186W, A202N, D301A, P312N | PRT |
| 334 | Lipase variant | K8E, K40M, E79S, L186W, A202N, D301A, P312N | NUC |
| 335 | Lipase variant | M3Q, C11A, L186W, A202N, L280A, P309C, P312N | PRT |
| 336 | Lipase variant | M3Q, C11A, L186W, A202N, L280A, P309C, P312N | NUC |
| 337 | Lipase variant | L17P, K40M, L186W, F192A, A202N, L280A, P309C | PRT |
| 338 | Lipase variant | L17P, K40M, L186W, F192A, A202N, L280A, P309C | NUC |
| 339 | Lipase variant | K8E, L17P, N29W, K40M, E79S, L186W, F192A, L280A | PRT |
| 340 | Lipase variant | K8E, L17P, N29W, K40M, E79S, L186W, F192A, L280A | NUC |
| 341 | Lipase variant | S4P, N29H, N84T, Q111E, L186Y, A202N, L280A, D301A | PRT |
| 342 | Lipase variant | S4P, N29H, N84T, Q111E, L186Y, A202N, L280A, D301A | NUC |
| 343 | Lipase variant | L17P, K40M, E79S, Q111E, F192A, L280A, P309C, P312N | PRT |
| 344 | Lipase variant | L17P, K40M, E79S, Q111E, F192A, L280A, P309C, P312N | NUC |
| 345 | Lipase variant | M3Q, L17P, V18C, L186Y, A202N, L280A, P309C, P312N | PRT |
| 346 | Lipase variant | M3Q, L17P, V18C, L186Y, A202N, L280A, P309C, P312N | NUC |
| 347 | Lipase variant | M3Q, S4P, K8E, C11A, E79S, W85H, L186Y, F192A, D301A | PRT |
| 348 | Lipase variant | M3Q, S4P, K8E, C11A, E79S, W85H, L186Y, F192A, D301A | NUC |
| 349 | Lipase variant | K8E, E79S, W85H, Q111E, L186Y, F192A, A202N, L280A, P309C | PRT |
| 350 | Lipase variant | K8E, E79S, W85H, Q111E, L186Y, F192A, A202N, L280A, P309C | NUC |
| 351 | Lipase variant | K8E, K40M, E79S, Q111E, L186W, L280A, D301A | PRT |
| 352 | Lipase variant | K8E, K40M, E79S, Q111E, L186W, L280A, D301A | NUC |
| 353 | Lipase variant | L17P, E79S, L186Y, F192A, A202N, L280A, D301A | PRT |
| 354 | Lipase variant | L17P, E79S, L186Y, F192A, A202N, L280A, D301A | NUC |
| 355 | Lipase variant | Q31W, E79S, Q111E, L186W, A202N, L280A, D301A | PRT |
| 356 | Lipase variant | Q31W, E79S, Q111E, L186W, A202N, L280A, D301A | NUC |
| 357 | Lipase variant | L17P, K40M, E79S, Q111E, L186W, F192A, D301A | PRT |
| 358 | Lipase variant | L17P, K40M, E79S, Q111E, L186W, F192A, D301A | NUC |
| 359 | Lipase variant | C11A, K40M, E79S, Q111E, L186W, F192A, D301A | PRT |
| 360 | Lipase variant | C11A, K40M, E79S, Q111E, L186W, F192A, D301A | NUC |
| 361 | Lipase variant | L17P, V18A, K40M, Q111E, L186Y, A202N, L280A | PRT |
| 362 | Lipase variant | L17P, V18A, K40M, Q111E, L186Y, A202N, L280A | NUC |
| 363 | Lipase variant | M3Q, L17P, E79S, L186W, A202N, D301A, P309C | PRT |
| 364 | Lipase variant | M3Q, L17P, E79S, L186W, A202N, D301A, P309C | NUC |
| 365 | Lipase variant | M3Q, S4P, C11A, L17P, E79S, Q111E, L186Y | PRT |
| 366 | Lipase variant | M3Q, S4P, C11A, L17P, E79S, Q111E, L186Y | NUC |
| 367 | Lipase variant | K40M, E79S, Q111E, L186Y, A202N, L280A, P309C, P312N | PRT |
| 368 | Lipase variant | K40M, E79S, Q111E, L186Y, A202N, L280A, P309C, P312N | NUC |
| 369 | Lipase variant | C11A, N29W, Q31W, E79S, L186Y, A202N, L280A, D301A | PRT |
| 370 | Lipase variant | C11A, N29W, Q31W, E79S, L186Y, A202N, L280A, D301A | NUC |
| 371 | Lipase variant | L17P, K40M, E79S, Q111E, L186W, F192A, L280A, P309C | PRT |
| 372 | Lipase variant | L17P, K40M, E79S, Q111E, L186W, F192A, L280A, P309C | NUC |
| 373 | Lipase variant | L17P, N29H, Q31W, K40M, E79S, Q111E, L186Y, L280A | PRT |
| 374 | Lipase variant | L17P, N29H, Q31W, K40M, E79S, Q111E, L186Y, L280A | NUC |
| 375 | Lipase variant | K8E, K40M, E79S, Q111E, L186W, A202N, L280A, P309C | PRT |
| 376 | Lipase variant | K8E, K40M, E79S, Q111E, L186W, A202N, L280A, P309C | NUC |
| 377 | Lipase variant | S4P, K40M, E79S, Q111E, L186Y, A202N, L280A, P309C | PRT |
| 378 | Lipase variant | S4P, K40M, E79S, Q111E, L186Y, A202N, L280A, P309C | NUC |
| 379 | Lipase variant | M3Q, L17P, N29W, Q31W, Q111E, L186Y, A202N, L280A, P309C | PRT |
| 380 | Lipase variant | M3Q, L17P, N29W, Q31W, Q111E, L186Y, A202N, L280A, P309C | NUC |
| 381 | Lipase variant | L17P, K40M, E79S, L186Y, A202N, L280A, D301A | PRT |
| 382 | Lipase variant | L17P, K40M, E79S, L186Y, A202N, L280A, D301A | NUC |
| 383 | Lipase variant | M3Q, L17P, E79S, Q111E, L186W, F192A, A202N, L280A | PRT |
| 384 | Lipase variant | M3Q, L17P, E79S, Q111E, L186W, F192A, A202N, L280A | NUC |
| 385 | Lipase variant | M3Q, L17P, V18A, E79S, Q111E, L186Y, L280A, D301A, P312N | PRT |
| 386 | Lipase variant | M3Q, L17P, V18A, E79S, Q111E, L186Y, L280A, D301A, P312N | NUC |
| 387 | Lipase variant | M3Q, L17P, V18A, K40M, E79S, N84T, L186Y, A202N, L280A | PRT |
| 388 | Lipase variant | M3Q, L17P, V18A, K40M, E79S, N84T, L186Y, A202N, L280A | NUC |
| 389 | Lipase variant | L17P, K40M, E79S, N84T, Q111E, L186Y, A202N, L280A, D301A | PRT |
| 390 | Lipase variant | L17P, K40M, E79S, N84T, Q111E, L186Y, A202N, L280A, D301A | NUC |
| 391 | Lipase variant | M3Q, S4P, L17P, K40M, E79S, L186Y, A202N, L280A, D301A | PRT |
| 392 | Lipase variant | M3Q, S4P, L17P, K40M, E79S, L186Y, A202N, L280A, D301A | NUC |
| 393 | Lipase variant | N29W, Q31W, L186W, P312N | PRT |
| 394 | Lipase variant | N29W, Q31W, L186W, P312N | NUC |
| 395 | Lipase variant | L186Y, F192A, P309C, P312N | PRT |
| 396 | Lipase variant | L186Y, F192A, P309C, P312N | NUC |
| 397 | Lipase variant | N29W, Q31W, W85H, L186Y, P312N | PRT |
| 398 | Lipase variant | N29W, Q31W, W85H, L186Y, P312N | NUC |
| 399 | Lipase variant | K40M, L42D, E79S, N84T, L186Y, F192A, D301A | PRT |
| 400 | Lipase variant | K40M, L42D, E79S, N84T, L186Y, F192A, D301A | NUC |
| 401 | Lipase variant | E79S, N84T, L186Y, F192V, Q217M, D301A | PRT |
| 402 | Lipase variant | E79S, N84T, L186Y, F192V, Q217M, D301A | NUC |

The protein having the activity of a lipase is preferably used in an amount of 0.1-50% by weight, based on the mixture (I). Preferred is an amount of 0.5-10% by weight. Especially preferred is an amount of 1-5% by weight.

Step 1 of the process according to the invention can be carried out in the absence of a solvent or in such a solvent. Preferred are solvents from the group consisting of methyl tert-butyl ether, heptane, toluene, xylenes, mesitylene, anisole, chlorobenzene, n-butanol, isopropanol, n-propanol and ethanol and also mixtures thereof. Particularly preferred are solvents from the group consisting of toluene, xylenes, mesitylene, n-butanol and ethanol and also mixtures thereof. Likewise particularly preferred is carrying out the reaction in the absence of a solvent.

Based on the molar amount of mixture (I) used, the acylation or carboxylation agent R—C(═O)R¹ is preferably used in an amount of 0.4-25 equivalents. Particularly preferably, it is used in an amount of 0.6-10 equivalents. Especially preferably, it is used in an amount of 1-5 equivalents.

The reaction as per step 1 is usually carried out at temperatures of 20-130° C. Preferably, the reaction is carried out at 70-130° C. Particularly preferably, the reaction is carried out at 80-120° C.

The separation of component (II) specified in step 2 of the process according to the invention is carried out by a crystallization known to a person skilled in the art.

The bases specified in step 3 of the process according to the invention usually come from the group consisting of lithium hydroxide, sodium hydroxide, potassium hydroxide, lithium carbonate, sodium carbonate, potassium carbonate, lithium methoxide, sodium methoxide, potassium methoxide, lithium ethoxide, sodium ethoxide and potassium ethoxide. Particular preferably, the bases lithium hydroxide, sodium hydroxide, potassium hydroxide, lithium ethoxide, sodium ethoxide and potassium ethoxide are used.

Very particularly preferably, the bases lithium hydroxide, sodium hydroxide and potassium hydroxide are used.

Usually, the base is used in a stoichiometry of 1.00-3.00 equivalents, based on the molar amount of component (II) used. Preferably, the base is used in an amount of 1.50-2.50 equivalents. Especially preferably, the base is used in an amount of 1.75 to 2.25 equivalents.

Preferably, ethanol, n-propanol, isopropanol, n-butanol, isobutanol, sec-butanol, tert-butanol and 1-methoxypropan-2-ol, and also toluene, xylenes and veratrole, are used as solvents. Particular preferably, ethanol, n-butanol, isopropanol, 1-methoxypropan-2-ol, toluene, xylenes and veratrole are used. Especially preferably, ethanol, n-butanol and xylenes are used.

The reaction as per step 3 is usually carried out at temperatures of 60-140° C. Preferably, the reaction is carried out at 80-120° C. Particularly preferably, the reaction is carried out at 90-110° C.

A further advantage of the process according to the invention is also that the mixture of 2,6-dimethyl-1-indanamines (III) remaining in step 1 and step 2 can be isomerized again under metal catalysis to form a mixture of the four stereoisomers of 2,6-dimethyl-1-indanamine (I) in a resource-saving manner.

This isomerization can be carried out as follows:

Preferably, a palladium on carbon (Pd/C), a palladium on calcium carbonate (Pd/CaCO₃) or a palladium on aluminium oxide (Pd/Al₂O₃) catalyst having a palladium load of 1-20% by weight is used as metal catalyst. Likewise preferably, use is made of the Shvo catalyst with the IUPAC name 1-hydroxytetraphenylcyclopentadienyl (tetraphenyl-2,4-cyclopentadien-1-one)-u-hydrotetracarbonyldiruthenium (II) in a stoichiometry of 1-10 mol %. Particularly preferably, a palladium on carbon (Pd/C) or a palladium on aluminium oxide (Pd/Al₂O₃) catalyst having a palladium load of 1-20% by weight is used. Especially preferably, a palladium on aluminium oxide (Pd/Al₂O₃) catalyst having a palladium load of 1-20% by weight is used. The supported catalysts are used in an amount of 0.1-10% by weight based on the compounds of formula (III); preference is given to using 0.5-5% by weight. The amount of catalysts used is calculated on the basis of the dry mass of the catalysts.

Preferably, pyridine, N-methylmorpholine, morpholine, cyclohexylamine, di-n-butylamine, tri-n-butylamine, triethylamine, diisopropylethylamine, piperidine, potassium carbonate, sodium carbonate, lithium carbonate, potassium ethoxide, sodium ethoxide and lithium ethoxide are used as bases, or no base is used. Particularly preferably, piperidine, morpholine, diisopropylethylamine, tri-n-butylamine, potassium carbonate and sodium ethoxide are used, or no base is used. Especially preferably, piperidine and sodium ethoxide are used, or no base is used.

Preferably, toluene, xylenes, mesitylene, anisole, chlorobenzene, n-butanol, isopropanol, n-propanol and ethanol are used as solvents, or the reaction is carried out in the absence of a solvent. Particularly preferably, toluene, xylenes, mesitylene, n-butanol and ethanol are used as solvents, or the reaction is carried out in the absence of a solvent. Especially preferably, the reaction is carried out in the absence of a solvent.

The reaction is preferably carried out at between 0-20 bar gauge pressure. Particularly preferably, the reaction is carried out at between 0-10 bar gauge pressure. Especially preferably, the reaction is carried out at between 1-6 bar gauge pressure.

The reaction pressure is substantially achieved by the injection of a gas/gas mixture. Preferably, hydrogen, nitrogen or argon or a mixture of hydrogen and nitrogen or hydrogen and argon is injected. Particularly preferably, hydrogen or a mixture of nitrogen and hydrogen is injected. Especially preferably, pure hydrogen is injected.

The reaction is preferably operated at temperatures of 80-150° C. Particularly preferably, the reaction is operated at 100-130° C. Especially preferably, the reaction is operated at 110-125° C.

The present invention thus also further provides a process for producing a mixture of all four stereoisomers of 2,6-dimethyl-1-indanamine (I) by metal-catalysed isomerization of a mixture of the three stereoisomers of (1R,2R)-, (1S,2R)- and (1S,2S)-2,6-dimethyl-1-indanamine:

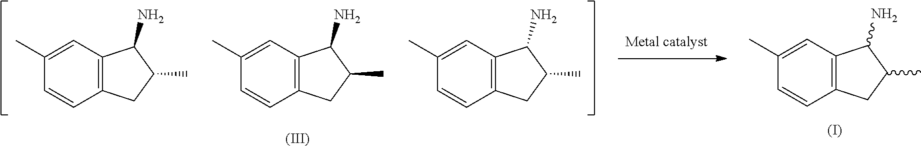

The examples which follow elucidate the invention more particularly:

Terrific broth (TB) culture media were prepared in demineralized water using 47.6 g/l granulated medium and 4 ml/l glycerol and sterilized at 121° C. for 20 minutes.

### Cloning of Lipases

Nucleotide sequences which encode lipases and lipase variants as described herein can be synthesized as known according to the prior art, for example as offered by relevant service providers, such as Eurofins Genomics GmbH (Eurofins Genomics GmbH, Anzinger Str. 7a, 85560 Ebersberg, Germany). In brief, nucleic acid sequences of wild-type lipase (SEQ ID No. 2) or related variants, as described herein, were cloned into an expression vector based on the vector pKA81a. Genetic elements were introduced into the modified pKA81a vector by means of commonly known methods. Wild-type lipase and lipase variants were expressed by introducing the expression vectors into electrocompetent Escherichia coli MG1655 cells.

### Generation of Enzyme Variants

Nucleotide substitutions (replacements) were introduced into the nucleic acid parent sequences, for example in order to exchange an amino acid for other amino acids. A number of molecular biology methods can be used in order to achieve these replacements. One useful method for producing a mutated nucleic acid and the corresponding mutated protein according to the invention is site-directed mutagenesis at codons encoding one or more amino acids, which are selected in advance. The methods for achieving these site-directed mutations are well known to those of ordinary skill in the art and adequately described in the literature (in particular: Directed Mutagenesis: A Practical Approach, 1991, edited by M. J. McPHERSON, IRL PRESS) or are methods for which commercial kits (e.g. the QUIKCHANGE™ Lightning Mutagenesis Kit from Qiagen or Stratagene) can be used. Site-directed mutagenesis was followed by transforming nucleic acids into the Escherichia coli MG1655 cells.

Transformed cells were tested in suitable biotransformation reactions in order to determine product yield and product selectivity. Suitable biotransformation reactions are described in what follows. Sequence verification was carried out as known according to the prior art.

Glycerol stocks of the E. coli cultures transformed with the respective expression plasmids were prepared by adding one volume of a 40% glycerol solution to one volume of an E. coli culture.

To isolate single bacterial colonies, suitable dilutions of E. coli cultures were plated onto LB agar plates containing suitable concentrations of kanamycin and were incubated at 37° C. until single colonies were obtained.

### Synthesis of ethyl [(1R,2S)-2,6-dimethyl-2,3-dihydro-1H-inden-1-yl]carbamate

### EXAMPLE 1

In a comparative experiment, the suitability of three different proteins having the activity of a lipase was tested: the wild-type lipase (SEQ ID No. 1) and the lipase variants having SEQ ID No. 233 and SEQ ID No. 399. To this end, 9.25 g of 2,6-dimethylindan-1-amine (racemic mixture of 82% trans- and 18% cis-2,6-dimethylindan-1-amine) were in each case initially charged under argon in a 25 mL three-necked flask having a magnetic stirrer and diluted with 13.6 g of diethyl carbonate in each case. This solution was heated to an internal temperature of 110° C. and then 250 mg of protein having the activity of a lipase were in each case added in one portion. The conversions were monitored in respect of their reaction progress by means of HPLC measurements. After complete conversion of the (1R,2S)-2,6-dimethylindan-1-amine isomer, the reaction was terminated by means of addition of 5 mL of diethyl carbonate, cooling to 100° C. and filtration of this reaction mixture across a glass frit (porosity 3).

Another 5 mL of diethyl carbonate were used in order to wash the filter cake. The combined orange filtrate was then subjected to distillation at 50° C. and a vacuum of 35-10 mbar. 50 mL of n-heptane were added to the distillation residue and the solution was stirred at 40° C. for 20 minutes. During cooling to 2° C., what was formed was a suspension, which was filtered by means of a glass frit (porosity 3). The filter cake was washed with 5 mL of n-heptane and then dried under reduced pressure. The white solid obtained was then tested for chemical purity by means of quantitative ¹H-NMR measurement and for stereoisomer purity by means of chiral HPLC measurement. The results are compared in Table 5 below.

TABLE 5

| | Wild-type lipase | Lipase variant | Lipase variant |
|---|---|---|---|
| | (SEQ ID No. 1) | (SEQ ID No. 233) | (SEQ ID No. 399) |
| Reaction time | 10 h | 13 h | 12 h |
| Conversion | 58% | 41% | 43% |
| Yield | 4.3 g (21% 1R,2S) | 4.6 g (27% 1R,2S) | 4.1 g (27% 1R,2S) |
| Chemical purity | 80% | 81% | 88% |
| Isomer purity | 81% 1R,2S | 97% 1R,2S | 99% 1R,2S |

The yields are based here on the content of pure ethyl [(1R,2S)-2,6-dimethyl-2,3-dihydro-1H-inden-1-yl]carbamate.

### EXAMPLE 2

In a 500 mL four-necked round-bottom flask equipped with an internal thermometer, a magnetic stirrer, an inert gas inlet and a bubble counter, 80.0 g of 2,6-dimethylindan-1-amine (racemic mixture of 82% trans- and 18% cis-2,6-dimethylindan-1-amine) were dissolved in 130.0 g of diethyl carbonate under argon. This solution was heated with stirring to an internal temperature of 110° C. Thereafter, 1.2 g of E. coli culture transformed with SEQ ID No. 234 were added in one portion and the reaction was continued at 110° C. with stirring and under argon. After a reaction time of 7 hours, a virtually complete conversion of the (1R,2S)-2,6-dimethylindan-1-amine was determined by means of an HPLC measurement and the reaction was terminated by means of cooling to 80° C. and subsequent filtration across a glass frit (porosity 3). The filter cake was then washed with 2×40 mL of diethyl carbonate and the combined filtrate was subjected to distillation at 50° C. and a vacuum of 35-10 mbar. This removed a total of 157.4 g of distillate from an initial 264.2 g of filtrate. 120 mL of methylcyclohexane were then added to the distillation residue and the mixture obtained was cooled to 2° C., yielding a suspension. The solid was removed by means of filtration across a glass frit (porosity 3) and washed with a small amount of methylcyclohexane. After drying under vacuum, 37.0 g of a white solid were obtained. A quantitative ¹H-NMR measurement confirmed a chemical purity of 85.4% for ethyl [(1R,2S)-2,6-dimethyl-2,3-dihydro-1H-inden-1-yl]carbamate. By means of a chiral HPLC measurement, the stereoisomer purity was determined as 98.5%. This corresponds to a yield of 27% pure ethyl [(1R,2S)-2,6-dimethyl-2,3-dihydro-1H-inden-1-yl]carbamate. ¹H-NMR (400 MHz; CDCl₃) δ=7.08-7.01 (m, 3H), 4.81-4.71 (m, 2H), 4.19 (q, J=8.0 Hz, 2H), 3.00 (dd, J=8.0, 14.0 Hz, 1H), 2.51-2.45 (m, 1H), 2.33 (s, 3H), 2.16 (dt, J=8.0, 14.0 Hz, 1H), 1.31-1.26 (m, 6H).

The derivative methyl[(1R,2S)-2,6-dimethyl-2,3-dihydro-1H-inden-1-yl]carbamate was prepared analogously using dimethyl carbonate and was obtained in the form of a white solid. ¹H-NMR (400 MHz; CDCl₃) δ=7.08-7.01 (m, 3H), 4.79-4.78 (m, 2H), 3.74 (s, 3H), 3.00 (dd, J=8.0, 16.0 Hz, 1H), 2.51-2.45 (m, 1H), 2.32 (s, 3H), 2.20-2.10 (m, 1H), 1.27 (d, J=8.0 Hz, 3H).

### EXAMPLE 3

In a 25 mL three-necked flask equipped with an internal thermometer, an inert gas inlet, a bubble counter and a magnetic stirrer, 14.65 g of diethyl carbonate were heated to an internal temperature of 110° C. under argon. Thereafter, both 10.00 g of 2,6-dimethylindan-1-amine (racemic mixture of 82% trans- and 18% cis-2,6-dimethylindan-1-amine) and 0.25 g of E. coli culture transformed with SEQ ID No. 234 were added in one portion and the reaction was continued with stirring at 110° C. for 10 hours under argon until an HPLC measurement indicated a virtually complete conversion of the (1R,2S)-2,6-dimethylindan-1-amine. The reaction mixture was subsequently diluted with another 5 mL of diethyl carbonate and then filtered by means of a glass frit (porosity 3) at 100° C. The filter cake was then washed with 5 mL of diethyl carbonate adjusted to a temperature of 80° C. The combined filtrate was then subjected to distillation at 50° C. and a vacuum of 35-10 mbar. Thereafter, 50 mL of n-heptane were added to the distillation residue, which resulted in a thick suspension. The suspension was heated to 40° C. and stirred at this temperature for 20 minutes. Thereafter, the suspension was cooled to 21° C. within 30 minutes. Subsequently, the suspension was cooled further to 2° C. and filtered by means of a glass frit (porosity 3). The filter cake was washed with 20 mL of n-heptane cooled to 5° C. and then dried at 40° C. and a vacuum of 10 mbar. 5.1 g of a white solid were obtained. A quantitative ¹H-NMR measurement confirmed a chemical purity of 99% for ethyl [(1R,2S)-2,6-dimethyl-2,3-dihydro-1H-inden-1-yl]carbamate. By means of a chiral HPLC measurement, the stereoisomer purity was determined as 98.5%. This corresponds to a yield of 34% pure ethyl [(1R,2S)-2,6-dimethyl-2,3-dihydro-1H-inden-1-yl]carbamate.

### EXAMPLE 4

In a 600 mL autoclave, 100.0 g of 2,6-dimethylindan-1-amine (racemic mixture of 82% trans- and 18% cis-2,6-dimethylindan-1-amine), 139.2 g of diethyl carbonate and 2 g of biomass Gö171-7-032 were stirred under argon at 110° C. for 7 h. After cooling to 80° C., filtration was carried out across a glass frit and the filter cake was washed with diethyl carbonate. The combined filtrate was concentrated under reduced pressure at 50° C. and the distillation residue was crystallized in an ice bath by addition of 150 mL of methylcyclohexane. The solid was removed by filtration across a glass frit and washed with a small amount of methylcyclohexane. After drying under vacuum, 47.6 g of a white solid were obtained. A quantitative ¹H-NMR measurement confirmed a chemical purity of 91.9% for ethyl [(1R,2S)-2,6-dimethyl-2,3-dihydro-1H-inden-1-yl]carbamate. By means of a chiral HPLC measurement, the stereoisomer purity was determined as 96.1%. This corresponds to a yield of 31.9% pure ethyl [(1R,2S)-2,6-dimethyl-2,3-dihydro-1H-inden-1-yl]carbamate. The filtrate was concentrated under reduced pressure at 50° C. and the content of 2,6-dimethylindan-1-amine isomers was determined by chiral GC at an isomer ratio of R,R:S,S:S,R:R,S=6.0:16.2:77.2:0.

### Synthesis of (1R,2S)-2,6-dimethylindan-1-amine from methyl[(1R,2S)-2,6-dimethyl-2,3-dihydro-1H-inden-1-yl]carbamate

### EXAMPLE 5

In a 250 mL jacketed reactor inertized with argon at 21° C., 25 g of methyl[(1R,2S)-2,6-dimethyl-2,3-dihydro-1H-inden-1-yl]carbamate (98% stereoisomer purity, 98% chemical purity) were suspended in 80 mL of n-butanol. The suspension was heated to an internal temperature of 65° C. and a clear solution was obtained. Thereafter, 15 g of potassium hydroxide (85% chemical purity) were added in one portion and the solution was heated further to an internal temperature of 100° C. The solution was stirred further at this temperature for one hour, while insoluble solids precipitated. Complete conversion was indicated by an HPLC measurement. Subsequently, a reduced pressure of 360 mbar was applied and 55 mL of solvent were distilled off. Thereafter, 100 mL of p-xylene were added and the reaction mixture was cooled to 20° C. The mixture was washed with 1×50 mL of deionized water. The organic phase was then distilled at 40° C. to a reduced pressure of 5 mbar and 17.8 g of (1R,2S)-2,6-dimethylindan-1-amine in the form of a pale yellow oil and a chemical purity of 95%, determined by a quantitative ¹H-NMR measurement, were obtained, which corresponds to a yield of 95%. The stereoisomer purity was determined at 98% by means of chiral GC measurement. 1H NMR (500 MHZ, CDCl₃): δ 7.12 (s, 1H), 7.05 (d, J=7.5 Hz, 1H), 6.99 (d, J=7.5 Hz, 1H), 3.74 (d, J=8.5 Hz, 1H), 2.98 (dd, J=7.5, 15.5 Hz, 1H), 2.42 (dd, J=9.5, 15.5 Hz, 1H), 2.34 (s, 3H), 1.98 (m, 1H), 1.72 (bs, 2H), 1.24 (d, J=6.5 Hz, 3H).

### EXAMPLE 6

### Synthesis of (1R,2S)-2,6-dimethylindan-1-amine hydrochloride from methyl[(1R,2S)-2,6-dimethyl-2,3-dihydro-1H-inden-1-yl]carbamate

In a 250 mL jacketed reactor inertized with argon at 21° C., 50 g of methyl[(1R,2S)-2,6-dimethyl-2,3-dihydro-1H-inden-1-yl]carbamate (98% stereoisomer purity, 98% chemical purity) were suspended in 160 mL of n-butanol. The suspension was heated to an internal temperature of 65° C. and a clear solution was obtained. Thereafter, 31.1 g of potassium hydroxide (85% chemical purity) were added in one portion and the solution was heated further to an internal temperature of 100° C. The solution was stirred further at this temperature for one hour, while insoluble solids precipitated. Complete conversion was indicated by an HPLC measurement. Subsequently, a reduced pressure of 360 mbar was applied and 110 mL of solvent were distilled off. Thereafter, 200 mL of p-xylene were added and the reaction mixture was cooled to 20° C. The mixture was washed with 1×100 mL of deionized water. The organic phase was then cooled to 10° C. and admixed with 22.8 g of concentrated hydrochloric acid (37% by weight in water) in such a way that the temperature did not rise above 25° C. 100 mL of solvent were then distilled off at 50° C. from the suspension obtained, and the solid was removed by filtration at 20° C. by means of a glass frit (porosity 3). The filter cake was washed with 1×100 mL of toluene and then dried at 40° C. and a reduced pressure of 10 mbar and 40 g of (1R,2S)-2,6-dimethylindan-1-amine hydrochloride in the form of a white solid and a chemical purity of 99%, determined by a quantitative ¹H-NMR measurement, was obtained, which corresponds to a yield of 95%. The stereoisomer purity was determined at 98% by means of chiral GC measurement. ¹H-NMR (400 MHz; DMSO-d6) δ=8.66 (bs, 3H), 7.45 (s, 1H), 7.14 (dd, J=8.0, 16.0 Hz, 2H), 4.20 (d, J=8.0 Hz, 1H), 3.18-3.12 (m, 1H), 2.50-2.40 (m, 1H), 2.30 (s, 3H), 1.22 (d, J=8.0 Hz, 3H).

### Isomerization of 2,6-dimethylindan-1-amine mixture (III) to form 2,6-dimethylindan-1-amine mixture (I)

### EXAMPLE 7

In a 25 ml autoclave, 10 g of the 2,6-dimethylindan-1-amine mixture (III) obtained (concentrated filtrate from Example 4) were admixed with 396 mg of palladium catalyst (5% Pd/Al₂O₃). The autoclave was purged three times with 5 bar argon followed by injection of 3 bar hydrogen and stirring at 120° C. for 10 h under 3 bar hydrogen. After cooling to room temperature and venting of the autoclave, the content of 2,6-dimethylindan-1-amine isomers of the mixture obtained was determined by chiral GC at an isomer ratio of R,R:S,S:S,R:R,S=9.2:8.9:43.0:39.0.

### Isomerization of (1R,2S)-2,6-dimethylindan-1-amine to 2,6-dimethylindan-1-amine (racemic mixture of 82% trans and 18% cis isomers)

### EXAMPLES 8-27

A 6 mL Wheaton screw-cap vial was filled with 1.0 g of (1R,2S)-2,6-dimethylindan-1-amine, 39.6 mg of palladium catalyst (5% Pd/Al₂O₃), 1.47 g of diethyl carbonate and 10-20 mol % of a base. After that, the vial was closed with a screw cap and shaken at 400 rpm at 90-110° C. for 6 hours. This was followed by cooling to 21° C. and analysis by means of chiral GC analysis and achiral HPLC measurement. The results are tabulated. Activity defines the isomerization progress proceeding from an isomer ratio of 0:0:0:100 right up to an equilibrium ratio of 9:9:41:41 and is primarily measured by the degradation progress of the 1R,2S isomer from 100% to 41%. Activity is thus determined from the measured proportion of not more than 59% degradation. Chemoselectivity describes the degree of desired components (all DMAI reactant isomers and all DMAI carbamate product isomers) in relation to the sum total of all components formed in the isomerization, including the secondary components. The associated data are shown in Table 6 below:

TABLE 6

| No. | T/° C. | Base | R,R:S,S:S,R:R,S | Activity | Chemoselect. |
|---|---|---|---|---|---|
| 8 | 110 | | 8.3:8.8:23.6:59.3 | 69% | 85% |
| 9 | 100 | -- | 0.3:0.1:0.5:99.1 | 0% | 86% |
| 10 | 90 | -- | 0.3:0.1:0.5:99.1 | 0% | 91% |
| 11 | 110 | 10 mol % diisopropylethylamine | 9.0:9.0:41.0:41.0 | 100% | 85% |
| 12 | 100 | 20 mol % diisopropylethylamine | 8.2:8.7:30.1:53.0 | 80% | 90% |
| 13 | 90 | 20 mol % diisopropylethylamine | 0.3:0.1:0.5:99.1 | 0% | 91% |
| 14 | 100 | 10 mol % tri-n-butylamine | 8.1:7.8:26.3:56.9 | 73% | 87% |
| 15 | 90 | 10 mol % tri-n-butylamine | 0.3:0.1:0.5:99.1 | 0% | 95% |
| 16 | 110 | 10 mol % piperidine | 8.9:8.9:41.1:41.1 | 100% | 88% |
| 17 | 100 | 10 mol % piperidine | 8.5:8.5:41.3:41.6 | 100% | 89% |
| 18 | 90 | 20 mol % piperidine | 7.3:8.6:27.9:56.2 | 74% | 92% |
| 19 | 110 | 10 mol % morpholine | 8.9:8.9:40.9:41.2 | 100% | 81% |
| 20 | 100 | 10 mol % morpholine | 8.1:8.7:28.7:54.4 | 77% | 86% |
| 21 | 90 | 10 mol % morpholine | 6.7:7.9:16.3:69.1 | 52% | 90% |
| 22 | 110 | 10 mol % potassium carbonate | 9.1:9.1:40.7:41.0 | 100% | 85% |
| 23 | 100 | 20 mol % potassium carbonate | 8.1:8.7:28.1:55.0 | 76% | 87% |
| 24 | 90 | 20 mol % potassium carbonate | 5.9:6.7:14.3:73.2 | 45% | 90% |
| 25 | 110 | 20 mol % sodium ethoxide | 9.0:9.0:41.0:41.0 | 100% | 81% |
| 26 | 100 | 20 mol % sodium ethoxide | 8.3:8.7:30.0:53.0 | 80% | 83% |
| 27 | 90 | 20 mol % sodium ethoxide | 7.4:8.2:20.4:64.0 | 71% | 85% |

1. A method for preparing (1R,2S)-2,6-dimethyl-1-indanamine, the method comprising:
a first step, wherein a mixture of four stereoisomers of 2,6-dimethyl-1-indanamine (I) is reacted with an acylation or carboxylation agent R—C(═O)R¹ in the presence of a protein having activity of a lipase to form a corresponding amide or carbamate (II) and a mixture (III) of unreacted stereoisomers of 2,6-dimethyl-1-indanamine: 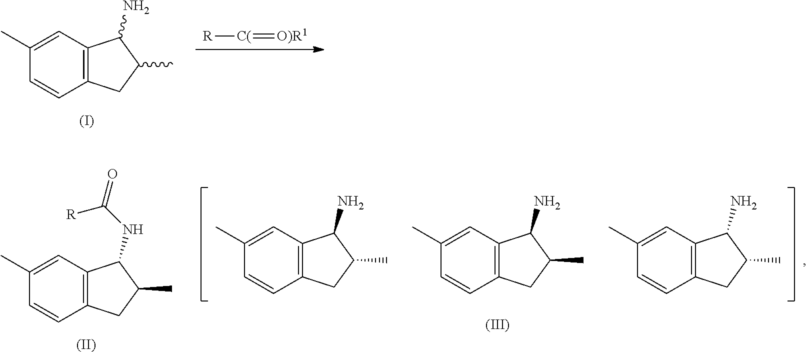
wherein the protein is encoded by an amino acid sequence selected from the group consisting of I. proteins having at least 80% identity to the amino acid sequence shown under SEQ ID No. 1, II. proteins having at least 80% identity to the amino acid sequence shown under SEQ ID No. 1 apart from the fact that wherein the amino acid sequence has a modification selected from the group consisting of:
i. an amino acid at position 186 different from L;
ii. an amino acid at position 280 different from L;
iii. an amino acid at position 312 different from P;
iv. an amino acid at position 3 different from M;
v. an amino acid at position 29 different from N;
vi. an amino acid at position 17 different from L;
vii. an amino acid at position 4 different from S;
viii. an amino acid at position 18 different from V;
ix. an amino acid at position 202 different from A;
x. an amino acid at position 301 different from D;
xi. an amino acid at position 309 different from P;
xii. an amino acid at position 31 different from Q;
xiii. an amino acid at position 111 different from Q;
xiv. an amino acid at position 85 different from W;
xv. an amino acid at position 8 different from K;
xvi. an amino acid at position 79 different from E;
xvii. an amino acid at position 40 different from K;
a second step, wherein the amide or carbamate (II) is separated from the unreacted 2,6-dimethyl-1-indanamines (III) and secondary components by crystallization; and
a third step, wherein the amide or carbamate (II) is converted to the (1R,2S)-2,6-dimethyl-1-indanamine (IV) using an acid or a base, 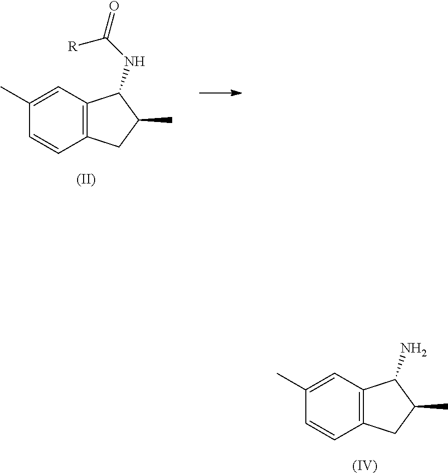
in which R means a radical from the group consisting of CH₂OCH₃, CH₂OCH₂CH₃, CH₃, OCH₃, OCH₂CH₃, OCH(CH₃)₂, OCH₂CH₂CH₂CH₃, OCH₂CHCH₂ and OCH₂(C₆H₅),
and
in which R¹ means a radical from the group consisting of OCH₃, OCH₂CH₃, OCH(CH₃)₂, OCH₂CH₂CH₂CH₃, OCH₂CHCH₂ and OCH₂(C₆H₅).

2. The method according to claim 1, in which R means a radical from the group consisting of OCH₃ and OCH₂CH₃,
and
R¹ means a radical from the group consisting of OCH₃ and OCH₂CH₃.

3. The method according to claim 1, characterized in that the protein is selected from the group consisting of
a) proteins comprising the amino acid sequence shown in SEQ ID No. 1 wherein the amino acid at position 186 is different from L;
b) proteins having an amino acid sequence having at least 80% identity to the amino acid sequence of a) provided that the amino acid at position 186 is different from L.

4. The method according to claim 3, characterized in that the protein has at least one substitution selected from the group consisting of:
(i) the amino acid at position 79 is different from E;
(ii) the amino acid at position 202 is different from A;
(iii) the amino acid at position 280 is different from L;
(iv) the amino acid at position 301 is different from D;
(v) the amino acid at position 3 is different from M;
(vi) an amino acid at position 11 is different from C;
(vii) the amino acid at position 17 is different from L;
(viii) the amino acid at position 40 is different from K;
(ix) the amino acid at position 111 is different from Q.

5. The method according to claim 3, characterized in that the protein has the amino acid substitutions at position 186 and at position 79 and at position 301 and at position 40, wherein the amino acid at position 186 is Y and the amino acid at position 79 is S and the amino acid at position 301 is A and the amino acid at position 40 is M.

6. The method according to claim 1, characterized in that the protein is used in an amount of 0.1-50% by weight, based on the mixture (I).

7. The method according to claim 6, characterized in that the protein is used in an amount of 0.5-10% by weight, based on the mixture (I).

8. The method according to claim 7, characterized in that the protein is used in an amount of 1-5% by weight, based on the mixture (I).

9. The method according to claim 1, characterized in that the first step is carried out either without solvent or in the presence of a solvent selected from the group consisting of toluene, xylenes, mesitylene, and n-butanol.

10. The method according to claim 1, characterized in that, in the first step, the acylation or carboxylation agent R—C(═O)R¹ is used in an amount of 0.4-25 equivalents, based on a molar amount of mixture (I) used.

11. The method according to claim 10, characterized in that, in the first step, the acylation or carboxylation agent R—C(═O)R¹ is preferably used in an amount of 1.0-5.0 equivalents, based on the molar amount of mixture (I) used.

12. The method according to claim 1, characterized in that, in the first step, the reaction is carried out at a temperature of 20-130° C.

13. The method according to claim 12, characterized in that, in the first step, the reaction is carried out at temperatures of the temperature is 80-120° C.

14. The method according to claim 1, characterized in that, in the third step, a base from the group consisting of lithium hydroxide, sodium hydroxide and potassium hydroxide is used.

15. The method according to claim 1, characterized in that, in step 3 the third step, the base is used in a stoichiometry of 1.00-3.00 equivalents, based on a molar amount of component (II).

16. A method for producing a mixture of all four stereoisomers of 2,6-dimethyl-1-indanamine (I) by metal-catalysed isomerization of a mixture of three stereoisomers of (1R,2R)-, (1S,2R)- and (1S,2S)-2,6-dimethyl-1-indanamine:

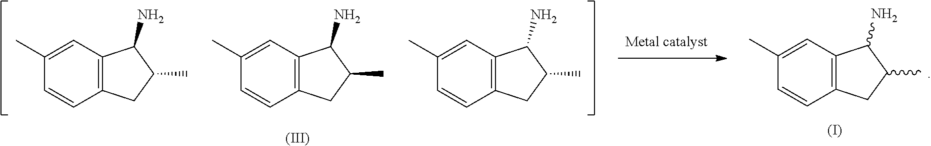

17. The method according to claim 16, characterized in that a palladium on carbon (Pd/C) or a palladium on aluminium oxide (Pd/Al₂O₃) catalyst having a palladium load of 0.5-10% by weight is used.

18. The method according to claim 16, characterized in that the reaction is carried out at between 1-6 bar gauge pressure under injection of a gas mixture comprising nitrogen and hydrogen.

19. The method according to claim 3, wherein the amino acid in the proteins according to a) or b) at position 186 is F, W, Y, E, D, Q, T, H, P, C, K, S, N, I or V.

20. The method according to claim 4, characterized in that the protein has at least one substitution selected from the group consisting of:
(i) the amino acid at position 79 is S;
(ii) the amino acid at position 202 is N;
(iii) the amino acid at position 280 is A;
(iv) the amino acid at position 301 is A;
(v) the amino acid at position 3 is Q;
(vi) the amino acid at position 11 is A;
(vii) the amino acid at position 17 is P;
(viii) the amino acid at position 40 is M;
(ix) the amino acid at position 111 is E.


SEQUENCE LISTING (402 sequences)

SEQ ID NO: 1 - protein, 319 aa, unidentified
MRMSRRAKLRCALPLALVAVVAAFAPAANAQLAVDYSLPKALAAWAANPNGSPPGANDWSCRPSSLHPYPVILVHGTFENQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTLSGLTDFARLLGPIGNAAVQLVTSFCPACGDQIRGSAFMQELNAGGDTVAGVAYTVIQTKYDEVVTPYDSAFLTPAASSHQVRNILVQKGCPIDLSEHLAIPFDRRALAYVLNALDPAHPRPVPCVPVAPLIGG

SEQ ID NO: 2 - DNA, 957 bp, unidentified
  source 1..957: mol_type = genomic DNA
atgcgtatgtctcgtcgtgccaaactgcgctgcgccttaccccttgcgctggtagctgtggttgctgcctttgcacccgcggcaaatgcacaactggcggtggactatagcctgccgaaagcgctcgctgcatgggcagccaatccgaatggcagcccaccgggtgcgaacgattggtcgtgtcgcccaagctccttgcacccttatccggtgattctggtccacggtacattcgagaaccagcgcttcaattggaatgccctggcgcctctgctgaagaacgcgggttactgcgtcttttcctttaactatggagcaacgcaaggtacgcaacttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaaccctgagtggcctgactgactttgctcgcttgctcggccctattggcaatgcagccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacttatcggagcatctggcgattccgtttgatcgtcgtgcgctggcttatgtcctgaacgcgttagatccggcccatccacgtccggtgccttgtgtcccggtagcgccgctgattggcggg

SEQ ID NO: 3 - protein, 319 aa, synthetic construct
MRMSRRAKLRCALPLALVAVVAAFAPAANAQLAVDYSLPKALAAWAANPNGSPPGANDWSCRPSSLHPYPVILVHGTFENQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTFSGLTDFARLLGPIGNAAVQLVTSFCPACGDQIRGSAFMQELNAGGDTVAGVAYTVIQTKYDEVVTPYDSAFLTPAASSHQVRNILVQKGCPIDLSEHLAIPFDRRALAYVLNALDPAHPRPVPCVPVAPLIGG

SEQ ID NO: 4 - DNA, 957 bp, synthetic construct
  source 1..957: mol_type = other DNA
atgcgtatgagccgtcgtgcaaaactgcgttgtgcactgccgctggcactggttgcagttgttgcagcatttgcaccggcagcaaatgcacagctggcagttgattatagcctgccgaaagcactggcagcatgggcagccaatccgaatggtagccctccgggtgccaatgattggagctgtcgtccgagcagcctgcatccgtatccggttattctggttcatggcacctttgaaaatcagcgctttaattggaatgcactggcaccgctgctgaaaaatgcaggttattgtgttttcagcttcaactatggtgcaacccagggcacccagctgaccggtggttttgcaaatggtctggcagcagttcgtgatagcgcacgtcaactggcagcctttgttgatcgtgttcgtgcagcaaccggtgcagcagaagttgatattgttggtcatagccaaggtggtatgatgcctcgttattataccaataaacttggtggtgcaagcaaagttcgtgccctggttggtctgagcccgagcaatcatggtacaacctttagcggtctgaccgattttgcacgtctgctgggtccgattggtaatgcagccgttcagctggttaccagtttttgtccggcatgtggtgatcagattcgtggtagcgcctttatgcaagaactgaatgccggtggtgataccgttgccggtgttgcatataccgttattcagaccaaatatgatgaagtggttaccccgtatgatagcgcgtttctgactccggcagccagcagccatcaggttcgtaatattctggtgcagaaaggttgtccgattgatctgagcgaacatctggcaattccgtttgatcgtcgtgcactggcctatgttctgaatgccctggatccggcacatccgcgtccggttccgtgtgttccggttgcaccgctgattggcggt

SEQ ID NO: 5 - protein, 319 aa, synthetic construct
MRMSRRAKLRCALPLALVAVVAAFAPAANAQLAVDYSLPKALAAWAANPNGSPPGANDWSCRPSSLHPYPVILVHGTFENQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTLSGLTDFARLLGPIGNAAVQLVTSFCPACGDQIRGSAFMQELNAGGDTVAGVAYTVIQTKYDEVVTPYDSAFLTPAASSHQVRNILVQKGCPIDESEHLAIPFDRRALAYVLNALDPAHPRPVPCVPVAPLIGG

SEQ ID NO: 6 - DNA, 957 bp, synthetic construct
  source 1..957: mol_type = other DNA
atgcgtatgagccgtcgtgcaaaactgcgttgtgcactgccgctggcactggttgcagttgttgcagcatttgcaccggcagcaaatgcacagctggcagttgattatagcctgccgaaagcactggcagcatgggcagccaatccgaatggtagccctccgggtgccaatgattggagctgtcgtccgagcagcctgcatccgtatccggttattctggttcatggcacctttgaaaatcagcgctttaattggaatgcactggcaccgctgctgaaaaatgcaggttattgtgttttcagcttcaactatggtgcaacccagggcacccagctgaccggtggttttgcaaatggtctggcagcagttcgtgatagcgcacgtcaactggcagcctttgttgatcgtgttcgtgcagcaaccggtgcagcagaagttgatattgttggtcatagccaaggtggtatgatgcctcgttattataccaataaacttggtggtgcaagcaaagttcgtgccctggttggtctgagcccgagcaatcatggtacaaccctgagcggtctgaccgattttgcacgtctgctgggtccgattggtaatgcagccgttcagctggttaccagtttttgtccggcatgtggtgatcagattcgtggtagcgcctttatgcaagaactgaatgccggtggtgataccgttgccggtgttgcatataccgttattcagaccaaatatgatgaagtggttaccccgtatgatagcgcgtttctgactccggcagccagcagccatcaggttcgtaatattctggtgcagaaaggttgtccgattgatgagagcgaacatctggcaattccgtttgatcgtcgtgcactggcctatgttctgaatgccctggatccggcacatccgcgtccggttccgtgtgttccggttgcaccgctgattggcggt

SEQ ID NO: 7 - protein, 319 aa, synthetic construct
MRMSRRAKLRCALPLALVAVVAAFAPAANAQLAVDYSLPKALAAWAANPNGSPPGANDWSCRPSSLHPYPVILVHGTFENQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTLSGLTDFARLLGPIGNAAVQLVTSFCPACGDQIRGSAFMQELNAGGDTVAGVAYTVIQTKYDEVVTPYDSAFLTPAASSHQVRNILVQKGCPIDSSEHLAIPFDRRALAYVLNALDPAHPRPVPCVPVAPLIGG

SEQ ID NO: 8 - DNA, 957 bp, synthetic construct
  source 1..957: mol_type = other DNA
atgcgtatgagccgtcgtgcaaaactgcgttgtgcactgccgctggcactggttgcagttgttgcagcatttgcaccggcagcaaatgcacagctggcagttgattatagcctgccgaaagcactggcagcatgggcagccaatccgaatggtagccctccgggtgccaatgattggagctgtcgtccgagcagcctgcatccgtatccggttattctggttcatggcacctttgaaaatcagcgctttaattggaatgcactggcaccgctgctgaaaaatgcaggttattgtgttttcagcttcaactatggtgcaacccagggcacccagctgaccggtggttttgcaaatggtctggcagcagttcgtgatagcgcacgtcaactggcagcctttgttgatcgtgttcgtgcagcaaccggtgcagcagaagttgatattgttggtcatagccaaggtggtatgatgcctcgttattataccaataaacttggtggtgcaagcaaagttcgtgccctggttggtctgagcccgagcaatcatggtacaaccctgagcggtctgaccgattttgcacgtctgctgggtccgattggtaatgcagccgttcagctggttaccagtttttgtccggcatgtggtgatcagattcgtggtagcgcctttatgcaagaactgaatgccggtggtgataccgttgccggtgttgcatataccgttattcagaccaaatatgatgaagtggttaccccgtatgatagcgcgtttctgactccggcagccagcagccatcaggttcgtaatattctggtgcagaaaggttgtccgattgattctagcgaacatctggcaattccgtttgatcgtcgtgcactggcctatgttctgaatgccctggatccggcacatccgcgtccggttccgtgtgttccggttgcaccgctgattggcggt

SEQ ID NO: 9 - protein, 319 aa, synthetic construct
MRMSRRAKLRCALPLALVAVVAAFAPAANAQLAVDYSLPKALAAWAANPNGSPPGANDWSCRPSSLHPYPVILVHGTFENQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTLSGLTDFARLLGPIGNAAVQLVTSFCPACGDQIRGSAFMQELNAGGDTVAGVAYTVIQTKYDEVVTPYDSAFLTPAASSHQVRNILVQKGCPIDKSEHLAIPFDRRALAYVLNALDPAHPRPVPCVPVAPLIGG

SEQ ID NO: 10 - DNA, 957 bp, synthetic construct
  source 1..957: mol_type = other DNA
atgcgtatgagccgtcgtgcaaaactgcgttgtgcactgccgctggcactggttgcagttgttgcagcatttgcaccggcagcaaatgcacagctggcagttgattatagcctgccgaaagcactggcagcatgggcagccaatccgaatggtagccctccgggtgccaatgattggagctgtcgtccgagcagcctgcatccgtatccggttattctggttcatggcacctttgaaaatcagcgctttaattggaatgcactggcaccgctgctgaaaaatgcaggttattgtgttttcagcttcaactatggtgcaacccagggcacccagctgaccggtggttttgcaaatggtctggcagcagttcgtgatagcgcacgtcaactggcagcctttgttgatcgtgttcgtgcagcaaccggtgcagcagaagttgatattgttggtcatagccaaggtggtatgatgcctcgttattataccaataaacttggtggtgcaagcaaagttcgtgccctggttggtctgagcccgagcaatcatggtacaaccctgagcggtctgaccgattttgcacgtctgctgggtccgattggtaatgcagccgttcagctggttaccagtttttgtccggcatgtggtgatcagattcgtggtagcgcctttatgcaagaactgaatgccggtggtgataccgttgccggtgttgcatataccgttattcagaccaaatatgatgaagtggttaccccgtatgatagcgcgtttctgactccggcagccagcagccatcaggttcgtaatattctggtgcagaaaggttgtccgattgataagagcgaacatctggcaattccgtttgatcgtcgtgcactggcctatgttctgaatgccctggatccggcacatccgcgtccggttccgtgtgttccggttgcaccgctgattggcggt

SEQ ID NO: 11 - protein, 319 aa, synthetic construct
MRMSRRAKLRCALPLALVAVVAAFAPAANAQLAVDYSLPKALAAWAANPNGSPPGANDWSCRPSSLHPYPVILVHGTFENQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTLSGLTDFARLLGPIGNAAVQLVTSFCPACGDQIRGSAFMQELNAGGDTVAGVAYTVIQTKYDEVVTPYDSAFLTPAASSHQVRNILVQKGCPIDLSEHLAIPFDRRALAYVLNALDPAHPRPVPCVNVAPLIGG

SEQ ID NO: 12 - DNA, 957 bp, synthetic construct
  source 1..957: mol_type = other DNA
atgcgtatgagccgtcgtgcaaaactgcgttgtgcactgccgctggcactggttgcagttgttgcagcatttgcaccggcagcaaatgcacagctggcagttgattatagcctgccgaaagcactggcagcatgggcagccaatccgaatggtagccctccgggtgccaatgattggagctgtcgtccgagcagcctgcatccgtatccggttattctggttcatggcacctttgaaaatcagcgctttaattggaatgcactggcaccgctgctgaaaaatgcaggttattgtgttttcagcttcaactatggtgcaacccagggcacccagctgaccggtggttttgcaaatggtctggcagcagttcgtgatagcgcacgtcaactggcagcctttgttgatcgtgttcgtgcagcaaccggtgcagcagaagttgatattgttggtcatagccaaggtggtatgatgcctcgttattataccaataaacttggtggtgcaagcaaagttcgtgccctggttggtctgagcccgagcaatcatggtacaaccctgagcggtctgaccgattttgcacgtctgctgggtccgattggtaatgcagccgttcagctggttaccagtttttgtccggcatgtggtgatcagattcgtggtagcgcctttatgcaagaactgaatgccggtggtgataccgttgccggtgttgcatataccgttattcagaccaaatatgatgaagtggttaccccgtatgatagcgcgtttctgactccggcagccagcagccatcaggttcgtaatattctggtgcagaaaggttgtccgattgatctgagcgaacatctggcaattccgtttgatcgtcgtgcactggcctatgttctgaatgccctggatccggcacatccgcgtccggttccgtgtgttaatgttgcaccgctgattggcggt

SEQ ID NO: 13 - protein, 319 aa, synthetic construct
MRMSRRAKLRCALPLALVAVVAAFAPAANAQLAVDYSLPKALAAWAANPNGSPPGANDWSCRPSSLHPYPVILVHGTFENQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTLSGLTDFARLLGPIGNAAVQLVTSFCPACGDQIRGSAFMQELNAGGDTVAGVAYTVIQTKYDEVVTPYDSAFLTPAASSHQVRNILVQKGCPIDLSEHLAIPFDRRALAYVLNALDPAHPRPVPCVFVAPLIGG

SEQ ID NO: 14 - DNA, 957 bp, synthetic construct
  source 1..957: mol_type = other DNA
atgcgtatgagccgtcgtgcaaaactgcgttgtgcactgccgctggcactggttgcagttgttgcagcatttgcaccggcagcaaatgcacagctggcagttgattatagcctgccgaaagcactggcagcatgggcagccaatccgaatggtagccctccgggtgccaatgattggagctgtcgtccgagcagcctgcatccgtatccggttattctggttcatggcacctttgaaaatcagcgctttaattggaatgcactggcaccgctgctgaaaaatgcaggttattgtgttttcagcttcaactatggtgcaacccagggcacccagctgaccggtggttttgcaaatggtctggcagcagttcgtgatagcgcacgtcaactggcagcctttgttgatcgtgttcgtgcagcaaccggtgcagcagaagttgatattgttggtcatagccaaggtggtatgatgcctcgttattataccaataaacttggtggtgcaagcaaagttcgtgccctggttggtctgagcccgagcaatcatggtacaacctggagcggtctgaccgattttgcacgtctgctgggtccgattggtaatgcagccgttcagctggttaccagtttttgtccggcatgtggtgatcagattcgtggtagcgcctttatgcaagaactgaatgccggtggtgataccgttgccggtgttgcatataccgttattcagaccaaatatgatgaagtggttaccccgtatgatagcgcgtttctgactccggcagccagcagccatcaggttcgtaatattctggtgcagaaaggttgtccgattgatctgagcgaacatctggcaattccgtttgatcgtcgtgcactggcctatgttctgaatgccctggatccggcacatccgcgtccggttccgtgtgttccggttgcaccgctgattggcggt

SEQ ID NO: 15 - protein, 319 aa, synthetic construct
MRMSRRAKLRCALPLALVAVVAAFAPAANAQLAVDYSLPKALAAWAANPNGSPPGANDWSCRPSSLHPYPVILVHGTFENQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTWSGLTDFARLLGPIGNAAVQLVTSFCPACGDQIRGSAFMQELNAGGDTVAGVAYTVIQTKYDEVVTPYDSAFLTPAASSHQVRNILVQKGCPIDLSEHLAIPFDRRALAYVLNALDPAHPRPVPCVPVAPLIGG

SEQ ID NO: 16 - DNA, 957 bp, synthetic construct; identical to SEQ ID NO: 14
  source 1..957: mol_type = other DNA

SEQ ID NO: 17 - protein, 319 aa, synthetic construct
MRLSRRAKLRCALPLALVAVVAAFAPAANAQLAVDYSLPKALAAWAANPNGSPPGANDWSCRPSSLHPYPVILVHGTFENQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTLSGLTDFARLLGPIGNAAVQLVTSFCPACGDQIRGSAFMQELNAGGDTVAGVAYTVIQTKYDEVVTPYDSAFLTPAASSHQVRNILVQKGCPIDLSEHLAIPFDRRALAYVLNALDPAHPRPVPCVPVAPLIGG

SEQ ID NO: 18 - DNA, 957 bp, synthetic construct
  source 1..957: mol_type = other DNA
atgcgtttgagccgtcgtgcaaaactgcgttgtgcactgccgctggcactggttgcagttgttgcagcatttgcaccggcagcaaatgcacagctggcagttgattatagcctgccgaaagcactggcagcatgggcagccaatccgaatggtagccctccgggtgccaatgattggagctgtcgtccgagcagcctgcatccgtatccggttattctggttcatggcacctttgaaaatcagcgctttaattggaatgcactggcaccgctgctgaaaaatgcaggttattgtgttttcagcttcaactatggtgcaacccagggcacccagctgaccggtggttttgcaaatggtctggcagcagttcgtgatagcgcacgtcaactggcagcctttgttgatcgtgttcgtgcagcaaccggtgcagcagaagttgatattgttggtcatagccaaggtggtatgatgcctcgttattataccaataaacttggtggtgcaagcaaagttcgtgccctggttggtctgagcccgagcaatcatggtacaaccctgagcggtctgaccgattttgcacgtctgctgggtccgattggtaatgcagccgttcagctggttaccagtttttgtccggcatgtggtgatcagattcgtggtagcgcctttatgcaagaactgaatgccggtggtgataccgttgccggtgttgcatataccgttattcagaccaaatatgatgaagtggttaccccgtatgatagcgcgtttctgactccggcagccagcagccatcaggttcgtaatattctggtgcagaaaggttgtccgattgatctgagcgaacatctggcaattccgtttgatcgtcgtgcactggcctatgttctgaatgccctggatccggcacatccgcgtccggttccgtgtgttccggttgcaccgctgattggcggt

SEQ ID NO: 19 - protein, 319 aa, synthetic construct
MRMSRRAKLRCALPLALVAVVAAFAPAAYAQLAVDYSLPKALAAWAANPNGSPPGANDWSCRPSSLHPYPVILVHGTFENQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTLSGLTDFARLLGPIGNAAVQLVTSFCPACGDQIRGSAFMQELNAGGDTVAGVAYTVIQTKYDEVVTPYDSAFLTPAASSHQVRNILVQKGCPIDLSEHLAIPFDRRALAYVLNALDPAHPRPVPCVPVAPLIGG

SEQ ID NO: 20 - DNA, 957 bp, synthetic construct
  source 1..957: mol_type = other DNA
atgcgtatgagccgtcgtgcaaaactgcgttgtgcactgccgctggcactggttgcagttgttgcagcatttgcaccggcagcatatgcacagctggcagttgattatagcctgccgaaagcactggcagcatgggcagccaatccgaatggtagccctccgggtgccaatgattggagctgtcgtccgagcagcctgcatccgtatccggttattctggttcatggcacctttgaaaatcagcgctttaattggaatgcactggcaccgctgctgaaaaatgcaggttattgtgttttcagcttcaactatggtgcaacccagggcacccagctgaccggtggttttgcaaatggtctggcagcagttcgtgatagcgcacgtcaactggcagcctttgttgatcgtgttcgtgcagcaaccggtgcagcagaagttgatattgttggtcatagccaaggtggtatgatgcctcgttattataccaataaacttggtggtgcaagcaaagttcgtgccctggttggtctgagcccgagcaatcatggtacaaccctgagcggtctgaccgattttgcacgtctgctgggtccgattggtaatgcagccgttcagctggttaccagtttttgtccggcatgtggtgatcagattcgtggtagcgcctttatgcaagaactgaatgccggtggtgataccgttgccggtgttgcatataccgttattcagaccaaatatgatgaagtggttaccccgtatgatagcgcgtttctgactccggcagccagcagccatcaggttcgtaatattctggtgcagaaaggttgtccgattgatctgagcgaacatctggcaattccgtttgatcgtcgtgcactggcctatgttctgaatgccctggatccggcacatccgcgtccggttccgtgtgttccggttgcaccgctgattggcggt

SEQ ID NO: 21 - protein, 319 aa, synthetic construct
MRMSRRAKLRCALPLAPVAVVAAFAPAANAQLAVDYSLPKALAAWAANPNGSPPGANDWSCRPSSLHPYPVILVHGTFENQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTLSGLTDFARLLGPIGNAAVQLVTSFCPACGDQIRGSAFMQELNAGGDTVAGVAYTVIQTKYDEVVTPYDSAFLTPAASSHQVRNILVQKGCPIDLSEHLAIPFDRRALAYVLNALDPAHPRPVPCVPVAPLIGG

SEQ ID NO: 22 - DNA, 957 bp, synthetic construct
  source 1..957: mol_type = other DNA
atgcgtatgagccgtcgtgcaaaactgcgttgtgcactgccgctggcaccggttgcagttgttgcagcatttgcaccggcagcaaatgcacagctggcagttgattatagcctgccgaaagcactggcagcatgggcagccaatccgaatggtagccctccgggtgccaatgattggagctgtcgtccgagcagcctgcatccgtatccggttattctggttcatggcacctttgaaaatcagcgctttaattggaatgcactggcaccgctgctgaaaaatgcaggttattgtgttttcagcttcaactatggtgcaacccagggcacccagctgaccggtggttttgcaaatggtctggcagcagttcgtgatagcgcacgtcaactggcagcctttgttgatcgtgttcgtgcagcaaccggtgcagcagaagttgatattgttggtcatagccaaggtggtatgatgcctcgttattataccaataaacttggtggtgcaagcaaagttcgtgccctggttggtctgagcccgagcaatcatggtacaaccctgagcggtctgaccgattttgcacgtctgctgggtccgattggtaatgcagccgttcagctggttaccagtttttgtccggcatgtggtgatcagattcgtggtagcgcctttatgcaagaactgaatgccggtggtgataccgttgccggtgttgcatataccgttattcagaccaaatatgatgaagtggttaccccgtatgatagcgcgtttctgactccggcagccagcagccatcaggttcgtaatattctggtgcagaaaggttgtccgattgatctgagcgaacatctggcaattccgtttgatcgtcgtgcactggcctatgttctgaatgccctggatccggcacatccgcgtccggttccgtgtgttccggttgcaccgctgattggcggt

SEQ ID NO: 23 - protein, 319 aa, synthetic construct
MRMPRRAKLRCALPLALVAVVAAFAPAANAQLAVDYSLPKALAAWAANPNGSPPGANDWSCRPSSLHPYPVILVHGTFENQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTLSGLTDFARLLGPIGNAAVQLVTSFCPACGDQIRGSAFMQELNAGGDTVAGVAYTVIQTKYDEVVTPYDSAFLTPAASSHQVRNILVQKGCPIDLSEHLAIPFDRRALAYVLNALDPAHPRPVPCVPVAPLIGG

SEQ ID NO: 24 - DNA, 957 bp, synthetic construct
  source 1..957: mol_type = other DNA
atgcgtatgccgcgtcgtgcaaaactgcgttgtgcactgccgctggcactggttgcagttgttgcagcatttgcaccggcagcaaatgcacagctggcagttgattatagcctgccgaaagcactggcagcatgggcagccaatccgaatggtagccctccgggtgccaatgattggagctgtcgtccgagcagcctgcatccgtatccggttattctggttcatggcacctttgaaaatcagcgctttaattggaatgcactggcaccgctgctgaaaaatgcaggttattgtgttttcagcttcaactatggtgcaacccagggcacccagctgaccggtggttttgcaaatggtctggcagcagttcgtgatagcgcacgtcaactggcagcctttgttgatcgtgttcgtgcagcaaccggtgcagcagaagttgatattgttggtcatagccaaggtggtatgatgcctcgttattataccaataaacttggtggtgcaagcaaagttcgtgccctggttggtctgagcccgagcaatcatggtacaaccctgagcggtctgaccgattttgcacgtctgctgggtccgattggtaatgcagccgttcagctggttaccagtttttgtccggcatgtggtgatcagattcgtggtagcgcctttatgcaagaactgaatgccggtggtgataccgttgccggtgttgcatataccgttattcagaccaaatatgatgaagtggttaccccgtatgatagcgcgtttctgactccggcagccagcagccatcaggttcgtaatattctggtgcagaaaggttgtccgattgatctgagcgaacatctggcaattccgtttgatcgtcgtgcactggcctatgttctgaatgccctggatccggcacatccgcgtccggttccgtgtgttccggttgcaccgctgattggcggt

SEQ ID NO: 25 - protein, 319 aa, synthetic construct
MRMSRRAKLRCALPLALVAVVAAFAPAANAQLAVDYSLPKALAAWAANPNGSPPGANDWSCRPSSLHPYPVILVHGTFENQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTLSGLTDFARLLGPIGNAAVQLVTSFCPACGDQIRGSAFMQELNAGGDTVAGVAYTVIQTKYDEVVTPYDSAFLTPAASSHQVRNILVQKGCPIDDSEHLAIPFDRRALAYVLNALDPAHPRPVPCVPVAPLIGG

SEQ ID NO: 26 - DNA, 957 bp, synthetic construct
  source 1..957: mol_type = other DNA
atgcgtatgagccgtcgtgcaaaactgcgttgtgcactgccgctggcactggttgcagttgttgcagcatttgcaccggcagcaaatgcacagctggcagttgattatagcctgccgaaagcactggcagcatgggcagccaatccgaatggtagccctccgggtgccaatgattggagctgtcgtccgagcagcctgcatccgtatccggttattctggttcatggcacctttgaaaatcagcgctttaattggaatgcactggcaccgctgctgaaaaatgcaggttattgtgttttcagcttcaactatggtgcaacccagggcacccagctgaccggtggttttgcaaatggtctggcagcagttcgtgatagcgcacgtcaactggcagcctttgttgatcgtgttcgtgcagcaaccggtgcagcagaagttgatattgttggtcatagccaaggtggtatgatgcctcgttattataccaataaacttggtggtgcaagcaaagttcgtgccctggttggtctgagcccgagcaatcatggtacaaccctgagcggtctgaccgattttgcacgtctgctgggtccgattggtaatgcagccgttcagctggttaccagtttttgtccggcatgtggtgatcagattcgtggtagcgcctttatgcaagaactgaatgccggtggtgataccgttgccggtgttgcatataccgttattcagaccaaatatgatgaagtggttaccccgtatgatagcgcgtttctgactccggcagccagcagccatcaggttcgtaatattctggtgcagaaaggttgtccgattgatgatagcgaacatctggcaattccgtttgatcgtcgtgcactggcctatgttctgaatgccctggatccggcacatccgcgtccggttccgtgtgttccggttgcaccgctgattggcggt

SEQ ID NO: 27 - protein, 319 aa, synthetic construct
MRMSRRAKLRCALPLALAAVVAAFAPAANAQLAVDYSLPKALAAWAANPNGSPPGANDWSCRPSSLHPYPVILVHGTFENQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTLSGLTDFARLLGPIGNAAVQLVTSFCPACGDQIRGSAFMQELNAGGDTVAGVAYTVIQTKYDEVVTPYDSAFLTPAASSHQVRNILVQKGCPIDLSEHLAIPFDRRALAYVLNALDPAHPRPVPCVPVAPLIGG

SEQ ID NO: 28 - DNA, 957 bp, synthetic construct
  source 1..957: mol_type = other DNA
atgcgtatgagccgtcgtgcaaaactgcgttgtgcactgccgctggcactggcggcagttgttgcagcatttgcaccggcagcaaatgcacagctggcagttgattatagcctgccgaaagcactggcagcatgggcagccaatccgaatggtagccctccgggtgccaatgattggagctgtcgtccgagcagcctgcatccgtatccggttattctggttcatggcacctttgaaaatcagcgctttaattggaatgcactggcaccgctgctgaaaaatgcaggttattgtgttttcagcttcaactatggtgcaacccagggcacccagctgaccggtggttttgcaaatggtctggcagcagttcgtgatagcgcacgtcaactggcagcctttgttgatcgtgttcgtgcagcaaccggtgcagcagaagttgatattgttggtcatagccaaggtggtatgatgcctcgttattataccaataaacttggtggtgcaagcaaagttcgtgccctggttggtctgagcccgagcaatcatggtacaaccctgagcggtctgaccgattttgcacgtctgctgggtccgattggtaatgcagccgttcagctggttaccagtttttgtccggcatgtggtgatcagattcgtggtagcgcctttatgcaagaactgaatgccggtggtgataccgttgccggtgttgcatataccgttattcagaccaaatatgatgaagtggttaccccgtatgatagcgcgtttctgactccggcagccagcagccatcaggttcgtaatattctggtgcagaaaggttgtccgattgatctgagcgaacatctggcaattccgtttgatcgtcgtgcactggcctatgttctgaatgccctggatccggcacatccgcgtccggttccgtgtgttccggttgcaccgctgattggcggt

SEQ ID NO: 29 - protein, 319 aa, synthetic construct
MRQSRRAKLRCALPLALVAVVAAFAPAANAQLAVDYSLPKALAAWAANPNGSPPGANDWSCRPSSLHPYPVILVHGTFENQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTLSGLTDFARLLGPIGNAAVQLVTSFCPACGDQIRGSAFMQELNAGGDTVAGVAYTVIQTKYDEVVTPYDSAFLTPAASSHQVRNILVQKGCPIDLSEHLAIPFDRRALAYVLNALDPAHPRPVPCVPVAPLIGG

SEQ ID NO: 30 - DNA, 957 bp, synthetic construct
  source 1..957: mol_type = other DNA
atgcgtcagagccgtcgtgcaaaactgcgttgtgcactgccgctggcactggttgcagttgttgcagcatttgcaccggcagcaaatgcacagctggcagttgattatagcctgccgaaagcactggcagcatgggcagccaatccgaatggtagccctccgggtgccaatgattggagctgtcgtccgagcagcctgcatccgtatccggttattctggttcatggcacctttgaaaatcagcgctttaattggaatgcactggcaccgctgctgaaaaatgcaggttattgtgttttcagcttcaactatggtgcaacccagggcacccagctgaccggtggttttgcaaatggtctggcagcagttcgtgatagcgcacgtcaactggcagcctttgttgatcgtgttcgtgcagcaaccggtgcagcagaagttgatattgttggtcatagccaaggtggtatgatgcctcgttattataccaataaacttggtggtgcaagcaaagttcgtgccctggttggtctgagcccgagcaatcatggtacaaccctgagcggtctgaccgattttgcacgtctgctgggtccgattggtaatgcagccgttcagctggttaccagtttttgtccggcatgtggtgatcagattcgtggtagcgcctttatgcaagaactgaatgccggtggtgataccgttgccggtgttgcatataccgttattcagaccaaatatgatgaagtggttaccccgtatgatagcgcgtttctgactccggcagccagcagccatcaggttcgtaatattctggtgcagaaaggttgtccgattgatctgagcgaacatctggcaattccgtttgatcgtcgtgcactggcctatgttctgaatgccctggatccggcacatccgcgtccggttccgtgtgttccggttgcaccgctgattggcggt

SEQ ID NO: 31 - protein, 319 aa, synthetic construct
MRMSRRAKLRCALPLALVAVVAAFAPAANAQLAVDYSLPKALAAWAANPNGSPPGANDWSCRPSSLHPYPVILVHGTFENQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTLSGLTDFARLLGPIGNAAVQLVTSFCPACGDQIRGSAFMQELNAGGDTVAGVAYTVIQTKYDEVVTPYDSAFLTPAASSHQVRNILVQKGCPIDLSEHLAIPFDRRALAYVLNALDPAHPRPVPCVDVAPLIGG

SEQ ID NO: 32 - DNA, 957 bp, synthetic construct
  source 1..957: mol_type = other DNA
atgcgtatgagccgtcgtgcaaaactgcgttgtgcactgccgctggcactggttgcagttgttgcagcatttgcaccggcagcaaatgcacagctggcagttgattatagcctgccgaaagcactggcagcatgggcagccaatccgaatggtagccctccgggtgccaatgattggagctgtcgtccgagcagcctgcatccgtatccggttattctggttcatggcacctttgaaaatcagcgctttaattggaatgcactggcaccgctgctgaaaaatgcaggttattgtgttttcagcttcaactatggtgcaacccagggcacccagctgaccggtggttttgcaaatggtctggcagcagttcgtgatagcgcacgtcaactggcagcctttgttgatcgtgttcgtgcagcaaccggtgcagcagaagttgatattgttggtcatagccaaggtggtatgatgcctcgttattataccaataaacttggtggtgcaagcaaagttcgtgccctggttggtctgagcccgagcaatcatggtacaaccctgagcggtctgaccgattttgcacgtctgctgggtccgattggtaatgcagccgttcagctggttaccagtttttgtccggcatgtggtgatcagattcgtggtagcgcctttatgcaagaactgaatgccggtggtgataccgttgccggtgttgcatataccgttattcagaccaaatatgatgaagtggttaccccgtatgatagcgcgtttctgactccggcagccagcagccatcaggttcgtaatattctggtgcagaaaggttgtccgattgatctgagcgaacatctggcaattccgtttgatcgtcgtgcactggcctatgttctgaatgccctggatccggcacatccgcgtccggttccgtgtgttgatgttgcaccgctgattggcggt

SEQ ID NO: 33 - protein, 319 aa, synthetic construct
MRMSRRAKLRCALPLALVAVVAAFAPAAWAQLAVDYSLPKALAAWAANPNGSPPGANDWSCRPSSLHPYPVILVHGTFENQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTLSGLTDFARLLGPIGNAAVQLVTSFCPACGDQIRGSAFMQELNAGGDTVAGVAYTVIQTKYDEVVTPYDSAFLTPAASSHQVRNILVQKGCPIDLSEHLAIPFDRRALAYVLNALDPAHPRPVPCVPVAPLIGG

SEQ ID NO: 34 - DNA, 957 bp, synthetic construct
  source 1..957: mol_type = other DNA
atgcgtatgagccgtcgtgcaaaactgcgttgtgcactgccgctggcactggttgcagttgttgcagcatttgcaccggcagcatgggcacagctggcagttgattatagcctgccgaaagcactggcagcatgggcagccaatccgaatggtagccctccgggtgccaatgattggagctgtcgtccgagcagcctgcatccgtatccggttattctggttcatggcacctttgaaaatcagcgctttaattggaatgcactggcaccgctgctgaaaaatgcaggttattgtgttttcagcttcaactatggtgcaacccagggcacccagctgaccggtggttttgcaaatggtctggcagcagttcgtgatagcgcacgtcaactggcagcctttgttgatcgtgttcgtgcagcaaccggtgcagcagaagttgatattgttggtcatagccaaggtggtatgatgcctcgttattataccaataaacttggtggtgcaagcaaagttcgtgccctggttggtctgagcccgagcaatcatggtacaaccctgagcggtctgaccgattttgcacgtctgctgggtccgattggtaatgcagccgttcagctggttaccagtttttgtccggcatgtggtgatcagattcgtggtagcgcctttatgcaagaactgaatgccggtggtgataccgttgccggtgttgcatataccgttattcagaccaaatatgatgaagtggttaccccgtatgatagcgcgtttctgactccggcagccagcagccatcaggttcgtaatattctggtgcagaaaggttgtccgattgatctgagcgaacatctggcaattccgtttgatcgtcgtgcactggcctatgttctgaatgccctggatccggcacatccgcgtccggttccgtgtgttccggttgcaccgctgattggcggt

SEQ ID NO: 35 - protein, 319 aa, synthetic construct
MRMSRRAKLRCALPLALVAVVAAFAPAANAQLAVDYSLPKALAAWAANPNGSPPGANDWSCRPSSLHPYPVILVHGTFENQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTYSGLTDFARLLGPIGNAAVQLVTSFCPACGDQIRGSAFMQELNAGGDTVAGVAYTVIQTKYDEVVTPYDSAFLTPAASSHQVRNILVQKGCPIDLSEHLAIPFDRRALAYVLNALDPAHPRPVPCVPVAPLIGG

SEQ ID NO: 36 - DNA, 957 bp, synthetic construct
  source 1..957: mol_type = other DNA
atgcgtatgagccgtcgtgcaaaactgcgttgtgcactgccgctggcactggttgcagttgttgcagcatttgcaccggcagcaaatgcacagctggcagttgattatagcctgccgaaagcactggcagcatgggcagccaatccgaatggtagccctccgggtgccaatgattggagctgtcgtccgagcagcctgcatccgtatccggttattctggttcatggcacctttgaaaatcagcgctttaattggaatgcactggcaccgctgctgaaaaatgcaggttattgtgttttcagcttcaactatggtgcaacccagggcacccagctgaccggtggttttgcaaatggtctggcagcagttcgtgatagcgcacgtcaactggcagcctttgttgatcgtgttcgtgcagcaaccggtgcagcagaagttgatattgttggtcatagccaaggtggtatgatgcctcgttattataccaataaacttggtggtgcaagcaaagttcgtgccctggttggtctgagcccgagcaatcatggtacaacctatagcggtctgaccgattttgcacgtctgctgggtccgattggtaatgcagccgttcagctggttaccagtttttgtccggcatgtggtgatcagattcgtggtagcgcctttatgcaagaactgaatgccggtggtgataccgttgccggtgttgcatataccgttattcagaccaaatatgatgaagtggttaccccgtatgatagcgcgtttctgactccggcagccagcagccatcaggttcgtaatattctggtgcagaaaggttgtccgattgatctgagcgaacatctggcaattccgtttgatcgtcgtgcactggcctatgttctgaatgccctggatccggcacatccgcgtccggttccgtgtgttccggttgcaccgctgattggcggt

SEQ ID NO: 37 - protein, 319 aa, synthetic construct
MRMSRRAKLRCALPLALTAVVAAFAPAANAQLAVDYSLPKALAAWAANPNGSPPGANDWSCRPSSLHPYPVILVHGTFENQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTLSGLTDFARLLGPIGNAAVQLVTSFCPACGDQIRGSAFMQELNAGGDTVAGVAYTVIQTKYDEVVTPYDSAFLTPAASSHQVRNILVQKGCPIDLSEHLAIPFDRRALAYVLNALDPAHPRPVPCVPVAPLIGG

SEQ ID NO: 38 - DNA, 957 bp, synthetic construct
  source 1..957: mol_type = other DNA
atgcgtatgagccgtcgtgcaaaactgcgttgtgcactgccgctggcactgacggcagttgttgcagcatttgcaccggcagcaaatgcacagctggcagttgattatagcctgccgaaagcactggcagcatgggcagccaatccgaatggtagccctccgggtgccaatgattggagctgtcgtccgagcagcctgcatccgtatccggttattctggttcatggcacctttgaaaatcagcgctttaattggaatgcactggcaccgctgctgaaaaatgcaggttattgtgttttcagcttcaactatggtgcaacccagggcacccagctgaccggtggttttgcaaatggtctggcagcagttcgtgatagcgcacgtcaactggcagcctttgttgatcgtgttcgtgcagcaaccggtgcagcagaagttgatattgttggtcatagccaaggtggtatgatgcctcgttattataccaataaacttggtggtgcaagcaaagttcgtgccctggttggtctgagcccgagcaatcatggtacaaccctgagcggtctgaccgattttgcacgtctgctgggtccgattggtaatgcagccgttcagctggttaccagtttttgtccggcatgtggtgatcagattcgtggtagcgcctttatgcaagaactgaatgccggtggtgataccgttgccggtgttgcatataccgttattcagaccaaatatgatgaagtggttaccccgtatgatagcgcgtttctgactccggcagccagcagccatcaggttcgtaatattctggtgcagaaaggttgtccgattgatctgagcgaacatctggcaattccgtttgatcgtcgtgcactggcctatgttctgaatgccctggatccggcacatccgcgtccggttccgtgtgttccggttgcaccgctgattggcggt

SEQ ID NO: 39 - protein, 319 aa, synthetic construct
MRMSRRAKLRCALPLALVAVVAAFAPAANAQLAVDYSLPKALAAWAANPNGSPPGANDWSCRPSSLHPYPVILVHGTFENQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTESGLTDFARLLGPIGNAAVQLVTSFCPACGDQIRGSAFMQELNAGGDTVAGVAYTVIQTKYDEVVTPYDSAFLTPAASSHQVRNILVQKGCPIDLSEHLAIPFDRRALAYVLNALDPAHPRPVPCVPVAPLIGG

SEQ ID NO: 40 - DNA, 957 bp, synthetic construct
  source 1..957: mol_type = other DNA
atgcgtatgagccgtcgtgcaaaactgcgttgtgcactgccgctggcactggttgcagttgttgcagcatttgcaccggcagcaaatgcacagctggcagttgattatagcctgccgaaagcactggcagcatgggcagccaatccgaatggtagccctccgggtgccaatgattggagctgtcgtccgagcagcctgcatccgtatccggttattctggttcatggcacctttgaaaatcagcgctttaattggaatgcactggcaccgctgctgaaaaatgcaggttattgtgttttcagcttcaactatggtgcaacccagggcacccagctgaccggtggttttgcaaatggtctggcagcagttcgtgatagcgcacgtcaactggcagcctttgttgatcgtgttcgtgcagcaaccggtgcagcagaagttgatattgttggtcatagccaaggtggtatgatgcctcgttattataccaataaacttggtggtgcaagcaaagttcgtgccctggttggtctgagcccgagcaatcatggtacaaccgagagcggtctgaccgattttgcacgtctgctgggtccgattggtaatgcagccgttcagctggttaccagtttttgtccggcatgtggtgatcagattcgtggtagcgcctttatgcaagaactgaatgccggtggtgataccgttgccggtgttgcatataccgttattcagaccaaatatgatgaagtggttaccccgtatgatagcgcgtttctgactccggcagccagcagccatcaggttcgtaatattctggtgcagaaaggttgtccgattgatctgagcgaacatctggcaattccgtttgatcgtcgtgcactggcctatgttctgaatgccctggatccggcacatccgcgtccggttccgtgtgttccggttgcaccgctgattggcggt

SEQ ID NO: 41 - protein, 319 aa, synthetic construct
MRMSRRAKLRCALPLALVAVVAAFAPAANAQLAVDYSLPKALAAWAANPNGSPPGANDWSCRPSSLHPYPVILVHGTFENQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTLSGLTDFARLLGPIGNQAVQLVTSFCPACGDQIRGSAFMQELNAGGDTVAGVAYTVIQTKYDEVVTPYDSAFLTPAASSHQVRNILVQKGCPIDLSEHLAIPFDRRALAYVLNALDPAHPRPVPCVPVAPLIGG

SEQ ID NO: 42 - DNA, 957 bp, synthetic construct
  source 1..957: mol_type = other DNA
atgcgtatgagccgtcgtgcaaaactgcgttgtgcactgccgctggcactggttgcagttgttgcagcatttgcaccggcagcaaatgcacagctggcagttgattatagcctgccgaaagcactggcagcatgggcagccaatccgaatggtagccctccgggtgccaatgattggagctgtcgtccgagcagcctgcatccgtatccggttattctggttcatggcacctttgaaaatcagcgctttaattggaatgcactggcaccgctgctgaaaaatgcaggttattgtgttttcagcttcaactatggtgcaacccagggcacccagctgaccggtggttttgcaaatggtctggcagcagttcgtgatagcgcacgtcaactggcagcctttgttgatcgtgttcgtgcagcaaccggtgcagcagaagttgatattgttggtcatagccaaggtggtatgatgcctcgttattataccaataaacttggtggtgcaagcaaagttcgtgccctggttggtctgagcccgagcaatcatggtacaaccctgagcggtctgaccgattttgcacgtctgctgggtccgattggtaatcaggccgttcagctggttaccagtttttgtccggcatgtggtgatcagattcgtggtagcgcctttatgcaagaactgaatgccggtggtgataccgttgccggtgttgcatataccgttattcagaccaaatatgatgaagtggttaccccgtatgatagcgcgtttctgactccggcagccagcagccatcaggttcgtaatattctggtgcagaaaggttgtccgattgatctgagcgaacatctggcaattccgtttgatcgtcgtgcactggcctatgttctgaatgccctggatccggcacatccgcgtccggttccgtgtgttccggttgcaccgctgattggcggt

SEQ ID NO: 43 - protein, 319 aa, synthetic construct
MRMSRRAKLRCALPLALVAVVAAFAPAANAQLAVDYSLPKALAAWAANPNGSPPGANDWSCRPSSLHPYPVILVHGTFENQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTLSGLTDFARLLGPIGNAAVQLVTSFCPACGDQIRGSAFMQELNAGGDTVAGVAYTVIQTKYDEVVTPYDSAFLTPAASSHQVRNILVQKGCPIDLSEHLAIPFDRRALAYVLNALAPAHPRPVPCVPVAPLIGG

SEQ ID NO: 44 - DNA, 957 bp, synthetic construct
  source 1..957: mol_type = other DNA
atgcgtatgagccgtcgtgcaaaactgcgttgtgcactgccgctggcactggttgcagttgttgcagcatttgcaccggcagcaaatgcacagctggcagttgattatagcctgccgaaagcactggcagcatgggcagccaatccgaatggtagccctccgggtgccaatgattggagctgtcgtccgagcagcctgcatccgtatccggttattctggttcatggcacctttgaaaatcagcgctttaattggaatgcactggcaccgctgctgaaaaatgcaggttattgtgttttcagcttcaactatggtgcaacccagggcacccagctgaccggtggttttgcaaatggtctggcagcagttcgtgatagcgcacgtcaactggcagcctttgttgatcgtgttcgtgcagcaaccggtgcagcagaagttgatattgttggtcatagccaaggtggtatgatgcctcgttattataccaataaacttggtggtgcaagcaaagttcgtgccctggttggtctgagcccgagcaatcatggtacaaccctgagcggtctgaccgattttgcacgtctgctgggtccgattggtaatgcagccgttcagctggttaccagtttttgtccggcatgtggtgatcagattcgtggtagcgcctttatgcaagaactgaatgccggtggtgataccgttgccggtgttgcatataccgttattcagaccaaatatgatgaagtggttaccccgtatgatagcgcgtttctgactccggcagccagcagccatcaggttcgtaatattctggtgcagaaaggttgtccgattgatctgagcgaacatctggcaattccgtttgatcgtcgtgcactggcctatgttctgaatgccctggcgccggcacatccgcgtccggttccgtgtgttccggttgcaccgctgattggcggt

SEQ ID NO: 45 - protein, 319 aa, synthetic construct
MRMSRRAKLRCALPLALVAVVAAFAPAANAQLAVDYSLPKALAAWAANPNGSPPGANDWSCRPSSLHPYPVILVHGTFENQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTLSGLTDFARLLGPIGNAAVQLVTSFCPACGDQIRGSAFMQELNAGGDTVAGVAYTVIQTKYDEVVTPYDSAFLTPAASSHQVRNILVQKGCPIDLSEHLAIPFDRRALAYVLNALDPAHPRPVCCVPVAPLIGG

SEQ ID NO: 46 - DNA, 957 bp, synthetic construct
  source 1..957: mol_type = other DNA
atgcgtatgagccgtcgtgcaaaactgcgttgtgcactgccgctggcactggttgcagttgttgcagcatttgcaccggcagcaaatgcacagctggcagttgattatagcctgccgaaagcactggcagcatgggcagccaatccgaatggtagccctccgggtgccaatgattggagctgtcgtccgagcagcctgcatccgtatccggttattctggttcatggcacctttgaaaatcagcgctttaattggaatgcactggcaccgctgctgaaaaatgcaggttattgtgttttcagcttcaactatggtgcaacccagggcacccagctgaccggtggttttgcaaatggtctggcagcagttcgtgatagcgcacgtcaactggcagcctttgttgatcgtgttcgtgcagcaaccggtgcagcagaagttgatattgttggtcatagccaaggtggtatgatgcctcgttattataccaataaacttggtggtgcaagcaaagttcgtgccctggttggtctgagcccgagcaatcatggtacaaccctgagcggtctgaccgattttgcacgtctgctgggtccgattggtaatgcagccgttcagctggttaccagtttttgtccggcatgtggtgatcagattcgtggtagcgcctttatgcaagaactgaatgccggtggtgataccgttgccggtgttgcatataccgttattcagaccaaatatgatgaagtggttaccccgtatgatagcgcgtttctgactccggcagccagcagccatcaggttcgtaatattctggtgcagaaaggttgtccgattgatctgagcgaacatctggcaattccgtttgatcgtcgtgcactggcctatgttctgaatgccctggatccggcacatccgcgtccggtttgttgtgttccggttgcaccgctgattggcggt

SEQ ID NO: 47 - protein, 319 aa, synthetic construct
MRMSRRAKLRCALPLALVAVVAAFAPAANAQLAVDYSLPKALAAWAANPNGSPPGANDWSCRPSSLHPYPVILVHGTFENQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTDSGLTDFARLLGPIGNAAVQLVTSFCPACGDQIRGSAFMQELNAGGDTVAGVAYTVIQTKYDEVVTPYDSAFLTPAASSHQVRNILVQKGCPIDLSEHLAIPFDRRALAYVLNALDPAHPRPVPCVPVAPLIGG

SEQ ID NO: 48 - DNA, 957 bp, synthetic construct
  source 1..957: mol_type = other DNA
atgcgtatgagccgtcgtgcaaaactgcgttgtgcactgccgctggcactggttgcagttgttgcagcatttgcaccggcagcaaatgcacagctggcagttgattatagcctgccgaaagcactggcagcatgggcagccaatccgaatggtagccctccgggtgccaatgattggagctgtcgtccgagcagcctgcatccgtatccggttattctggttcatggcacctttgaaaatcagcgctttaattggaatgcactggcaccgctgctgaaaaatgcaggttattgtgttttcagcttcaactatggtgcaacccagggcacccagctgaccggtggttttgcaaatggtctggcagcagttcgtgatagcgcacgtcaactggcagcctttgttgatcgtgttcgtgcagcaaccggtgcagcagaagttgatattgttggtcatagccaaggtggtatgatgcctcgttattataccaataaacttggtggtgcaagcaaagttcgtgccctggttggtctgagcccgagcaatcatggtacaaccgatagcggtctgaccgattttgcacgtctgctgggtccgattggtaatgcagccgttcagctggttaccagtttttgtccggcatgtggtgatcagattcgtggtagcgcctttatgcaagaactgaatgccggtggtgataccgttgccggtgttgcatataccgttattcagaccaaatatgatgaagtggttaccccgtatgatagcgcgtttctgactccggcagccagcagccatcaggttcgtaatattctggtgcagaaaggttgtccgattgatctgagcgaacatctggcaattccgtttgatcgtcgtgcactggcctatgttctgaatgccctggatccggcacatccgcgtccggttccgtgtgttccggttgcaccgctgattggcggt

SEQ ID NO: 49 - protein, 319 aa, synthetic construct
MRMSRRAKLRCALPLALVAVVAAFAPAANAWLAVDYSLPKALAAWAANPNGSPPGANDWSCRPSSLHPYPVILVHGTFENQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTLSGLTDFARLLGPIGNAAVQLVTSFCPACGDQIRGSAFMQELNAGGDTVAGVAYTVIQTKYDEVVTPYDSAFLTPAASSHQVRNILVQKGCPIDLSEHLAIPFDRRALAYVLNALDPAHPRPVPCVPVAPLIGG

SEQ ID NO: 50 - DNA, 957 bp, synthetic construct
  source 1..957: mol_type = other DNA
atgcgtatgagccgtcgtgcaaaactgcgttgtgcactgccgctggcactggttgcagttgttgcagcatttgcaccggcagcaaatgcatggctggcagttgattatagcctgccgaaagcactggcagcatgggcagccaatccgaatggtagccctccgggtgccaatgattggagctgtcgtccgagcagcctgcatccgtatccggttattctggttcatggcacctttgaaaatcagcgctttaattggaatgcactggcaccgctgctgaaaaatgcaggttattgtgttttcagcttcaactatggtgcaacccagggcacccagctgaccggtggttttgcaaatggtctggcagcagttcgtgatagcgcacgtcaactggcagcctttgttgatcgtgttcgtgcagcaaccggtgcagcagaagttgatattgttggtcatagccaaggtggtatgatgcctcgttattataccaataaacttggtggtgcaagcaaagttcgtgccctggttggtctgagcccgagcaatcatggtacaaccctgagcggtctgaccgattttgcacgtctgctgggtccgattggtaatgcagccgttcagctggttaccagtttttgtccggcatgtggtgatcagattcgtggtagcgcctttatgcaagaactgaatgccggtggtgataccgttgccggtgttgcatataccgttattcagaccaaatatgatgaagtggttaccccgtatgatagcgcgtttctgactccggcagccagcagccatcaggttcgtaatattctggtgcagaaaggttgtccgattgatctgagcgaacatctggcaattccgtttgatcgtcgtgcactggcctatgttctgaatgccctggatccggcacatccgcgtccggttccgtgtgttccggttgcaccgctgattggcggt

SEQ ID NO: 51 - protein, 319 aa, synthetic construct
MRMSRRAKLRCALPLALVAVVAAFAPAANAQLAVDYSLPKALAAWAANPNGSPPGANDWSCRPSSLHPYPVILVHGTFENQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTLSGLTDFARLLGPIGNNAVQLVTSFCPACGDQIRGSAFMQELNAGGDTVAGVAYTVIQTKYDEVVTPYDSAFLTPAASSHQVRNILVQKGCPIDLSEHLAIPFDRRALAYVLNALDPAHPRPVPCVPVAPLIGG

SEQ ID NO: 52 - DNA, 957 bp, synthetic construct
  source 1..957: mol_type = other DNA
atgcgtatgagccgtcgtgcaaaactgcgttgtgcactgccgctggcactggttgcagttgttgcagcatttgcaccggcagcaaatgcacagctggcagttgattatagcctgccgaaagcactggcagcatgggcagccaatccgaatggtagccctccgggtgccaatgattggagctgtcgtccgagcagcctgcatccgtatccggttattctggttcatggcacctttgaaaatcagcgctttaattggaatgcactggcaccgctgctgaaaaatgcaggttattgtgttttcagcttcaactatggtgcaacccagggcacccagctgaccggtggttttgcaaatggtctggcagcagttcgtgatagcgcacgtcaactggcagcctttgttgatcgtgttcgtgcagcaaccggtgcagcagaagttgatattgttggtcatagccaaggtggtatgatgcctcgttattataccaataaacttggtggtgcaagcaaagttcgtgccctggttggtctgagcccgagcaatcatggtacaaccctgagcggtctgaccgattttgcacgtctgctgggtccgattggtaataatgccgttcagctggttaccagtttttgtccggcatgtggtgatcagattcgtggtagcgcctttatgcaagaactgaatgccggtggtgataccgttgccggtgttgcatataccgttattcagaccaaatatgatgaagtggttaccccgtatgatagcgcgtttctgactccggcagccagcagccatcaggttcgtaatattctggtgcagaaaggttgtccgattgatctgagcgaacatctggcaattccgtttgatcgtcgtgcactggcctatgttctgaatgccctggatccggcacatccgcgtccggttccgtgtgttccggttgcaccgctgattggcggt

SEQ ID NO: 53 - protein, 319 aa, synthetic construct
MRMSRRAKLRCALPLALVAVVAAFAPAANAQLAVDYSLPKALAAWAANPNGSPPGANDWSCRPSSLHPYPVILVHGTFENQRFNWNALAPLLKNAGYCVFSFNYGATQGTELTGGFANGLAAVRDSARQLAAFVDRVRAATGAAEVDIVGHSQGGMMPRYYTNKLGGASKVRALVGLSPSNHGTTLSGLTDFARLLGPIGNAAVQLVTSFCPACGDQIRGSAFMQELNAGGDTVAGVAYTVIQTKYDEVVTPYDSAFLTPAASSHQVRNILVQKGCPIDLSEHLAIPFDRRALAYVLNALDPAHPRPVPCVPVAPLIGG

SEQ ID NO: 54 - DNA, 957 bp, synthetic construct
  source 1..957: mol_type = other DNA
atgcgtatgagccgtcgtgcaaaactgcgttgtgcactgccgctggcactggttgcagttgttgcagcatttgcaccggcagcaaatgcacagctggcagttgattatagcctgccgaaagcactggcagcatgggcagccaatccgaatggtagccctccgggtgccaatgattggagctgtcgtccgagcagcctgcatccgtatccggttattctggttcatggcacctttgaaaatcagcgctttaattggaatgcactggcaccgctgctgaaaaatgcaggttattgtgttttcagcttcaactatggtgcaacccagggcaccgagctgaccggtggttttgcaaatggtctggcagcagttcgtgatagcgcacgtcaactggcagcctttgttgatcgtgttcgtgcagcaaccggtgcagcagaagttgatattgttggtcatagccaaggtggtatgatgcctcgttattataccaataaacttggtggtgcaagcaaagttcgtgccctggttggtctgagcccgagcaatcatggtacaaccctgagcggtctgaccgattttgcacgtctgctgggtccgattggtaatgcagccgttcagctggttaccagtttttgtccggcatgtggtgatcagattcgtggtagcgcctttatgcaagaactgaatgccggtggtgataccgttgccggtgttgcatataccgttattcagaccaaatatgatgaagtggttaccccgtatgatagcgcgtttctgactccggcagccagcagccatcaggttcgtaatattctggtgcagaaaggttgtccgattgatctgagcgaacatctggcaattccgtttgatcgtcgtgcactggcctatgttctgaatgccctggatccggcacatccgcgtccggttccgtgtgttccggttgcaccgctgattggcggt

SEQ ID NO: 55 - protein, 319 aa, synthetic construct
MRMSRRAKLRCALPLALVAVVAAFAPAANAQLAVDYSLPKALAAWAANPNGSPPGANDWSCRPSSLHPYPVILVHGTFENQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTQSGLTDFARLLGPIGNAAVQLVTSFCPACGDQIRGSAFMQELNAGGDTVAGVAYTVIQTKYDEVVTPYDSAFLTPAASSHQVRNILVQKGCPIDLSEHLAIPFDRRALAYVLNALDPAHPRPVPCVPVAPLIGG

SEQ ID NO: 56 - DNA, 957 bp, synthetic construct
  source 1..957: mol_type = other DNA
atgcgtatgagccgtcgtgcaaaactgcgttgtgcactgccgctggcactggttgcagttgttgcagcatttgcaccggcagcaaatgcacagctggcagttgattatagcctgccgaaagcactggcagcatgggcagccaatccgaatggtagccctccgggtgccaatgattggagctgtcgtccgagcagcctgcatccgtatccggttattctggttcatggcacctttgaaaatcagcgctttaattggaatgcactggcaccgctgctgaaaaatgcaggttattgtgttttcagcttcaactatggtgcaacccagggcacccagctgaccggtggttttgcaaatggtctggcagcagttcgtgatagcgcacgtcaactggcagcctttgttgatcgtgttcgtgcagcaaccggtgcagcagaagttgatattgttggtcatagccaaggtggtatgatgcctcgttattataccaataaacttggtggtgcaagcaaagttcgtgccctggttggtctgagcccgagcaatcatggtacaacccagagcggtctgaccgattttgcacgtctgctgggtccgattggtaatgcagccgttcagctggttaccagtttttgtccggcatgtggtgatcagattcgtggtagcgcctttatgcaagaactgaatgccggtggtgataccgttgccggtgttgcatataccgttattcagaccaaatatgatgaagtggttaccccgtatgatagcgcgtttctgactccggcagccagcagccatcaggttcgtaatattctggtgcagaaaggttgtccgattgatctgagcgaacatctggcaattccgtttgatcgtcgtgcactggcctatgttctgaatgccctggatccggcacatccgcgtccggttccgtgtgttccggttgcaccgctgattggcggt

SEQ ID NO: 57 - protein, 319 aa, synthetic construct
MRMSRRAKLRCALPLALVAVVAAFAPAANAQLAVDYSLPKALAAWAANPNGSPPGANDWSCRPSSLHPYPVILVHGTFENQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTTSGLTDFARLLGPIGNAAVQLVTSFCPACGDQIRGSAFMQELNAGGDTVAGVAYTVIQTKYDEVVTPYDSAFLTPAASSHQVRNILVQKGCPIDLSEHLAIPFDRRALAYVLNALDPAHPRPVPCVPVAPLIGG

SEQ ID NO: 58 - DNA, 957 bp, synthetic construct
  source 1..957: mol_type = other DNA
atgcgtatgagccgtcgtgcaaaactgcgttgtgcactgccgctggcactggttgcagttgttgcagcatttgcaccggcagcaaatgcacagctggcagttgattatagcctgccgaaagcactggcagcatgggcagccaatccgaatggtagccctccgggtgccaatgattggagctgtcgtccgagcagcctgcatccgtatccggttattctggttcatggcacctttgaaaatcagcgctttaattggaatgcactggcaccgctgctgaaaaatgcaggttattgtgttttcagcttcaactatggtgcaacccagggcacccagctgaccggtggttttgcaaatggtctggcagcagttcgtgatagcgcacgtcaactggcagcctttgttgatcgtgttcgtgcagcaaccggtgcagcagaagttgatattgttggtcatagccaaggtggtatgatgcctcgttattataccaataaacttggtggtgcaagcaaagttcgtgccctggttggtctgagcccgagcaatcatggtacaaccaccagcggtctgaccgattttgcacgtctgctgggtccgattggtaatgcagccgttcagctggttaccagtttttgtccggcatgtggtgatcagattcgtggtagcgcctttatgcaagaactgaatgccggtggtgataccgttgccggtgttgcatataccgttattcagaccaaatatgatgaagtggttaccccgtatgatagcgcgtttctgactccggcagccagcagccatcaggttcgtaatattctggtgcagaaaggttgtccgattgatctgagcgaacatctggcaattccgtttgatcgtcgtgcactggcctatgttctgaatgccctggatccggcacatccgcgtccggttccgtgtgttccggttgcaccgctgattggcggt

SEQ ID NO: 59 - protein, 319 aa, synthetic construct
MRCSRRAKLRCALPLALVAVVAAFAPAANAQLAVDYSLPKALAAWAANPNGSPPGANDWSCRPSSLHPYPVILVHGTFENQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTLSGLTDFARLLGPIGNAAVQLVTSFCPACGDQIRGSAFMQELNAGGDTVAGVAYTVIQTKYDEVVTPYDSAFLTPAASSHQVRNILVQKGCPIDLSEHLAIPFDRRALAYVLNALDPAHPRPVPCVPVAPLIGG

SEQ ID NO: 60 - DNA, 957 bp, synthetic construct
  source 1..957: mol_type = other DNA
atgcgttgtagccgtcgtgcaaaactgcgttgtgcactgccgctggcactggttgcagttgttgcagcatttgcaccggcagcaaatgcacagctggcagttgattatagcctgccgaaagcactggcagcatgggcagccaatccgaatggtagccctccgggtgccaatgattggagctgtcgtccgagcagcctgcatccgtatccggttattctggttcatggcacctttgaaaatcagcgctttaattggaatgcactggcaccgctgctgaaaaatgcaggttattgtgttttcagcttcaactatggtgcaacccagggcacccagctgaccggtggttttgcaaatggtctggcagcagttcgtgatagcgcacgtcaactggcagcctttgttgatcgtgttcgtgcagcaaccggtgcagcagaagttgatattgttggtcatagccaaggtggtatgatgcctcgttattataccaataaacttggtggtgcaagcaaagttcgtgccctggttggtctgagcccgagcaatcatggtacaaccctgagcggtctgaccgattttgcacgtctgctgggtccgattggtaatgcagccgttcagctggttaccagtttttgtccggcatgtggtgatcagattcgtggtagcgcctttatgcaagaactgaatgccggtggtgataccgttgccggtgttgcatataccgttattcagaccaaatatgatgaagtggttaccccgtatgatagcgcgtttctgactccggcagccagcagccatcaggttcgtaatattctggtgcagaaaggttgtccgattgatctgagcgaacatctggcaattccgtttgatcgtcgtgcactggcctatgttctgaatgccctggatccggcacatccgcgtccggttccgtgtgttccggttgcaccgctgattggcggt

SEQ ID NO: 61 - protein, 319 aa, synthetic construct; identical to SEQ ID NO: 49

SEQ ID NO: 62 - DNA, 957 bp, synthetic construct; identical to SEQ ID NO: 50
  source 1..957: mol_type = other DNA

SEQ ID NO: 63 - protein, 319 aa, synthetic construct
MRMSRRAKLRCALPLALVAVVAAFAPAAHAQLAVDYSLPKALAAWAANPNGSPPGANDWSCRPSSLHPYPVILVHGTFENQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTLSGLTDFARLLGPIGNAAVQLVTSFCPACGDQIRGSAFMQELNAGGDTVAGVAYTVIQTKYDEVVTPYDSAFLTPAASSHQVRNILVQKGCPIDLSEHLAIPFDRRALAYVLNALDPAHPRPVPCVPVAPLIGG

SEQ ID NO: 64 - DNA, 957 bp, synthetic construct
  source 1..957: mol_type = other DNA
atgcgtatgagccgtcgtgcaaaactgcgttgtgcactgccgctggcactggttgcagttgttgcagcatttgcaccggcagcacatgcacagctggcagttgattatagcctgccgaaagcactggcagcatgggcagccaatccgaatggtagccctccgggtgccaatgattggagctgtcgtccgagcagcctgcatccgtatccggttattctggttcatggcacctttgaaaatcagcgctttaattggaatgcactggcaccgctgctgaaaaatgcaggttattgtgttttcagcttcaactatggtgcaacccagggcacccagctgaccggtggttttgcaaatggtctggcagcagttcgtgatagcgcacgtcaactggcagcctttgttgatcgtgttcgtgcagcaaccggtgcagcagaagttgatattgttggtcatagccaaggtggtatgatgcctcgttattataccaataaacttggtggtgcaagcaaagttcgtgccctggttggtctgagcccgagcaatcatggtacaaccctgagcggtctgaccgattttgcacgtctgctgggtccgattggtaatgcagccgttcagctggttaccagtttttgtccggcatgtggtgatcagattcgtggtagcgcctttatgcaagaactgaatgccggtggtgataccgttgccggtgttgcatataccgttattcagaccaaatatgatgaagtggttaccccgtatgatagcgcgtttctgactccggcagccagcagccatcaggttcgtaatattctggtgcagaaaggttgtccgattgatctgagcgaacatctggcaattccgtttgatcgtcgtgcactggcctatgttctgaatgccctggatccggcacatccgcgtccggttccgtgtgttccggttgcaccgctgattggcggt

SEQ ID NO: 65 - protein, 319 aa, synthetic construct
MRMSRRAELRCALPLALVAVVAAFAPAANAQLAVDYSLPKALAAWAANPNGSPPGANDWSCRPSSLHPYPVILVHGTFENQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTLSGLTDFARLLGPIGNAAVQLVTSFCPACGDQIRGSAFMQELNAGGDTVAGVAYTVIQTKYDEVVTPYDSAFLTPAASSHQVRNILVQKGCPIDLSEHLAIPFDRRALAYVLNALDPAHPRPVPCVPVAPLIGG

SEQ ID NO: 66 - DNA, 957 bp, synthetic construct
  source 1..957: mol_type = other DNA
atgcgtatgagccgtcgtgcagagctgcgttgtgcactgccgctggcactggttgcagttgttgcagcatttgcaccggcagcaaatgcacagctggcagttgattatagcctgccgaaagcactggcagcatgggcagccaatccgaatggtagccctccgggtgccaatgattggagctgtcgtccgagcagcctgcatccgtatccggttattctggttcatggcacctttgaaaatcagcgctttaattggaatgcactggcaccgctgctgaaaaatgcaggttattgtgttttcagcttcaactatggtgcaacccagggcacccagctgaccggtggttttgcaaatggtctggcagcagttcgtgatagcgcacgtcaactggcagcctttgttgatcgtgttcgtgcagcaaccggtgcagcagaagttgatattgttggtcatagccaaggtggtatgatgcctcgttattataccaataaacttggtggtgcaagcaaagttcgtgccctggttggtctgagcccgagcaatcatggtacaaccctgagcggtctgaccgattttgcacgtctgctgggtccgattggtaatgcagccgttcagctggttaccagtttttgtccggcatgtggtgatcagattcgtggtagcgcctttatgcaagaactgaatgccggtggtgataccgttgccggtgttgcatataccgttattcagaccaaatatgatgaagtggttaccccgtatgatagcgcgtttctgactccggcagccagcagccatcaggttcgtaatattctggtgcagaaaggttgtccgattgatctgagcgaacatctggcaattccgtttgatcgtcgtgcactggcctatgttctgaatgccctggatccggcacatccgcgtccggttccgtgtgttccggttgcaccgctgattggcggt

SEQ ID NO: 67 - protein, 319 aa, synthetic construct
MRMSRRAKLRCALPLALVAVVAAFAPAANAQLAVDYSLPKALAAWAANPNGSPPGANDWSCRPSSLHPYPVILVHGTFENQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTHSGLTDFARLLGPIGNAAVQLVTSFCPACGDQIRGSAFMQELNAGGDTVAGVAYTVIQTKYDEVVTPYDSAFLTPAASSHQVRNILVQKGCPIDLSEHLAIPFDRRALAYVLNALDPAHPRPVPCVPVAPLIGG

SEQ ID NO: 68 - DNA, 957 bp, synthetic construct
  source 1..957: mol_type = other DNA
atgcgtatgagccgtcgtgcaaaactgcgttgtgcactgccgctggcactggttgcagttgttgcagcatttgcaccggcagcaaatgcacagctggcagttgattatagcctgccgaaagcactggcagcatgggcagccaatccgaatggtagccctccgggtgccaatgattggagctgtcgtccgagcagcctgcatccgtatccggttattctggttcatggcacctttgaaaatcagcgctttaattggaatgcactggcaccgctgctgaaaaatgcaggttattgtgttttcagcttcaactatggtgcaacccagggcacccagctgaccggtggttttgcaaatggtctggcagcagttcgtgatagcgcacgtcaactggcagcctttgttgatcgtgttcgtgcagcaaccggtgcagcagaagttgatattgttggtcatagccaaggtggtatgatgcctcgttattataccaataaacttggtggtgcaagcaaagttcgtgccctggttggtctgagcccgagcaatcatggtacaacccatagcggtctgaccgattttgcacgtctgctgggtccgattggtaatgcagccgttcagctggttaccagtttttgtccggcatgtggtgatcagattcgtggtagcgcctttatgcaagaactgaatgccggtggtgataccgttgccggtgttgcatataccgttattcagaccaaatatgatgaagtggttaccccgtatgatagcgcgtttctgactccggcagccagcagccatcaggttcgtaatattctggtgcagaaaggttgtccgattgatctgagcgaacatctggcaattccgtttgatcgtcgtgcactggcctatgttctgaatgccctggatccggcacatccgcgtccggttccgtgtgttccggttgcaccgctgattggcggt

SEQ ID NO: 69 - protein, 319 aa, synthetic construct
MRMSRRAKLRCALPLALVAVVAAFAPAANAQLAVDYSLPKALAAWAANPNGSPPGANDWSCRPSSLHPYPVILVHGTFINQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTLSGLTDFARLLGPIGNAAVQLVTSFCPACGDQIRGSAFMQELNAGGDTVAGVAYTVIQTKYDEVVTPYDSAFLTPAASSHQVRNILVQKGCPIDLSEHLAIPFDRRALAYVLNALDPAHPRPVPCVPVAPLIGG

SEQ ID NO: 70 - DNA, 957 bp, synthetic construct
  source 1..957: mol_type = other DNA
atgcgtatgagccgtcgtgcaaaactgcgttgtgcactgccgctggcactggttgcagttgttgcagcatttgcaccggcagcaaatgcacagctggcagttgattatagcctgccgaaagcactggcagcatgggcagccaatccgaatggtagccctccgggtgccaatgattggagctgtcgtccgagcagcctgcatccgtatccggttattctggttcatggcacctttattaatcagcgctttaattggaatgcactggcaccgctgctgaaaaatgcaggttattgtgttttcagcttcaactatggtgcaacccagggcacccagctgaccggtggttttgcaaatggtctggcagcagttcgtgatagcgcacgtcaactggcagcctttgttgatcgtgttcgtgcagcaaccggtgcagcagaagttgatattgttggtcatagccaaggtggtatgatgcctcgttattataccaataaacttggtggtgcaagcaaagttcgtgccctggttggtctgagcccgagcaatcatggtacaaccctgagcggtctgaccgattttgcacgtctgctgggtccgattggtaatgcagccgttcagctggttaccagtttttgtccggcatgtggtgatcagattcgtggtagcgcctttatgcaagaactgaatgccggtggtgataccgttgccggtgttgcatataccgttattcagaccaaatatgatgaagtggttaccccgtatgatagcgcgtttctgactccggcagccagcagccatcaggttcgtaatattctggtgcagaaaggttgtccgattgatctgagcgaacatctggcaattccgtttgatcgtcgtgcactggcctatgttctgaatgccctggatccggcacatccgcgtccggttccgtgtgttccggttgcaccgctgattggcggt

SEQ ID NO: 71 - protein, 319 aa, synthetic construct
MRMSRRAKLRCALPLALVAVVAAFAPAANAQLAVDYSLPKALAAWAANPNGSPPGANDWSCRPSSLHPYPVILVHGTFWNQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTLSGLTDFARLLGPIGNAAVQLVTSFCPACGDQIRGSAFMQELNAGGDTVAGVAYTVIQTKYDEVVTPYDSAFLTPAASSHQVRNILVQKGCPIDLSEHLAIPFDRRALAYVLNALDPAHPRPVPCVPVAPLIGG

SEQ ID NO: 72 - DNA, 957 bp, synthetic construct
  source 1..957: mol_type = other DNA
atgcgtatgagccgtcgtgcaaaactgcgttgtgcactgccgctggcactggttgcagttgttgcagcatttgcaccggcagcaaatgcacagctggcagttgattatagcctgccgaaagcactggcagcatgggcagccaatccgaatggtagccctccgggtgccaatgattggagctgtcgtccgagcagcctgcatccgtatccggttattctggttcatggcaccttttggaatcagcgctttaattggaatgcactggcaccgctgctgaaaaatgcaggttattgtgttttcagcttcaactatggtgcaacccagggcacccagctgaccggtggttttgcaaatggtctggcagcagttcgtgatagcgcacgtcaactggcagcctttgttgatcgtgttcgtgcagcaaccggtgcagcagaagttgatattgttggtcatagccaaggtggtatgatgcctcgttattataccaataaacttggtggtgcaagcaaagttcgtgccctggttggtctgagcccgagcaatcatggtacaaccctgagcggtctgaccgattttgcacgtctgctgggtccgattggtaatgcagccgttcagctggttaccagtttttgtccggcatgtggtgatcagattcgtggtagcgcctttatgcaagaactgaatgccggtggtgataccgttgccggtgttgcatataccgttattcagaccaaatatgatgaagtggttaccccgtatgatagcgcgtttctgactccggcagccagcagccatcaggttcgtaatattctggtgcagaaaggttgtccgattgatctgagcgaacatctggcaattccgtttgatcgtcgtgcactggcctatgttctgaatgccctggatccggcacatccgcgtccggttccgtgtgttccggttgcaccgctgattggcggt

SEQ ID NO: 73 - protein, 319 aa, synthetic construct
MRMSRRAKLRCALPLATVAVVAAFAPAANAQLAVDYSLPKALAAWAANPNGSPPGANDWSCRPSSLHPYPVILVHGTFENQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTLSGLTDFARLLGPIGNAAVQLVTSFCPACGDQIRGSAFMQELNAGGDTVAGVAYTVIQTKYDEVVTPYDSAFLTPAASSHQVRNILVQKGCPIDLSEHLAIPFDRRALAYVLNALDPAHPRPVPCVPVAPLIGG

SEQ ID NO: 74 - DNA, 957 bp, synthetic construct
  source 1..957: mol_type = other DNA
atgcgtatgagccgtcgtgcaaaactgcgttgtgcactgccgctggcaacggttgcagttgttgcagcatttgcaccggcagcaaatgcacagctggcagttgattatagcctgccgaaagcactggcagcatgggcagccaatccgaatggtagccctccgggtgccaatgattggagctgtcgtccgagcagcctgcatccgtatccggttattctggttcatggcacctttgaaaatcagcgctttaattggaatgcactggcaccgctgctgaaaaatgcaggttattgtgttttcagcttcaactatggtgcaacccagggcacccagctgaccggtggttttgcaaatggtctggcagcagttcgtgatagcgcacgtcaactggcagcctttgttgatcgtgttcgtgcagcaaccggtgcagcagaagttgatattgttggtcatagccaaggtggtatgatgcctcgttattataccaataaacttggtggtgcaagcaaagttcgtgccctggttggtctgagcccgagcaatcatggtacaaccctgagcggtctgaccgattttgcacgtctgctgggtccgattggtaatgcagccgttcagctggttaccagtttttgtccggcatgtggtgatcagattcgtggtagcgcctttatgcaagaactgaatgccggtggtgataccgttgccggtgttgcatataccgttattcagaccaaatatgatgaagtggttaccccgtatgatagcgcgtttctgactccggcagccagcagccatcaggttcgtaatattctggtgcagaaaggttgtccgattgatctgagcgaacatctggcaattccgtttgatcgtcgtgcactggcctatgttctgaatgccctggatccggcacatccgcgtccggttccgtgtgttccggttgcaccgctgattggcggt

SEQ ID NO: 75 - protein, 319 aa, synthetic construct
MRMSRRAKLRCALPLALCAVVAAFAPAANAQLAVDYSLPKALAAWAANPNGSPPGANDWSCRPSSLHPYPVILVHGTFENQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTLSGLTDFARLLGPIGNAAVQLVTSFCPACGDQIRGSAFMQELNAGGDTVAGVAYTVIQTKYDEVVTPYDSAFLTPAASSHQVRNILVQKGCPIDLSEHLAIPFDRRALAYVLNALDPAHPRPVPCVPVAPLIGG

SEQ ID NO: 76 - DNA, 957 bp, synthetic construct
  source 1..957: mol_type = other DNA
atgcgtatgagccgtcgtgcaaaactgcgttgtgcactgccgctggcactgtgtgcagttgttgcagcatttgcaccggcagcaaatgcacagctggcagttgattatagcctgccgaaagcactggcagcatgggcagccaatccgaatggtagccctccgggtgccaatgattggagctgtcgtccgagcagcctgcatccgtatccggttattctggttcatggcacctttgaaaatcagcgctttaattggaatgcactggcaccgctgctgaaaaatgcaggttattgtgttttcagcttcaactatggtgcaacccagggcacccagctgaccggtggttttgcaaatggtctggcagcagttcgtgatagcgcacgtcaactggcagcctttgttgatcgtgttcgtgcagcaaccggtgcagcagaagttgatattgttggtcatagccaaggtggtatgatgcctcgttattataccaataaacttggtggtgcaagcaaagttcgtgccctggttggtctgagcccgagcaatcatggtacaaccctgagcggtctgaccgattttgcacgtctgctgggtccgattggtaatgcagccgttcagctggttaccagtttttgtccggcatgtggtgatcagattcgtggtagcgcctttatgcaagaactgaatgccggtggtgataccgttgccggtgttgcatataccgttattcagaccaaatatgatgaagtggttaccccgtatgatagcgcgtttctgactccggcagccagcagccatcaggttcgtaatattctggtgcagaaaggttgtccgattgatctgagcgaacatctggcaattccgtttgatcgtcgtgcactggcctatgttctgaatgccctggatccggcacatccgcgtccggttccgtgtgttccggttgcaccgctgattggcggt

SEQ ID NO: 77 - protein, 319 aa, synthetic construct
MRMSRRAKLRCALPLALVAVVAAFAPAANAQLAVDYSLPKALAAWAANPNGSPPGANDWSCRPSSLHPYPVILVHGTFENQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTPSGLTDFARLLGPIGNAAVQLVTSFCPACGDQIRGSAFMQELNAGGDTVAGVAYTVIQTKYDEVVTPYDSAFLTPAASSHQVRNILVQKGCPIDLSEHLAIPFDRRALAYVLNALDPAHPRPVPCVPVAPLIGG

SEQ ID NO: 78 - DNA, 957 bp, synthetic construct
  source 1..957: mol_type = other DNA
atgcgtatgagccgtcgtgcaaaactgcgttgtgcactgccgctggcactggttgcagttgttgcagcatttgcaccggcagcaaatgcacagctggcagttgattatagcctgccgaaagcactggcagcatgggcagccaatccgaatggtagccctccgggtgccaatgattggagctgtcgtccgagcagcctgcatccgtatccggttattctggttcatggcacctttgaaaatcagcgctttaattggaatgcactggcaccgctgctgaaaaatgcaggttattgtgttttcagcttcaactatggtgcaacccagggcacccagctgaccggtggttttgcaaatggtctggcagcagttcgtgatagcgcacgtcaactggcagcctttgttgatcgtgttcgtgcagcaaccggtgcagcagaagttgatattgttggtcatagccaaggtggtatgatgcctcgttattataccaataaacttggtggtgcaagcaaagttcgtgccctggttggtctgagcccgagcaatcatggtacaacccctagcggtctgaccgattttgcacgtctgctgggtccgattggtaatgcagccgttcagctggttaccagtttttgtccggcatgtggtgatcagattcgtggtagcgcctttatgcaagaactgaatgccggtggtgataccgttgccggtgttgcatataccgttattcagaccaaatatgatgaagtggttaccccgtatgatagcgcgtttctgactccggcagccagcagccatcaggttcgtaatattctggtgcagaaaggttgtccgattgatctgagcgaacatctggcaattccgtttgatcgtcgtgcactggcctatgttctgaatgccctggatccggcacatccgcgtccggttccgtgtgttccggttgcaccgctgattggcggt

SEQ ID NO: 79 - protein, 319 aa, synthetic construct
MRMSRRAKLRCALPLALVAVVAAFAPAANAQLAVDYSLPKALAAWAANPNGSPPGANDWSCRPSSLHPYPVILVHGTFENQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTLSGLTDFARLLGPIGNAAVQLVTSFCPACGDQIRGSAFMQELNAGGDTVAGVAYTVIQTKYDEVVTPYDSAFLTPAASSHQVRNILVQKGCPIDLSEHLAIPFDRRALAYVLNALDPAHPRPVPCVQVAPLIGG

SEQ ID NO: 80 - DNA, 957 bp, synthetic construct
  source 1..957: mol_type = other DNA
atgcgtatgagccgtcgtgcaaaactgcgttgtgcactgccgctggcactggttgcagttgttgcagcatttgcaccggcagcaaatgcacagctggcagttgattatagcctgccgaaagcactggcagcatgggcagccaatccgaatggtagccctccgggtgccaatgattggagctgtcgtccgagcagcctgcatccgtatccggttattctggttcatggcacctttgaaaatcagcgctttaattggaatgcactggcaccgctgctgaaaaatgcaggttattgtgttttcagcttcaactatggtgcaacccagggcacccagctgaccggtggttttgcaaatggtctggcagcagttcgtgatagcgcacgtcaactggcagcctttgttgatcgtgttcgtgcagcaaccggtgcagcagaagttgatattgttggtcatagccaaggtggtatgatgcctcgttattataccaataaacttggtggtgcaagcaaagttcgtgccctggttggtctgagcccgagcaatcatggtacaaccctgagcggtctgaccgattttgcacgtctgctgggtccgattggtaatgcagccgttcagctggttaccagtttttgtccggcatgtggtgatcagattcgtggtagcgcctttatgcaagaactgaatgccggtggtgataccgttgccggtgttgcatataccgttattcagaccaaatatgatgaagtggttaccccgtatgatagcgcgtttctgactccggcagccagcagccatcaggttcgtaatattctggtgcagaaaggttgtccgattgatctgagcgaacatctggcaattccgtttgatcgtcgtgcactggcctatgttctgaatgccctggatccggcacatccgcgtccggttccgtgtgttcaggttgcaccgctgattggcggt

SEQ ID NO: 81 - protein, 319 aa, synthetic construct
MRMSRRAKLRCALPLALVAVVAAFAPAANAQLAVDYSLPMALAAWAANPNGSPPGANDWSCRPSSLHPYPVILVHGTFENQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTLSGLTDFARLLGPIGNAAVQLVTSFCPACGDQIRGSAFMQELNAGGDTVAGVAYTVIQTKYDEVVTPYDSAFLTPAASSHQVRNILVQKGCPIDLSEHLAIPFDRRALAYVLNALDPAHPRPVPCVPVAPLIGG

SEQ ID NO: 82 - DNA, 957 bp, synthetic construct
  source 1..957: mol_type = other DNA
atgcgtatgagccgtcgtgcaaaactgcgttgtgcactgccgctggcactggttgcagttgttgcagcatttgcaccggcagcaaatgcacagctggcagttgattatagcctgccgatggcactggcagcatgggcagccaatccgaatggtagccctccgggtgccaatgattggagctgtcgtccgagcagcctgcatccgtatccggttattctggttcatggcacctttgaaaatcagcgctttaattggaatgcactggcaccgctgctgaaaaatgcaggttattgtgttttcagcttcaactatggtgcaacccagggcacccagctgaccggtggttttgcaaatggtctggcagcagttcgtgatagcgcacgtcaactggcagcctttgttgatcgtgttcgtgcagcaaccggtgcagcagaagttgatattgttggtcatagccaaggtggtatgatgcctcgttattataccaataaacttggtggtgcaagcaaagttcgtgccctggttggtctgagcccgagcaatcatggtacaaccctgagcggtctgaccgattttgcacgtctgctgggtccgattggtaatgcagccgttcagctggttaccagtttttgtccggcatgtggtgatcagattcgtggtagcgcctttatgcaagaactgaatgccggtggtgataccgttgccggtgttgcatataccgttattcagaccaaatatgatgaagtggttaccccgtatgatagcgcgtttctgactccggcagccagcagccatcaggttcgtaatattctggtgcagaaaggttgtccgattgatctgagcgaacatctggcaattccgtttgatcgtcgtgcactggcctatgttctgaatgccctggatccggcacatccgcgtccggttccgtgtgttccggttgcaccgctgattggcggt

SEQ ID NO: 83 - protein, 319 aa, synthetic construct
MRMSRRAKLRCALPLALVAVVAAFAPAANAQLAVDYSLPKALAAWAANPNGSPPGANDWSCRPSSLHPYPVILVHGTFENQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTLSGLTDFARLLGPIGNAAVQLVTSFCPACGDQIRGSAFMQELNAGGDTVAGVAYTVIQTKYDEVVTPYDSAFLTPAASSHQVRNILVQKGCPIDLSEHLAIPFDRRALAYVLNALDPAHPRPVPCVKVAPLIGG

SEQ ID NO: 84 - DNA, 957 bp, synthetic construct
  source 1..957: mol_type = other DNA
atgcgtatgagccgtcgtgcaaaactgcgttgtgcactgccgctggcactggttgcagttgttgcagcatttgcaccggcagcaaatgcacagctggcagttgattatagcctgccgaaagcactggcagcatgggcagccaatccgaatggtagccctccgggtgccaatgattggagctgtcgtccgagcagcctgcatccgtatccggttattctggttcatggcacctttgaaaatcagcgctttaattggaatgcactggcaccgctgctgaaaaatgcaggttattgtgttttcagcttcaactatggtgcaacccagggcacccagctgaccggtggttttgcaaatggtctggcagcagttcgtgatagcgcacgtcaactggcagcctttgttgatcgtgttcgtgcagcaaccggtgcagcagaagttgatattgttggtcatagccaaggtggtatgatgcctcgttattataccaataaacttggtggtgcaagcaaagttcgtgccctggttggtctgagcccgagcaatcatggtacaaccctgagcggtctgaccgattttgcacgtctgctgggtccgattggtaatgcagccgttcagctggttaccagtttttgtccggcatgtggtgatcagattcgtggtagcgcctttatgcaagaactgaatgccggtggtgataccgttgccggtgttgcatataccgttattcagaccaaatatgatgaagtggttaccccgtatgatagcgcgtttctgactccggcagccagcagccatcaggttcgtaatattctggtgcagaaaggttgtccgattgatctgagcgaacatctggcaattccgtttgatcgtcgtgcactggcctatgttctgaatgccctggatccggcacatccgcgtccggttccgtgtgttaaggttgcaccgctgattggcggt

SEQ ID NO: 85 - protein, 319 aa, synthetic construct
MRMSRRAKLRCALPLALVAVVAAFAPAANAQLAVDYSLPKALAAWAANPNGSPPGANDWSCRPSSLHPYPVILVHGTFENQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTCSGLTDFARLLGPIGNAAVQLVTSFCPACGDQIRGSAFMQELNAGGDTVAGVAYTVIQTKYDEVVTPYDSAFLTPAASSHQVRNILVQKGCPIDLSEHLAIPFDRRALAYVLNALDPAHPRPVPCVPVAPLIGG

SEQ ID NO: 86 - DNA, 957 bp, synthetic construct
  source 1..957: mol_type = other DNA
atgcgtatgagccgtcgtgcaaaactgcgttgtgcactgccgctggcactggttgcagttgttgcagcatttgcaccggcagcaaatgcacagctggcagttgattatagcctgccgaaagcactggcagcatgggcagccaatccgaatggtagccctccgggtgccaatgattggagctgtcgtccgagcagcctgcatccgtatccggttattctggttcatggcacctttgaaaatcagcgctttaattggaatgcactggcaccgctgctgaaaaatgcaggttattgtgttttcagcttcaactatggtgcaacccagggcacccagctgaccggtggttttgcaaatggtctggcagcagttcgtgatagcgcacgtcaactggcagcctttgttgatcgtgttcgtgcagcaaccggtgcagcagaagttgatattgttggtcatagccaaggtggtatgatgcctcgttattataccaataaacttggtggtgcaagcaaagttcgtgccctggttggtctgagcccgagcaatcatggtacaacctgcagcggtctgaccgattttgcacgtctgctgggtccgattggtaatgcagccgttcagctggttaccagtttttgtccggcatgtggtgatcagattcgtggtagcgcctttatgcaagaactgaatgccggtggtgataccgttgccggtgttgcatataccgttattcagaccaaatatgatgaagtggttaccccgtatgatagcgcgtttctgactccggcagccagcagccatcaggttcgtaatattctggtgcagaaaggttgtccgattgatctgagcgaacatctggcaattccgtttgatcgtcgtgcactggcctatgttctgaatgccctggatccggcacatccgcgtccggttccgtgtgttccggttgcaccgctgattggcggt

SEQ ID NO: 87 - protein, 319 aa, synthetic construct
MRMSRRAKLRCALPLALVAVVAAFAPAANAQLAVDYSLPKALAAWAANPNGSPPGANDWSCRPSSLHPYPVILVHGTFENQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTLSGLTDFARLLGPIGNAAVQLVTSFCPACGDQIRGSAFMQELNAGGDTVAGVAYTVIQTKYDEVVTPYDSAFLTPAASSHQVRNILVQKGCPIDASEHLAIPFDRRALAYVLNALDPAHPRPVPCVPVAPLIGG

SEQ ID NO: 88 - DNA, 957 bp, synthetic construct
  source 1..957: mol_type = other DNA
atgcgtatgagccgtcgtgcaaaactgcgttgtgcactgccgctggcactggttgcagttgttgcagcatttgcaccggcagcaaatgcacagctggcagttgattatagcctgccgaaagcactggcagcatgggcagccaatccgaatggtagccctccgggtgccaatgattggagctgtcgtccgagcagcctgcatccgtatccggttattctggttcatggcacctttgaaaatcagcgctttaattggaatgcactggcaccgctgctgaaaaatgcaggttattgtgttttcagcttcaactatggtgcaacccagggcacccagctgaccggtggttttgcaaatggtctggcagcagttcgtgatagcgcacgtcaactggcagcctttgttgatcgtgttcgtgcagcaaccggtgcagcagaagttgatattgttggtcatagccaaggtggtatgatgcctcgttattataccaataaacttggtggtgcaagcaaagttcgtgccctggttggtctgagcccgagcaatcatggtacaaccctgagcggtctgaccgattttgcacgtctgctgggtccgattggtaatgcagccgttcagctggttaccagtttttgtccggcatgtggtgatcagattcgtggtagcgcctttatgcaagaactgaatgccggtggtgataccgttgccggtgttgcatataccgttattcagaccaaatatgatgaagtggttaccccgtatgatagcgcgtttctgactccggcagccagcagccatcaggttcgtaatattctggtgcagaaaggttgtccgattgatgctagcgaacatctggcaattccgtttgatcgtcgtgcactggcctatgttctgaatgccctggatccggcacatccgcgtccggttccgtgtgttccggttgcaccgctgattggcggt

SEQ ID NO: 89 - protein, 319 aa, synthetic construct
MRMLRRAKLRCALPLALVAVVAAFAPAANAQLAVDYSLPKALAAWAANPNGSPPGANDWSCRPSSLHPYPVILVHGTFENQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTLSGLTDFARLLGPIGNAAVQLVTSFCPACGDQIRGSAFMQELNAGGDTVAGVAYTVIQTKYDEVVTPYDSAFLTPAASSHQVRNILVQKGCPIDLSEHLAIPFDRRALAYVLNALDPAHPRPVPCVPVAPLIGG

SEQ ID NO: 90 - DNA, 957 bp, synthetic construct
  source 1..957: mol_type = other DNA
atgcgtatgctgcgtcgtgcaaaactgcgttgtgcactgccgctggcactggttgcagttgttgcagcatttgcaccggcagcaaatgcacagctggcagttgattatagcctgccgaaagcactggcagcatgggcagccaatccgaatggtagccctccgggtgccaatgattggagctgtcgtccgagcagcctgcatccgtatccggttattctggttcatggcacctttgaaaatcagcgctttaattggaatgcactggcaccgctgctgaaaaatgcaggttattgtgttttcagcttcaactatggtgcaacccagggcacccagctgaccggtggttttgcaaatggtctggcagcagttcgtgatagcgcacgtcaactggcagcctttgttgatcgtgttcgtgcagcaaccggtgcagcagaagttgatattgttggtcatagccaaggtggtatgatgcctcgttattataccaataaacttggtggtgcaagcaaagttcgtgccctggttggtctgagcccgagcaatcatggtacaaccctgagcggtctgaccgattttgcacgtctgctgggtccgattggtaatgcagccgttcagctggttaccagtttttgtccggcatgtggtgatcagattcgtggtagcgcctttatgcaagaactgaatgccggtggtgataccgttgccggtgttgcatataccgttattcagaccaaatatgatgaagtggttaccccgtatgatagcgcgtttctgactccggcagccagcagccatcaggttcgtaatattctggtgcagaaaggttgtccgattgatctgagcgaacatctggcaattccgtttgatcgtcgtgcactggcctatgttctgaatgccctggatccggcacatccgcgtccggttccgtgtgttccggttgcaccgctgattggcggt

SEQ ID NO: 91 - protein, 319 aa, synthetic construct
MRMSRRAKLRCALPLALSAVVAAFAPAANAQLAVDYSLPKALAAWAANPNGSPPGANDWSCRPSSLHPYPVILVHGTFENQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTLSGLTDFARLLGPIGNAAVQLVTSFCPACGDQIRGSAFMQELNAGGDTVAGVAYTVIQTKYDEVVTPYDSAFLTPAASSHQVRNILVQKGCPIDLSEHLAIPFDRRALAYVLNALDPAHPRPVPCVPVAPLIGG

SEQ ID NO: 92 - DNA, 957 bp, synthetic construct
  source 1..957: mol_type = other DNA
atgcgtatgagccgtcgtgcaaaactgcgttgtgcactgccgctggcactgtcggcagttgttgcagcatttgcaccggcagcaaatgcacagctggcagttgattatagcctgccgaaagcactggcagcatgggcagccaatccgaatggtagccctccgggtgccaatgattggagctgtcgtccgagcagcctgcatccgtatccggttattctggttcatggcacctttgaaaatcagcgctttaattggaatgcactggcaccgctgctgaaaaatgcaggttattgtgttttcagcttcaactatggtgcaacccagggcacccagctgaccggtggttttgcaaatggtctggcagcagttcgtgatagcgcacgtcaactggcagcctttgttgatcgtgttcgtgcagcaaccggtgcagcagaagttgatattgttggtcatagccaaggtggtatgatgcctcgttattataccaataaacttggtggtgcaagcaaagttcgtgccctggttggtctgagcccgagcaatcatggtacaaccctgagcggtctgaccgattttgcacgtctgctgggtccgattggtaatgcagccgttcagctggttaccagtttttgtccggcatgtggtgatcagattcgtggtagcgcctttatgcaagaactgaatgccggtggtgataccgttgccggtgttgcatataccgttattcagaccaaatatgatgaagtggttaccccgtatgatagcgcgtttctgactccggcagccagcagccatcaggttcgtaatattctggtgcagaaaggttgtccgattgatctgagcgaacatctggcaattccgtttgatcgtcgtgcactggcctatgttctgaatgccctggatccggcacatccgcgtccggttccgtgtgttccggttgcaccgctgattggcggt

SEQ ID NO: 93 - protein, 319 aa, synthetic construct
MRMSRRAKLRCALPLALVAVVAAFAPAANAQLAVDYSLPKALAAWAANPNGSPPGANDWSCRPSSLHPYPVILVHGTFENQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTKSGLTDFARLLGPIGNAAVQLVTSFCPACGDQIRGSAFMQELNAGGDTVAGVAYTVIQTKYDEVVTPYDSAFLTPAASSHQVRNILVQKGCPIDLSEHLAIPFDRRALAYVLNALDPAHPRPVPCVPVAPLIGG

SEQ ID NO: 94 - DNA, 957 bp, synthetic construct
  source 1..957: mol_type = other DNA
atgcgtatgagccgtcgtgcaaaactgcgttgtgcactgccgctggcactggttgcagttgttgcagcatttgcaccggcagcaaatgcacagctggcagttgattatagcctgccgaaagcactggcagcatgggcagccaatccgaatggtagccctccgggtgccaatgattggagctgtcgtccgagcagcctgcatccgtatccggttattctggttcatggcacctttgaaaatcagcgctttaattggaatgcactggcaccgctgctgaaaaatgcaggttattgtgttttcagcttcaactatggtgcaacccagggcacccagctgaccggtggttttgcaaatggtctggcagcagttcgtgatagcgcacgtcaactggcagcctttgttgatcgtgttcgtgcagcaaccggtgcagcagaagttgatattgttggtcatagccaaggtggtatgatgcctcgttattataccaataaacttggtggtgcaagcaaagttcgtgccctggttggtctgagcccgagcaatcatggtacaaccaagagcggtctgaccgattttgcacgtctgctgggtccgattggtaatgcagccgttcagctggttaccagtttttgtccggcatgtggtgatcagattcgtggtagcgcctttatgcaagaactgaatgccggtggtgataccgttgccggtgttgcatataccgttattcagaccaaatatgatgaagtggttaccccgtatgatagcgcgtttctgactccggcagccagcagccatcaggttcgtaatattctggtgcagaaaggttgtccgattgatctgagcgaacatctggcaattccgtttgatcgtcgtgcactggcctatgttctgaatgccctggatccggcacatccgcgtccggttccgtgtgttccggttgcaccgctgattggcggt

SEQ ID NO: 95 - protein, 319 aa, synthetic construct
MRMSRRAKLRCALPLALVAVVAAFAPAANAQLAVDYSLPKALAAWAANPNGSPPGANDWSCRPSSLHPYPVILVHGTFENQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTSSGLTDFARLLGPIGNAAVQLVTSFCPACGDQIRGSAFMQELNAGGDTVAGVAYTVIQTKYDEVVTPYDSAFLTPAASSHQVRNILVQKGCPIDLSEHLAIPFDRRALAYVLNALDPAHPRPVPCVPVAPLIGG

SEQ ID NO: 96 - DNA, 957 bp, synthetic construct
  source 1..957: mol_type = other DNA
atgcgtatgagccgtcgtgcaaaactgcgttgtgcactgccgctggcactggttgcagttgttgcagcatttgcaccggcagcaaatgcacagctggcagttgattatagcctgccgaaagcactggcagcatgggcagccaatccgaatggtagccctccgggtgccaatgattggagctgtcgtccgagcagcctgcatccgtatccggttattctggttcatggcacctttgaaaatcagcgctttaattggaatgcactggcaccgctgctgaaaaatgcaggttattgtgttttcagcttcaactatggtgcaacccagggcacccagctgaccggtggttttgcaaatggtctggcagcagttcgtgatagcgcacgtcaactggcagcctttgttgatcgtgttcgtgcagcaaccggtgcagcagaagttgatattgttggtcatagccaaggtggtatgatgcctcgttattataccaataaacttggtggtgcaagcaaagttcgtgccctggttggtctgagcccgagcaatcatggtacaacctcgagcggtctgaccgattttgcacgtctgctgggtccgattggtaatgcagccgttcagctggttaccagtttttgtccggcatgtggtgatcagattcgtggtagcgcctttatgcaagaactgaatgccggtggtgataccgttgccggtgttgcatataccgttattcagaccaaatatgatgaagtggttaccccgtatgatagcgcgtttctgactccggcagccagcagccatcaggttcgtaatattctggtgcagaaaggttgtccgattgatctgagcgaacatctggcaattccgtttgatcgtcgtgcactggcctatgttctgaatgccctggatccggcacatccgcgtccggttccgtgtgttccggttgcaccgctgattggcggt

SEQ ID NO: 97 - protein, 319 aa, synthetic construct
MRMSRRAKLRCALPLALVAVVAAFAPAANAQLAVDYSLPKALAAWAANPNGSPPGANDWSCRPSSLHPYPVILVHGTFTNQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTLSGLTDFARLLGPIGNAAVQLVTSFCPACGDQIRGSAFMQELNAGGDTVAGVAYTVIQTKYDEVVTPYDSAFLTPAASSHQVRNILVQKGCPIDLSEHLAIPFDRRALAYVLNALDPAHPRPVPCVPVAPLIGG

SEQ ID NO: 98 - DNA, 957 bp, synthetic construct
  source 1..957: mol_type = other DNA
atgcgtatgagccgtcgtgcaaaactgcgttgtgcactgccgctggcactggttgcagttgttgcagcatttgcaccggcagcaaatgcacagctggcagttgattatagcctgccgaaagcactggcagcatgggcagccaatccgaatggtagccctccgggtgccaatgattggagctgtcgtccgagcagcctgcatccgtatccggttattctggttcatggcacctttactaatcagcgctttaattggaatgcactggcaccgctgctgaaaaatgcaggttattgtgttttcagcttcaactatggtgcaacccagggcacccagctgaccggtggttttgcaaatggtctggcagcagttcgtgatagcgcacgtcaactggcagcctttgttgatcgtgttcgtgcagcaaccggtgcagcagaagttgatattgttggtcatagccaaggtggtatgatgcctcgttattataccaataaacttggtggtgcaagcaaagttcgtgccctggttggtctgagcccgagcaatcatggtacaaccctgagcggtctgaccgattttgcacgtctgctgggtccgattggtaatgcagccgttcagctggttaccagtttttgtccggcatgtggtgatcagattcgtggtagcgcctttatgcaagaactgaatgccggtggtgataccgttgccggtgttgcatataccgttattcagaccaaatatgatgaagtggttaccccgtatgatagcgcgtttctgactccggcagccagcagccatcaggttcgtaatattctggtgcagaaaggttgtccgattgatctgagcgaacatctggcaattccgtttgatcgtcgtgcactggcctatgttctgaatgccctggatccggcacatccgcgtccggttccgtgtgttccggttgcaccgctgattggcggt

SEQ ID NO: 99 - protein, 319 aa, synthetic construct
MRMSRRAKLRCALPLALVAVVAAFAPAANAQLAVDYSLPKALAAWAANPNGSPPGANDWSCRPSSLHPYPVILVHGTFENQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTNSGLTDFARLLGPIGNAAVQLVTSFCPACGDQIRGSAFMQELNAGGDTVAGVAYTVIQTKYDEVVTPYDSAFLTPAASSHQVRNILVQKGCPIDLSEHLAIPFDRRALAYVLNALDPAHPRPVPCVPVAPLIGG

SEQ ID NO: 100 - DNA, 957 bp, synthetic construct
  source 1..957: mol_type = other DNA
atgcgtatgagccgtcgtgcaaaactgcgttgtgcactgccgctggcactggttgcagttgttgcagcatttgcaccggcagcaaatgcacagctggcagttgattatagcctgccgaaagcactggcagcatgggcagccaatccgaatggtagccctccgggtgccaatgattggagctgtcgtccgagcagcctgcatccgtatccggttattctggttcatggcacctttgaaaatcagcgctttaattggaatgcactggcaccgctgctgaaaaatgcaggttattgtgttttcagcttcaactatggtgcaacccagggcacccagctgaccggtggttttgcaaatggtctggcagcagttcgtgatagcgcacgtcaactggcagcctttgttgatcgtgttcgtgcagcaaccggtgcagcagaagttgatattgttggtcatagccaaggtggtatgatgcctcgttattataccaataaacttggtggtgcaagcaaagttcgtgccctggttggtctgagcccgagcaatcatggtacaaccaatagcggtctgaccgattttgcacgtctgctgggtccgattggtaatgcagccgttcagctggttaccagtttttgtccggcatgtggtgatcagattcgtggtagcgcctttatgcaagaactgaatgccggtggtgataccgttgccggtgttgcatataccgttattcagaccaaatatgatgaagtggttaccccgtatgatagcgcgtttctgactccggcagccagcagccatcaggttcgtaatattctggtgcagaaaggttgtccgattgatctgagcgaacatctggcaattccgtttgatcgtcgtgcactggcctatgttctgaatgccctggatccggcacatccgcgtccggttccgtgtgttccggttgcaccgctgattggcggt

SEQ ID NO: 101 - protein, 319 aa, synthetic construct
MRMSRRAKLRCALPLALVAVVAAFAPAANAQLAVDYSLPKALAAWAANPNGSPPGANDWSCRPSSLHPYPVILVHGTFENQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTISGLTDFARLLGPIGNAAVQLVTSFCPACGDQIRGSAFMQELNAGGDTVAGVAYTVIQTKYDEVVTPYDSAFLTPAASSHQVRNILVQKGCPIDLSEHLAIPFDRRALAYVLNALDPAHPRPVPCVPVAPLIGG

SEQ ID NO: 102 - DNA, 957 bp, synthetic construct
  source 1..957: mol_type = other DNA
atgcgtatgagccgtcgtgcaaaactgcgttgtgcactgccgctggcactggttgcagttgttgcagcatttgcaccggcagcaaatgcacagctggcagttgattatagcctgccgaaagcactggcagcatgggcagccaatccgaatggtagccctccgggtgccaatgattggagctgtcgtccgagcagcctgcatccgtatccggttattctggttcatggcacctttgaaaatcagcgctttaattggaatgcactggcaccgctgctgaaaaatgcaggttattgtgttttcagcttcaactatggtgcaacccagggcacccagctgaccggtggttttgcaaatggtctggcagcagttcgtgatagcgcacgtcaactggcagcctttgttgatcgtgttcgtgcagcaaccggtgcagcagaagttgatattgttggtcatagccaaggtggtatgatgcctcgttattataccaataaacttggtggtgcaagcaaagttcgtgccctggttggtctgagcccgagcaatcatggtacaaccattagcggtctgaccgattttgcacgtctgctgggtccgattggtaatgcagccgttcagctggttaccagtttttgtccggcatgtggtgatcagattcgtggtagcgcctttatgcaagaactgaatgccggtggtgataccgttgccggtgttgcatataccgttattcagaccaaatatgatgaagtggttaccccgtatgatagcgcgtttctgactccggcagccagcagccatcaggttcgtaatattctggtgcagaaaggttgtccgattgatctgagcgaacatctggcaattccgtttgatcgtcgtgcactggcctatgttctgaatgccctggatccggcacatccgcgtccggttccgtgtgttccggttgcaccgctgattggcggt

SEQ ID NO: 103 - protein, 319 aa, synthetic construct
MRMSRRAKLRCALPLALVAVVAAFAPAANAQLAVDYSLPKALAAWAANPNGSPPGANDWSCRPSSLHPYPVILVHGTFENQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTVSGLTDFARLLGPIGNAAVQLVTSFCPACGDQIRGSAFMQELNAGGDTVAGVAYTVIQTKYDEVVTPYDSAFLTPAASSHQVRNILVQKGCPIDLSEHLAIPFDRRALAYVLNALDPAHPRPVPCVPVAPLIGG

SEQ ID NO: 104 - DNA, 957 bp, synthetic construct
  source 1..957: mol_type = other DNA
atgcgtatgagccgtcgtgcaaaactgcgttgtgcactgccgctggcactggttgcagttgttgcagcatttgcaccggcagcaaatgcacagctggcagttgattatagcctgccgaaagcactggcagcatgggcagccaatccgaatggtagccctccgggtgccaatgattggagctgtcgtccgagcagcctgcatccgtatccggttattctggttcatggcacctttgaaaatcagcgctttaattggaatgcactggcaccgctgctgaaaaatgcaggttattgtgttttcagcttcaactatggtgcaacccagggcacccagctgaccggtggttttgcaaatggtctggcagcagttcgtgatagcgcacgtcaactggcagcctttgttgatcgtgttcgtgcagcaaccggtgcagcagaagttgatattgttggtcatagccaaggtggtatgatgcctcgttattataccaataaacttggtggtgcaagcaaagttcgtgccctggttggtctgagcccgagcaatcatggtacaaccgtgagcggtctgaccgattttgcacgtctgctgggtccgattggtaatgcagccgttcagctggttaccagtttttgtccggcatgtggtgatcagattcgtggtagcgcctttatgcaagaactgaatgccggtggtgataccgttgccggtgttgcatataccgttattcagaccaaatatgatgaagtggttaccccgtatgatagcgcgtttctgactccggcagccagcagccatcaggttcgtaatattctggtgcagaaaggttgtccgattgatctgagcgaacatctggcaattccgtttgatcgtcgtgcactggcctatgttctgaatgccctggatccggcacatccgcgtccggttccgtgtgttccggttgcaccgctgattggcggt

SEQ ID NO: 105 - protein, 319 aa, synthetic construct
MRMSRRAKLRCALPLALVAVVAAFAPAANAQLAVDYSLPKALAAWAANPNGSPPGANDWSCRPSSLHPYPVILVHGTFSNQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTYSGLTDFARLLGPIGNAAVQLVTSFCPACGDQIRGSAFMQELNAGGDTVAGVAYTVIQTKYDEVVTPYDSAFLTPAASSHQVRNILVQKGCPIDLSEHLAIPFDRRALAYVLNALDPAHPRPVPCVPVAPLIGG

SEQ ID NO: 106 - DNA, 957 bp, synthetic construct
  source 1..957: mol_type = other DNA
atgcgtatgtctcgtcgtgccaaactgcgctgcgccttaccccttgcgcttgtagctgtggttgctgcctttgcacccgcggcaaatgcacaactggcggtggactatagcctgccgaaagcgctcgctgcatgggcagccaatccgaatggcagcccaccgggtgcgaacgattggtcgtgtcgcccaagctccttgcacccttatccggtgattctggtccacggtacattcagcaaccagcgcttcaattggaatgccctggcgcctctgctgaagaacgcgggttactgcgtcttttcctttaactatggagcaacgcaaggtacgcaacttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctacagtggcctgactgactttgctcgcttgctcggccctattggcaatgcagccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacttatcggagcatctggcgattccgtttgatcgtcgtgcgctggcttatgtcctgaacgcgttagatccggcccatccacgtccggtgccttgtgtcccggtagcgccgctgattggcggg

SEQ ID NO: 107 - protein, 319 aa, synthetic construct
MRMSRRAKLRAALPLALVAVVAAFAPAANAQLAVDYSLPKALAAWAANPNGSPPGANDWSCRPSSLHPYPVILVHGTFENQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTYSGLTDFARLLGPIGNAAVQLVTSFCPACGDQIRGSAFMQELNAGGDTVAGVAYTVIQTKYDEVVTPYDSAFLTPAASSHQVRNILVQKGCPIDLSEHLAIPFDRRALAYVLNALDPAHPRPVPCVNVAPLIGG

SEQ ID NO: 108 - DNA, 957 bp, synthetic construct
  source 1..957: mol_type = other DNA
atgcgtatgtctcgtcgtgccaaactgcgcgcagccttaccccttgcgctggtagctgtggttgctgcctttgcacccgcggcaaatgcacaactggcggtggactatagcctgccgaaagcgctcgctgcatgggcagccaatccgaatggcagcccaccgggtgcgaacgattggtcgtgtcgcccaagctccttgcacccttatccggtgattctggtccacggtacattcgagaaccagcgcttcaattggaatgccctggcgcctctgctgaagaacgcgggttactgcgtcttttcctttaactatggagcaacgcaaggtacgcaacttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctacagtggcctgactgactttgctcgcttgctcggccctattggcaatgcagccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacttatcggagcatctggcgattccgtttgatcgtcgtgcgctggcttatgtcctgaacgcgttagatccggcccatccacgtccggtgccttgtgtcaacgtagcgccgctgattggcggg

SEQ ID NO: 109 - protein, 319 aa, synthetic construct
MRMSRRAKLRCALPLALVAVVAAFAPAANAQLAVDYSLPKALAAWAANPNGSPPGANDWSCRPSSLHPYPVILVHGTFENQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTYSGLTDFARLLGPIGNAAVQLVTSFCPACGDQIRGSAFMQELNAGGDTVAGVAYTVIQTKYDEVVTPYDSAFLTPAASSHQVRNILVQKGCPIDASEHLAIPFDRRALAYVLNALDPAHPRPVPCVNVAPLIGG

SEQ ID NO: 110 - DNA, 957 bp, synthetic construct
  source 1..957: mol_type = other DNA
atgcgtatgtctcgtcgtgccaaactgcgctgcgccttaccccttgcgcttgtagctgtggttgctgcctttgcacccgcggcaaatgcacaactggcggtggactatagcctgccgaaagcgctcgctgcatgggcagccaatccgaatggcagcccaccgggtgcgaacgattggtcgtgtcgcccaagctccttgcacccttatccggtgattctggtccacggtacattcgagaaccagcgcttcaattggaatgccctggcgcctctgctgaagaacgcgggttactgcgtcttttcctttaactatggagcaacgcaaggtacgcaacttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctacagtggcctgactgactttgctcgcttgctcggccctattggcaatgcagccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacgcgtcggagcatctggcgattccgtttgatcgtcgtgcgctggcttatgtcctgaacgcgttagatccggcccatccacgtccggtgccttgtgtcaacgtagcgccgctgattggcggg

SEQ ID NO: 111 - protein, 319 aa, synthetic construct
MRMSRRAKLRCALPLAPVAVVAAFAPAANAQLAVDYSLPKALAAWAANPNGSPPGANDWSCRPSSLHPYPVILVHGTFENQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTYSGLTDFARLLGPIGNAAVQLVTSFCPACGDQIRGSAFMQELNAGGDTVAGVAYTVIQTKYDEVVTPYDSAFLTPAASSHQVRNILVQKGCPIDLSEHLAIPFDRRALAYVLNALDPAHPRPVPCVNVAPLIGG

SEQ ID NO: 112 - DNA, 957 bp, synthetic construct
  source 1..957: mol_type = other DNA
atgcgtatgtctcgtcgtgccaaactgcgctgcgccttaccccttgcgcctgtagctgtggttgctgcctttgcacccgcggcaaatgcacaactggcggtggactatagcctgccgaaagcgctcgctgcatgggcagccaatccgaatggcagcccaccgggtgcgaacgattggtcgtgtcgcccaagctccttgcacccttatccggtgattctggtccacggtacattcgagaaccagcgcttcaattggaatgccctggcgcctctgctgaagaacgcgggttactgcgtcttttcctttaactatggagcaacgcaaggtacgcaacttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctacagtggcctgactgactttgctcgcttgctcggccctattggcaatgcagccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacttatcggagcatctggcgattccgtttgatcgtcgtgcgctggcttatgtcctgaacgcgttagatccggcccatccacgtccggtgccttgtgtcaacgtagcgccgctgattggcggg

SEQ ID NO: 113 - protein, 319 aa, synthetic construct
MRMSRRAKLRCALPLALVAVVAAFAPAANAQLAVDYSLPKALAAWAANPNGSPPGANDWSCRPSSLHPYPVILVHGTFENQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTWSGLTDFARLLGPIGNAAVQLVTSFCPACGDQIRGSAFMQELNAGGDTVAGVAYTVIQTKYDEVVTPYDSAFLTPAASSHQVRNILVQKGCPIDASEHLAIPFDRRALAYVLNALDPAHPRPVPCVNVAPLIGG

SEQ ID NO: 114 - DNA, 957 bp, synthetic construct
  source 1..957: mol_type = other DNA
atgcgtatgtctcgtcgtgccaaactgcgctgcgccttaccccttgcgctggtagctgtggttgctgcctttgcacccgcggcaaatgcacaactggcggtggactatagcctgccgaaagcgctcgctgcatgggcagccaatccgaatggcagcccaccgggtgcgaacgattggtcgtgtcgcccaagctccttgcacccttatccggtgattctggtccacggtacattcgagaaccagcgcttcaattggaatgccctggcgcctctgctgaagaacgcgggttactgcgtcttttcctttaactatggagcaacgcaaggtacgcaacttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctggagtggcctgactgactttgctcgcttgctcggccctattggcaatgcagccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacgcgtcggagcatctggcgattccgtttgatcgtcgtgcgctggcttatgtcctgaacgcgttagatccggcccatccacgtccggtgccttgtgtcaacgtagcgccgctgattggcggg

SEQ ID NO: 115 - protein, 319 aa, synthetic construct
MRMSRRAKLRCALPLALVAVVAAFAPAAHAQLAVDYSLPKALAAWAANPNGSPPGANDWSCRPSSLHPYPVILVHGTFENQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTWSGLTDFARLLGPIGNAAVQLVTSFCPACGDQIRGSAFMQELNAGGDTVAGVAYTVIQTKYDEVVTPYDSAFLTPAASSHQVRNILVQKGCPIDASEHLAIPFDRRALAYVLNALDPAHPRPVPCVPVAPLIGG

SEQ ID NO: 116 - DNA, 957 bp, synthetic construct
  source 1..957: mol_type = other DNA
atgcgtatgtctcgtcgtgccaaactgcgctgcgccttaccccttgcgctggtagctgtggttgctgcctttgcacccgcggcacatgcacaactggcggtggactatagcctgccgaaagcgctcgctgcatgggcagccaatccgaatggcagcccaccgggtgcgaacgattggtcgtgtcgcccaagctccttgcacccttatccggtgattctggtccacggtacattcgagaaccagcgcttcaattggaatgccctggcgcctctgctgaagaacgcgggttactgcgtcttttcctttaactatggagcaacgcaaggtacgcaacttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctggagtggcctgactgactttgctcgcttgctcggccctattggcaatgcagccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacgcgtcggagcatctggcgattccgtttgatcgtcgtgcgctggcttatgtcctgaacgcgttagatccggcccatccacgtccggtgccttgtgtcccggtagcgccgctgattggcggg

SEQ ID NO: 117 - protein, 319 aa, synthetic construct
MRMSRRAKLRCALPLAPVAVVAAFAPAANAQLAVDYSLPKALAAWAANPNGSPPGANDWSCRPSSLHPYPVILVHGTFENQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTYSGLTDFARLLGPIGNAAVQLVTSFCPACGDQIRGSAFMQELNAGGDTVAGVAYTVIQTKYDEVVTPYDSAFLTPAASSHQVRNILVQKGCPIDLSEHLAIPFDRRALAYVLNALDPAHPRPVCCVPVAPLIGG

SEQ ID NO: 118 - DNA, 957 bp, synthetic construct
  source 1..957: mol_type = other DNA
atgcgtatgtctcgtcgtgccaaactgcgctgcgccttaccccttgcgcctgtagctgtggttgctgcctttgcacccgcggcaaatgcacaactggcggtggactatagcctgccgaaagcgctcgctgcatgggcagccaatccgaatggcagcccaccgggtgcgaacgattggtcgtgtcgcccaagctccttgcacccttatccggtgattctggtccacggtacattcgagaaccagcgcttcaattggaatgccctggcgcctctgctgaagaacgcgggttactgcgtcttttcctttaactatggagcaacgcaaggtacgcaacttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctacagtggcctgactgactttgctcgcttgctcggccctattggcaatgcagccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacttatcggagcatctggcgattccgtttgatcgtcgtgcgctggcttatgtcctgaacgcgttagatccggcccatccacgtccggtgtgttgtgtcccggtagcgccgctgattggcggg

SEQ ID NO: 119 - protein, 319 aa, synthetic construct
MRMPRRAKLRCALPLALVAVVAAFAPAANAQLAVDYSLPKALAAWAANPNGSPPGANDWSCRPSSLHPYPVILVHGTFENQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTWSGLTDFARLLGPIGNNAVQLVTSFCPACGDQIRGSAFMQELNAGGDTVAGVAYTVIQTKYDEVVTPYDSAFLTPAASSHQVRNILVQKGCPIDLSEHLAIPFDRRALAYVLNALDPAHPRPVPCVPVAPLIGG

SEQ ID NO: 120 - DNA, 957 bp, synthetic construct
  source 1..957: mol_type = other DNA
atgcgtatgcctcgtcgtgccaaactgcgctgcgccttaccccttgcgctggtagctgtggttgctgcctttgcacccgcggcaaatgcacaactggcggtggactatagcctgccgaaagcgctcgctgcatgggcagccaatccgaatggcagcccaccgggtgcgaacgattggtcgtgtcgcccaagctccttgcacccttatccggtgattctggtccacggtacattcgagaaccagcgcttcaattggaatgccctggcgcctctgctgaagaacgcgggttactgcgtcttttcctttaactatggagcaacgcaaggtacgcaacttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctggagtggcctgactgactttgctcgcttgctcggccctattggcaataacgccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacttatcggagcatctggcgattccgtttgatcgtcgtgcgctggcttatgtcctgaacgcgttagatccggcccatccacgtccggtgccttgtgtcccggtagcgccgctgattggcggg

SEQ ID NO: 121 - protein, 319 aa, synthetic construct
MRMSRRAKLRCALPLALVAVVAAFAPAANAQLAVDYSLPKALAAWAANPNGSPPGANDWSCRPSSLHPYPVILVHGTFENQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTWSGLTDFARLLGPIGNNAVQLVTSFCPACGDQIRGSAFMQELNAGGDTVAGVAYTVIQTKYDEVVTPYDSAFLTPAASSHQVRNILVQKGCPIDLSEHLAIPFDRRALAYVLNALDPAHPRPVPCVNVAPLIGG

SEQ ID NO: 122 - DNA, 957 bp, synthetic construct
  source 1..957: mol_type = other DNA
atgcgtatgtctcgtcgtgccaaactgcgctgcgccttaccccttgcgctggtagctgtggttgctgcctttgcacccgcggcaaatgcacaactggcggtggactatagcctgccgaaagcgctcgctgcatgggcagccaatccgaatggcagcccaccgggtgcgaacgattggtcgtgtcgcccaagctccttgcacccttatccggtgattctggtccacggtacattcgagaaccagcgcttcaattggaatgccctggcgcctctgctgaagaacgcgggttactgcgtcttttcctttaactatggagcaacgcaaggtacgcaacttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctggagtggcctgactgactttgctcgcttgctcggccctattggcaataacgccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacttatcggagcatctggcgattccgtttgatcgtcgtgcgctggcttatgtcctgaacgcgttagatccggcccatccacgtccggtgccttgtgtcaacgtagcgccgctgattggcggg

SEQ ID NO: 123 - protein, 319 aa, synthetic construct
MRMSRRAKLRCALPLALVAVVAAFAPAANAQLAVDYSLPKALAAWAANPNGSPPGANDWSCRPSSLHPYPVILVHGTFSNQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTWSGLTDFARLLGPIGNAAVQLVTSFCPACGDQIRGSAFMQELNAGGDTVAGVAYTVIQTKYDEVVTPYDSAFLTPAASSHQVRNILVQKGCPIDLSEHLAIPFDRRALAYVLNALDPAHPRPVCCVNVAPLIGG

SEQ ID NO: 124 - DNA, 957 bp, synthetic construct
  source 1..957: mol_type = other DNA
atgcgtatgtctcgtcgtgccaaactgcgctgcgccttaccccttgcgctggtagctgtggttgctgcctttgcacccgcggcaaatgcacaactggcggtggactatagcctgccgaaagcgctcgctgcatgggcagccaatccgaatggcagcccaccgggtgcgaacgattggtcgtgtcgcccaagctccttgcacccttatccggtgattctggtccacggtacattcagcaaccagcgcttcaattggaatgccctggcgcctctgctgaagaacgcgggttactgcgtcttttcctttaactatggagcaacgcaaggtacgcaacttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctggagtggcctgactgactttgctcgcttgctcggccctattggcaatgcagccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacttatcggagcatctggcgattccgtttgatcgtcgtgcgctggcttatgtcctgaacgcgttagatccggcccatccacgtccggtgtgttgtgtcaacgtagcgccgctgattggcggg

SEQ ID NO: 125 - protein, 319 aa, synthetic construct
MRMSRRAKLRCALPLALVAVVAAFAPAANAQLAVDYSLPKALAAWAANPNGSPPGANDWSCRPSSLHPYPVILVHGTFENQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTYSGLTDAARLLGPIGNNAVQLVTSFCPACGDQIRGSAFMQELNAGGDTVAGVAYTVIQTKYDEVVTPYDSAFLTPAASSHQVRNILVQKGCPIDLSEHLAIPFDRRALAYVLNALDPAHPRPVPCVNVAPLIGG

SEQ ID NO: 126 - DNA, 957 bp, synthetic construct
  source 1..957: mol_type = other DNA
atgcgtatgtctcgtcgtgccaaactgcgctgcgccttaccccttgcgctggtagctgtggttgctgcctttgcacccgcggcaaatgcacaactggcggtggactatagcctgccgaaagcgctcgctgcatgggcagccaatccgaatggcagcccaccgggtgcgaacgattggtcgtgtcgcccaagctccttgcacccttatccggtgattctggtccacggtacattcgagaaccagcgcttcaattggaatgccctggcgcctctgctgaagaacgcgggttactgcgtcttttcctttaactatggagcaacgcaaggtacgcaacttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctacagtggcctgactgacgctgctcgcttgctcggccctattggcaataacgccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacttatcggagcatctggcgattccgtttgatcgtcgtgcgctggcttatgtcctgaacgcgttagatccggcccatccacgtccggtgccttgtgtcaacgtagcgccgctgattggcggg

SEQ ID NO: 127 - protein, 319 aa, synthetic construct
MRMSRRAELRAALPLALVAVVAAFAPAANAQLAVDYSLPKALAAWAANPNGSPPGANDWSCRPSSLHPYPVILVHGTFENQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTWSGLTDFARLLGPIGNAAVQLVTSFCPACGDQIRGSAFMQELNAGGDTVAGVAYTVIQTKYDEVVTPYDSAFLTPAASSHQVRNILVQKGCPIDLSEHLAIPFDRRALAYVLNALDPAHPRPVPCVNVAPLIGG

SEQ ID NO: 128 - DNA, 957 bp, synthetic construct
  source 1..957: mol_type = other DNA
atgcgtatgtcccgtcgtgccgaactgcgcgcagccttaccccttgcgctggtagctgtggttgctgcctttgcacccgcggcaaatgcacaactggcggtggactatagcctgccgaaagcgctcgctgcatgggcagccaatccgaatggcagcccaccgggtgcgaacgattggtcgtgtcgcccaagctccttgcacccttatccggtgattctggtccacggtacattcgagaaccagcgcttcaattggaatgccctggcgcctctgctgaagaacgcgggttactgcgtcttttcctttaactatggagcaacgcaaggtacgcaacttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctggagtggcctgactgactttgctcgcttgctcggccctattggcaatgcagccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacttatcggagcatctggcgattccgtttgatcgtcgtgcgctggcttatgtcctgaacgcgttagatccggcccatccacgtccggtgccttgtgtcaacgtagcgccgctgattggcggg

SEQ ID NO: 129 - protein, 319 aa, synthetic construct
MRMSRRAKLRCALPLALVAVVAAFAPAANAQLAVDYSLPKALAAWAANPNGSPPGANDWSCRPSSLHPYPVILVHGTFSNQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTYSGLTDFARLLGPIGNAAVQLVTSFCPACGDQIRGSAFMQELNAGGDTVAGVAYTVIQTKYDEVVTPYDSAFLTPAASSHQVRNILVQKGCPIDLSEHLAIPFDRRALAYVLNALDPAHPRPVCCVNVAPLIGG

SEQ ID NO: 130 - DNA, 957 bp, synthetic construct
  source 1..957: mol_type = other DNA
atgcgtatgtctcgtcgtgccaaactgcgctgcgccttaccccttgcgctggtagctgtggttgctgcctttgcacccgcggcaaatgcacaactggcggtggactatagcctgccgaaagcgctcgctgcatgggcagccaatccgaatggcagcccaccgggtgcgaacgattggtcgtgtcgcccaagctccttgcacccttatccggtgattctggtccacggtacattcagcaaccagcgcttcaattggaatgccctggcgcctctgctgaagaacgcgggttactgcgtcttttcctttaactatggagcaacgcaaggtacgcaacttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctacagtggcctgactgactttgctcgcttgctcggccctattggcaatgcagccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacttatcggagcatctggcgattccgtttgatcgtcgtgcgctggcttatgtcctgaacgcgttagatccggcccatccacgtccggtgtgttgtgtcaacgtagcgccgctgattggcggg

SEQ ID NO: 131 - protein, 319 aa, synthetic construct
MRMSRRAKLRCALPLALVAVVAAFAPAANAQLAVDYSLPKALAAWAANPNGSPPGANDWSCRPSSLHPYPVILVHGTFSNQRFTWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTWSGLTDFARLLGPIGNAAVQLVTSFCPACGDQIRGSAFMQELNAGGDTVAGVAYTVIQTKYDEVVTPYDSAFLTPAASSHQVRNILVQKGCPIDLSEHLAIPFDRRALAYVLNALDPAHPRPVCCVPVAPLIGG

SEQ ID NO: 132 - DNA, 957 bp, synthetic construct
  source 1..957: mol_type = other DNA
atgcgtatgtctcgtcgtgccaaactgcgctgcgccttaccccttgcgctggtagctgtggttgctgcctttgcacccgcggcaaatgcacaactggcggtggactatagcctgccgaaagcgctcgctgcatgggcagccaatccgaatggcagcccaccgggtgcgaacgattggtcgtgtcgcccaagctccttgcacccttatccggtgattctggtccacggtacattcagcaaccagcgcttcacttggaatgccctggcgcctctgctgaagaacgcgggttactgcgtcttttcctttaactatggagcaacgcaaggtacgcaacttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctggagtggcctgactgactttgctcgcttgctcggccctattggcaatgcagccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacttatcggagcatctggcgattccgtttgatcgtcgtgcgctggcttatgtcctgaacgcgttagatccggcccatccacgtccggtgtgttgtgtcccggtagcgccgctgattggcggg

SEQ ID NO: 133 - protein, 319 aa, synthetic construct
MRMSRRAKLRCALPLALVAVVAAFAPAANAQLAVDYSLPKALAAWAANPNGSPPGANDWSCRPSSLHPYPVILVHGTFSNQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTWSGLTDAARLLGPIGNAAVQLVTSFCPACGDQIRGSAFMQELNAGGDTVAGVAYTVIQTKYDEVVTPYDSAFLTPAASSHQVRNILVQKGCPIDLSEHLAIPFDRRALAYVLNALDPAHPRPVPCVNVAPLIGG

SEQ ID NO: 134 - DNA, 957 bp, synthetic construct
  source 1..957: mol_type = other DNA
atgcgtcagtcccgtcgtgccaaactgcgctgcgccttaccccttgcgcttgtagctgtggttgctgcctttgcacccgcggcaaatgcacaactggcggtggactatagcctgccgaaagcgctcgctgcatgggcagccaatccgaatggcagcccaccgggtgcgaacgattggtcgtgtcgcccaagctccttgcacccttatccggtgattctggtccacggtacattcagcaaccagcgcttcaattggaatgccctggcgcctctgctgaagaacgcgggttactgcgtcttttcctttaactatggagcaacgcaaggtacgcaacttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctacagtggcctgactgactttgctcgcttgctcggccctattggcaatgcagccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacttatcggagcatctggcgattccgtttgatcgtcgtgcgctggcttatgtcctgaacgcgttagcgccggcccatccacgtccggtgccttgtgtcccggtagcgccgctgattggcggg

SEQ ID NO: 135 - protein, 319 aa, synthetic construct
MRMSRRAKLRCALPLALVAVVAAFAPAAHAQLAVDYSLPKALAAWAANPNGSPPGANDWSCRPSSLHPYPVILVHGTFENQRFTWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTYSGLTDFARLLGPIGNNAVQLVTSFCPACGDQIRGSAFMQELNAGGDTVAGVAYTVIQTKYDEVVTPYDSAFLTPAASSHQVRNILVQKGCPIDLSEHLAIPFDRRALAYVLNALDPAHPRPVPCVPVAPLIGG

SEQ ID NO: 136 - DNA, 957 bp, synthetic construct
  source 1..957: mol_type = other DNA
atgcgtatgtctcgtcgtgccaaactgcgctgcgccttaccccttgcgctggtagctgtggttgctgcctttgcacccgcggcacatgcacaactggcggtggactatagcctgccgaaagcgctcgctgcatgggcagccaatccgaatggcagcccaccgggtgcgaacgattggtcgtgtcgcccaagctccttgcacccttatccggtgattctggtccacggtacattcgagaaccagcgcttcacttggaatgccctggcgcctctgctgaagaacgcgggttactgcgtcttttcctttaactatggagcaacgcaaggtacgcaacttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctacagtggcctgactgactttgctcgcttgctcggccctattggcaataacgccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacttatcggagcatctggcgattccgtttgatcgtcgtgcgctggcttatgtcctgaacgcgttagatccggcccatccacgtccggtgccttgtgtcccggtagcgccgctgattggcggg

SEQ ID NO: 137 - protein, 319 aa, synthetic construct
MRMSRRAKLRCALPLALCAVVAAFAPAANAQLAVDYSLPMALAAWAANPNGSPPGANDWSCRPSSLHPYPVILVHGTFENQRFTWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTWSGLTDFARLLGPIGNAAVQLVTSFCPACGDQIRGSAFMQELNAGGDTVAGVAYTVIQTKYDEVVTPYDSAFLTPAASSHQVRNILVQKGCPIDLSEHLAIPFDRRALAYVLNALDPAHPRPVPCVNVAPLIGG

SEQ ID NO: 138 - DNA, 957 bp, synthetic construct
  source 1..957: mol_type = other DNA
atgcgtatgtctcgtcgtgccaaactgcgctgcgccttaccccttgcgctttgcgctgtggttgctgcctttgcacccgcggcaaatgcacaactggcggtggactatagcctgccgatggcgctcgctgcatgggcagccaatccgaatggcagcccaccgggtgcgaacgattggtcgtgtcgcccaagctccttgcacccttatccggtgattctggtccacggtacattcgagaaccagcgcttcacttggaatgccctggcgcctctgctgaagaacgcgggttactgcgtcttttcctttaactatggagcaacgcaaggtacgcaacttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctggagtggcctgactgactttgctcgcttgctcggccctattggcaatgcagccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacttatcggagcatctggcgattccgtttgatcgtcgtgcgctggcttatgtcctgaacgcgttagatccggcccatccacgtccggtgccttgtgtcaacgtagcgccgctgattggcggg

SEQ ID NO: 139 - protein, 319 aa, synthetic construct
MRMPRRAKLRCALPLALVAVVAAFAPAAHAQLAVDYSLPKALAAWAANPNGSPPGANDWSCRPSSLHPYPVILVHGTFENQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTYSGLTDFARLLGPIGNNAVQLVTSFCPACGDQIRGSAFMQELNAGGDTVAGVAYTVIQTKYDEVVTPYDSAFLTPAASSHQVRNILVQKGCPIDLSEHLAIPFDRRALAYVLNALDPAHPRPVPCVNVAPLIGG

SEQ ID NO: 140 - DNA, 957 bp, synthetic construct
  source 1..957: mol_type = other DNA
atgcgtatgcctcgtcgtgccaaactgcgctgcgccttaccccttgcgcttgtagctgtggttgctgcctttgcacccgcggcacatgcacaactggcggtggactatagcctgccgaaagcgctcgctgcatgggcagccaatccgaatggcagcccaccgggtgcgaacgattggtcgtgtcgcccaagctccttgcacccttatccggtgattctggtccacggtacattcgagaaccagcgcttcaattggaatgccctggcgcctctgctgaagaacgcgggttactgcgtcttttcctttaactatggagcaacgcaaggtacgcaacttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctacagtggcctgactgactttgctcgcttgctcggccctattggcaataacgccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacttatcggagcatctggcgattccgtttgatcgtcgtgcgctggcttatgtcctgaacgcgttagatccggcccatccacgtccggtgccttgtgtcaacgtagcgccgctgattggcggg

SEQ ID NO: 141 - protein, 319 aa, synthetic construct
MRMSRRAKLRCALPLALAAVVAAFAPAANAQLAVDYSLPKALAAWAANPNGSPPGANDWSCRPSSLHPYPVILVHGTFENQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTYSGLTDAARLLGPIGNAAVQLVTSFCPACGDQIRGSAFMQELNAGGDTVAGVAYTVIQTKYDEVVTPYDSAFLTPAASSHQVRNILVQKGCPIDLSEHLAIPFDRRALAYVLNALAPAHPRPVPCVNVAPLIGG

SEQ ID NO: 142 - DNA, 957 bp, synthetic construct
  source 1..957: mol_type = other DNA
atgcgtatgtctcgtcgtgccaaactgcgctgcgccttaccccttgcgcttgccgctgtggttgctgcctttgcacccgcggcaaatgcacaactggcggtggactatagcctgccgaaagcgctcgctgcatgggcagccaatccgaatggcagcccaccgggtgcgaacgattggtcgtgtcgcccaagctccttgcacccttatccggtgattctggtccacggtacattcgagaaccagcgcttcaattggaatgccctggcgcctctgctgaagaacgcgggttactgcgtcttttcctttaactatggagcaacgcaaggtacgcaacttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctacagtggcctgactgacgctgctcgcttgctcggccctattggcaatgcagccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacttatcggagcatctggcgattccgtttgatcgtcgtgcgctggcttatgtcctgaacgcgttagcgccggcccatccacgtccggtgccttgtgtcaacgtagcgccgctgattggcggg

SEQ ID NO: 143 - protein, 319 aa, synthetic construct
MRMSRRAKLRCALPLALVAVVAAFAPAAHAQLAVDYSLPKALAAWAANPNGSPPGANDWSCRPSSLHPYPVILVHGTFENQRFNWNALAPLLKNAGYCVFSFNYGATQGTELTGGFANGLAAVRDSARQLAAFVDRVRAATGAAEVDIVGHSQGGMMPRYYTNKLGGASKVRALVGLSPSNHGTTWSGLTDFARLLGPIGNAAVQLVTSFCPACGDQIRGSAFMQELNAGGDTVAGVAYTVIQTKYDEVVTPYDSAFLTPAASSHQVRNILVQKGCPIDLSEHLAIPFDRRALAYVLNALDPAHPRPVCCVNVAPLIGG

SEQ ID NO: 144 - DNA, 957 bp, synthetic construct
  source 1..957: mol_type = other DNA
atgcgtatgtctcgtcgtgccaaactgcgctgcgccttaccccttgcgctggtagctgtggttgctgcctttgcacccgcggcacatgcacaactggcggtggactatagcctgccgaaagcgctcgctgcatgggcagccaatccgaatggcagcccaccgggtgcgaacgattggtcgtgtcgcccaagctccttgcacccttatccggtgattctggtccacggtacattcgagaaccagcgcttcaattggaatgccctggcgcctctgctgaagaacgcgggttactgcgtcttttcctttaactatggagcaacgcaaggtacggagcttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctggagtggcctgactgactttgctcgcttgctcggccctattggcaatgcagccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacttatcggagcatctggcgattccgtttgatcgtcgtgcgctggcttatgtcctgaacgcgttagatccggcccatccacgtccggtgtgttgtgtcaacgtagcgccgctgattggcggg

SEQ ID NO: 145 - protein, 319 aa, synthetic construct
MRQPRRAELRCALPLALVAVVAAFAPAANAQLAVDYSLPKALAAWAANPNGSPPGANDWSCRPSSLHPYPVILVHGTFENQRFTWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTWSGLTDFARLLGPIGNAAVQLVTSFCPACGDQIRGSAFMQELNAGGDTVAGVAYTVIQTKYDEVVTPYDSAFLTPAASSHQVRNILVQKGCPIDLSEHLAIPFDRRALAYVLNALDPAHPRPVPCVNVAPLIGG

SEQ ID NO: 146 - DNA, 957 bp, synthetic construct
  source 1..957: mol_type = other DNA
atgcgtcagccccgtcgtgccgaactgcgctgcgccttaccccttgcgcttgtagctgtggttgctgcctttgcacccgcggcaaatgcacaactggcggtggactatagcctgccgaaagcgctcgctgcatgggcagccaatccgaatggcagcccaccgggtgcgaacgattggtcgtgtcgcccaagctccttgcacccttatccggtgattctggtccacggtacattcgagaaccagcgcttcacttggaatgccctggcgcctctgctgaagaacgcgggttactgcgtcttttcctttaactatggagcaacgcaaggtacgcaacttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctggagtggcctgactgactttgctcgcttgctcggccctattggcaatgcagccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacttatcggagcatctggcgattccgtttgatcgtcgtgcgctggcttatgtcctgaacgcgttagatccggcccatccacgtccggtgccttgtgtcaacgtagcgccgctgattggcggg

SEQ ID NO: 147 - protein, 319 aa, synthetic construct
MRMSRRAKLRCALPLALVAVVAAFAPAANAQLAVDYSLPKALAAWAANPNGSPPGANDWSCRPSSLHPYPVILVHGTFSNQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTYSGLTDFARLLGPIGNAAVQLVTSFCPACGDQIRGSAFMQELNAGGDTVAGVAYTVIQTKYDEVVTPYDSAFLTPAASSHQVRNILVQKGCPIDLSEHLAIPFDRRALAYVLNALAPAHPRPVPCVPVAPLIGG

SEQ ID NO: 148 - DNA, 957 bp, synthetic construct
  source 1..957: mol_type = other DNA
atgcgtatgtcccgtcgtgccaaactgcgctgcgccttaccccttgcgctggtagctgtggttgctgcctttgcacccgcggcaaatgcacaactggcggtggactatagcctgccgaaagcgctcgctgcatgggcagccaatccgaatggcagcccaccgggtgcgaacgattggtcgtgtcgcccaagctccttgcacccttatccggtgattctggtccacggtacattcagcaaccagcgcttcaattggaatgccctggcgcctctgctgaagaacgcgggttactgcgtcttttcctttaactatggagcaacgcaaggtacgcaacttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctacagtggcctgactgactttgctcgcttgctcggccctattggcaatgcagccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacttatcggagcatctggcgattccgtttgatcgtcgtgcgctggcttatgtcctgaacgcgttagcgccggcccatccacgtccggtgccttgtgtcccggtagcgccgctgattggcggg

SEQ ID NO: 149 - protein, 319 aa, synthetic construct
MRMSRRAKLRCALPLALVAVVAAFAPAANAQLAVDYSLPKALAAWAANPNGSPPGANDWSCRPSSLHPYPVILVHGTFENQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTYSGLTDFARLLGPIGNNAVQLVTSFCPACGDQIRGSAFMQELNAGGDTVAGVAYTVIQTKYDEVVTPYDSAFLTPAASSHQVRNILVQKGCPIDLSEHLAIPFDRRALAYVLNALAPAHPRPVPCVPVAPLIGG

SEQ ID NO: 150 - DNA, 957 bp, synthetic construct
  source 1..957: mol_type = other DNA
atgcgtatgtctcgtcgtgccaaactgcgctgcgccttaccccttgcgcttgtagctgtggttgctgcctttgcacccgcggcaaatgcacaactggcggtggactatagcctgccgaaagcgctcgctgcatgggcagccaatccgaatggcagcccaccgggtgcgaacgattggtcgtgtcgcccaagctccttgcacccttatccggtgattctggtccacggtacattcgagaaccagcgcttcaattggaatgccctggcgcctctgctgaagaacgcgggttactgcgtcttttcctttaactatggagcaacgcaaggtacgcaacttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctacagtggcctgactgactttgctcgcttgctcggccctattggcaataacgccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacttatcggagcatctggcgattccgtttgatcgtcgtgcgctggcttatgtcctgaacgcgttagcgccggcccatccacgtccggtgccttgtgtcccggtagcgccgctgattggcggg

SEQ ID NO: 151 - protein, 319 aa, synthetic construct
MRMSRRAKLRCALPLAPVAVVAAFAPAANAQLAVDYSLPKALAAWAANPNGSPPGANDWSCRPSSLHPYPVILVHGTFENQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTWSGLTDFARLLGPIGNNAVQLVTSFCPACGDQIRGSAFMQELNAGGDTVAGVAYTVIQTKYDEVVTPYDSAFLTPAASSHQVRNILVQKGCPIDLSEHLAIPFDRRALAYVLNALDPAHPRPVPCVPVAPLIGG

SEQ ID NO: 152 - DNA, 957 bp, synthetic construct
  source 1..957: mol_type = other DNA
atgcgtatgtctcgtcgtgccaaactgcgctgcgccttaccccttgcgcctgtagctgtggttgctgcctttgcacccgcggcaaatgcacaactggcggtggactatagcctgccgaaagcgctcgctgcatgggcagccaatccgaatggcagcccaccgggtgcgaacgattggtcgtgtcgcccaagctccttgcacccttatccggtgattctggtccacggtacattcgagaaccagcgcttcaattggaatgccctggcgcctctgctgaagaacgcgggttactgcgtcttttcctttaactatggagcaacgcaaggtacgcaacttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctggagtggcctgactgactttgctcgcttgctcggccctattggcaataacgccgttcagcttgtcaccagcttctgtccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacttatcggagcatctggcgattccgtttgatcgtcgtgcgctggcttatgtcctgaacgcgttagatccggcccatccacgtccggtgccttgtgtcccggtagcgccgctgattggcggg

SEQ ID NO: 153 - protein, 319 aa, synthetic construct
MRQSRRAKLRCALPLALVAVVAAFAPAANAQLAVDYSLPKALAAWAANPNGSPPGANDWSCRPSSLHPYPVILVHGTFSNQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTYSGLTDFARLLGPIGNAAVQLVTSFCPACGDQIRGSAFMQELNAGGDTVAGVAYTVIQTKYDEVVTPYDSAFLTPAASSHQVRNILVQKGCPIDLSEHLAIPFDRRALAYVLNALDPAHPRPVPCVNVAPLIGG

SEQ ID NO: 154 - DNA, 957 bp, synthetic construct
  source 1..957: mol_type = other DNA
atgcgtcagtctcgtcgtgccaaactgcgctgcgccttaccccttgcgctggtagctgtggttgctgcctttgcacccgcggcaaatgcacaactggcggtggactatagcctgccgaaagcgctcgctgcatgggcagccaatccgaatggcagcccaccgggtgcgaacgattggtcgtgtcgcccaagctccttgcacccttatccggtgattctggtccacggtacattcagcaaccagcgcttcaattggaatgccctggcgcctctgctgaagaacgcgggttactgcgtcttttcctttaactatggagcaacgcaaggtacgcaacttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctacagtggcctgactgactttgctcgcttgctcggccctattggcaatgcagccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacttatcggagcatctggcgattccgtttgatcgtcgtgcgctggcttatgtcctgaacgcgttagatccggcccatccacgtccggtgccttgtgtcaacgtagcgccgctgattggcggg

SEQ ID NO: 155 - protein, 319 aa, synthetic construct
MRMSRRAKLRCALPLALVAVVAAFAPAANAQLAVDYSLPMALAAWAANPNGSPPGANDWSCRPSSLHPYPVILVHGTFSNQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTYSGLTDFARLLGPIGNAAVQLVTSFCPACGDQIRGSAFMQELNAGGDTVAGVAYTVIQTKYDEVVTPYDSAFLTPAASSHQVRNILVQKGCPIDLSEHLAIPFDRRALAYVLNALDPAHPRPVPCVNVAPLIGG

SEQ ID NO: 156 - DNA, 957 bp, synthetic construct
  source 1..957: mol_type = other DNA
atgcgtatgtctcgtcgtgccaaactgcgctgcgccttaccccttgcgctggtagctgtggttgctgcctttgcacccgcggcaaatgcacaactggcggtggactatagcctgccgatggcgctcgctgcatgggcagccaatccgaatggcagcccaccgggtgcgaacgattggtcgtgtcgcccaagctccttgcacccttatccggtgattctggtccacggtacattcagcaaccagcgcttcaattggaatgccctggcgcctctgctgaagaacgcgggttactgcgtcttttcctttaactatggagcaacgcaaggtacgcaacttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctacagtggcctgactgactttgctcgcttgctcggccctattggcaatgcagccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacttatcggagcatctggcgattccgtttgatcgtcgtgcgctggcttatgtcctgaacgcgttagatccggcccatccacgtccggtgccttgtgtcaacgtagcgccgctgattggcggg

SEQ ID NO: 157 - protein, 319 aa, synthetic construct
MRMSRRAELRCALPLALVAVVAAFAPAANAQLAVDYSLPKALAAWAANPNGSPPGANDWSCRPSSLHPYPVILVHGTFENQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTYSGLTDFARLLGPIGNAAVQLVTSFCPACGDQIRGSAFMQELNAGGDTVAGVAYTVIQTKYDEVVTPYDSAFLTPAASSHQVRNILVQKGCPIDASEHLAIPFDRRALAYVLNALAPAHPRPVPCVPVAPLIGG

SEQ ID NO: 158 - DNA, 957 bp, synthetic construct
  source 1..957: mol_type = other DNA
atgcgtatgtctcgtcgtgccgaactgcgctgcgccttaccccttgcgctggtagctgtggttgctgcctttgcacccgcggcaaatgcacaactggcggtggactatagcctgccgaaagcgctcgctgcatgggcagccaatccgaatggcagcccaccgggtgcgaacgattggtcgtgtcgcccaagctccttgcacccttatccggtgattctggtccacggtacattcgagaaccagcgcttcaattggaatgccctggcgcctctgctgaagaacgcgggttactgcgtcttttcctttaactatggagcaacgcaaggtacgcaacttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctacagtggcctgactgactttgctcgcttgctcggccctattggcaatgcagccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacgcgtcggagcatctggcgattccgtttgatcgtcgtgcgctggcttatgtcctgaacgcgttagcgccggcccatccacgtccggtgccttgtgtcccggtagcgccgctgattggcggg

SEQ ID NO: 159 - protein, 319 aa, synthetic construct
MRMSRRAKLRCALPLAPVAVVAAFAPAANAQLAVDYSLPKALAAWAANPNGSPPGANDWSCRPSSLHPYPVILVHGTFSNQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTYSGLTDFARLLGPIGNAAVQLVTSFCPACGDQIRGSAFMQELNAGGDTVAGVAYTVIQTKYDEVVTPYDSAFLTPAASSHQVRNILVQKGCPIDLSEHLAIPFDRRALAYVLNALDPAHPRPVCCVPVAPLIGG

SEQ ID NO: 160 - DNA, 957 bp, synthetic construct
  source 1..957: mol_type = other DNA
atgcgtatgtctcgtcgtgccaaactgcgctgcgccttaccccttgcgcctgtagctgtggttgctgcctttgcacccgcggcaaatgcacaactggcggtggactatagcctgccgaaagcgctcgctgcatgggcagccaatccgaatggcagcccaccgggtgcgaacgattggtcgtgtcgcccaagctccttgcacccttatccggtgattctggtccacggtacattcagcaaccagcgcttcaattggaatgccctggcgcctctgctgaagaacgcgggttactgcgtcttttcctttaactatggagcaacgcaaggtacgcaacttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctacagtggcctgactgactttgctcgcttgctcggccctattggcaatgcagccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacttatcggagcatctggcgattccgtttgatcgtcgtgcgctggcttatgtcctgaacgcgttagatccggcccatccacgtccggtgtgttgtgtcccggtagcgccgctgattggcggg

SEQ ID NO: 161 - protein, 319 aa, synthetic construct
MRMSRRAKLRCALPLALVAVVAAFAPAANAQLAVDYSLPKALAAWAANPNGSPPGANDWSCRPSSLHPYPVILVHGTFENQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTYSGLTDAARLLGPIGNAAVQLVTSFCPACGDQIRGSAFMQELNAGGDTVAGVAYTVIQTKYDEVVTPYDSAFLTPAASSHQVRNILVQKGCPIDASEHLAIPFDRRALAYVLNALAPAHPRPVPCVPVAPLIGG

SEQ ID NO: 162 - DNA, 957 bp, synthetic construct
  source 1..957: mol_type = other DNA
atgcgtatgtctcgtcgtgccaaactgcgctgcgccttaccccttgcgctggtagctgtggttgctgcctttgcacccgcggcaaatgcacaactggcggtggactatagcctgccgaaagcgctcgctgcatgggcagccaatccgaatggcagcccaccgggtgcgaacgattggtcgtgtcgcccaagctccttgcacccttatccggtgattctggtccacggtacattcgagaaccagcgcttcaattggaatgccctggcgcctctgctgaagaacgcgggttactgcgtcttttcctttaactatggagcaacgcaaggtacgcaacttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctacagtggcctgactgacgctgctcgcttgctcggccctattggcaatgcagccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacgcgtcggagcatctggcgattccgtttgatcgtcgtgcgctggcttatgtcctgaacgcgttagcgccggcccatccacgtccggtgccttgtgtcccggtagcgccgctgattggcggg

SEQ ID NO: 163 - protein, 319 aa, synthetic construct
MRMSRRAKLRCALPLALVAVVAAFAPAANAQLAVDYSLPKALAAWAANPNGSPPGANDWSCRPSSLHPYPVILVHGTFENQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTYSGLTDFARLLGPIGNNAVQLVTSFCPACGDQIRGSAFMQELNAGGDTVAGVAYTVIQTKYDEVVTPYDSAFLTPAASSHQVRNILVQKGCPIDASEHLAIPFDRRALAYVLNALDPAHPRPVPCVNVAPLIGG

SEQ ID NO: 164 - DNA, 957 bp, synthetic construct
  source 1..957: mol_type = other DNA
atgcgtatgtctcgtcgtgccaaactgcgctgcgccttaccccttgcgctggtagctgtggttgctgcctttgcacccgcggcaaatgcacaactggcggtggactatagcctgccgaaagcgctcgctgcatgggcagccaatccgaatggcagcccaccgggtgcgaacgattggtcgtgtcgcccaagctccttgcacccttatccggtgattctggtccacggtacattcgagaaccagcgcttcaattggaatgccctggcgcctctgctgaagaacgcgggttactgcgtcttttcctttaactatggagcaacgcaaggtacgcaacttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctacagtggcctgactgactttgctcgcttgctcggccctattggcaataacgccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacgcgtcggagcatctggcgattccgtttgatcgtcgtgcgctggcttatgtcctgaacgcgttagatccggcccatccacgtccggtgccttgtgtcaacgtagcgccgctgattggcggg

SEQ ID NO: 165 - protein, 319 aa, synthetic construct
MRMSRRAKLRCALPLALVAVVAAFAPAANAQLAVDYSLPMALAAWAANPNGSPPGANDWSCRPSSLHPYPVILVHGTFENQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTWSGLTDFARLLGPIGNAAVQLVTSFCPACGDQIRGSAFMQELNAGGDTVAGVAYTVIQTKYDEVVTPYDSAFLTPAASSHQVRNILVQKGCPIDASEHLAIPFDRRALAYVLNALDPAHPRPVCCVPVAPLIGG

SEQ ID NO: 166 - DNA, 957 bp, synthetic construct
  source 1..957: mol_type = other DNA
atgcgtatgtctcgtcgtgccaaactgcgctgcgccttaccccttgcgctggtagctgtggttgctgcctttgcacccgcggcaaatgcacaactggcggtggactatagcctgccgatggcgctcgctgcatgggcagccaatccgaatggcagcccaccgggtgcgaacgattggtcgtgtcgcccaagctccttgcacccttatccggtgattctggtccacggtacattcgagaaccagcgcttcaattggaatgccctggcgcctctgctgaagaacgcgggttactgcgtcttttcctttaactatggagcaacgcaaggtacgcaacttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctggagtggcctgactgactttgctcgcttgctcggccctattggcaatgcagccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacgcgtcggagcatctggcgattccgtttgatcgtcgtgcgctggcttatgtcctgaacgcgttagatccggcccatccacgtccggtgtgttgtgtcccggtagcgccgctgattggcggg

SEQ ID NO: 167 - protein, 319 aa, synthetic construct
MRMSRRAKLRAALPLALVAVVAAFAPAAWAQLAVDYSLPKALAAWAANPNGSPPGANDWSCRPSSLHPYPVILVHGTFSNQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTYSGLTDFARLLGPIGNAAVQLVTSFCPACGDQIRGSAFMQELNAGGDTVAGVAYTVIQTKYDEVVTPYDSAFLTPAASSHQVRNILVQKGCPIDLSEHLAIPFDRRALAYVLNALDPAHPRPVPCVPVAPLIGG

SEQ ID NO: 168 - DNA, 957 bp, synthetic construct
  source 1..957: mol_type = other DNA
atgcgtatgtctcgtcgtgccaaactgcgcgcagccttaccccttgcgctggtagctgtggttgctgcctttgcacccgcggcatgggcacaactggcggtggactatagcctgccgaaagcgctcgctgcatgggcagccaatccgaatggcagcccaccgggtgcgaacgattggtcgtgtcgcccaagctccttgcacccttatccggtgattctggtccacggtacattcagcaaccagcgcttcaattggaatgccctggcgcctctgctgaagaacgcgggttactgcgtcttttcctttaactatggagcaacgcaaggtacgcaacttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctacagtggcctgactgactttgctcgcttgctcggccctattggcaatgcagccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacttatcggagcatctggcgattccgtttgatcgtcgtgcgctggcttatgtcctgaacgcgttagatccggcccatccacgtccggtgccttgtgtcccggtagcgccgctgattggcggg

SEQ ID NO: 169 - protein, 319 aa, synthetic construct
MRMSRRAKLRCALPLALVAVVAAFAPAAWAQLAVDYSLPKALAAWAANPNGSPPGANDWSCRPSSLHPYPVILVHGTFENQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTYSGLTDFARLLGPIGNNAVQLVTSFCPACGDQIRGSAFMQELNAGGDTVAGVAYTVIQTKYDEVVTPYDSAFLTPAASSHQVRNILVQKGCPIDLSEHLAIPFDRRALAYVLNALAPAHPRPVPCVPVAPLIGG

SEQ ID NO: 170 - DNA, 957 bp, synthetic construct
  source 1..957: mol_type = other DNA
atgcgtatgtctcgtcgtgccaaactgcgctgcgccttaccccttgcgctggtagctgtggttgctgcctttgcacccgcggcatgggcacaactggcggtggactatagcctgccgaaagcgctcgctgcatgggcagccaatccgaatggcagcccaccgggtgcgaacgattggtcgtgtcgcccaagctccttgcacccttatccggtgattctggtccacggtacattcgagaaccagcgcttcaattggaatgccctggcgcctctgctgaagaacgcgggttactgcgtcttttcctttaactatggagcaacgcaaggtacgcaacttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctacagtggcctgactgactttgctcgcttgctcggccctattggcaataacgccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacttatcggagcatctggcgattccgtttgatcgtcgtgcgctggcttatgtcctgaacgcgttagcgccggcccatccacgtccggtgccttgtgtcccggtagcgccgctgattggcggg

SEQ ID NO: 171 - protein, 319 aa, synthetic construct
MRMSRRAKLRCALPLAPVAVVAAFAPAAHAQLAVDYSLPKALAAWAANPNGSPPGANDWSCRPSSLHPYPVILVHGTFSNQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTYSGLTDFARLLGPIGNAAVQLVTSFCPACGDQIRGSAFMQELNAGGDTVAGVAYTVIQTKYDEVVTPYDSAFLTPAASSHQVRNILVQKGCPIDLSEHLAIPFDRRALAYVLNALDPAHPRPVPCVPVAPLIGG

SEQ ID NO: 172 - DNA, 957 bp, synthetic construct
  source 1..957: mol_type = other DNA
atgcgtatgtctcgtcgtgccaaactgcgctgcgccttaccccttgcgcctgtagctgtggttgctgcctttgcacccgcggcacatgcacaactggcggtggactatagcctgccgaaagcgctcgctgcatgggcagccaatccgaatggcagcccaccgggtgcgaacgattggtcgtgtcgcccaagctccttgcacccttatccggtgattctggtccacggtacattcagcaaccagcgcttcaattggaatgccctggcgcctctgctgaagaacgcgggttactgcgtcttttcctttaactatggagcaacgcaaggtacgcaacttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctacagtggcctgactgactttgctcgcttgctcggccctattggcaatgcagccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacttatcggagcatctggcgattccgtttgatcgtcgtgcgctggcttatgtcctgaacgcgttagatccggcccatccacgtccggtgccttgtgtcccggtagcgccgctgattggcggg

SEQ ID NO: 173 - protein, 319 aa, synthetic construct
MRMSRRAELRAALPLALVAVVAAFAPAANAQLAVDYSLPKALAAWAANPNGSPPGANDWSCRPSSLHPYPVILVHGTFENQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTYSGLTDFARLLGPIGNAAVQLVTSFCPACGDQIRGSAFMQELNAGGDTVAGVAYTVIQTKYDEVVTPYDSAFLTPAASSHQVRNILVQKGCPIDLSEHLAIPFDRRALAYVLNALAPAHPRPVPCVPVAPLIGG

SEQ ID NO: 174 - DNA, 957 bp, synthetic construct
  source 1..957: mol_type = other DNA
atgcgtatgtctcgtcgtgccgaactgcgcgcagccttaccccttgcgctggtagctgtggttgctgcctttgcacccgcggcaaatgcacaactggcggtggactatagcctgccgaaagcgctcgctgcatgggcagccaatccgaatggcagcccaccgggtgcgaacgattggtcgtgtcgcccaagctccttgcacccttatccggtgattctggtccacggtacattcgagaaccagcgcttcaattggaatgccctggcgcctctgctgaagaacgcgggttactgcgtcttttcctttaactatggagcaacgcaaggtacgcaacttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctacagtggcctgactgactttgctcgcttgctcggccctattggcaatgcagccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacttatcggagcatctggcgattccgtttgatcgtcgtgcgctggcttatgtcctgaacgcgttagcgccggcccatccacgtccggtgccttgtgtcccggtagcgccgctgattggcggg

SEQ ID NO: 175 - protein, 319 aa, synthetic construct
MRMPRRAKLRCALPLALVAVVAAFAPAANAQLAVDYSLPKALAAWAANPNGSPPGANDWSCRPSSLHPYPVILVHGTFSNQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTWSGLTDFARLLGPIGNNAVQLVTSFCPACGDQIRGSAFMQELNAGGDTVAGVAYTVIQTKYDEVVTPYDSAFLTPAASSHQVRNILVQKGCPIDLSEHLAIPFDRRALAYVLNALDPAHPRPVCCVPVAPLIGG

SEQ ID NO: 176 - DNA, 957 bp, synthetic construct
  source 1..957: mol_type = other DNA
atgcgtatgcctcgtcgtgccaaactgcgctgcgccttaccccttgcgctggtagctgtggttgctgcctttgcacccgcggcaaatgcacaactggcggtggactatagcctgccgaaagcgctcgctgcatgggcagccaatccgaatggcagcccaccgggtgcgaacgattggtcgtgtcgcccaagctccttgcacccttatccggtgattctggtccacggtacattcagcaaccagcgcttcaattggaatgccctggcgcctctgctgaagaacgcgggttactgcgtcttttcctttaactatggagcaacgcaaggtacgcaacttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctggagtggcctgactgactttgctcgcttgctcggccctattggcaataacgccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacttatcggagcatctggcgattccgtttgatcgtcgtgcgctggcttatgtcctgaacgcgttagatccggcccatccacgtccggtgtgttgtgtcccggtagcgccgctgattggcggg

SEQ ID NO: 177 - protein, 319 aa, synthetic construct
MRMSRRAKLRCALPLALVAVVAAFAPAANAWLAVDYSLPKALAAWAANPNGSPPGANDWSCRPSSLHPYPVILVHGTFSNQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTWSGLTDFARLLGPIGNNAVQLVTSFCPACGDQIRGSAFMQELNAGGDTVAGVAYTVIQTKYDEVVTPYDSAFLTPAASSHQVRNILVQKGCPIDLSEHLAIPFDRRALAYVLNALDPAHPRPVCCVPVAPLIGG

SEQ ID NO: 178 - DNA, 957 bp, synthetic construct
  source 1..957: mol_type = other DNA
atgcgtatgtctcgtcgtgccaaactgcgctgcgccttaccccttgcgctggtagctgtggttgctgcctttgcacccgcggcaaatgcatggctggcggtggactatagcctgccgaaagcgctcgctgcatgggcagccaatccgaatggcagcccaccgggtgcgaacgattggtcgtgtcgcccaagctccttgcacccttatccggtgattctggtccacggtacattcagcaaccagcgcttcaattggaatgccctggcgcctctgctgaagaacgcgggttactgcgtcttttcctttaactatggagcaacgcaaggtacgcaacttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctggagtggcctgactgactttgctcgcttgctcggccctattggcaataacgccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacttatcggagcatctggcgattccgtttgatcgtcgtgcgctggcttatgtcctgaacgcgttagatccggcccatccacgtccggtgtgttgtgtcccggtagcgccgctgattggcggg

SEQ ID NO: 179 - protein, 319 aa, synthetic construct
MRMSRRAKLRCALPLALVAVVAAFAPAANAQLAVDYSLPKALAAWAANPNGSPPGANDWSCRPSSLHPYPVILVHGTFSNQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTWSGLTDFARLLGPIGNAAVQLVTSFCPACGDQIRGSAFMQELNAGGDTVAGVAYTVIQTKYDEVVTPYDSAFLTPAASSHQVRNILVQKGCPIDASEHLAIPFDRRALAYVLNALDPAHPRPVCCVNVAPLIGG

SEQ ID NO: 180 - DNA, 957 bp, synthetic construct
  source 1..957: mol_type = other DNA
atgcgtatgtctcgtcgtgccaaactgcgctgcgccttaccccttgcgctggtagctgtggttgctgcctttgcacccgcggcaaatgcacaactggcggtggactatagcctgccgaaagcgctcgctgcatgggcagccaatccgaatggcagcccaccgggtgcgaacgattggtcgtgtcgcccaagctccttgcacccttatccggtgattctggtccacggtacattcagcaaccagcgcttcaattggaatgccctggcgcctctgctgaagaacgcgggttactgcgtcttttcctttaactatggagcaacgcaaggtacgcaacttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctggagtggcctgactgactttgctcgcttgctcggccctattggcaatgcagccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacgcgtcggagcatctggcgattccgtttgatcgtcgtgcgctggcttatgtcctgaacgcgttagatccggcccatccacgtccggtgtgttgtgtcaacgtagcgccgctgattggcggg

SEQ ID NO: 181 - protein, 319 aa, synthetic construct
MRMSRRAKLRCALPLALVAVVAAFAPAANAWLAVDYSLPKALAAWAANPNGSPPGANDWSCRPSSLHPYPVILVHGTFSNQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTWSGLTDAARLLGPIGNAAVQLVTSFCPACGDQIRGSAFMQELNAGGDTVAGVAYTVIQTKYDEVVTPYDSAFLTPAASSHQVRNILVQKGCPIDLSEHLAIPFDRRALAYVLNALAPAHPRPVPCVPVAPLIGG

SEQ ID NO: 182 - DNA, 957 bp, synthetic construct
  source 1..957: mol_type = other DNA
atgcgtatgtcccgtcgtgccaaactgcgctgcgccttaccccttgcgctggtagctgtggttgctgcctttgcacccgcggcaaatgcatggctggcggtggactatagcctgccgaaagcgctcgctgcatgggcagccaatccgaatggcagcccaccgggtgcgaacgattggtcgtgtcgcccaagctccttgcacccttatccggtgattctggtccacggtacattcagcaaccagcgcttcaattggaatgccctggcgcctctgctgaagaacgcgggttactgcgtcttttcctttaactatggagcaacgcaaggtacgcaacttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctggagtggcctgactgacgctgctcgcttgctcggccctattggcaatgcagccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacttatcggagcatctggcgattccgtttgatcgtcgtgcgctggcttatgtcctgaacgcgttagcgccggcccatccacgtccggtgccttgtgtcccggtagcgccgctgattggcggg

SEQ ID NO: 183 - protein, 319 aa, synthetic construct
MRMSRRAKLRCALPLALVAVVAAFAPAANAWLAVDYSLPKALAAWAANPNGSPPGANDWSCRPSSLHPYPVILVHGTFSNQRFTWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTWSGLTDFARLLGPIGNNAVQLVTSFCPACGDQIRGSAFMQELNAGGDTVAGVAYTVIQTKYDEVVTPYDSAFLTPAASSHQVRNILVQKGCPIDLSEHLAIPFDRRALAYVLNALDPAHPRPVPCVPVAPLIGG

SEQ ID NO: 184 - DNA, 957 bp, synthetic construct
  source 1..957: mol_type = other DNA
atgcgtatgtctcgtcgtgccaaactgcgctgcgccttaccccttgcgctggtagctgtggttgctgcctttgcacccgcggcaaatgcatggctggcggtggactatagcctgccgaaagcgctcgctgcatgggcagccaatccgaatggcagcccaccgggtgcgaacgattggtcgtgtcgcccaagctccttgcacccttatccggtgattctggtccacggtacattcagcaaccagcgcttcacttggaatgccctggcgcctctgctgaagaacgcgggttactgcgtcttttcctttaactatggagcaacgcaaggtacgcaacttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctggagtggcctgactgactttgctcgcttgctcggccctattggcaataacgccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacttatcggagcatctggcgattccgtttgatcgtcgtgcgctggcttatgtcctgaacgcgttagatccggcccatccacgtccggtgccttgtgtcccggtagcgccgctgattggcggg

SEQ ID NO: 185 - protein, 319 aa, synthetic construct
MRMSRRAKLRCALPLALVAVVAAFAPAANAQLAVDYSLPKALAAWAANPNGSPPGANDWSCRPSSLHPYPVILVHGTFSNQRFTWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTYSGLTDAARLLGPIGNAAVQLVTSFCPACGDQIRGSAFMQELNAGGDTVAGVAYTVIQTKYDEVVTPYDSAFLTPAASSHQVRNILVQKGCPIDASEHLAIPFDRRALAYVLNALDPAHPRPVPCVPVAPLIGG

SEQ ID NO: 186 - DNA, 957 bp, synthetic construct
  source 1..957: mol_type = other DNA
atgcgtatgtctcgtcgtgccaaactgcgctgcgccttaccccttgcgctggtagctgtggttgctgcctttgcacccgcggcaaatgcacaactggcggtggactatagcctgccgaaagcgctcgctgcatgggcagccaatccgaatggcagcccaccgggtgcgaacgattggtcgtgtcgcccaagctccttgcacccttatccggtgattctggtccacggtacattcagcaaccagcgcttcacttggaatgccctggcgcctctgctgaagaacgcgggttactgcgtcttttcctttaactatggagcaacgcaaggtacgcaacttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctacagtggcctgactgacgctgctcgcttgctcggccctattggcaatgcagccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacgcgtcggagcatctggcgattccgtttgatcgtcgtgcgctggcttatgtcctgaacgcgttagatccggcccatccacgtccggtgccttgtgtcccggtagcgccgctgattggcggg

SEQ ID NO: 187 - protein, 319 aa, synthetic construct
MRMSRRAKLRCALPLALVAVVAAFAPAANAQLAVDYSLPKALAAWAANPNGSPPGANDWSCRPSSLHPYPVILVHGTFSNQRFTWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTWSGLTDAARLLGPIGNAAVQLVTSFCPACGDQIRGSAFMQELNAGGDTVAGVAYTVIQTKYDEVVTPYDSAFLTPAASSHQVRNILVQKGCPIDASEHLAIPFDRRALAYVLNALDPAHPRPVPCVPVAPLIGG

SEQ ID NO: 188 - DNA, 957 bp, synthetic construct
  source 1..957: mol_type = other DNA
atgcgtatgtctcgtcgtgccaaactgcgctgcgccttaccccttgcgctggtagctgtggttgctgcctttgcacccgcggcaaatgcacaactggcggtggactatagcctgccgaaagcgctcgctgcatgggcagccaatccgaatggcagcccaccgggtgcgaacgattggtcgtgtcgcccaagctccttgcacccttatccggtgattctggtccacggtacattcagcaaccagcgcttcacttggaatgccctggcgcctctgctgaagaacgcgggttactgcgtcttttcctttaactatggagcaacgcaaggtacgcaacttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctggagtggcctgactgacgctgctcgcttgctcggccctattggcaatgcagccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacgcgtcggagcatctggcgattccgtttgatcgtcgtgcgctggcttatgtcctgaacgcgttagatccggcccatccacgtccggtgccttgtgtcccggtagcgccgctgattggcggg

SEQ ID NO: 189 - protein, 319 aa, synthetic construct
MRMPRRAKLRCALPLAPVAVVAAFAPAAWAQLAVDYSLPKALAAWAANPNGSPPGANDWSCRPSSLHPYPVILVHGTFENQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTYSGLTDFARLLGPIGNAAVQLVTSFCPACGDQIRGSAFMQELNAGGDTVAGVAYTVIQTKYDEVVTPYDSAFLTPAASSHQVRNILVQKGCPIDLSEHLAIPFDRRALAYVLNALAPAHPRPVPCVPVAPLIGG

SEQ ID NO: 190 - DNA, 957 bp, synthetic construct
  source 1..957: mol_type = other DNA
atgcgtatgcctcgtcgtgccaaactgcgctgcgccttaccccttgcgcctgtagctgtggttgctgcctttgcacccgcggcatgggcacaactggcggtggactatagcctgccgaaagcgctcgctgcatgggcagccaatccgaatggcagcccaccgggtgcgaacgattggtcgtgtcgcccaagctccttgcacccttatccggtgattctggtccacggtacattcgagaaccagcgcttcaattggaatgccctggcgcctctgctgaagaacgcgggttactgcgtcttttcctttaactatggagcaacgcaaggtacgcaacttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctacagtggcctgactgactttgctcgcttgctcggccctattggcaatgcagccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacttatcggagcatctggcgattccgtttgatcgtcgtgcgctggcttatgtcctgaacgcgttagcgccggcccatccacgtccggtgccttgtgtcccggtagcgccgctgattggcggg

SEQ ID NO: 191 - protein, 319 aa, synthetic construct
MRMSRRAKLRCALPLALVAVVAAFAPAAHAQLAVDYSLPKALAAWAANPNGSPPGANDWSCRPSSLHPYPVILVHGTFSNQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTWSGLTDAARLLGPIGNNAVQLVTSFCPACGDQIRGSAFMQELNAGGDTVAGVAYTVIQTKYDEVVTPYDSAFLTPAASSHQVRNILVQKGCPIDLSEHLAIPFDRRALAYVLNALDPAHPRPVPCVPVAPLIGG

SEQ ID NO: 192 - DNA, 957 bp, synthetic construct
  source 1..957: mol_type = other DNA
atgcgtatgtctcgtcgtgccaaactgcgctgcgccttaccccttgcgctggtagctgtggttgctgcctttgcacccgcggcacatgcacaactggcggtggactatagcctgccgaaagcgctcgctgcatgggcagccaatccgaatggcagcccaccgggtgcgaacgattggtcgtgtcgcccaagctccttgcacccttatccggtgattctggtccacggtacattcagcaaccagcgcttcaattggaatgccctggcgcctctgctgaagaacgcgggttactgcgtcttttcctttaactatggagcaacgcaaggtacgcaacttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctggagtggcctgactgacgctgctcgcttgctcggccctattggcaataacgccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacttatcggagcatctggcgattccgtttgatcgtcgtgcgctggcttatgtcctgaacgcgttagatccggcccatccacgtccggtgccttgtgtcccggtagcgccgctgattggcggg

SEQ ID NO: 193 - protein, 319 aa, synthetic construct
MRMSRRAKLRCALPLALVAVVAAFAPAANAQLAVDYSLPKALAAWAANPNGSPPGANDWSCRPSSLHPYPVILVHGTFSNQRFNHNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTYSGLTDAARLLGPIGNAAVQLVTSFCPACGDQIRGSAFMQELNAGGDTVAGVAYTVIQTKYDEVVTPYDSAFLTPAASSHQVRNILVQKGCPIDLSEHLAIPFDRRALAYVLNALAPAHPRPVPCVPVAPLIGG

SEQ ID NO: 194 - DNA, 957 bp, synthetic construct
  source 1..957: mol_type = other DNA
atgcgtatgtctcgtcgtgccaaactgcgctgcgccttaccccttgcgctggtagctgtggttgctgcctttgcacccgcggcaaatgcacaactggcggtggactatagcctgccgaaagcgctcgctgcatgggcagccaatccgaatggcagcccaccgggtgcgaacgattggtcgtgtcgcccaagctccttgcacccttatccggtgattctggtccacggtacattcagcaaccagcgcttcaatcataatgccctggcgcctctgctgaagaacgcgggttactgcgtcttttcctttaactatggagcaacgcaaggtacgcaacttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctacagtggcctgactgacgctgctcgcttgctcggccctattggcaatgcagccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacttatcggagcatctggcgattccgtttgatcgtcgtgcgctggcttatgtcctgaacgcgttagcgccggcccatccacgtccggtgccttgtgtcccggtagcgccgctgattggcggg

SEQ ID NO: 195 - protein, 319 aa, synthetic construct
MRMSRRAKLRCALPLAPVAVVAAFAPAAHAQLAVDYSLPKALAAWAANPNGSPPGANDWSCRPSSLHPYPVILVHGTFSNQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTYSGLTDFARLLGPIGNAAVQLVTSFCPACGDQIRGSAFMQELNAGGDTVAGVAYTVIQTKYDEVVTPYDSAFLTPAASSHQVRNILVQKGCPIDLSEHLAIPFDRRALAYVLNALDPAHPRPVCCVPVAPLIGG

SEQ ID NO: 196 - DNA, 957 bp, synthetic construct
  source 1..957: mol_type = other DNA
atgcgtatgtctcgtcgtgccaaactgcgctgcgccttaccccttgcgcctgtagctgtggttgctgcctttgcacccgcggcacatgcacaactggcggtggactatagcctgccgaaagcgctcgctgcatgggcagccaatccgaatggcagcccaccgggtgcgaacgattggtcgtgtcgcccaagctccttgcacccttatccggtgattctggtccacggtacattcagcaaccagcgcttcaattggaatgccctggcgcctctgctgaagaacgcgggttactgcgtcttttcctttaactatggagcaacgcaaggtacgcaacttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctacagtggcctgactgactttgctcgcttgctcggccctattggcaatgcagccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacttatcggagcatctggcgattccgtttgatcgtcgtgcgctggcttatgtcctgaacgcgttagatccggcccatccacgtccggtgtgttgtgtcccggtagcgccgctgattggcggg

SEQ ID NO: 197 - protein, 319 aa, synthetic construct
MRMSRRAKLRCALPLALVAVVAAFAPAAHAWLAVDYSLPKALAAWAANPNGSPPGANDWSCRPSSLHPYPVILVHGTFENQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTYSGLTDFARLLGPIGNAAVQLVTSFCPACGDQIRGSAFMQELNAGGDTVAGVAYTVIQTKYDEVVTPYDSAFLTPAASSHQVRNILVQKGCPIDASEHLAIPFDRRALAYVLNALAPAHPRPVPCVPVAPLIGG

SEQ ID NO: 198 - DNA, 957 bp, synthetic construct
  source 1..957: mol_type = other DNA
atgcgtatgtctcgtcgtgccaaactgcgctgcgccttaccccttgcgctggtagctgtggttgctgcctttgcacccgcggcacatgcatggctggcggtggactatagcctgccgaaagcgctcgctgcatgggcagccaatccgaatggcagcccaccgggtgcgaacgattggtcgtgtcgcccaagctccttgcacccttatccggtgattctggtccacggtacattcgagaaccagcgcttcaattggaatgccctggcgcctctgctgaagaacgcgggttactgcgtcttttcctttaactatggagcaacgcaaggtacgcaacttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctacagtggcctgactgactttgctcgcttgctcggccctattggcaatgcagccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacgcgtcggagcatctggcgattccgtttgatcgtcgtgcgctggcttatgtcctgaacgcgttagcgccggcccatccacgtccggtgccttgtgtcccggtagcgccgctgattggcggg

SEQ ID NO: 199 - protein, 319 aa, synthetic construct
MRMPRRAKLRAALPLALVAVVAAFAPAANAQLAVDYSLPKALAAWAANPNGSPPGANDWSCRPSSLHPYPVILVHGTFENQRFNHNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTYSGLTDFARLLGPIGNNAVQLVTSFCPACGDQIRGSAFMQELNAGGDTVAGVAYTVIQTKYDEVVTPYDSAFLTPAASSHQVRNILVQKGCPIDLSEHLAIPFDRRALAYVLNALDPAHPRPVPCVPVAPLIGG

SEQ ID NO: 200 - DNA, 957 bp, synthetic construct
  source 1..957: mol_type = other DNA
atgcgtatgcctcgtcgtgccaaactgcgcgcagccttaccccttgcgctggtagctgtggttgctgcctttgcacccgcggcaaatgcacaactggcggtggactatagcctgccgaaagcgctcgctgcatgggcagccaatccgaatggcagcccaccgggtgcgaacgattggtcgtgtcgcccaagctccttgcacccttatccggtgattctggtccacggtacattcgagaaccagcgcttcaatcataatgccctggcgcctctgctgaagaacgcgggttactgcgtcttttcctttaactatggagcaacgcaaggtacgcaacttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctacagtggcctgactgactttgctcgcttgctcggccctattggcaataacgccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacttatcggagcatctggcgattccgtttgatcgtcgtgcgctggcttatgtcctgaacgcgttagatccggcccatccacgtccggtgccttgtgtcccggtagcgccgctgattggcggg

SEQ ID NO: 201 - protein, 319 aa, synthetic construct
MRMPRRAKLRCALPLALVAVVAAFAPAANAQLAVDYSLPKALAAWAANPNGSPPGANDWSCRPSSLHPYPVILVHGTFSNQRFNHNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTYSGLTDFARLLGPIGNAAVQLVTSFCPACGDQIRGSAFMQELNAGGDTVAGVAYTVIQTKYDEVVTPYDSAFLTPAASSHQVRNILVQKGCPIDASEHLAIPFDRRALAYVLNALDPAHPRPVPCVPVAPLIGG

SEQ ID NO: 202 - DNA, 957 bp, synthetic construct
  source 1..957: mol_type = other DNA
atgcgtatgcctcgtcgtgccaaactgcgctgcgccttaccccttgcgcttgtagctgtggttgctgcctttgcacccgcggcaaatgcacaactggcggtggactatagcctgccgaaagcgctcgctgcatgggcagccaatccgaatggcagcccaccgggtgcgaacgattggtcgtgtcgcccaagctccttgcacccttatccggtgattctggtccacggtacattcagcaaccagcgcttcaatcataatgccctggcacctctgctgaagaacgcgggttactgcgtcttttcctttaactatggagcaacgcaaggtacgcaacttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctacagtggcctgactgactttgctcgcttgctcggccctattggcaatgcagccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacgcgtcggagcatctggcgattccgtttgatcgtcgtgcgctggcttatgtcctgaacgcgttagatccggcccatccacgtccggtgccttgtgtcccggtagcgccgctgattggcggg

SEQ ID NO: 203 - protein, 319 aa, synthetic construct
MRMSRRAELRCALPLALVAVVAAFAPAANAQLAVDYSLPMALAAWAANPNGSPPGANDWSCRPSSLHPYPVILVHGTFSNQRFTWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTYSGLTDFARLLGPIGNAAVQLVTSFCPACGDQIRGSAFMQELNAGGDTVAGVAYTVIQTKYDEVVTPYDSAFLTPAASSHQVRNILVQKGCPIDLSEHLAIPFDRRALAYVLNALDPAHPRPVPCVNVAPLIGG

SEQ ID NO: 204 - DNA, 957 bp, synthetic construct
  source 1..957: mol_type = other DNA
atgcgtatgtctcgtcgtgccgaactgcgctgcgccttaccccttgcgctggtagctgtggttgctgcctttgcacccgcggcaaatgcacaactggcggtggactatagcctgccgatggcgctcgctgcatgggcagccaatccgaatggcagcccaccgggtgcgaacgattggtcgtgtcgcccaagctccttgcacccttatccggtgattctggtccacggtacattcagcaaccagcgcttcacttggaatgccctggcgcctctgctgaagaacgcgggttactgcgtcttttcctttaactatggagcaacgcaaggtacgcaacttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctacagtggcctgactgactttgctcgcttgctcggccctattggcaatgcagccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacttatcggagcatctggcgattccgtttgatcgtcgtgcgctggcttatgtcctgaacgcgttagatccggcccatccacgtccggtgccttgtgtcaacgtagcgccgctgattggcggg

SEQ ID NO: 205 - protein, 319 aa, synthetic construct
MRMSRRAKLRCALPLALVAVVAAFAPAANAWLAVDYSLPKALAAWAANPNGSPPGANDWSCRPSSLHPYPVILVHGTFENQRFTWNALAPLLKNAGYCVFSFNYGATQGTELTGGFANGLAAVRDSARQLAAFVDRVRAATGAAEVDIVGHSQGGMMPRYYTNKLGGASKVRALVGLSPSNHGTTYSGLTDFARLLGPIGNAAVQLVTSFCPACGDQIRGSAFMQELNAGGDTVAGVAYTVIQTKYDEVVTPYDSAFLTPAASSHQVRNILVQKGCPIDASEHLAIPFDRRALAYVLNALDPAHPRPVPCVNVAPLIGG

SEQ ID NO: 206 - DNA, 957 bp, synthetic construct
  source 1..957: mol_type = other DNA
atgcgtatgtctcgtcgtgccaaactgcgctgcgccttaccccttgcgctggtagctgtggttgctgcctttgcacccgcggcaaatgcatggctggcggtggactatagcctgccgaaagcgctcgctgcatgggcagccaatccgaatggcagcccaccgggtgcgaacgattggtcgtgtcgcccaagctccttgcacccttatccggtgattctggtccacggtacattcgagaaccagcgcttcacttggaatgccctggcgcctctgctgaagaacgcgggttactgcgtcttttcctttaactatggagcaacgcaaggtacggagcttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctacagtggcctgactgactttgctcgcttgctcggccctattggcaatgcagccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacgcgtcggagcatctggcgattccgtttgatcgtcgtgcgctggcttatgtcctgaacgcgttagatccggcccatccacgtccggtgccttgtgtcaacgtagcgccgctgattggcggg

SEQ ID NO: 207 - protein, 319 aa, synthetic construct
MRMSRRAKLRCALPLALVAVVAAFAPAANAWLAVDYSLPKALAAWAANPNGSPPGANDWSCRPSSLHPYPVILVHGTFENQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTYSGLTDAARLLGPIGNAAVQLVTSFCPACGDQIRGSAFMQELNAGGDTVAGVAYTVIQTKYDEVVTPYDSAFLTPAASSHQVRNILVQKGCPIDASEHLAIPFDRRALAYVLNALAPAHPRPVPCVNVAPLIGG

SEQ ID NO: 208 - DNA, 957 bp, synthetic construct
  source 1..957: mol_type = other DNA
atgcgtatgtctcgtcgtgccaaactgcgctgcgccttaccccttgcgctggtagctgtggttgctgcctttgcacccgcggcaaatgcatggctggcggtggactatagcctgccgaaagcgctcgctgcatgggcagccaatccgaatggcagcccaccgggtgcgaacgattggtcgtgtcgcccaagctccttgcacccttatccggtgattctggtccacggtacattcgagaaccagcgcttcaattggaatgccctggcgcctctgctgaagaacgcgggttactgcgtcttttcctttaactatggagcaacgcaaggtacgcaacttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctacagtggcctgactgacgctgctcgcttgctcggccctattggcaatgcagccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacgcgtcggagcatctggcgattccgtttgatcgtcgtgcgctggcttatgtcctgaacgcgttagcgccggcccatccacgtccggtgccttgtgtcaacgtagcgccgctgattggcggg

SEQ ID NO: 209 - protein, 319 aa, synthetic construct
MRMPRRAELRCALPLALVAVVAAFAPAAWAQLAVDYSLPKALAAWAANPNGSPPGANDWSCRPSSLHPYPVILVHGTFSNQRFNWNALAPLLKNAGYCVFSFNYGATQGTELTGGFANGLAAVRDSARQLAAFVDRVRAATGAAEVDIVGHSQGGMMPRYYTNKLGGASKVRALVGLSPSNHGTTWSGLTDFARLLGPIGNAAVQLVTSFCPACGDQIRGSAFMQELNAGGDTVAGVAYTVIQTKYDEVVTPYDSAFLTPAASSHQVRNILVQKGCPIDLSEHLAIPFDRRALAYVLNALDPAHPRPVPCVPVAPLIGG

SEQ ID NO: 210 - DNA, 957 bp, synthetic construct
  source 1..957: mol_type = other DNA
atgcgtatgccccgtcgtgccgaactgcgctgcgccttaccccttgcgctggtagctgtggttgctgcctttgcacccgcggcatgggcacaactggcggtggactatagcctgccgaaagcgctcgctgcatgggcagccaatccgaatggcagcccaccgggtgcgaacgattggtcgtgtcgcccaagctccttgcacccttatccggtgattctggtccacggtacattcagcaaccagcgcttcaattggaatgccctggcgcctctgctgaagaacgcgggttactgcgtcttttcctttaactatggagcaacgcaaggtacggagcttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctggagtggcctgactgactttgctcgcttgctcggccctattggcaatgcagccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacttatcggagcatctggcgattccgtttgatcgtcgtgcgctggcttatgtcctgaacgcgttagatccggcccatccacgtccggtgccttgtgtcccggtagcgccgctgattggcggg

SEQ ID NO: 211 - protein, 319 aa, synthetic construct
MRMSRRAKLRCALPLAPAAVVAAFAPAANAQLAVDYSLPKALAAWAANPNGSPPGANDWSCRPSSLHPYPVILVHGTFENQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTWSGLTDFARLLGPIGNAAVQLVTSFCPACGDQIRGSAFMQELNAGGDTVAGVAYTVIQTKYDEVVTPYDSAFLTPAASSHQVRNILVQKGCPIDASEHLAIPFDRRALAYVLNALDPAHPRPVCCVNVAPLIGG

SEQ ID NO: 212 - DNA, 957 bp, synthetic construct
  source 1..957: mol_type = other DNA
atgcgtatgtctcgtcgtgccaaactgcgctgcgccttaccccttgcgcctgccgctgtggttgctgcctttgcacccgcggcaaatgcacaactggcggtggactatagcctgccgaaagcgctcgctgcatgggcagccaatccgaatggcagcccaccgggtgcgaacgattggtcgtgtcgcccaagctccttgcacccttatccggtgattctggtccacggtacattcgagaaccagcgcttcaattggaatgccctggcgcctctgctgaagaacgcgggttactgcgtcttttcctttaactatggagcaacgcaaggtacgcaacttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctggagtggcctgactgactttgctcgcttgctcggccctattggcaatgcagccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacgcgtcggagcatctggcgattccgtttgatcgtcgtgcgctggcttatgtcctgaacgcgttagatccggcccatccacgtccggtgtgttgtgtcaacgtagcgccgctgattggcggg

SEQ ID NO: 213 - protein, 319 aa, synthetic construct
MRMSRRAKLRCALPLALVAVVAAFAPAANAQLAVDYSLPKALAAWAANPNGSPPGANDWSCRPSSLHPYPVILVHGTFSNQRFTWNALAPLLKNAGYCVFSFNYGATQGTELTGGFANGLAAVRDSARQLAAFVDRVRAATGAAEVDIVGHSQGGMMPRYYTNKLGGASKVRALVGLSPSNHGTTYSGLTDFARLLGPIGNAAVQLVTSFCPACGDQIRGSAFMQELNAGGDTVAGVAYTVIQTKYDEVVTPYDSAFLTPAASSHQVRNILVQKGCPIDLSEHLAIPFDRRALAYVLNALDPAHPRPVCCVNVAPLIGG

SEQ ID NO: 214 - DNA, 957 bp, synthetic construct
  source 1..957: mol_type = other DNA
atgcgtatgtctcgtcgtgccaaactgcgctgcgccttaccccttgcgctggtagctgtggttgctgcctttgcacccgcggcaaatgcacaactggcggtggactatagcctgccgaaagcgctcgctgcatgggcagccaatccgaatggcagcccaccgggtgcgaacgattggtcgtgtcgcccaagctccttgcacccttatccggtgattctggtccacggtacattcagcaaccagcgcttcacttggaatgccctggcgcctctgctgaagaacgcgggttactgcgtcttttcctttaactatggagcaacgcaaggtacggagcttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctacagtggcctgactgactttgctcgcttgctcggccctattggcaatgcagccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacttatcggagcatctggcgattccgtttgatcgtcgtgcgctggcttatgtcctgaacgcgttagatccggcccatccacgtccggtgtgttgtgtcaacgtagcgccgctgattggcggg

SEQ ID NO: 215 - protein, 319 aa, synthetic construct
MRMSRRAKLRCALPLALVAVVAAFAPAAHAQLAVDYSLPKALAAWAANPNGSPPGANDWSCRPSSLHPYPVILVHGTFENQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTYSGLTDAARLLGPIGNAAVQLVTSFCPACGDQIRGSAFMQELNAGGDTVAGVAYTVIQTKYDEVVTPYDSAFLTPAASSHQVRNILVQKGCPIDASEHLAIPFDRRALAYVLNALAPAHPRPVPCVNVAPLIGG

SEQ ID NO: 216 - DNA, 957 bp, synthetic construct
  source 1..957: mol_type = other DNA
atgcgtatgtctcgtcgtgccaaactgcgctgcgccttaccccttgcgctggtagctgtggttgctgcctttgcacccgcggcacatgcacaactggcggtggactatagcctgccgaaagcgctcgctgcatgggcagccaatccgaatggcagcccaccgggtgcgaacgattggtcgtgtcgcccaagctccttgcacccttatccggtgattctggtccacggtacattcgagaaccagcgcttcaattggaatgccctggcgcctctgctgaagaacgcgggttactgcgtcttttcctttaactatggagcaacgcaaggtacgcaacttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctacagtggcctgactgacgctgctcgcttgctcggccctattggcaatgcagccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacgcgtcggagcatctggcgattccgtttgatcgtcgtgcgctggcttatgtcctgaacgcgttagcgccggcccatccacgtccggtgccttgtgtcaacgtagcgccgctgattggcggg

SEQ ID NO: 217 - protein, 319 aa, synthetic construct
MRMSRRAELRAALPLALVAVVAAFAPAAHAWLAVDYSLPKALAAWAANPNGSPPGANDWSCRPSSLHPYPVILVHGTFENQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTYSGLTDFARLLGPIGNAAVQLVTSFCPACGDQIRGSAFMQELNAGGDTVAGVAYTVIQTKYDEVVTPYDSAFLTPAASSHQVRNILVQKGCPIDLSEHLAIPFDRRALAYVLNALAPAHPRPVPCVPVAPLIGG

SEQ ID NO: 218 - DNA, 957 bp, synthetic construct
  source 1..957: mol_type = other DNA
atgcgtatgtctcgtcgtgccgaactgcgcgcagccttaccccttgcgctggtagctgtggttgctgcctttgcacccgcggcacatgcatggctggcggtggactatagcctgccgaaagcgctcgctgcatgggcagccaatccgaatggcagcccaccgggtgcgaacgattggtcgtgtcgcccaagctccttgcacccttatccggtgattctggtccacggtacattcgagaaccagcgcttcaattggaatgccctggcgcctctgctgaagaacgcgggttactgcgtcttttcctttaactatggagcaacgcaaggtacgcaacttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctacagtggcctgactgactttgctcgcttgctcggccctattggcaatgcagccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacttatcggagcatctggcgattccgtttgatcgtcgtgcgctggcttatgtcctgaacgcgttagcgccggcccatccacgtccggtgccttgtgtcccggtagcgccgctgattggcggg

SEQ ID NO: 219 - protein, 319 aa, synthetic construct
MRMPRRAKLRCALPLALVAVVAAFAPAAHAWLAVDYSLPKALAAWAANPNGSPPGANDWSCRPSSLHPYPVILVHGTFSNQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTLSGLTDFARLLGPIGNAAVQLVTSFCPACGDQIRGSAFMQELNAGGDTVAGVAYTVIQTKYDEVVTPYDSAFLTPAASSHQVRNILVQKGCPIDASEHLAIPFDRRALAYVLNALAPAHPRPVPCVPVAPLIGG

SEQ ID NO: 220 - DNA, 957 bp, synthetic construct
  source 1..957: mol_type = other DNA
atgcgtatgcctcgtcgtgccaaactgcgctgcgccttaccccttgcgctggtagctgtggttgctgcctttgcacccgcggcacatgcatggctggcggtggactatagcctgccgaaagcgctcgctgcatgggcagccaatccgaatggcagcccaccgggtgcgaacgattggtcgtgtcgcccaagctccttgcacccttatccggtgattctggtccacggtacattcagcaaccagcgcttcaattggaatgccctggcgcctctgctgaagaacgcgggttactgcgtcttttcctttaactatggagcaacgcaaggtacgcaacttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaaccctgagtggcctgactgactttgctcgcttgctcggccctattggcaatgcagccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacgcgtcggagcatctggcgattccgtttgatcgtcgtgcgctggcttatgtcctgaacgcgttagcgccggcccatccacgtccggtgccttgtgtcccggtagcgccgctgattggcggg

SEQ ID NO: 221 - protein, 319 aa, synthetic construct
MRMSRRAELRCALPLALVAVVAAFAPAAHAWLAVDYSLPKALAAWAANPNGSPPGANDWSCRPSSLHPYPVILVHGTFSNQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTWSGLTDFARLLGPIGNAAVQLVTSFCPACGDQIRGSAFMQELNAGGDTVAGVAYTVIQTKYDEVVTPYDSAFLTPAASSHQVRNILVQKGCPIDLSEHLAIPFDRRALAYVLNALAPAHPRPVPCVPVAPLIGG

SEQ ID NO: 222 - DNA, 957 bp, synthetic construct
  source 1..957: mol_type = other DNA
atgcgtatgtctcgtcgtgccgaactgcgctgcgccttaccccttgcgctggtagctgtggttgctgcctttgcacccgcggcacatgcatggctggcggtggactatagcctgccgaaagcgctcgctgcatgggcagccaatccgaatggcagcccaccgggtgcgaacgattggtcgtgtcgcccaagctccttgcacccttatccggtgattctggtccacggtacattcagcaaccagcgcttcaattggaatgccctggcgcctctgctgaagaacgcgggttactgcgtcttttcctttaactatggagcaacgcaaggtacgcaacttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctggagtggcctgactgactttgctcgcttgctcggccctattggcaatgcagccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacttatcggagcatctggcgattccgtttgatcgtcgtgcgctggcttatgtcctgaacgcgttagcgccggcccatccacgtccggtgccttgtgtcccggtagcgccgctgattggcggg

SEQ ID NO: 223 - protein, 319 aa, synthetic construct
MRMSRRAELRCALPLAPAAVVAAFAPAANAQLAVDYSLPKALAAWAANPNGSPPGANDWSCRPSSLHPYPVILVHGTFENQRFNWNALAPLLKNAGYCVFSFNYGATQGTELTGGFANGLAAVRDSARQLAAFVDRVRAATGAAEVDIVGHSQGGMMPRYYTNKLGGASKVRALVGLSPSNHGTTWSGLTDFARLLGPIGNAAVQLVTSFCPACGDQIRGSAFMQELNAGGDTVAGVAYTVIQTKYDEVVTPYDSAFLTPAASSHQVRNILVQKGCPIDLSEHLAIPFDRRALAYVLNALDPAHPRPVCCVPVAPLIGG

SEQ ID NO: 224 - DNA, 957 bp, synthetic construct
  source 1..957: mol_type = other DNA
atgcgtatgtctcgtcgtgccgaactgcgctgcgccttaccccttgcgcctgccgctgtggttgctgcctttgcacccgcggcaaatgcacaactggcggtggactatagcctgccgaaagcgctcgctgcatgggcagccaatccgaatggcagcccaccgggtgcgaacgattggtcgtgtcgcccaagctccttgcacccttatccggtgattctggtccacggtacattcgagaaccagcgcttcaattggaatgccctggcgcctctgctgaagaacgcgggttactgcgtcttttcctttaactatggagcaacgcaaggtacggagcttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctggagtggcctgactgactttgctcgcttgctcggccctattggcaatgcagccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacttatcggagcatctggcgattccgtttgatcgtcgtgcgctggcttatgtcctgaacgcgttagatccggcccatccacgtccggtgtgttgtgtcccggtagcgccgctgattggcggg

SEQ ID NO: 225 - protein, 319 aa, synthetic construct
MRMSRRAKLRAALPLALVAVVAAFAPAAWAQLAVDYSLPKALAAWAANPNGSPPGANDWSCRPSSLHPYPVILVHGTFSNQRFTHNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTYSGLTDFARLLGPIGNAAVQLVTSFCPACGDQIRGSAFMQELNAGGDTVAGVAYTVIQTKYDEVVTPYDSAFLTPAASSHQVRNILVQKGCPIDLSEHLAIPFDRRALAYVLNALDPAHPRPVCCVPVAPLIGG

SEQ ID NO: 226 - DNA, 957 bp, synthetic construct
  source 1..957: mol_type = other DNA
atgcgtatgtctcgtcgtgccaaactgcgcgcagccttaccccttgcgctggtagctgtggttgctgcctttgcacccgcggcatgggcacaactggcggtggactatagcctgccgaaagcgctcgctgcatgggcagccaatccgaatggcagcccaccgggtgcgaacgattggtcgtgtcgcccaagctccttgcacccttatccggtgattctggtccacggtacattcagcaaccagcgcttcactcataatgccctggcgcctctgctgaagaacgcgggttactgcgtcttttcctttaactatggagcaacgcaaggtacgcaacttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctacagtggcctgactgactttgctcgcttgctcggccctattggcaatgcagccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacttatcggagcatctggcgattccgtttgatcgtcgtgcgctggcttatgtcctgaacgcgttagatccggcccatccacgtccggtgtgttgtgtcccggtagcgccgctgattggcggg

SEQ ID NO: 227 - protein, 319 aa, synthetic construct
MRMSRRAKLRCALPLALVAVVAAFAPAAHAQLAVDYSLPKALAAWAANPNGSPPGANDWSCRPSSLHPYPVILVHGTFENQRFTWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTYSGLTDFARLLGPIGNNAVQLVTSFCPACGDQIRGSAFMQELNAGGDTVAGVAYTVIQTKYDEVVTPYDSAFLTPAASSHQVRNILVQKGCPIDASEHLAIPFDRRALAYVLNALDPAHPRPVCCVNVAPLIGG

SEQ ID NO: 228 - DNA, 957 bp, synthetic construct
  source 1..957: mol_type = other DNA
atgcgtatgtctcgtcgtgccaaactgcgctgcgccttaccccttgcgctggtagctgtggttgctgcctttgcacccgcggcacatgcacaactggcggtggactatagcctgccgaaagcgctcgctgcatgggcagccaatccgaatggcagcccaccgggtgcgaacgattggtcgtgtcgcccaagctccttgcacccttatccggtgattctggtccacggtacattcgagaaccagcgcttcacttggaatgccctggcgcctctgctgaagaacgcgggttactgcgtcttttcctttaactatggagcaacgcaaggtacgcaacttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctacagtggcctgactgactttgctcgcttgctcggccctattggcaataacgccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacgcgtcggagcatctggcgattccgtttgatcgtcgtgcgctggcttatgtcctgaacgcgttagatccggcccatccacgtccggtgtgttgtgtcaacgtagcgccgctgattggcggg

SEQ ID NO: 229 - protein, 319 aa, synthetic construct
MRMSRRAKLRCALPLALVAVVAAFAPAAHAWLAVDYSLPKALAAWAANPNGSPPGANDWSCRPSSLHPYPVILVHGTFSNQRFTHNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTYSGLTDAARLLGPIGNAAVQLVTSFCPACGDQIRGSAFMQELNAGGDTVAGVAYTVIQTKYDEVVTPYDSAFLTPAASSHQVRNILVQKGCPIDASEHLAIPFDRRALAYVLNALDPAHPRPVCCVPVAPLIGG

SEQ ID NO: 230 - DNA, 957 bp, synthetic construct
  source 1..957: mol_type = other DNA
atgcgtatgtcccgtcgtgccaaactgcgctgcgccttaccccttgcgcttgtagctgtggttgctgcctttgcacccgcggcacatgcatggctggcggtggactatagcctgccgaaagcgctcgctgcatgggcagccaatccgaatggcagcccaccgggtgcgaacgattggtcgtgtcgcccaagctccttgcacccttatccggtgattctggtccacggtacattcagcaaccagcgcttcactcataatgccctggcgcctctgctgaagaacgcgggttactgcgtcttttcctttaactatggagcaacgcaaggtacgcaacttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctacagtggcctgactgacgctgctcgcttgctcggccctattggcaatgcagccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacgcgtcggagcatctggcgattccgtttgatcgtcgtgcgctggcttatgtcctgaacgcgttagatccggcccatccacgtccggtgtgttgtgtcccggtagcgccgctgattggcggg

SEQ ID NO: 231 - protein, 319 aa, synthetic construct
MRMSRRAKLRCALPLAPVAVVAAFAPAANAQLAVDYSLPKALAAWAANPNGSPPGANDWSCRPSSLHPYPVILVHGTFSNQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTWSGLTDFARLLGPIGNNAVQLVTSFCPACGDQIRGSAFMQELNAGGDTVAGVAYTVIQTKYDEVVTPYDSAFLTPAASSHQVRNILVQKGCPIDLSEHLAIPFDRRALAYVLNALDPAHPRPVPCVPVAPLIGG

SEQ ID NO: 232 - DNA, 957 bp, synthetic construct
  source 1..957: mol_type = other DNA
atgcgtatgtctcgtcgtgccaaactgcgctgcgccttaccccttgcgcctgtagctgtggttgctgcctttgcacccgcggcaaatgcacaactggcggtggactatagcctgccgaaagcgctcgctgcatgggcagccaatccgaatggcagcccaccgggtgcgaacgattggtcgtgtcgcccaagctccttgcacccttatccggtgattctggtccacggtacattcagcaaccagcgcttcaattggaatgccctggcgcctctgctgaagaacgcgggttactgcgtcttttcctttaactatggagcaacgcaaggtacgcaacttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctggagtggcctgactgactttgctcgcttgctcggccctattggcaataacgccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacttatcggagcatctggcgattccgtttgatcgtcgtgcgctggcttatgtcctgaacgcgttagatccggcccatccacgtccggtgccttgtgtcccggtagcgccgctgattggcggg

SEQ ID NO: 233 - protein, 319 aa, synthetic construct
MRMSRRAKLRCALPLALVAVVAAFAPAANAQLAVDYSLPMALAAWAANPNGSPPGANDWSCRPSSLHPYPVILVHGTFSNQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTYSGLTDFARLLGPIGNAAVQLVTSFCPACGDQIRGSAFMQELNAGGDTVAGVAYTVIQTKYDEVVTPYDSAFLTPAASSHQVRNILVQKGCPIDLSEHLAIPFDRRALAYVLNALAPAHPRPVPCVPVAPLIGG

SEQ ID NO: 234 - DNA, 957 bp, synthetic construct
  source 1..957: mol_type = other DNA
atgcgtatgtctcgtcgtgccaaactgcgctgcgccttaccccttgcgctggtagctgtggttgctgcctttgcacccgcggcaaatgcacaactggcggtggactatagcctgccgatggcgctcgctgcatgggcagccaatccgaatggcagcccaccgggtgcgaacgattggtcgtgtcgcccaagctccttgcacccttatccggtgattctggtccacggtacattcagcaaccagcgcttcaattggaatgccctggcgcctctgctgaagaacgcgggttactgcgtcttttcctttaactatggagcaacgcaaggtacgcaacttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctacagtggcctgactgactttgctcgcttgctcggccctattggcaatgcagccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacttatcggagcatctggcgattccgtttgatcgtcgtgcgctggcttatgtcctgaacgcgttagcgccggcccatccacgtccggtgccttgtgtcccggtagcgccgctgattggcggg

SEQ ID NO: 235 - protein, 319 aa, synthetic construct
MRMSRRAKLRCALPLALVAVVAAFAPAANAQLAVDYSLPKALAAWAANPNGSPPGANDWSCRPSSLHPYPVILVHGTFSNQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTWSGLTDFARLLGPIGNNAVQLVTSFCPACGDQIRGSAFMQELNAGGDTVAGVAYTVIQTKYDEVVTPYDSAFLTPAASSHQVRNILVQKGCPIDASEHLAIPFDRRALAYVLNALDPAHPRPVPCVPVAPLIGG

SEQ ID NO: 236 - DNA, 957 bp, synthetic construct
  source 1..957: mol_type = other DNA
atgcgtatgtctcgtcgtgccaaactgcgctgcgccttaccccttgcgcttgtagctgtggttgctgcctttgcacccgcggcaaatgcacaactggcggtggactatagcctgccgaaagcgctcgctgcatgggcagccaatccgaatggcagcccaccgggtgcgaacgattggtcgtgtcgcccaagctccttgcacccttatccggtgattctggtccacggtacattcagcaaccagcgcttcaattggaatgccctggcgcctctgctgaagaacgcgggttactgcgtcttttcctttaactatggagcaacgcaaggtacgcaacttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctggagtggcctgactgactttgctcgcttgctcggccctattggcaataacgccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacgcgtcggagcatctggcgattccgtttgatcgtcgtgcgctggcttatgtcctgaacgcgttagatccggcccatccacgtccggtgccttgtgtcccggtagcgccgctgattggcggg

SEQ ID NO: 237 - protein, 319 aa, synthetic construct
MRMSRRAKLRCALPLAPVAVVAAFAPAANAQLAVDYSLPKALAAWAANPNGSPPGANDWSCRPSSLHPYPVILVHGTFENQRFNWNALAPLLKNAGYCVFSFNYGATQGTELTGGFANGLAAVRDSARQLAAFVDRVRAATGAAEVDIVGHSQGGMMPRYYTNKLGGASKVRALVGLSPSNHGTTYSGLTDFARLLGPIGNAAVQLVTSFCPACGDQIRGSAFMQELNAGGDTVAGVAYTVIQTKYDEVVTPYDSAFLTPAASSHQVRNILVQKGCPIDLSEHLAIPFDRRALAYVLNALAPAHPRPVPCVPVAPLIGG

SEQ ID NO: 238 - DNA, 957 bp, synthetic construct
  source 1..957: mol_type = other DNA
atgcgtatgtctcgtcgtgccaaactgcgctgcgccttaccccttgcgcctgtagctgtggttgctgcctttgcacccgcggcaaatgcacaactggcggtggactatagcctgccgaaagcgctcgctgcatgggcagccaatccgaatggcagcccaccgggtgcgaacgattggtcgtgtcgcccaagctccttgcacccttatccggtgattctggtccacggtacattcgagaaccagcgcttcaattggaatgccctggcgcctctgctgaagaacgcgggttactgcgtcttttcctttaactatggagcaacgcaaggtacggagcttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctacagtggcctgactgactttgctcgcttgctcggccctattggcaatgcagccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacttatcggagcatctggcgattccgtttgatcgtcgtgcgctggcttatgtcctgaacgcgttagcgccggcccatccacgtccggtgccttgtgtcccggtagcgccgctgattggcggg

SEQ ID NO: 239 - protein, 319 aa, synthetic construct
MRMSRRAKLRCALPLAPVAVVAAFAPAANAQLAVDYSLPKALAAWAANPNGSPPGANDWSCRPSSLHPYPVILVHGTFSNQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTYSGLTDFARLLGPIGNNAVQLVTSFCPACGDQIRGSAFMQELNAGGDTVAGVAYTVIQTKYDEVVTPYDSAFLTPAASSHQVRNILVQKGCPIDLSEHLAIPFDRRALAYVLNALAPAHPRPVPCVPVAPLIGG

SEQ ID NO: 240 - DNA, 957 bp, synthetic construct
  source 1..957: mol_type = other DNA
atgcgtatgtctcgtcgtgccaaactgcgctgcgccttaccccttgcgcctgtagctgtggttgctgcctttgcacccgcggcaaatgcacaactggcggtggactatagcctgccgaaagcgctcgctgcatgggcagccaatccgaatggcagcccaccgggtgcgaacgattggtcgtgtcgcccaagctccttgcacccttatccggtgattctggtccacggtacattcagcaaccagcgcttcaattggaatgccctggcgcctctgctgaagaacgcgggttactgcgtcttttcctttaactatggagcaacgcaaggtacgcaacttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctacagtggcctgactgactttgctcgcttgctcggccctattggcaataacgccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacttatcggagcatctggcgattccgtttgatcgtcgtgcgctggcttatgtcctgaacgcgttagcgccggcccatccacgtccggtgccttgtgtcccggtagcgccgctgattggcggg

SEQ ID NO: 241 - protein, 319 aa, synthetic construct
MRQSRRAKLRCALPLALVAVVAAFAPAANAQLAVDYSLPKALAAWAANPNGSPPGANDWSCRPSSLHPYPVILVHGTFSNQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTYSGLTDFARLLGPIGNAAVQLVTSFCPACGDQIRGSAFMQELNAGGDTVAGVAYTVIQTKYDEVVTPYDSAFLTPAASSHQVRNILVQKGCPIDLSEHLAIPFDRRALAYVLNALAPAHPRPVPCVPVAPLIGG

SEQ ID NO: 242 - DNA, 957 bp, synthetic construct; identical to SEQ ID NO: 134
  source 1..957: mol_type = other DNA

SEQ ID NO: 243 - protein, 319 aa, synthetic construct
MRMSRRAKLRCALPLAPVAVVAAFAPAANAQLAVDYSLPKALAAWAANPNGSPPGANDWSCRPSSLHPYPVILVHGTFSNQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTWSGLTDFARLLGPIGNAAVQLVTSFCPACGDQIRGSAFMQELNAGGDTVAGVAYTVIQTKYDEVVTPYDSAFLTPAASSHQVRNILVQKGCPIDASEHLAIPFDRRALAYVLNALAPAHPRPVPCVPVAPLIGG

SEQ ID NO: 244 - DNA, 957 bp, synthetic construct
  source 1..957: mol_type = other DNA
atgcgtatgtctcgtcgtgccaaactgcgctgcgccttaccccttgcgcctgtagctgtggttgctgcctttgcacccgcggcaaatgcacaactggcggtggactatagcctgccgaaagcgctcgctgcatgggcagccaatccgaatggcagcccaccgggtgcgaacgattggtcgtgtcgcccaagctccttgcacccttatccggtgattctggtccacggtacattcagcaaccagcgcttcaattggaatgccctggcgcctctgctgaagaacgcgggttactgcgtcttttcctttaactatggagcaacgcaaggtacgcaacttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctggagtggcctgactgactttgctcgcttgctcggccctattggcaatgcagccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacgcgtcggagcatctggcgattccgtttgatcgtcgtgcgctggcttatgtcctgaacgcgttagcgccggcccatccacgtccggtgccttgtgtcccggtagcgccgctgattggcggg

SEQ ID NO: 245 - protein, 319 aa, synthetic construct
MRMSRRAKLRCALPLAPVAVVAAFAPAANAQLAVDYSLPKALAAWAANPNGSPPGANDWSCRPSSLHPYPVILVHGTFSNQRFNWNALAPLLKNAGYCVFSFNYGATQGTELTGGFANGLAAVRDSARQLAAFVDRVRAATGAAEVDIVGHSQGGMMPRYYTNKLGGASKVRALVGLSPSNHGTTWSGLTDFARLLGPIGNAAVQLVTSFCPACGDQIRGSAFMQELNAGGDTVAGVAYTVIQTKYDEVVTPYDSAFLTPAASSHQVRNILVQKGCPIDLSEHLAIPFDRRALAYVLNALDPAHPRPVCCVPVAPLIGG

SEQ ID NO: 246 - DNA, 957 bp, synthetic construct
  source 1..957: mol_type = other DNA
atgcgtatgtctcgtcgtgccaaactgcgctgcgccttaccccttgcgcctgtagctgtggttgctgcctttgcacccgcggcaaatgcacaactggcggtggactatagcctgccgaaagcgctcgctgcatgggcagccaatccgaatggcagcccaccgggtgcgaacgattggtcgtgtcgcccaagctccttgcacccttatccggtgattctggtccacggtacattcagcaaccagcgcttcaattggaatgccctggcgcctctgctgaagaacgcgggttactgcgtcttttcctttaactatggagcaacgcaaggtacggagcttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctggagtggcctgactgactttgctcgcttgctcggccctattggcaatgcagccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacttatcggagcatctggcgattccgtttgatcgtcgtgcgctggcttatgtcctgaacgcgttagatccggcccatccacgtccggtgtgttgtgtcccggtagcgccgctgattggcggg

SEQ ID NO: 247 - protein, 319 aa, synthetic construct
MRMSRRAKLRCALPLAPCAVVAAFAPAANAQLAVDYSLPKALAAWAANPNGSPPGANDWSCRPSSLHPYPVILVHGTFSNQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTWSGLTDFARLLGPIGNAAVQLVTSFCPACGDQIRGSAFMQELNAGGDTVAGVAYTVIQTKYDEVVTPYDSAFLTPAASSHQVRNILVQKGCPIDASEHLAIPFDRRALAYVLNALDPAHPRPVPCVPVAPLIGG

SEQ ID NO: 248 - DNA, 957 bp, synthetic construct
  source 1..957: mol_type = other DNA
atgcgtatgtcccgtcgtgccaaactgcgctgcgccttaccccttgcgccttgcgctgtggttgctgcctttgcacccgcggcaaatgcacaactggcggtggactatagcctgccgaaagcgctcgctgcatgggcagccaatccgaatggcagcccaccgggtgcgaacgattggtcgtgtcgcccaagctccttgcacccttatccggtgattctggtccacggtacattcagcaaccagcgcttcaattggaatgccctggcgcctctgctgaagaacgcgggttactgcgtcttttcctttaactatggagcaacgcaaggtacgcaacttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctggagtggcctgactgactttgctcgcttgctcggccctattggcaatgcagccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacgcgtcggagcatctggcgattccgtttgatcgtcgtgcgctggcttatgtcctgaacgcgttagatccggcccatccacgtccggtgccttgtgtcccggtagcgccgctgattggcggg

SEQ ID NO: 249 - protein, 319 aa, synthetic construct
MRMSRRAKLRCALPLALVAVVAAFAPAAHAQLAVDYSLPMALAAWAANPNGSPPGANDWSCRPSSLHPYPVILVHGTFSNQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTWSGLTDFARLLGPIGNAAVQLVTSFCPACGDQIRGSAFMQELNAGGDTVAGVAYTVIQTKYDEVVTPYDSAFLTPAASSHQVRNILVQKGCPIDLSEHLAIPFDRRALAYVLNALAPAHPRPVPCVPVAPLIGG

SEQ ID NO: 250 - DNA, 957 bp, synthetic construct
  source 1..957: mol_type = other DNA
atgcgtatgtctcgtcgtgccaaactgcgctgcgccttaccccttgcgctggtagctgtggttgctgcctttgcacccgcggcacatgcacaactggcggtggactatagcctgccgatggcgctcgctgcatgggcagccaatccgaatggcagcccaccgggtgcgaacgattggtcgtgtcgcccaagctccttgcacccttatccggtgattctggtccacggtacattcagcaaccagcgcttcaattggaatgccctggcgcctctgctgaagaacgcgggttactgcgtcttttcctttaactatggagcaacgcaaggtacgcaacttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctggagtggcctgactgactttgctcgcttgctcggccctattggcaatgcagccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacttatcggagcatctggcgattccgtttgatcgtcgtgcgctggcttatgtcctgaacgcgttagcgccggcccatccacgtccggtgccttgtgtcccggtagcgccgctgattggcggg

SEQ ID NO: 251 - protein, 319 aa, synthetic construct
MRMSRRAKLRCALPLALVAVVAAFAPAAHAQLAVDYSLPKALAAWAANPNGSPPGANDWSCRPSSLHPYPVILVHGTFSNQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTWSGLTDFARLLGPIGNNAVQLVTSFCPACGDQIRGSAFMQELNAGGDTVAGVAYTVIQTKYDEVVTPYDSAFLTPAASSHQVRNILVQKGCPIDASEHLAIPFDRRALAYVLNALDPAHPRPVPCVPVAPLIGG

SEQ ID NO: 252 - DNA, 957 bp, synthetic construct
  source 1..957: mol_type = other DNA
atgcgtatgtctcgtcgtgccaaactgcgctgcgccttaccccttgcgctggtagctgtggttgctgcctttgcacccgcggcacatgcacaactggcggtggactatagcctgccgaaagcgctcgctgcatgggcagccaatccgaatggcagcccaccgggtgcgaacgattggtcgtgtcgcccaagctccttgcacccttatccggtgattctggtccacggtacattcagcaaccagcgcttcaattggaatgccctggcgcctctgctgaagaacgcgggttactgcgtcttttcctttaactatggagcaacgcaaggtacgcaacttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctggagtggcctgactgactttgctcgcttgctcggccctattggcaataacgccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacgcgtcggagcatctggcgattccgtttgatcgtcgtgcgctggcttatgtcctgaacgcgttagatccggcccatccacgtccggtgccttgtgtcccggtagcgccgctgattggcggg

SEQ ID NO: 253 - protein, 319 aa, synthetic construct
MRMSRRAKLRCALPLALVAVVAAFAPAAHAQLAVDYSLPKALAAWAANPNGSPPGANDWSCRPSSLHPYPVILVHGTFSNQRFNWNALAPLLKNAGYCVFSFNYGATQGTELTGGFANGLAAVRDSARQLAAFVDRVRAATGAAEVDIVGHSQGGMMPRYYTNKLGGASKVRALVGLSPSNHGTTWSGLTDFARLLGPIGNAAVQLVTSFCPACGDQIRGSAFMQELNAGGDTVAGVAYTVIQTKYDEVVTPYDSAFLTPAASSHQVRNILVQKGCPIDASEHLAIPFDRRALAYVLNALDPAHPRPVPCVPVAPLIGG

SEQ ID NO: 254 - DNA, 957 bp, synthetic construct
  source 1..957: mol_type = other DNA
atgcgtatgtctcgtcgtgccaaactgcgctgcgccttaccccttgcgctggtagctgtggttgctgcctttgcacccgcggcacatgcacaactggcggtggactatagcctgccgaaagcgctcgctgcatgggcagccaatccgaatggcagcccaccgggtgcgaacgattggtcgtgtcgcccaagctccttgcacccttatccggtgattctggtccacggtacattcagcaaccagcgcttcaattggaatgccctggcgcctctgctgaagaacgcgggttactgcgtcttttcctttaactatggagcaacgcaaggtacggagcttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctggagtggcctgactgactttgctcgcttgctcggccctattggcaatgcagccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacgcgtcggagcatctggcgattccgtttgatcgtcgtgcgctggcttatgtcctgaacgcgttagatccggcccatccacgtccggtgccttgtgtcccggtagcgccgctgattggcggg

SEQ ID NO: 255 - protein, 319 aa, synthetic construct
MRMSRRAKLRCALPLALVAVVAAFAPAAHAQLAVDYSLPKALAAWAANPNGSPPGANDWSCRPSSLHPYPVILVHGTFSNQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTWSGLTDFARLLGPIGNNAVQLVTSFCPACGDQIRGSAFMQELNAGGDTVAGVAYTVIQTKYDEVVTPYDSAFLTPAASSHQVRNILVQKGCPIDLSEHLAIPFDRRALAYVLNALAPAHPRPVPCVPVAPLIGG

SEQ ID NO: 256 - DNA, 957 bp, synthetic construct
  source 1..957: mol_type = other DNA
atgcgtatgtctcgtcgtgccaaactgcgctgcgccttaccccttgcgctggtagctgtggttgctgcctttgcacccgcggcacatgcacaactggcggtggactatagcctgccgaaagcgctcgctgcatgggcagccaatccgaatggcagcccaccgggtgcgaacgattggtcgtgtcgcccaagctccttgcacccttatccggtgattctggtccacggtacattcagcaaccagcgcttcaattggaatgccctggcgcctctgctgaagaacgcgggttactgcgtcttttcctttaactatggagcaacgcaaggtacgcaacttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctggagtggcctgactgactttgctcgcttgctcggccctattggcaataacgccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacttatcggagcatctggcgattccgtttgatcgtcgtgcgctggcttatgtcctgaacgcgttagcgccggcccatccacgtccggtgccttgtgtcccggtagcgccgctgattggcggg

SEQ ID NO: 257 - protein, 319 aa, synthetic construct
MRMSRRAKLRAALPLALVAVVAAFAPAANAQLAVDYSLPKALAAWAANPNGSPPGANDWSCRPSSLHPYPVILVHGTFENQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTYSGLTDFARLLGPIGNAAVQLVTSFCPACGDQIRGSAFMQELNAGGDTVAGVAYTVIQTKYDEVVTPYDSAFLTPAASSHQVRNILVQKGCPIDASEHLAIPFDRRALAYVLNALAPAHPRPVCCVPVAPLIGG

SEQ ID NO: 258 - DNA, 957 bp, synthetic construct
  source 1..957: mol_type = other DNA
atgcgtatgtctcgtcgtgccaaactgcgcgcagccttaccccttgcgctggtagctgtggttgctgcctttgcacccgcggcaaatgcacaactggcggtggactatagcctgccgaaagcgctcgctgcatgggcagccaatccgaatggcagcccaccgggtgcgaacgattggtcgtgtcgcccaagctccttgcacccttatccggtgattctggtccacggtacattcgagaaccagcgcttcaattggaatgccctggcgcctctgctgaagaacgcgggttactgcgtcttttcctttaactatggagcaacgcaaggtacgcaacttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctacagtggcctgactgactttgctcgcttgctcggccctattggcaatgcagccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacgcgtcggagcatctggcgattccgtttgatcgtcgtgcgctggcttatgtcctgaacgcgttagcgccggcccatccacgtccggtgtgttgtgtcccggtagcgccgctgattggcggg

SEQ ID NO: 259 - protein, 319 aa, synthetic construct
MRMSRRAELRCALPLALVAVVAAFAPAANAQLAVDYSLPKALAAWAANPNGSPPGANDWSCRPSSLHPYPVILVHGTFSNQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTYSGLTDFARLLGPIGNAAVQLVTSFCPACGDQIRGSAFMQELNAGGDTVAGVAYTVIQTKYDEVVTPYDSAFLTPAASSHQVRNILVQKGCPIDASEHLAIPFDRRALAYVLNALAPAHPRPVPCVPVAPLIGG

SEQ ID NO: 260 - DNA, 957 bp, synthetic construct
  source 1..957: mol_type = other DNA
atgcgtatgtctcgtcgtgccgaactgcgctgcgccttaccccttgcgctggtagctgtggttgctgcctttgcacccgcggcaaatgcacaactggcggtggactatagcctgccgaaagcgctcgctgcatgggcagccaatccgaatggcagcccaccgggtgcgaacgattggtcgtgtcgcccaagctccttgcacccttatccggtgattctggtccacggtacattcagcaaccagcgcttcaattggaatgccctggcgcctctgctgaagaacgcgggttactgcgtcttttcctttaactatggagcaacgcaaggtacgcaacttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctacagtggcctgactgactttgctcgcttgctcggccctattggcaatgcagccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacgcgtcggagcatctggcgattccgtttgatcgtcgtgcgctggcttatgtcctgaacgcgttagcgccggcccatccacgtccggtgccttgtgtcccggtagcgccgctgattggcggg

SEQ ID NO: 261 - protein, 319 aa, synthetic construct
MRMSRRAKLRCALPLAPVAVVAAFAPAAWAQLAVDYSLPKALAAWAANPNGSPPGANDWSCRPSSLHPYPVILVHGTFENQRFNWNALAPLLKNAGYCVFSFNYGATQGTELTGGFANGLAAVRDSARQLAAFVDRVRAATGAAEVDIVGHSQGGMMPRYYTNKLGGASKVRALVGLSPSNHGTTWSGLTDFARLLGPIGNNAVQLVTSFCPACGDQIRGSAFMQELNAGGDTVAGVAYTVIQTKYDEVVTPYDSAFLTPAASSHQVRNILVQKGCPIDLSEHLAIPFDRRALAYVLNALDPAHPRPVPCVPVAPLIGG

SEQ ID NO: 262 - DNA, 957 bp, synthetic construct
  source 1..957: mol_type = other DNA
atgcgtatgtctcgtcgtgccaaactgcgctgcgccttaccccttgcgcctgtagctgtggttgctgcctttgcacccgcggcatgggcacaactggcggtggactatagcctgccgaaagcgctcgctgcatgggcagccaatccgaatggcagcccaccgggtgcgaacgattggtcgtgtcgcccaagctccttgcacccttatccggtgattctggtccacggtacattcgagaaccagcgcttcaattggaatgccctggcgcctctgctgaagaacgcgggttactgcgtcttttcctttaactatggagcaacgcaaggtacggagcttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctggagtggcctgactgactttgctcgcttgctcggccctattggcaataacgccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacttatcggagcatctggcgattccgtttgatcgtcgtgcgctggcttatgtcctgaacgcgttagatccggcccatccacgtccggtgccttgtgtcccggtagcgccgctgattggcggg

SEQ ID NO: 263 - protein, 319 aa, synthetic construct
MRMSRRAKLRCALPLAPVAVVAAFAPAANAQLAVDYSLPKALAAWAANPNGSPPGANDWSCRPSSLHPYPVILVHGTFENQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTYSGLTDFARLLGPIGNAAVQLVTSFCPACGDQIRGSAFMQELNAGGDTVAGVAYTVIQTKYDEVVTPYDSAFLTPAASSHQVRNILVQKGCPIDASEHLAIPFDRRALAYVLNALAPAHPRPVCCVPVAPLIGG

SEQ ID NO: 264 - DNA, 957 bp, synthetic construct
  source 1..957: mol_type = other DNA
atgcgtatgtctcgtcgtgccaaactgcgctgcgccttaccccttgcgcctgtagctgtggttgctgcctttgcacccgcggcaaatgcacaactggcggtggactatagcctgccgaaagcgctcgctgcatgggcagccaatccgaatggcagcccaccgggtgcgaacgattggtcgtgtcgcccaagctccttgcacccttatccggtgattctggtccacggtacattcgagaaccagcgcttcaattggaatgccctggcgcctctgctgaagaacgcgggttactgcgtcttttcctttaactatggagcaacgcaaggtacgcaacttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctacagtggcctgactgactttgctcgcttgctcggccctattggcaatgcagccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacgcgtcggagcatctggcgattccgtttgatcgtcgtgcgctggcttatgtcctgaacgcgttagcgccggcccatccacgtccggtgtgttgtgtcccggtagcgccgctgattggcggg

SEQ ID NO: 265 - protein, 319 aa, synthetic construct
MRMSRRAKLRCALPLALVAVVAAFAPAANAQLAVDYSLPKALAAWAANPNGSPPGANDWSCRPSSLHPYPVILVHGTFENQRFNWNALAPLLKNAGYCVFSFNYGATQGTELTGGFANGLAAVRDSARQLAAFVDRVRAATGAAEVDIVGHSQGGMMPRYYTNKLGGASKVRALVGLSPSNHGTTWSGLTDFARLLGPIGNNAVQLVTSFCPACGDQIRGSAFMQELNAGGDTVAGVAYTVIQTKYDEVVTPYDSAFLTPAASSHQVRNILVQKGCPIDASEHLAIPFDRRALAYVLNALDPAHPRPVPCVNVAPLIGG

SEQ ID NO: 266 - DNA, 957 bp, synthetic construct
  source 1..957: mol_type = other DNA
atgcgtatgtctcgtcgtgccaaactgcgctgcgccttaccccttgcgctggtagctgtggttgctgcctttgcacccgcggcaaatgcacaactggcggtggactatagcctgccgaaagcgctcgctgcatgggcagccaatccgaatggcagcccaccgggtgcgaacgattggtcgtgtcgcccaagctccttgcacccttatccggtgattctggtccacggtacattcgagaaccagcgcttcaattggaatgccctggcgcctctgctgaagaacgcgggttactgcgtcttttcctttaactatggagcaacgcaaggtacggagcttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctggagtggcctgactgactttgctcgcttgctcggccctattggcaataacgccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacgcgtcggagcatctggcgattccgtttgatcgtcgtgcgctggcttatgtcctgaacgcgttagatccggcccatccacgtccggtgccttgtgtcaacgtagcgccgctgattggcggg

SEQ ID NO: 267 - protein, 319 aa, synthetic construct
MRMSRRAKLRCALPLALVAVVAAFAPAAHAQLAVDYSLPKALAAWAANPNGSPPGANDWSCRPSSLHPYPVILVHGTFSNQRFNWNALAPLLKNAGYCVFSFNYGATQGTELTGGFANGLAAVRDSARQLAAFVDRVRAATGAAEVDIVGHSQGGMMPRYYTNKLGGASKVRALVGLSPSNHGTTYSGLTDFARLLGPIGNAAVQLVTSFCPACGDQIRGSAFMQELNAGGDTVAGVAYTVIQTKYDEVVTPYDSAFLTPAASSHQVRNILVQKGCPIDLSEHLAIPFDRRALAYVLNALAPAHPRPVPCVPVAPLIGG

SEQ ID NO: 268 - DNA, 957 bp, synthetic construct
  source 1..957: mol_type = other DNA
atgcgtatgtctcgtcgtgccaaactgcgctgcgccttaccccttgcgctggtagctgtggttgctgcctttgcacccgcggcacatgcacaactggcggtggactatagcctgccgaaagcgctcgctgcatgggcagccaatccgaatggcagcccaccgggtgcgaacgattggtcgtgtcgcccaagctccttgcacccttatccggtgattctggtccacggtacattcagcaaccagcgcttcaattggaatgccctggcgcctctgctgaagaacgcgggttactgcgtcttttcctttaactatggagcaacgcaaggtacggagcttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctacagtggcctgactgactttgctcgcttgctcggccctattggcaatgcagccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacttatcggagcatctggcgattccgtttgatcgtcgtgcgctggcttatgtcctgaacgcgttagcgccggcccatccacgtccggtgccttgtgtcccggtagcgccgctgattggcggg

SEQ ID NO: 269 - protein, 319 aa, synthetic construct
MRMSRRAKLRCALPLAPVAVVAAFAPAAWAQLAVDYSLPKALAAWAANPNGSPPGANDWSCRPSSLHPYPVILVHGTFSNQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTYSGLTDFARLLGPIGNNAVQLVTSFCPACGDQIRGSAFMQELNAGGDTVAGVAYTVIQTKYDEVVTPYDSAFLTPAASSHQVRNILVQKGCPIDLSEHLAIPFDRRALAYVLNALDPAHPRPVPCVNVAPLIGG

SEQ ID NO: 270 - DNA, 957 bp, synthetic construct
  source 1..957: mol_type = other DNA
atgcgtatgtctcgtcgtgccaaactgcgctgcgccttaccccttgcgcctgtagctgtggttgctgcctttgcacccgcggcatgggcacaactggcggtggactatagcctgccgaaagcgctcgctgcatgggcagccaatccgaatggcagcccaccgggtgcgaacgattggtcgtgtcgcccaagctccttgcacccttatccggtgattctggtccacggtacattcagcaaccagcgcttcaattggaatgccctggcgcctctgctgaagaacgcgggttactgcgtcttttcctttaactatggagcaacgcaaggtacgcaacttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctacagtggcctgactgactttgctcgcttgctcggccctattggcaataacgccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacttatcggagcatctggcgattccgtttgatcgtcgtgcgctggcttatgtcctgaacgcgttagatccggcccatccacgtccggtgccttgtgtcaacgtagcgccgctgattggcggg

SEQ ID NO: 271 - protein, 319 aa, synthetic construct
MRMSRRAKLRCALPLAPVAVVAAFAPAANAQLAVDYSLPKALAAWAANPNGSPPGANDWSCRPSSLHPYPVILVHGTFSNQRFTWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTYSGLTDFARLLGPIGNAAVQLVTSFCPACGDQIRGSAFMQELNAGGDTVAGVAYTVIQTKYDEVVTPYDSAFLTPAASSHQVRNILVQKGCPIDASEHLAIPFDRRALAYVLNALDPAHPRPVCCVPVAPLIGG

SEQ ID NO: 272 - DNA, 957 bp, synthetic construct
  source 1..957: mol_type = other DNA
atgcgtatgtctcgtcgtgccaaactgcgctgcgccttaccccttgcgcctgtagctgtggttgctgcctttgcacccgcggcaaatgcacaactggcggtggactatagcctgccgaaagcgctcgctgcatgggcagccaatccgaatggcagcccaccgggtgcgaacgattggtcgtgtcgcccaagctccttgcacccttatccggtgattctggtccacggtacattcagcaaccagcgcttcacttggaatgccctggcgcctctgctgaagaacgcgggttactgcgtcttttcctttaactatggagcaacgcaaggtacgcaacttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctacagtggcctgactgactttgctcgcttgctcggccctattggcaatgcagccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacgcgtcggagcatctggcgattccgtttgatcgtcgtgcgctggcttatgtcctgaacgcgttagatccggcccatccacgtccggtgtgttgtgtcccggtagcgccgctgattggcggg

SEQ ID NO: 273 - protein, 319 aa, synthetic construct
MRMSRRAKLRCALPLAPVAVVAAFAPAAHAQLAVDYSLPKALAAWAANPNGSPPGANDWSCRPSSLHPYPVILVHGTFSNQRFNHNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTWSGLTDFARLLGPIGNAAVQLVTSFCPACGDQIRGSAFMQELNAGGDTVAGVAYTVIQTKYDEVVTPYDSAFLTPAASSHQVRNILVQKGCPIDASEHLAIPFDRRALAYVLNALDPAHPRPVPCVPVAPLIGG

SEQ ID NO: 274 - DNA, 957 bp, synthetic construct
  source 1..957: mol_type = other DNA
atgcgtatgtctcgtcgtgccaaactgcgctgcgccttaccccttgcgcctgtagctgtggttgctgcctttgcacccgcggcacatgcacaactggcggtggactatagcctgccgaaagcgctcgctgcatgggcagccaatccgaatggcagcccaccgggtgcgaacgattggtcgtgtcgcccaagctccttgcacccttatccggtgattctggtccacggtacattcagcaaccagcgcttcaatcataatgccctggcgcctctgctgaagaacgcgggttactgcgtcttttcctttaactatggagcaacgcaaggtacgcaacttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctggagtggcctgactgactttgctcgcttgctcggccctattggcaatgcagccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacgcgtcggagcatctggcgattccgtttgatcgtcgtgcgctggcttatgtcctgaacgcgttagatccggcccatccacgtccggtgccttgtgtcccggtagcgccgctgattggcggg

SEQ ID NO: 275 - protein, 319 aa, synthetic construct
MRMSRRAKLRCALPLAPVAVVAAFAPAANAQLAVDYSLPKALAAWAANPNGSPPGANDWSCRPSSLHPYPVILVHGTFSNQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTYSGLTDAARLLGPIGNNAVQLVTSFCPACGDQIRGSAFMQELNAGGDTVAGVAYTVIQTKYDEVVTPYDSAFLTPAASSHQVRNILVQKGCPIDLSEHLAIPFDRRALAYVLNALDPAHPRPVCCVPVAPLIGG

SEQ ID NO: 276 - DNA, 957 bp, synthetic construct
  source 1..957: mol_type = other DNA
atgcgtatgtctcgtcgtgccaaactgcgctgcgccttaccccttgcgcctgtagctgtggttgctgcctttgcacccgcggcaaatgcacaactggcggtggactatagcctgccgaaagcgctcgctgcatgggcagccaatccgaatggcagcccaccgggtgcgaacgattggtcgtgtcgcccaagctccttgcacccttatccggtgattctggtccacggtacattctccaaccagcgcttcaattggaatgccctggcgcctctgctgaagaacgcgggttactgcgtcttttcctttaactatggagcaacgcaaggtacgcaacttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctacagtggcctgactgacgctgctcgcttgctcggccctattggcaataacgccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacttatcggagcatctggcgattccgtttgatcgtcgtgcgctggcttatgtcctgaacgcgttagatccggcccatccacgtccggtgtgttgtgtcccggtagcgccgctgattggcggg

SEQ ID NO: 277 - protein, 319 aa, synthetic construct
MRMSRRAKLRCALPLALAAVVAAFAPAANAQLAVDYSLPMALAAWAANPNGSPPGANDWSCRPSSLHPYPVILVHGTFSNQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTWSGLTDAARLLGPIGNAAVQLVTSFCPACGDQIRGSAFMQELNAGGDTVAGVAYTVIQTKYDEVVTPYDSAFLTPAASSHQVRNILVQKGCPIDLSEHLAIPFDRRALAYVLNALAPAHPRPVPCVPVAPLIGG

SEQ ID NO: 278 - DNA, 957 bp, synthetic construct
  source 1..957: mol_type = other DNA
atgcgtatgtcccgtcgtgccaaactgcgctgcgccttaccccttgcgcttgccgctgtggttgctgcctttgcacccgcggcaaatgcacaactggcggtggactatagcctgccgatggcgctcgctgcatgggcagccaatccgaatggcagcccaccgggtgcgaacgattggtcgtgtcgcccaagctccttgcacccttatccggtgattctggtccacggtacattcagcaaccagcgcttcaattggaatgccctggcgcctctgctgaagaacgcgggttactgcgtcttttcctttaactatggagcaacgcaaggtacgcaacttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctggagtggcctgactgacgctgctcgcttgctcggccctattggcaatgcagccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacttatcggagcatctggcgattccgtttgatcgtcgtgcgctggcttatgtcctgaacgcgttagcgccggcccatccacgtccggtgccttgtgtcccggtagcgccgctgattggcggg

SEQ ID NO: 279 - protein, 319 aa, synthetic construct
MRMSRRAKLRCALPLAPVAVVAAFAPAAHAWLAVDYSLPKALAAWAANPNGSPPGANDWSCRPSSLHPYPVILVHGTFSNQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTWSGLTDFARLLGPIGNAAVQLVTSFCPACGDQIRGSAFMQELNAGGDTVAGVAYTVIQTKYDEVVTPYDSAFLTPAASSHQVRNILVQKGCPIDLSEHLAIPFDRRALAYVLNALAPAHPRPVPCVPVAPLIGG

SEQ ID NO: 280 - DNA, 957 bp, synthetic construct
  source 1..957: mol_type = other DNA
atgcgtatgtctcgtcgtgccaaactgcgctgcgccttaccccttgcgcctgtagctgtggttgctgcctttgcacccgcggcacatgcatggctggcggtggactatagcctgccgaaagcgctcgctgcatgggcagccaatccgaatggcagcccaccgggtgcgaacgattggtcgtgtcgcccaagctccttgcacccttatccggtgattctggtccacggtacattcagcaaccagcgcttcaattggaatgccctggcgcctctgctgaagaacgcgggttactgcgtcttttcctttaactatggagcaacgcaaggtacgcaacttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctggagtggcctgactgactttgctcgcttgctcggccctattggcaatgcagccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacttatcggagcatctggcgattccgtttgatcgtcgtgcgctggcttatgtcctgaacgcgttagcgccggcccatccacgtccggtgccttgtgtcccggtagcgccgctgattggcggg

SEQ ID NO: 281 - protein, 319 aa, synthetic construct
MRMSRRAELRCALPLALVAVVAAFAPAANAQLAVDYSLPMALAAWAANPNGSPPGANDWSCRPSSLHPYPVILVHGTFENQRFNWNALAPLLKNAGYCVFSFNYGATQGTELTGGFANGLAAVRDSARQLAAFVDRVRAATGAAEVDIVGHSQGGMMPRYYTNKLGGASKVRALVGLSPSNHGTTWSGLTDFARLLGPIGNAAVQLVTSFCPACGDQIRGSAFMQELNAGGDTVAGVAYTVIQTKYDEVVTPYDSAFLTPAASSHQVRNILVQKGCPIDASEHLAIPFDRRALAYVLNALDPAHPRPVCCVPVAPLIGG

SEQ ID NO: 282 - DNA, 957 bp, synthetic construct
  source 1..957: mol_type = other DNA
atgcgtatgtctcgtcgtgccgaactgcgctgcgccttaccccttgcgctggtagctgtggttgctgcctttgcacccgcggcaaatgcacaactggcggtggactatagcctgccgatggcgctcgctgcatgggcagccaatccgaatggcagcccaccgggtgcgaacgattggtcgtgtcgcccaagctccttgcacccttatccggtgattctggtccacggtacattcgagaaccagcgcttcaattggaatgccctggcgcctctgctgaagaacgcgggttactgcgtcttttcctttaactatggagcaacgcaaggtacggagcttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctggagtggcctgactgactttgctcgcttgctcggccctattggcaatgcagccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacgcgtcggagcatctggcgattccgtttgatcgtcgtgcgctggcttatgtcctgaacgcgttagatccggcccatccacgtccggtgtgttgtgtcccggtagcgccgctgattggcggg

SEQ ID NO: 283 - protein, 319 aa, synthetic construct
MRQSRRAELRCALPLAPVAVVAAFAPAANAQLAVDYSLPKALAAWAANPNGSPPGANDWSCRPSSLHPYPVILVHGTFENQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTYSGLTDFARLLGPIGNAAVQLVTSFCPACGDQIRGSAFMQELNAGGDTVAGVAYTVIQTKYDEVVTPYDSAFLTPAASSHQVRNILVQKGCPIDLSEHLAIPFDRRALAYVLNALAPAHPRPVPCVNVAPLIGG

SEQ ID NO: 284 - DNA, 957 bp, synthetic construct
  source 1..957: mol_type = other DNA
atgcgtcagtcccgtcgtgccgaactgcgctgcgccttaccccttgcgcctgtagctgtggttgctgcctttgcacccgcggcaaatgcacaactggcggtggactatagcctgccgaaagcgctcgctgcatgggcagccaatccgaatggcagcccaccgggtgcgaacgattggtcgtgtcgcccaagctccttgcacccttatccggtgattctggtccacggtacattcgagaaccagcgcttcaattggaatgccctggcgcctctgctgaagaacgcgggttactgcgtcttttcctttaactatggagcaacgcaaggtacgcaacttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctacagtggcctgactgactttgctcgcttgctcggccctattggcaatgcagccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacttatcggagcatctggcgattccgtttgatcgtcgtgcgctggcttatgtcctgaacgcgttagcgccggcccatccacgtccggtgccttgtgtcaacgtagcgccgctgattggcggg

SEQ ID NO: 285 - protein, 319 aa, synthetic construct
MRMSRRAKLRCALPLAPVAVVAAFAPAAWAQLAVDYSLPKALAAWAANPNGSPPGANDWSCRPSSLHPYPVILVHGTFENQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTWSGLTDFARLLGPIGNNAVQLVTSFCPACGDQIRGSAFMQELNAGGDTVAGVAYTVIQTKYDEVVTPYDSAFLTPAASSHQVRNILVQKGCPIDASEHLAIPFDRRALAYVLNALDPAHPRPVCCVPVAPLIGG

SEQ ID NO: 286 - DNA, 957 bp, synthetic construct
  source 1..957: mol_type = other DNA
atgcgtatgtctcgtcgtgccaaactgcgctgcgccttaccccttgcgcctgtagctgtggttgctgcctttgcacccgcggcatgggcacaactggcggtggactatagcctgccgaaagcgctcgctgcatgggcagccaatccgaatggcagcccaccgggtgcgaacgattggtcgtgtcgcccaagctccttgcacccttatccggtgattctggtccacggtacattcgagaaccagcgcttcaattggaatgccctggcgcctctgctgaagaacgcgggttactgcgtcttttcctttaactatggagcaacgcaaggtacgcaacttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctggagtggcctgactgactttgctcgcttgctcggccctattggcaataacgccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacgcgtcggagcatctggcgattccgtttgatcgtcgtgcgctggcttatgtcctgaacgcgttagatccggcccatccacgtccggtgtgttgtgtcccggtagcgccgctgattggcggg

SEQ ID NO: 287 - protein, 319 aa, synthetic construct
MRMSRRAELRAALPLALVAVVAAFAPAANAQLAVDYSLPKALAAWAANPNGSPPGANDWSCRPSSLHPYPVILVHGTFENQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTWSGLTDFARLLGPIGNNAVQLVTSFCPACGDQIRGSAFMQELNAGGDTVAGVAYTVIQTKYDEVVTPYDSAFLTPAASSHQVRNILVQKGCPIDASEHLAIPFDRRALAYVLNALDPAHPRPVCCVPVAPLIGG

SEQ ID NO: 288 - DNA, 957 bp, synthetic construct
  source 1..957: mol_type = other DNA
atgcgtatgtctcgtcgtgccgaactgcgcgcagccttaccccttgcgctggtagctgtggttgctgcctttgcacccgcggcaaatgcacaactggcggtggactatagcctgccgaaagcgctcgctgcatgggcagccaatccgaatggcagcccaccgggtgcgaacgattggtcgtgtcgcccaagctccttgcacccttatccggtgattctggtccacggtacattcgagaaccagcgcttcaattggaatgccctggcgcctctgctgaagaacgcgggttactgcgtcttttcctttaactatggagcaacgcaaggtacgcaacttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctggagtggcctgactgactttgctcgcttgctcggccctattggcaataacgccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacgcgtcggagcatctggcgattccgtttgatcgtcgtgcgctggcttatgtcctgaacgcgttagatccggcccatccacgtccggtgtgttgtgtcccggtagcgccgctgattggcggg

SEQ ID NO: 289 - protein, 319 aa, synthetic construct
MRMPRRAKLRCALPLALVAVVAAFAPAAWAQLAVDYSLPKALAAWAANPNGSPPGANDWSCRPSSLHPYPVILVHGTFSNQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTWSGLTDFARLLGPIGNNAVQLVTSFCPACGDQIRGSAFMQELNAGGDTVAGVAYTVIQTKYDEVVTPYDSAFLTPAASSHQVRNILVQKGCPIDASEHLAIPFDRRALAYVLNALDPAHPRPVCCVPVAPLIGG

SEQ ID NO: 290 - DNA, 962 bp, synthetic construct
  source 1..962: mol_type = other DNA
atcatatgcgtatgccccgtcgtgccaaactgcgctgcgccttaccccttgcgctggtagctgtggttgctgcctttgcacccgcggcatgggcacaactggcggtggactatagcctgccgaaagcgctcgctgcatgggcagccaatccgaatggcagcccaccgggtgcgaacgattggtcgtgtcgcccaagctccttgcacccttatccggtgattctggtccacggtacattcagcaaccagcgcttcaattggaatgccctggcgcctctgctgaagaacgcgggttactgcgtcttttcctttaactatggagcaacgcaaggtacgcaacttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctggagtggcctgactgactttgctcgcttgctcggccctattggcaataacgccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacgcgtcggagcatctggcgattccgtttgatcgtcgtgcgctggcttatgtcctgaacgcgttagatccggcccatccacgtccggtgtgttgtgtcccggtagcgccgctgattggcggg

SEQ ID NO: 291 - protein, 319 aa, synthetic construct
MRMSRRAKLRCALPLALVAVVAAFAPAAWAQLAVDYSLPKALAAWAANPNGSPPGANDWSCRPSSLHPYPVILVHGTFSNQRFNWNALAPLLKNAGYCVFSFNYGATQGTELTGGFANGLAAVRDSARQLAAFVDRVRAATGAAEVDIVGHSQGGMMPRYYTNKLGGASKVRALVGLSPSNHGTTWSGLTDFARLLGPIGNAAVQLVTSFCPACGDQIRGSAFMQELNAGGDTVAGVAYTVIQTKYDEVVTPYDSAFLTPAASSHQVRNILVQKGCPIDASEHLAIPFDRRALAYVLNALDPAHPRPVCCVNVAPLIGG

SEQ ID NO: 292 - DNA, 957 bp, synthetic construct
  source 1..957: mol_type = other DNA
atgcgtatgtctcgtcgtgccaaactgcgctgcgccttaccccttgcgctggtagctgtggttgctgcctttgcacccgcggcatgggcacaactggcggtggactatagcctgccgaaagcgctcgctgcatgggcagccaatccgaatggcagcccaccgggtgcgaacgattggtcgtgtcgcccaagctccttgcacccttatccggtgattctggtccacggtacattcagcaaccagcgcttcaattggaatgccctggcgcctctgctgaagaacgcgggttactgcgtcttttcctttaactatggagcaacgcaaggtacggagcttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctggagtggcctgactgactttgctcgcttgctcggccctattggcaatgcagccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacgcgtcggagcatctggcgattccgtttgatcgtcgtgcgctggcttatgtcctgaacgcgttagatccggcccatccacgtccggtgtgttgtgtcaacgtagcgccgctgattggcggg

SEQ ID NO: 293 - protein, 319 aa, synthetic construct
MRMSRRAELRAALPLALVAVVAAFAPAANAQLAVDYSLPMALAAWAANPNGSPPGANDWSCRPSSLHPYPVILVHGTFSNQRFNHNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTWSGLTDFARLLGPIGNAAVQLVTSFCPACGDQIRGSAFMQELNAGGDTVAGVAYTVIQTKYDEVVTPYDSAFLTPAASSHQVRNILVQKGCPIDLSEHLAIPFDRRALAYVLNALDPAHPRPVCCVPVAPLIGG

SEQ ID NO: 294 - DNA, 957 bp, synthetic construct
  source 1..957: mol_type = other DNA
atgcgtatgtcccgtcgtgccgaactgcgcgcagccttaccccttgcgctggtagctgtggttgctgcctttgcacccgcggcaaatgcacaactggcggtggactatagcctgccgatggcgctcgctgcatgggcagccaatccgaatggcagcccaccgggtgcgaacgattggtcgtgtcgcccaagctccttgcacccttatccggtgattctggtccacggtacattcagcaaccagcgcttcaatcataatgccctggcgcctctgctgaagaacgcgggttactgcgtcttttcctttaactatggagcaacgcaaggtacgcaacttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctggagtggcctgactgactttgctcgcttgctcggccctattggcaatgcagccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacttatcggagcatctggcgattccgtttgatcgtcgtgcgctggcttatgtcctgaacgcgttagatccggcccatccacgtccggtgtgttgtgtcccggtagcgccgctgattggcggg

SEQ ID NO: 295 - protein, 319 aa, synthetic construct
MRQPRRAKLRCALPLALVAVVAAFAPAAHAQLAVDYSLPMALAAWAANPNGSPPGANDWSCRPSSLHPYPVILVHGTFENQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTYSGLTDFARLLGPIGNNAVQLVTSFCPACGDQIRGSAFMQELNAGGDTVAGVAYTVIQTKYDEVVTPYDSAFLTPAASSHQVRNILVQKGCPIDLSEHLAIPFDRRALAYVLNALDPAHPRPVCCVPVAPLIGG

SEQ ID NO: 296 - DNA, 957 bp, synthetic construct
  source 1..957: mol_type = other DNA
atgcgtcagccccgtcgtgccaaactgcgctgcgccttaccccttgcgcttgtagctgtggttgctgcctttgcacccgcggcacatgcacaactggcggtggactatagcctgccgatggcgctcgctgcatgggcagccaatccgaatggcagcccaccgggtgcgaacgattggtcgtgtcgcccaagctccttgcacccttatccggtgattctggtccacggtacattcgagaaccagcgcttcaattggaatgccctggcgcctctgctgaagaacgcgggttactgcgtcttttcctttaactatggagcaacgcaaggtacgcaacttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctacagtggcctgactgactttgctcgcttgctcggccctattggcaataacgccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacttatcggagcatctggcgattccgtttgatcgtcgtgcgctggcttatgtcctgaacgcgttagatccggcccatccacgtccggtgtgttgtgtcccggtagcgccgctgattggcggg

SEQ ID NO: 297 - protein, 319 aa, synthetic construct
MRMSRRAKLRCALPLALAAVVAAFAPAANAQLAVDYSLPMALAAWAANPNGSPPGANDWSCRPSSLHPYPVILVHGTFENQRFTWNALAPLLKNAGYCVFSFNYGATQGTELTGGFANGLAAVRDSARQLAAFVDRVRAATGAAEVDIVGHSQGGMMPRYYTNKLGGASKVRALVGLSPSNHGTTYSGLTDFARLLGPIGNAAVQLVTSFCPACGDQIRGSAFMQELNAGGDTVAGVAYTVIQTKYDEVVTPYDSAFLTPAASSHQVRNILVQKGCPIDASEHLAIPFDRRALAYVLNALDPAHPRPVCCVNVAPLIGG

SEQ ID NO: 298 - DNA, 957 bp, synthetic construct
  source 1..957: mol_type = other DNA
atgcgtatgtctcgtcgtgccaaactgcgctgcgccttaccccttgcgcttgccgctgtggttgctgcctttgcacccgcggcaaatgcacaactggcggtggactatagcctgccgatggcgctcgctgcatgggcagccaatccgaatggcagcccaccgggtgcgaacgattggtcgtgtcgcccaagctccttgcacccttatccggtgattctggtccacggtacattcgagaaccagcgcttcacttggaatgccctggcgcctctgctgaagaacgcgggttactgcgtcttttcctttaactatggagcaacgcaaggtacggagcttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctacagtggcctgactgactttgctcgcttgctcggccctattggcaatgcagccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacgcgtcggagcatctggcgattccgtttgatcgtcgtgcgctggcttatgtcctgaacgcgttagatccggcccatccacgtccggtgtgttgtgtcaacgtagcgccgctgattggcggg

SEQ ID NO: 299 - protein, 319 aa, synthetic construct
MRMPRRAKLRAALPLALAAVVAAFAPAANAQLAVDYSLPMALAAWAANPNGSPPGANDWSCRPSSLHPYPVILVHGTFENQRFTWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTYSGLTDFARLLGPIGNAAVQLVTSFCPACGDQIRGSAFMQELNAGGDTVAGVAYTVIQTKYDEVVTPYDSAFLTPAASSHQVRNILVQKGCPIDLSEHLAIPFDRRALAYVLNALAPAHPRPVPCVNVAPLIGG

SEQ ID NO: 300 - DNA, 957 bp, synthetic construct
  source 1..957: mol_type = other DNA
atgcgtatgccccgtcgtgccaaactgcgcgcagccttaccccttgcgcttgccgctgtggttgctgcctttgcacccgcggcaaatgcacaactggcggtggactatagcctgccgatggcgctcgctgcatgggcagccaatccgaatggcagcccaccgggtgcgaacgattggtcgtgtcgcccaagctccttgcacccttatccggtgattctggtccacggtacattcgagaaccagcgcttcacttggaatgccctggcgcctctgctgaagaacgcgggttactgcgtcttttcctttaactatggagcaacgcaaggtacgcaacttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctacagtggcctgactgactttgctcgcttgctcggccctattggcaatgcagccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacttatcggagcatctggcgattccgtttgatcgtcgtgcgctggcttatgtcctgaacgcgttagcgccggcccatccacgtccggtgccttgtgtcaacgtagcgccgctgattggcggg

SEQ ID NO: 301 - protein, 319 aa, synthetic construct
MRMPRRAELRAALPLALVAVVAAFAPAANAQLAVDYSLPKALAAWAANPNGSPPGANDWSCRPSSLHPYPVILVHGTFENQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTYSGLTDAARLLGPIGNNAVQLVTSFCPACGDQIRGSAFMQELNAGGDTVAGVAYTVIQTKYDEVVTPYDSAFLTPAASSHQVRNILVQKGCPIDASEHLAIPFDRRALAYVLNALDPAHPRPVCCVPVAPLIGG

SEQ ID NO: 302 - DNA, 957 bp, synthetic construct
  source 1..957: mol_type = other DNA
atgcgtatgccccgtcgtgccgaactgcgcgcagccttaccccttgcgcttgtagctgtggttgctgcctttgcacccgcggcaaatgcacaactggcggtggactatagcctgccgaaagcgctcgctgcatgggcagccaatccgaatggcagcccaccgggtgcgaacgattggtcgtgtcgcccaagctccttgcacccttatccggtgattctggtccacggtacattcgagaaccagcgcttcaattggaatgccctggcgcctctgctgaagaacgcgggttactgcgtcttttcctttaactatggagcaacgcaaggtacgcaacttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctacagtggcctgactgacgctgctcgcttgctcggccctattggcaataacgccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacgcgtcggagcatctggcgattccgtttgatcgtcgtgcgctggcttatgtcctgaacgcgttagatccggcccatccacgtccggtgtgttgtgtcccggtagcgccgctgattggcggg

SEQ ID NO: 303 - protein, 319 aa, synthetic construct
MRMSRRAKLRCALPLALVAVVAAFAPAANAQLAVDYSLPKALAAWAANPNGSPPGANDWSCRPSSLHPYPVILVHGTFSNQRFNWNALAPLLKNAGYCVFSFNYGATQGTELTGGFANGLAAVRDSARQLAAFVDRVRAATGAAEVDIVGHSQGGMMPRYYTNKLGGASKVRALVGLSPSNHGTTWSGLTDFARLLGPIGNAAVQLVTSFCPACGDQIRGSAFMQELNAGGDTVAGVAYTVIQTKYDEVVTPYDSAFLTPAASSHQVRNILVQKGCPIDASEHLAIPFDRRALAYVLNALAPAHPRPVPCVPVAPLIGG

SEQ ID NO: 304 - DNA, 957 bp, synthetic construct
  source 1..957: mol_type = other DNA
atgcgtatgtctcgtcgtgccaaactgcgctgcgccttaccccttgcgcttgtagctgtggttgctgcctttgcacccgcggcaaatgcacaactggcggtggactatagcctgccgaaagcgctcgctgcatgggcagccaatccgaatggcagcccaccgggtgcgaacgattggtcgtgtcgcccaagctccttgcacccttatccggtgattctggtccacggtacattcagcaaccagcgcttcaattggaatgccctggcgcctctgctgaagaacgcgggttactgcgtcttttcctttaactatggagcaacgcaaggtacggagcttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctggagtggcctgactgactttgctcgcttgctcggccctattggcaatgcagccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacgcgtcggagcatctggcgattccgtttgatcgtcgtgcgctggcttatgtcctgaacgcgttagcgccggcccatccacgtccggtgccttgtgtcccggtagcgccgctgattggcggg

SEQ ID NO: 305 - protein, 319 aa, synthetic construct
MRQSRRAKLRCALPLAPVAVVAAFAPAANAQLAVDYSLPMALAAWAANPNGSPPGANDWSCRPSSLHPYPVILVHGTFSNQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTYSGLTDFARLLGPIGNAAVQLVTSFCPACGDQIRGSAFMQELNAGGDTVAGVAYTVIQTKYDEVVTPYDSAFLTPAASSHQVRNILVQKGCPIDLSEHLAIPFDRRALAYVLNALDPAHPRPVPCVPVAPLIGG

SEQ ID NO: 306 - DNA, 957 bp, synthetic construct
  source 1..957: mol_type = other DNA
atgcgtcagtctcgtcgtgccaaactgcgctgcgccttaccccttgcgcctgtagctgtggttgctgcctttgcacccgcggcaaatgcacaactggcggtggactatagcctgccgatggcgctcgctgcatgggcagccaatccgaatggcagcccaccgggtgcgaacgattggtcgtgtcgcccaagctccttgcacccttatccggtgattctggtccacggtacattcagcaaccagcgcttcaattggaatgccctggcgcctctgctgaagaacgcgggttactgcgtcttttcctttaactatggagcaacgcaaggtacgcaacttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctacagtggcctgactgactttgctcgcttgctcggccctattggcaatgcagccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacttatcggagcatctggcgattccgtttgatcgtcgtgcgctggcttatgtcctgaacgcgttagatccggcccatccacgtccggtgccttgtgtcccggtagcgccgctgattggcggg

SEQ ID NO: 307 - protein, 319 aa, synthetic construct
MRMSRRAKLRCALPLAPVAVVAAFAPAANAQLAVDYSLPMALAAWAANPNGSPPGANDWSCRPSSLHPYPVILVHGTFENQRFNWNALAPLLKNAGYCVFSFNYGATQGTELTGGFANGLAAVRDSARQLAAFVDRVRAATGAAEVDIVGHSQGGMMPRYYTNKLGGASKVRALVGLSPSNHGTTWSGLTDFARLLGPIGNAAVQLVTSFCPACGDQIRGSAFMQELNAGGDTVAGVAYTVIQTKYDEVVTPYDSAFLTPAASSHQVRNILVQKGCPIDASEHLAIPFDRRALAYVLNALDPAHPRPVPCVPVAPLIGG

SEQ ID NO: 308 - DNA, 957 bp, synthetic construct
  source 1..957: mol_type = other DNA
atgcgtatgtctcgtcgtgccaaactgcgctgcgccttaccccttgcgcctgtagctgtggttgctgcctttgcacccgcggcaaatgcacaactggcggtggactatagcctgccgatggcgctcgctgcatgggcagccaatccgaatggcagcccaccgggtgcgaacgattggtcgtgtcgcccaagctccttgcacccttatccggtgattctggtccacggtacattcgagaaccagcgcttcaattggaatgccctggcgcctctgctgaagaacgcgggttactgcgtcttttcctttaactatggagcaacgcaaggtacggagcttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctggagtggcctgactgactttgctcgcttgctcggccctattggcaatgcagccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacgcgtcggagcatctggcgattccgtttgatcgtcgtgcgctggcttatgtcctgaacgcgttagatccggcccatccacgtccggtgccttgtgtcccggtagcgccgctgattggcggg

SEQ ID NO: 309 - protein, 319 aa, synthetic construct
MRMSRRAKLRAALPLALVAVVAAFAPAANAQLAVDYSLPKALAAWAANPNGSPPGANDWSCRPSSLHPYPVILVHGTFSNQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTYSGLTDFARLLGPIGNNAVQLVTSFCPACGDQIRGSAFMQELNAGGDTVAGVAYTVIQTKYDEVVTPYDSAFLTPAASSHQVRNILVQKGCPIDLSEHLAIPFDRRALAYVLNALAPAHPRPVPCVPVAPLIGG

SEQ ID NO: 310 - DNA, 957 bp, synthetic construct
  source 1..957: mol_type = other DNA
atgcgtatgtctcgtcgtgccaaactgcgcgcagccttaccccttgcgctggtagctgtggttgctgcctttgcacccgcggcaaatgcacaactggcggtggactatagcctgccgaaagcgctcgctgcatgggcagccaatccgaatggcagcccaccgggtgcgaacgattggtcgtgtcgcccaagctccttgcacccttatccggtgattctggtccacggtacattcagcaaccagcgcttcaattggaatgccctggcgcctctgctgaagaacgcgggttactgcgtcttttcctttaactatggagcaacgcaaggtacgcaacttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctacagtggcctgactgactttgctcgcttgctcggccctattggcaataacgccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacttatcggagcatctggcgattccgtttgatcgtcgtgcgctggcttatgtcctgaacgcgttagcgccggcccatccacgtccggtgccttgtgtcccggtagcgccgctgattggcggg

SEQ ID NO: 311 - protein, 319 aa, synthetic construct
MRMSRRAKLRCALPLAPVAVVAAFAPAANAQLAVDYSLPMALAAWAANPNGSPPGANDWSCRPSSLHPYPVILVHGTFSNQRFNWNALAPLLKNAGYCVFSFNYGATQGTELTGGFANGLAAVRDSARQLAAFVDRVRAATGAAEVDIVGHSQGGMMPRYYTNKLGGASKVRALVGLSPSNHGTTYSGLTDFARLLGPIGNAAVQLVTSFCPACGDQIRGSAFMQELNAGGDTVAGVAYTVIQTKYDEVVTPYDSAFLTPAASSHQVRNILVQKGCPIDLSEHLAIPFDRRALAYVLNALDPAHPRPVPCVPVAPLIGG

SEQ ID NO: 312 - DNA, 957 bp, synthetic construct
  source 1..957: mol_type = other DNA
atgcgtatgtctcgtcgtgccaaactgcgctgcgccttaccccttgcgcctgtagctgtggttgctgcctttgcacccgcggcaaatgcacaactggcggtggactatagcctgccgatggcgctcgctgcatgggcagccaatccgaatggcagcccaccgggtgcgaacgattggtcgtgtcgcccaagctccttgcacccttatccggtgattctggtccacggtacattcagcaaccagcgcttcaattggaatgccctggcgcctctgctgaagaacgcgggttactgcgtcttttcctttaactatggagcaacgcaaggtacggagcttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctacagtggcctgactgactttgctcgcttgctcggccctattggcaatgcagccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacttatcggagcatctggcgattccgtttgatcgtcgtgcgctggcttatgtcctgaacgcgttagatccggcccatccacgtccggtgccttgtgtcccggtagcgccgctgattggcggg

SEQ ID NO: 313 - protein, 319 aa, synthetic construct
MRMSRRAKLRAALPLAPVAVVAAFAPAANAQLAVDYSLPMALAAWAANPNGSPPGANDWSCRPSSLHPYPVILVHGTFSNQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTYSGLTDFARLLGPIGNAAVQLVTSFCPACGDQIRGSAFMQELNAGGDTVAGVAYTVIQTKYDEVVTPYDSAFLTPAASSHQVRNILVQKGCPIDLSEHLAIPFDRRALAYVLNALDPAHPRPVPCVPVAPLIGG

SEQ ID NO: 314 - DNA, 957 bp, synthetic construct
  source 1..957: mol_type = other DNA
atgcgtatgtctcgtcgtgccaaactgcgcgcagccttaccccttgcgcctgtagctgtggttgctgcctttgcacccgcggcaaatgcacaactggcggtggactatagcctgccgatggcgctcgctgcatgggcagccaatccgaatggcagcccaccgggtgcgaacgattggtcgtgtcgcccaagctccttgcacccttatccggtgattctggtccacggtacattcagcaaccagcgcttcaattggaatgccctggcgcctctgctgaagaacgcgggttactgcgtcttttcctttaactatggagcaacgcaaggtacgcaacttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctacagtggcctgactgactttgctcgcttgctcggccctattggcaatgcagccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacttatcggagcatctggcgattccgtttgatcgtcgtgcgctggcttatgtcctgaacgcgttagatccggcccatccacgtccggtgccttgtgtcccggtagcgccgctgattggcggg

SEQ ID NO: 315 - protein, 319 aa, synthetic construct
MRMSRRAKLRCALPLALVAVVAAFAPAANAQLAVDYSLPMALAAWAANPNGSPPGANDWSCRPSSLHPYPVILVHGTFENQRFNWNALAPLLKNAGYCVFSFNYGATQGTELTGGFANGLAAVRDSARQLAAFVDRVRAATGAAEVDIVGHSQGGMMPRYYTNKLGGASKVRALVGLSPSNHGTTYSGLTDFARLLGPIGNAAVQLVTSFCPACGDQIRGSAFMQELNAGGDTVAGVAYTVIQTKYDEVVTPYDSAFLTPAASSHQVRNILVQKGCPIDASEHLAIPFDRRALAYVLNALAPAHPRPVPCVPVAPLIGG

SEQ ID NO: 316 - DNA, 957 bp, synthetic construct
  source 1..957: mol_type = other DNA
atgcgtatgtctcgtcgtgccaaactgcgctgcgccttaccccttgcgctggtagctgtggttgctgcctttgcacccgcggcaaatgcacaactggcggtggactatagcctgccgatggcgctcgctgcatgggcagccaatccgaatggcagcccaccgggtgcgaacgattggtcgtgtcgcccaagctccttgcacccttatccggtgattctggtccacggtacattcgagaaccagcgcttcaattggaatgccctggcgcctctgctgaagaacgcgggttactgcgtcttttcctttaactatggagcaacgcaaggtacggagcttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctacagtggcctgactgactttgctcgcttgctcggccctattggcaatgcagccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacgcgtcggagcatctggcgattccgtttgatcgtcgtgcgctggcttatgtcctgaacgcgttagcgccggcccatccacgtccggtgccttgtgtcccggtagcgccgctgattggcggg

SEQ ID NO: 317 - protein, 319 aa, synthetic construct
MRMSRRAKLRCALPLALVAVVAAFAPAANAQLAVDYSLPKALAAWAANPNGSPPGANDWSCRPSSLHPYPVILVHGTFSNQRFTWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTWSGLTDFARLLGPIGNNAVQLVTSFCPACGDQIRGSAFMQELNAGGDTVAGVAYTVIQTKYDEVVTPYDSAFLTPAASSHQVRNILVQKGCPIDASEHLAIPFDRRALAYVLNALAPAHPRPVPCVPVAPLIGG

SEQ ID NO: 318 - DNA, 957 bp, synthetic construct
  source 1..957: mol_type = other DNA
atgcgtatgtctcgtcgtgccaaactgcgctgcgccttaccccttgcgctggtagctgtggttgctgcctttgcacccgcggcaaatgcacaactggcggtggactatagcctgccgaaagcgctcgctgcatgggcagccaatccgaatggcagcccaccgggtgcgaacgattggtcgtgtcgcccaagctccttgcacccttatccggtgattctggtccacggtacattcagcaaccagcgcttcacttggaatgccctggcgcctctgctgaagaacgcgggttactgcgtcttttcctttaactatggagcaacgcaaggtacgcaacttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctggagtggcctgactgactttgctcgcttgctcggccctattggcaataacgccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacgcgtcggagcatctggcgattccgtttgatcgtcgtgcgctggcttatgtcctgaacgcgttagcgccggcccatccacgtccggtgccttgtgtcccggtagcgccgctgattggcggg

SEQ ID NO: 319 - protein, 319 aa, synthetic construct
MRQSRRAKLRCALPLAPVAVVAAFAPAAHAQLAVDYSLPKALAAWAANPNGSPPGANDWSCRPSSLHPYPVILVHGTFSNQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTYSGLTDFARLLGPIGNAAVQLVTSFCPACGDQIRGSAFMQELNAGGDTVAGVAYTVIQTKYDEVVTPYDSAFLTPAASSHQVRNILVQKGCPIDLSEHLAIPFDRRALAYVLNALAPAHPRPVPCVPVAPLIGG

SEQ ID NO: 320 - DNA, 957 bp, synthetic construct
  source 1..957: mol_type = other DNA
atgcgtcagtcccgtcgtgccaaactgcgctgcgccttaccccttgcgcctgtagctgtggttgctgcctttgcacccgcggcacatgcacaactggcggtggactatagcctgccgaaagcgctcgctgcatgggcagccaatccgaatggcagcccaccgggtgcgaacgattggtcgtgtcgcccaagctccttgcacccttatccggtgattctggtccacggtacattcagcaaccagcgcttcaattggaatgccctggcgcctctgctgaagaacgcgggttactgcgtcttttcctttaactatggagcaacgcaaggtacgcaacttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctacagtggcctgactgactttgctcgcttgctcggccctattggcaatgcagccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacttatcggagcatctggcgattccgtttgatcgtcgtgcgctggcttatgtcctgaacgcgttagcgccggcccatccacgtccggtgccttgtgtcccggtagcgccgctgattggcggg

SEQ ID NO: 321 - protein, 319 aa, synthetic construct
MRMSRRAKLRCALPLAPVAVVAAFAPAANAQLAVDYSLPKALAAWAANPNGSPPGANDWSCRPSSLHPYPVILVHGTFENQRFNWNALAPLLKNAGYCVFSFNYGATQGTELTGGFANGLAAVRDSARQLAAFVDRVRAATGAAEVDIVGHSQGGMMPRYYTNKLGGASKVRALVGLSPSNHGTTYSGLTDFARLLGPIGNNAVQLVTSFCPACGDQIRGSAFMQELNAGGDTVAGVAYTVIQTKYDEVVTPYDSAFLTPAASSHQVRNILVQKGCPIDASEHLAIPFDRRALAYVLNALDPAHPRPVPCVNVAPLIGG

SEQ ID NO: 322 - DNA, 957 bp, synthetic construct
  source 1..957: mol_type = other DNA
atgcgtatgtctcgtcgtgccaaactgcgctgcgccttaccccttgcgcctgtagctgtggttgctgcctttgcacccgcggcaaatgcacaactggcggtggactatagcctgccgaaagcgctcgctgcatgggcagccaatccgaatggcagcccaccgggtgcgaacgattggtcgtgtcgcccaagctccttgcacccttatccggtgattctggtccacggtacattcgagaaccagcgcttcaattggaatgccctggcgcctctgctgaagaacgcgggttactgcgtcttttcctttaactatggagcaacgcaaggtacggagcttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctacagtggcctgactgactttgctcgcttgctcggccctattggcaataacgccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacgcgtcggagcatctggcgattccgtttgatcgtcgtgcgctggcttatgtcctgaacgcgttagatccggcccatccacgtccggtgccttgtgtcaacgtagcgccgctgattggcggg

SEQ ID NO: 323 - protein, 319 aa, synthetic construct
MRQSRRAKLRAALPLALVAVVAAFAPAANAQLAVDYSLPKALAAWAANPNGSPPGANDWSCRPSSLHPYPVILVHGTFENQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTYSGLTDFARLLGPIGNNAVQLVTSFCPACGDQIRGSAFMQELNAGGDTVAGVAYTVIQTKYDEVVTPYDSAFLTPAASSHQVRNILVQKGCPIDASEHLAIPFDRRALAYVLNALDPAHPRPVPCVNVAPLIGG

SEQ ID NO: 324 - DNA, 957 bp, synthetic construct
  source 1..957: mol_type = other DNA
atgcgtcagtcccgtcgtgccaaactgcgcgcagccttaccccttgcgctggtagctgtggttgctgcctttgcacccgcggcaaatgcacaactggcggtggactatagcctgccgaaagcgctcgctgcatgggcagccaatccgaatggcagcccaccgggtgcgaacgattggtcgtgtcgcccaagctccttgcacccttatccggtgattctggtccacggtacattcgagaaccagcgcttcaattggaatgccctggcgcctctgctgaagaacgcgggttactgcgtcttttcctttaactatggagcaacgcaaggtacgcaacttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctacagtggcctgactgactttgctcgcttgctcggccctattggcaataacgccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacgcgtcggagcatctggcgattccgtttgatcgtcgtgcgctggcttatgtcctgaacgcgttagatccggcccatccacgtccggtgccttgtgtcaacgtagcgccgctgattggcggg

SEQ ID NO: 325 - protein, 319 aa, synthetic construct
MRMSRRAKLRCALPLAPVAVVAAFAPAANAQLAVDYSLPKALAAWAANPNGSPPGANDWSCRPSSLHPYPVILVHGTFSNQRFNWNALAPLLKNAGYCVFSFNYGATQGTELTGGFANGLAAVRDSARQLAAFVDRVRAATGAAEVDIVGHSQGGMMPRYYTNKLGGASKVRALVGLSPSNHGTTYSGLTDAARLLGPIGNAAVQLVTSFCPACGDQIRGSAFMQELNAGGDTVAGVAYTVIQTKYDEVVTPYDSAFLTPAASSHQVRNILVQKGCPIDLSEHLAIPFDRRALAYVLNALAPAHPRPVPCVPVAPLIGG

SEQ ID NO: 326 - DNA, 957 bp, synthetic construct
  source 1..957: mol_type = other DNA
atgcgtatgtctcgtcgtgccaaactgcgctgcgccttaccccttgcgcctgtagctgtggttgctgcctttgcacccgcggcaaatgcacaactggcggtggactatagcctgccgaaagcgctcgctgcatgggcagccaatccgaatggcagcccaccgggtgcgaacgattggtcgtgtcgcccaagctccttgcacccttatccggtgattctggtccacggtacattcagcaaccagcgcttcaattggaatgccctggcgcctctgctgaagaacgcgggttactgcgtcttttcctttaactatggagcaacgcaaggtacggagcttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctacagtggcctgactgacgctgctcgcttgctcggccctattggcaatgcagccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacttatcggagcatctggcgattccgtttgatcgtcgtgcgctggcttatgtcctgaacgcgttagcgccggcccatccacgtccggtgccttgtgtcccggtagcgccgctgattggcggg

SEQ ID NO: 327 - protein, 319 aa, synthetic construct
MRMSRRAKLRAALPLALVAVVAAFAPAANAQLAVDYSLPKALAAWAANPNGSPPGANDWSCRPSSLHPYPVILVHGTFSNQRFNHNALAPLLKNAGYCVFSFNYGATQGTELTGGFANGLAAVRDSARQLAAFVDRVRAATGAAEVDIVGHSQGGMMPRYYTNKLGGASKVRALVGLSPSNHGTTYSGLTDFARLLGPIGNAAVQLVTSFCPACGDQIRGSAFMQELNAGGDTVAGVAYTVIQTKYDEVVTPYDSAFLTPAASSHQVRNILVQKGCPIDASEHLAIPFDRRALAYVLNALDPAHPRPVPCVNVAPLIGG

SEQ ID NO: 328 - DNA, 957 bp, synthetic construct
  source 1..957: mol_type = other DNA
atgcgtatgtctcgtcgtgccaaactgcgcgcagccttaccccttgcgctggtagctgtggttgctgcctttgcacccgcggcaaatgcacaactggcggtggactatagcctgccgaaagcgctcgctgcatgggcagccaatccgaatggcagcccaccgggtgcgaacgattggtcgtgtcgcccaagctccttgcacccttatccggtgattctggtccacggtacattcagcaaccagcgcttcaatcataatgccctggcgcctctgctgaagaacgcgggttactgcgtcttttcctttaactatggagcaacgcaaggtacggagcttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctacagtggcctgactgactttgctcgcttgctcggccctattggcaatgcagccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacgcgtcggagcatctggcgattccgtttgatcgtcgtgcgctggcttatgtcctgaacgcgttagatccggcccatccacgtccggtgccttgtgtcaacgtagcgccgctgattggcggg

SEQ ID NO: 329 - protein, 319 aa, synthetic construct
MRMSRRAKLRCALPLAPVAVVAAFAPAANAQLAVDYSLPMALAAWAANPNGSPPGANDWSCRPSSLHPYPVILVHGTFSNQRFTWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTYSGLTDFARLLGPIGNNAVQLVTSFCPACGDQIRGSAFMQELNAGGDTVAGVAYTVIQTKYDEVVTPYDSAFLTPAASSHQVRNILVQKGCPIDLSEHLAIPFDRRALAYVLNALDPAHPRPVCCVPVAPLIGG

SEQ ID NO: 330 - DNA, 957 bp, synthetic construct
  source 1..957: mol_type = other DNA
atgcgtatgtctcgtcgtgccaaactgcgctgcgccttaccccttgcgcctgtagctgtggttgctgcctttgcacccgcggcaaatgcacaactggcggtggactatagcctgccgatggcgctcgctgcatgggcagccaatccgaatggcagcccaccgggtgcgaacgattggtcgtgtcgcccaagctccttgcacccttatccggtgattctggtccacggtacattcagcaaccagcgcttcacttggaatgccctggcgcctctgctgaagaacgcgggttactgcgtcttttcctttaactatggagcaacgcaaggtacgcaacttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctacagtggcctgactgactttgctcgcttgctcggccctattggcaataacgccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacttatcggagcatctggcgattccgtttgatcgtcgtgcgctggcttatgtcctgaacgcgttagatccggcccatccacgtccggtgtgttgtgtcccggtagcgccgctgattggcggg

SEQ ID NO: 331 - protein, 319 aa, synthetic construct
MRMSRRAKLRAALPLALVAVVAAFAPAANAWLAVDYSLPKALAAWAANPNGSPPGANDWSCRPSSLHPYPVILVHGTFSNQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTYSGLTDFARLLGPIGNAAVQLVTSFCPACGDQIRGSAFMQELNAGGDTVAGVAYTVIQTKYDEVVTPYDSAFLTPAASSHQVRNILVQKGCPIDASEHLAIPFDRRALAYVLNALAPAHPRPVCCVPVAPLIGG

SEQ ID NO: 332 - DNA, 957 bp, synthetic construct
  source 1..957: mol_type = other DNA
atgcgtatgtctcgtcgtgccaaactgcgcgcagccttaccccttgcgctggtagctgtggttgctgcctttgcacccgcggcaaatgcatggctggcggtggactatagcctgccgaaagcgctcgctgcatgggcagccaatccgaatggcagcccaccgggtgcgaacgattggtcgtgtcgcccaagctccttgcacccttatccggtgattctggtccacggtacattcagcaaccagcgcttcaattggaatgccctggcgcctctgctgaagaacgcgggttactgcgtcttttcctttaactatggagcaacgcaaggtacgcaacttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctacagtggcctgactgactttgctcgcttgctcggccctattggcaatgcagccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacgcgtcggagcatctggcgattccgtttgatcgtcgtgcgctggcttatgtcctgaacgcgttagcgccggcccatccacgtccggtgtgttgtgtcccggtagcgccgctgattggcggg

SEQ ID NO: 333 - protein, 319 aa, synthetic construct
MRMSRRAELRCALPLALVAVVAAFAPAANAQLAVDYSLPMALAAWAANPNGSPPGANDWSCRPSSLHPYPVILVHGTFSNQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTWSGLTDFARLLGPIGNNAVQLVTSFCPACGDQIRGSAFMQELNAGGDTVAGVAYTVIQTKYDEVVTPYDSAFLTPAASSHQVRNILVQKGCPIDLSEHLAIPFDRRALAYVLNALAPAHPRPVPCVNVAPLIGG

SEQ ID NO: 334 - DNA, 957 bp, synthetic construct
  source 1..957: mol_type = other DNA
atgcgtatgtcccgtcgtgccgaactgcgctgcgccttaccccttgcgctggtagctgtggttgctgcctttgcacccgcggcaaatgcacaactggcggtggactatagcctgccgatggcgctcgctgcatgggcagccaatccgaatggcagcccaccgggtgcgaacgattggtcgtgtcgcccaagctccttgcacccttatccggtgattctggtccacggtacattcagcaaccagcgcttcaattggaatgccctggcgcctctgctgaagaacgcgggttactgcgtcttttcctttaactatggagcaacgcaaggtacgcaacttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctggagtggcctgactgactttgctcgcttgctcggccctattggcaataacgccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacttatcggagcatctggcgattccgtttgatcgtcgtgcgctggcttatgtcctgaacgcgttagcgccggcccatccacgtccggtgccttgtgtcaacgtagcgccgctgattggcggg

SEQ ID NO: 335 - protein, 319 aa, synthetic construct
MRQSRRAKLRAALPLALVAVVAAFAPAANAQLAVDYSLPKALAAWAANPNGSPPGANDWSCRPSSLHPYPVILVHGTFENQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTWSGLTDFARLLGPIGNNAVQLVTSFCPACGDQIRGSAFMQELNAGGDTVAGVAYTVIQTKYDEVVTPYDSAFLTPAASSHQVRNILVQKGCPIDASEHLAIPFDRRALAYVLNALDPAHPRPVCCVNVAPLIGG

SEQ ID NO: 336 - DNA, 957 bp, synthetic construct
  source 1..957: mol_type = other DNA
atgcgtcagtctcgtcgtgccaaactgcgcgcagccttaccccttgcgctggtagctgtggttgctgcctttgcacccgcggcaaatgcacaactggcggtggactatagcctgccgaaagcgctcgctgcatgggcagccaatccgaatggcagcccaccgggtgcgaacgattggtcgtgtcgcccaagctccttgcacccttatccggtgattctggtccacggtacattcgagaaccagcgcttcaattggaatgccctggcgcctctgctgaagaacgcgggttactgcgtcttttcctttaactatggagcaacgcaaggtacgcaacttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctggagtggcctgactgactttgctcgcttgctcggccctattggcaataacgccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacgcgtcggagcatctggcgattccgtttgatcgtcgtgcgctggcttatgtcctgaacgcgttagatccggcccatccacgtccggtgtgttgtgtcaacgtagcgccgctgattggcggg

SEQ ID NO: 337 - protein, 319 aa, synthetic construct
MRMSRRAKLRCALPLAPVAVVAAFAPAANAQLAVDYSLPMALAAWAANPNGSPPGANDWSCRPSSLHPYPVILVHGTFENQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTWSGLTDAARLLGPIGNNAVQLVTSFCPACGDQIRGSAFMQELNAGGDTVAGVAYTVIQTKYDEVVTPYDSAFLTPAASSHQVRNILVQKGCPIDASEHLAIPFDRRALAYVLNALDPAHPRPVCCVPVAPLIGG

SEQ ID NO: 338 - DNA, 957 bp, synthetic construct
  source 1..957: mol_type = other DNA
atgcgtatgtctcgtcgtgccaaactgcgctgcgccttaccccttgcgcctgtagctgtggttgctgcctttgcacccgcggcaaatgcacaactggcggtggactatagcctgccgatggcgctcgctgcatgggcagccaatccgaatggcagcccaccgggtgcgaacgattggtcgtgtcgcccaagctccttgcacccttatccggtgattctggtccacggtacattcgagaaccagcgcttcaattggaatgccctggcgcctctgctgaagaacgcgggttactgcgtcttttcctttaactatggagcaacgcaaggtacgcaacttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctggagtggcctgactgacgctgctcgcttgctcggccctattggcaataacgccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacgcgtcggagcatctggcgattccgtttgatcgtcgtgcgctggcttatgtcctgaacgcgttagatccggcccatccacgtccggtgtgttgtgtcccggtagcgccgctgattggcggg

SEQ ID NO: 339 - protein, 319 aa, synthetic construct
MRMSRRAELRCALPLAPVAVVAAFAPAAWAQLAVDYSLPMALAAWAANPNGSPPGANDWSCRPSSLHPYPVILVHGTFSNQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTWSGLTDAARLLGPIGNAAVQLVTSFCPACGDQIRGSAFMQELNAGGDTVAGVAYTVIQTKYDEVVTPYDSAFLTPAASSHQVRNILVQKGCPIDASEHLAIPFDRRALAYVLNALDPAHPRPVPCVPVAPLIGG

SEQ ID NO: 340 - DNA, 957 bp, synthetic construct
  source 1..957: mol_type = other DNA
atgcgtatgtctcgtcgtgccgaactgcgctgcgccttaccccttgcgcctgtagctgtggttgctgcctttgcacccgcggcatgggcacaactggcggtggactatagcctgccgatggcgctcgctgcatgggcagccaatccgaatggcagcccaccgggtgcgaacgattggtcgtgtcgcccaagctccttgcacccttatccggtgattctggtccacggtacattcagcaaccagcgcttcaattggaatgccctggcgcctctgctgaagaacgcgggttactgcgtcttttcctttaactatggagcaacgcaaggtacgcaacttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctggagtggcctgactgacgctgctcgcttgctcggccctattggcaatgcagccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacgcgtcggagcatctggcgattccgtttgatcgtcgtgcgctggcttatgtcctgaacgcgttagatccggcccatccacgtccggtgccttgtgtcccggtagcgccgctgattggcggg

SEQ ID NO: 341 - protein, 319 aa, synthetic construct
MRMPRRAKLRCALPLALVAVVAAFAPAAHAQLAVDYSLPKALAAWAANPNGSPPGANDWSCRPSSLHPYPVILVHGTFENQRFTWNALAPLLKNAGYCVFSFNYGATQGTELTGGFANGLAAVRDSARQLAAFVDRVRAATGAAEVDIVGHSQGGMMPRYYTNKLGGASKVRALVGLSPSNHGTTYSGLTDFARLLGPIGNNAVQLVTSFCPACGDQIRGSAFMQELNAGGDTVAGVAYTVIQTKYDEVVTPYDSAFLTPAASSHQVRNILVQKGCPIDASEHLAIPFDRRALAYVLNALAPAHPRPVPCVPVAPLIGG

SEQ ID NO: 342 - DNA, 957 bp, synthetic construct
  source 1..957: mol_type = other DNA
atgcgtatgcctcgtcgtgccaaactgcgctgcgccttaccccttgcgctggtagctgtggttgctgcctttgcacccgcggcacatgcacaactggcggtggactatagcctgccgaaagcgctcgctgcatgggcagccaatccgaatggcagcccaccgggtgcgaacgattggtcgtgtcgcccaagctccttgcacccttatccggtgattctggtccacggtacattcgagaaccagcgcttcacttggaatgccctggcgcctctgctgaagaacgcgggttactgcgtcttttcctttaactatggagcaacgcaaggtacggagcttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctacagtggcctgactgactttgctcgcttgctcggccctattggcaataacgccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacgcgtcggagcatctggcgattccgtttgatcgtcgtgcgctggcttatgtcctgaacgcgttagcgccggcccatccacgtccggtgccttgtgtcccggtagcgccgctgattggcggg

SEQ ID NO: 343 - protein, 319 aa, synthetic construct
MRMSRRAKLRCALPLAPVAVVAAFAPAANAQLAVDYSLPMALAAWAANPNGSPPGANDWSCRPSSLHPYPVILVHGTFSNQRFNWNALAPLLKNAGYCVFSFNYGATQGTELTGGFANGLAAVRDSARQLAAFVDRVRAATGAAEVDIVGHSQGGMMPRYYTNKLGGASKVRALVGLSPSNHGTTLSGLTDAARLLGPIGNAAVQLVTSFCPACGDQIRGSAFMQELNAGGDTVAGVAYTVIQTKYDEVVTPYDSAFLTPAASSHQVRNILVQKGCPIDASEHLAIPFDRRALAYVLNALDPAHPRPVCCVNVAPLIGG

SEQ ID NO: 344 - DNA, 957 bp, synthetic construct
  source 1..957: mol_type = other DNA
atgcgtatgtctcgtcgtgccaaactgcgctgcgccttaccccttgcgcctgtagctgtggttgctgcctttgcacccgcggcaaatgcacaactggcggtggactatagcctgccgatggcgctcgctgcatgggcagccaatccgaatggcagcccaccgggtgcgaacgattggtcgtgtcgcccaagctccttgcacccttatccggtgattctggtccacggtacattcagcaaccagcgcttcaattggaatgccctggcgcctctgctgaagaacgcgggttactgcgtcttttcctttaactatggagcaacgcaaggtacggagcttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaaccctgagtggcctgactgacgctgctcgcttgctcggccctattggcaatgcagccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacgcgtcggagcatctggcgattccgtttgatcgtcgtgcgctggcttatgtcctgaacgcgttagatccggcccatccacgtccggtgtgttgtgtcaacgtagcgccgctgattggcggg

SEQ ID NO: 345 - protein, 319 aa, synthetic construct
MRQSRRAKLRCALPLAPCAVVAAFAPAANAQLAVDYSLPKALAAWAANPNGSPPGANDWSCRPSSLHPYPVILVHGTFENQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTYSGLTDFARLLGPIGNNAVQLVTSFCPACGDQIRGSAFMQELNAGGDTVAGVAYTVIQTKYDEVVTPYDSAFLTPAASSHQVRNILVQKGCPIDASEHLAIPFDRRALAYVLNALDPAHPRPVCCVNVAPLIGG

SEQ ID NO: 346 - DNA, 957 bp, synthetic construct
  source 1..957: mol_type = other DNA
atgcgtcagtctcgtcgtgccaaactgcgctgcgccttaccccttgcgccttgcgctgtggttgctgcctttgcacccgcggcaaatgcacaactggcggtggactatagcctgccgaaagcgctcgctgcatgggcagccaatccgaatggcagcccaccgggtgcgaacgattggtcgtgtcgcccaagctccttgcacccttatccggtgattctggtccacggtacattcgagaaccagcgcttcaattggaatgccctggcgcctctgctgaagaacgcgggttactgcgtcttttcctttaactatggagcaacgcaaggtacgcaacttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctacagtggcctgactgactttgctcgcttgctcggccctattggcaataacgccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacgcgtcggagcatctggcgattccgtttgatcgtcgtgcgctggcttatgtcctgaacgcgttagatccggcccatccacgtccggtgtgttgtgtcaacgtagcgccgctgattggcggg

SEQ ID NO: 347 - protein, 319 aa, synthetic construct
MRQPRRAELRAALPLALVAVVAAFAPAANAQLAVDYSLPKALAAWAANPNGSPPGANDWSCRPSSLHPYPVILVHGTFSNQRFNHNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTYSGLTDAARLLGPIGNAAVQLVTSFCPACGDQIRGSAFMQELNAGGDTVAGVAYTVIQTKYDEVVTPYDSAFLTPAASSHQVRNILVQKGCPIDLSEHLAIPFDRRALAYVLNALAPAHPRPVPCVPVAPLIGG

SEQ ID NO: 348 - DNA, 957 bp, synthetic construct
  source 1..957: mol_type = other DNA
atgcgtcagccccgtcgtgccgaactgcgcgcagccttaccccttgcgcttgtagctgtggttgctgcctttgcacccgcggcaaatgcacaactggcggtggactatagcctgccgaaagcgctcgctgcatgggcagccaatccgaatggcagcccaccgggtgcgaacgattggtcgtgtcgcccaagctccttgcacccttatccggtgattctggtccacggtacattcagcaaccagcgcttcaatcataatgccctggcgcctctgctgaagaacgcgggttactgcgtcttttcctttaactatggagcaacgcaaggtacgcaacttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctacagtggcctgactgacgctgctcgcttgctcggccctattggcaatgcagccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacttatcggagcatctggcgattccgtttgatcgtcgtgcgctggcttatgtcctgaacgcgttagcgccggcccatccacgtccggtgccttgtgtcccggtagcgccgctgattggcggg

SEQ ID NO: 349 - protein, 319 aa, synthetic construct
MRMSRRAELRCALPLALVAVVAAFAPAANAQLAVDYSLPKALAAWAANPNGSPPGANDWSCRPSSLHPYPVILVHGTFSNQRFNHNALAPLLKNAGYCVFSFNYGATQGTELTGGFANGLAAVRDSARQLAAFVDRVRAATGAAEVDIVGHSQGGMMPRYYTNKLGGASKVRALVGLSPSNHGTTYSGLTDAARLLGPIGNNAVQLVTSFCPACGDQIRGSAFMQELNAGGDTVAGVAYTVIQTKYDEVVTPYDSAFLTPAASSHQVRNILVQKGCPIDASEHLAIPFDRRALAYVLNALDPAHPRPVCCVPVAPLIGG

SEQ ID NO: 350 - DNA, 957 bp, synthetic construct
  source 1..957: mol_type = other DNA
atgcgtatgtctcgtcgtgccgaactgcgctgcgccttaccccttgcgctggtagctgtggttgctgcctttgcacccgcggcaaatgcacaactggcggtggactatagcctgccgaaagcgctcgctgcatgggcagccaatccgaatggcagcccaccgggtgcgaacgattggtcgtgtcgcccaagctccttgcacccttatccggtgattctggtccacggtacattcagcaaccagcgcttcaatcataatgccctggcgcctctgctgaagaacgcgggttactgcgtcttttcctttaactatggagcaacgcaaggtacggagcttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctacagtggcctgactgacgctgctcgcttgctcggccctattggcaataacgccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacgcgtcggagcatctggcgattccgtttgatcgtcgtgcgctggcttatgtcctgaacgcgttagatccggcccatccacgtccggtgtgttgtgtcccggtagcgccgctgattggcggg

SEQ ID NO: 351 - protein, 319 aa, synthetic construct
MRMSRRAELRCALPLALVAVVAAFAPAANAQLAVDYSLPMALAAWAANPNGSPPGANDWSCRPSSLHPYPVILVHGTFSNQRFNWNALAPLLKNAGYCVFSFNYGATQGTELTGGFANGLAAVRDSARQLAAFVDRVRAATGAAEVDIVGHSQGGMMPRYYTNKLGGASKVRALVGLSPSNHGTTWSGLTDFARLLGPIGNAAVQLVTSFCPACGDQIRGSAFMQELNAGGDTVAGVAYTVIQTKYDEVVTPYDSAFLTPAASSHQVRNILVQKGCPIDASEHLAIPFDRRALAYVLNALAPAHPRPVPCVPVAPLIGG

SEQ ID NO: 352 - DNA, 957 bp, synthetic construct
  source 1..957: mol_type = other DNA
atgcgtatgtctcgtcgtgccgaactgcgctgcgccttaccccttgcgctggtagctgtggttgctgcctttgcacccgcggcaaatgcacaactggcggtggactatagcctgccgatggcgctcgctgcatgggcagccaatccgaatggcagcccaccgggtgcgaacgattggtcgtgtcgcccaagctccttgcacccttatccggtgattctggtccacggtacattcagcaaccagcgcttcaattggaatgccctggcgcctctgctgaagaacgcgggttactgcgtcttttcctttaactatggagcaacgcaaggtacggagcttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctggagtggcctgactgactttgctcgcttgctcggccctattggcaatgcagccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacgcgtcggagcatctggcgattccgtttgatcgtcgtgcgctggcttatgtcctgaacgcgttagcgccggcccatccacgtccggtgccttgtgtcccggtagcgccgctgattggcggg

SEQ ID NO: 353 - protein, 319 aa, synthetic construct
MRMSRRAKLRCALPLAPVAVVAAFAPAANAQLAVDYSLPKALAAWAANPNGSPPGANDWSCRPSSLHPYPVILVHGTFSNQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTYSGLTDAARLLGPIGNNAVQLVTSFCPACGDQIRGSAFMQELNAGGDTVAGVAYTVIQTKYDEVVTPYDSAFLTPAASSHQVRNILVQKGCPIDASEHLAIPFDRRALAYVLNALAPAHPRPVPCVPVAPLIGG

SEQ ID NO: 354 - DNA, 957 bp, synthetic construct
  source 1..957: mol_type = other DNA
atgcgtatgtcccgtcgtgccaaactgcgctgcgccttaccccttgcgcctgtagctgtggttgctgcctttgcacccgcggcaaatgcacaactggcggtggactatagcctgccgaaagcgctcgctgcatgggcagccaatccgaatggcagcccaccgggtgcgaacgattggtcgtgtcgcccaagctccttgcacccttatccggtgattctggtccacggtacattcagcaaccagcgcttcaattggaatgccctggcgcctctgctgaagaacgcgggttactgcgtcttttcctttaactatggagcaacgcaaggtacgcaacttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctacagtggcctgactgacgctgctcgcttgctcggccctattggcaataacgccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacgcgtcggagcatctggcgattccgtttgatcgtcgtgcgctggcttatgtcctgaacgcgttagcgccggcccatccacgtccggtgccttgtgtcccggtagcgccgctgattggcggg

SEQ ID NO: 355 - protein, 319 aa, synthetic construct
MRMSRRAKLRCALPLALVAVVAAFAPAANAWLAVDYSLPKALAAWAANPNGSPPGANDWSCRPSSLHPYPVILVHGTFSNQRFNWNALAPLLKNAGYCVFSFNYGATQGTELTGGFANGLAAVRDSARQLAAFVDRVRAATGAAEVDIVGHSQGGMMPRYYTNKLGGASKVRALVGLSPSNHGTTWSGLTDFARLLGPIGNNAVQLVTSFCPACGDQIRGSAFMQELNAGGDTVAGVAYTVIQTKYDEVVTPYDSAFLTPAASSHQVRNILVQKGCPIDASEHLAIPFDRRALAYVLNALAPAHPRPVPCVPVAPLIGG

SEQ ID NO: 356 - DNA, 957 bp, synthetic construct
  source 1..957: mol_type = other DNA
atgcgtatgtctcgtcgtgccaaactgcgctgcgccttaccccttgcgctggtagctgtggttgctgcctttgcacccgcggcaaatgcatggctggcggtggactatagcctgccgaaagcgctcgctgcatgggcagccaatccgaatggcagcccaccgggtgcgaacgattggtcgtgtcgcccaagctccttgcacccttatccggtgattctggtccacggtacattcagcaaccagcgcttcaattggaatgccctggcgcctctgctgaagaacgcgggttactgcgtcttttcctttaactatggagcaacgcaaggtacggagcttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctggagtggcctgactgactttgctcgcttgctcggccctattggcaataacgccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacgcgtcggagcatctggcgattccgtttgatcgtcgtgcgctggcttatgtcctgaacgcgttagcgccggcccatccacgtccggtgccttgtgtcccggtagcgccgctgattggcggg

SEQ ID NO: 357 - protein, 319 aa, synthetic construct
MRMSRRAKLRCALPLAPVAVVAAFAPAANAQLAVDYSLPMALAAWAANPNGSPPGANDWSCRPSSLHPYPVILVHGTFSNQRFNWNALAPLLKNAGYCVFSFNYGATQGTELTGGFANGLAAVRDSARQLAAFVDRVRAATGAAEVDIVGHSQGGMMPRYYTNKLGGASKVRALVGLSPSNHGTTWSGLTDAARLLGPIGNAAVQLVTSFCPACGDQIRGSAFMQELNAGGDTVAGVAYTVIQTKYDEVVTPYDSAFLTPAASSHQVRNILVQKGCPIDLSEHLAIPFDRRALAYVLNALAPAHPRPVPCVPVAPLIGG

SEQ ID NO: 358 - DNA, 957 bp, synthetic construct
  source 1..957: mol_type = other DNA
atgcgtatgtcccgtcgtgccaaactgcgctgcgccttaccccttgcgcctgtagctgtggttgctgcctttgcacccgcggcaaatgcacaactggcggtggactatagcctgccgatggcgctcgctgcatgggcagccaatccgaatggcagcccaccgggtgcgaacgattggtcgtgtcgcccaagctccttgcacccttatccggtgattctggtccacggtacattcagcaaccagcgcttcaattggaatgccctggcgcctctgctgaagaacgcgggttactgcgtcttttcctttaactatggagcaacgcaaggtacggagcttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctggagtggcctgactgacgctgctcgcttgctcggccctattggcaatgcagccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacttatcggagcatctggcgattccgtttgatcgtcgtgcgctggcttatgtcctgaacgcgttagcgccggcccatccacgtccggtgccttgtgtcccggtagcgccgctgattggcggg

SEQ ID NO: 359 - protein, 319 aa, synthetic construct
MRMSRRAKLRAALPLALVAVVAAFAPAANAQLAVDYSLPMALAAWAANPNGSPPGANDWSCRPSSLHPYPVILVHGTFSNQRFNWNALAPLLKNAGYCVFSFNYGATQGTELTGGFANGLAAVRDSARQLAAFVDRVRAATGAAEVDIVGHSQGGMMPRYYTNKLGGASKVRALVGLSPSNHGTTWSGLTDAARLLGPIGNAAVQLVTSFCPACGDQIRGSAFMQELNAGGDTVAGVAYTVIQTKYDEVVTPYDSAFLTPAASSHQVRNILVQKGCPIDLSEHLAIPFDRRALAYVLNALAPAHPRPVPCVPVAPLIGG

SEQ ID NO: 360 - DNA, 957 bp, synthetic construct
  source 1..957: mol_type = other DNA
atgcgtatgtctcgtcgtgccaaactgcgcgcagccttaccccttgcgctggtagctgtggttgctgcctttgcacccgcggcaaatgcacaactggcggtggactatagcctgccgatggcgctcgctgcatgggcagccaatccgaatggcagcccaccgggtgcgaacgattggtcgtgtcgcccaagctccttgcacccttatccggtgattctggtccacggtacattcagcaaccagcgcttcaattggaatgccctggcgcctctgctgaagaacgcgggttactgcgtcttttcctttaactatggagcaacgcaaggtacggagcttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctggagtggcctgactgacgctgctcgcttgctcggccctattggcaatgcagccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacttatcggagcatctggcgattccgtttgatcgtcgtgcgctggcttatgtcctgaacgcgttagcgccggcccatccacgtccggtgccttgtgtcccggtagcgccgctgattggcggg

SEQ ID NO: 361 - protein, 319 aa, synthetic construct
MRMSRRAKLRCALPLAPAAVVAAFAPAANAQLAVDYSLPMALAAWAANPNGSPPGANDWSCRPSSLHPYPVILVHGTFENQRFNWNALAPLLKNAGYCVFSFNYGATQGTELTGGFANGLAAVRDSARQLAAFVDRVRAATGAAEVDIVGHSQGGMMPRYYTNKLGGASKVRALVGLSPSNHGTTYSGLTDFARLLGPIGNNAVQLVTSFCPACGDQIRGSAFMQELNAGGDTVAGVAYTVIQTKYDEVVTPYDSAFLTPAASSHQVRNILVQKGCPIDASEHLAIPFDRRALAYVLNALDPAHPRPVPCVPVAPLIGG

SEQ ID NO: 362 - DNA, 957 bp, synthetic construct
  source 1..957: mol_type = other DNA
atgcgtatgtctcgtcgtgccaaactgcgctgcgccttaccccttgcgcctgccgctgtggttgctgcctttgcacccgcggcaaatgcacaactggcggtggactatagcctgccgatggcgctcgctgcatgggcagccaatccgaatggcagcccaccgggtgcgaacgattggtcgtgtcgcccaagctccttgcacccttatccggtgattctggtccacggtacattcgagaaccagcgcttcaattggaatgccctggcgcctctgctgaagaacgcgggttactgcgtcttttcctttaactatggagcaacgcaaggtacggagcttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctacagtggcctgactgactttgctcgcttgctcggccctattggcaataacgccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacgcgtcggagcatctggcgattccgtttgatcgtcgtgcgctggcttatgtcctgaacgcgttagatccggcccatccacgtccggtgccttgtgtcccggtagcgccgctgattggcggg

SEQ ID NO: 363 - protein, 319 aa, synthetic construct
MRQSRRAKLRCALPLAPVAVVAAFAPAANAQLAVDYSLPKALAAWAANPNGSPPGANDWSCRPSSLHPYPVILVHGTFSNQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTWSGLTDFARLLGPIGNNAVQLVTSFCPACGDQIRGSAFMQELNAGGDTVAGVAYTVIQTKYDEVVTPYDSAFLTPAASSHQVRNILVQKGCPIDLSEHLAIPFDRRALAYVLNALAPAHPRPVCCVPVAPLIGG

SEQ ID NO: 364 - DNA, 957 bp, synthetic construct
  source 1..957: mol_type = other DNA
atgcgtcagtctcgtcgtgccaaactgcgctgcgccttaccccttgcgcctgtagctgtggttgctgcctttgcacccgcggcaaatgcacaactggcggtggactatagcctgccgaaagcgctcgctgcatgggcagccaatccgaatggcagcccaccgggtgcgaacgattggtcgtgtcgcccaagctccttgcacccttatccggtgattctggtccacggtacattcagcaaccagcgcttcaattggaatgccctggcgcctctgctgaagaacgcgggttactgcgtcttttcctttaactatggagcaacgcaaggtacgcaacttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctggagtggcctgactgactttgctcgcttgctcggccctattggcaataacgccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacttatcggagcatctggcgattccgtttgatcgtcgtgcgctggcttatgtcctgaacgcgttagcgccggcccatccacgtccggtgtgttgtgtcccggtagcgccgctgattggcggg

SEQ ID NO: 365 - protein, 319 aa, synthetic construct
MRQPRRAKLRAALPLAPVAVVAAFAPAANAQLAVDYSLPKALAAWAANPNGSPPGANDWSCRPSSLHPYPVILVHGTFSNQRFNWNALAPLLKNAGYCVFSFNYGATQGTELTGGFANGLAAVRDSARQLAAFVDRVRAATGAAEVDIVGHSQGGMMPRYYTNKLGGASKVRALVGLSPSNHGTTYSGLTDFARLLGPIGNAAVQLVTSFCPACGDQIRGSAFMQELNAGGDTVAGVAYTVIQTKYDEVVTPYDSAFLTPAASSHQVRNILVQKGCPIDLSEHLAIPFDRRALAYVLNALDPAHPRPVPCVPVAPLIGG

SEQ ID NO: 366 - DNA, 957 bp, synthetic construct
  source 1..957: mol_type = other DNA
atgcgtcagccccgtcgtgccaaactgcgcgcagccttaccccttgcgcctgtagctgtggttgctgcctttgcacccgcggcaaatgcacaactggcggtggactatagcctgccgaaagcgctcgctgcatgggcagccaatccgaatggcagcccaccgggtgcgaacgattggtcgtgtcgcccaagctccttgcacccttatccggtgattctggtccacggtacattcagcaaccagcgcttcaattggaatgccctggcgcctctgctgaagaacgcgggttactgcgtcttttcctttaactatggagcaacgcaaggtacggagcttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctacagtggcctgactgactttgctcgcttgctcggccctattggcaatgcagccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacttatcggagcatctggcgattccgtttgatcgtcgtgcgctggcttatgtcctgaacgcgttagatccggcccatccacgtccggtgccttgtgtcccggtagcgccgctgattggcggg

SEQ ID NO: 367 - protein, 319 aa, synthetic construct
MRMSRRAKLRCALPLALVAVVAAFAPAANAQLAVDYSLPMALAAWAANPNGSPPGANDWSCRPSSLHPYPVILVHGTFSNQRFNWNALAPLLKNAGYCVFSFNYGATQGTELTGGFANGLAAVRDSARQLAAFVDRVRAATGAAEVDIVGHSQGGMMPRYYTNKLGGASKVRALVGLSPSNHGTTYSGLTDFARLLGPIGNNAVQLVTSFCPACGDQIRGSAFMQELNAGGDTVAGVAYTVIQTKYDEVVTPYDSAFLTPAASSHQVRNILVQKGCPIDASEHLAIPFDRRALAYVLNALDPAHPRPVCCVNVAPLIGG

SEQ ID NO: 368 - DNA, 957 bp, synthetic construct
  source 1..957: mol_type = other DNA
atgcgtatgtctcgtcgtgccaaactgcgctgcgccttaccccttgcgctggtagctgtggttgctgcctttgcacccgcggcaaatgcacaactggcggtggactatagcctgccgatggcgctcgctgcatgggcagccaatccgaatggcagcccaccgggtgcgaacgattggtcgtgtcgcccaagctccttgcacccttatccggtgattctggtccacggtacattcagcaaccagcgcttcaattggaatgccctggcgcctctgctgaagaacgcgggttactgcgtcttttcctttaactatggagcaacgcaaggtacggagcttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctacagtggcctgactgactttgctcgcttgctcggccctattggcaataacgccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacgcgtcggagcatctggcgattccgtttgatcgtcgtgcgctggcttatgtcctgaacgcgttagatccggcccatccacgtccggtgtgttgtgtcaacgtagcgccgctgattggcggg

SEQ ID NO: 369 - protein, 319 aa, synthetic construct
MRMSRRAKLRAALPLALVAVVAAFAPAAWAWLAVDYSLPKALAAWAANPNGSPPGANDWSCRPSSLHPYPVILVHGTFSNQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTYSGLTDFARLLGPIGNNAVQLVTSFCPACGDQIRGSAFMQELNAGGDTVAGVAYTVIQTKYDEVVTPYDSAFLTPAASSHQVRNILVQKGCPIDASEHLAIPFDRRALAYVLNALAPAHPRPVPCVPVAPLIGG

SEQ ID NO: 370 - DNA, 957 bp, synthetic construct
  source 1..957: mol_type = other DNA
atgcgtatgtctcgtcgtgccaaactgcgcgcagccttaccccttgcgctggtagctgtggttgctgcctttgcacccgcggcatgggcatggctggcggtggactatagcctgccgaaagcgctcgctgcatgggcagccaatccgaatggcagcccaccgggtgcgaacgattggtcgtgtcgcccaagctccttgcacccttatccggtgattctggtccacggtacattcagcaaccagcgcttcaattggaatgccctggcgcctctgctgaagaacgcgggttactgcgtcttttcctttaactatggagcaacgcaaggtacgcaacttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctacagtggcctgactgactttgctcgcttgctcggccctattggcaataacgccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacgcgtcggagcatctggcgattccgtttgatcgtcgtgcgctggcttatgtcctgaacgcgttagcgccggcccatccacgtccggtgccttgtgtcccggtagcgccgctgattggcggg

SEQ ID NO: 371 - protein, 319 aa, synthetic construct
MRMSRRAKLRCALPLAPVAVVAAFAPAANAQLAVDYSLPMALAAWAANPNGSPPGANDWSCRPSSLHPYPVILVHGTFSNQRFNWNALAPLLKNAGYCVFSFNYGATQGTELTGGFANGLAAVRDSARQLAAFVDRVRAATGAAEVDIVGHSQGGMMPRYYTNKLGGASKVRALVGLSPSNHGTTWSGLTDAARLLGPIGNAAVQLVTSFCPACGDQIRGSAFMQELNAGGDTVAGVAYTVIQTKYDEVVTPYDSAFLTPAASSHQVRNILVQKGCPIDASEHLAIPFDRRALAYVLNALDPAHPRPVCCVPVAPLIGG

SEQ ID NO: 372 - DNA, 957 bp, synthetic construct
  source 1..957: mol_type = other DNA
atgcgtatgtctcgtcgtgccaaactgcgctgcgccttaccccttgcgcctgtagctgtggttgctgcctttgcacccgcggcaaatgcacaactggcggtggactatagcctgccgatggcgctcgctgcatgggcagccaatccgaatggcagcccaccgggtgcgaacgattggtcgtgtcgcccaagctccttgcacccttatccggtgattctggtccacggtacattcagcaaccagcgcttcaattggaatgccctggcgcctctgctgaagaacgcgggttactgcgtcttttcctttaactatggagcaacgcaaggtacggagcttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctggagtggcctgactgacgctgctcgcttgctcggccctattggcaatgcagccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacgcgtcggagcatctggcgattccgtttgatcgtcgtgcgctggcttatgtcctgaacgcgttagatccggcccatccacgtccggtgtgttgtgtcccggtagcgccgctgattggcggg

SEQ ID NO: 373 - protein, 319 aa, synthetic construct
MRMSRRAKLRCALPLAPVAVVAAFAPAAHAWLAVDYSLPMALAAWAANPNGSPPGANDWSCRPSSLHPYPVILVHGTFSNQRFNWNALAPLLKNAGYCVFSFNYGATQGTELTGGFANGLAAVRDSARQLAAFVDRVRAATGAAEVDIVGHSQGGMMPRYYTNKLGGASKVRALVGLSPSNHGTTYSGLTDFARLLGPIGNAAVQLVTSFCPACGDQIRGSAFMQELNAGGDTVAGVAYTVIQTKYDEVVTPYDSAFLTPAASSHQVRNILVQKGCPIDASEHLAIPFDRRALAYVLNALDPAHPRPVPCVPVAPLIGG

SEQ ID NO: 374 - DNA, 957 bp, synthetic construct
  source 1..957: mol_type = other DNA
atgcgtatgtctcgtcgtgccaaactgcgctgcgccttaccccttgcgcctgtagctgtggttgctgcctttgcacccgcggcacatgcatggctggcggtggactatagcctgccgatggcgctcgctgcatgggcagccaatccgaatggcagcccaccgggtgcgaacgattggtcgtgtcgcccaagctccttgcacccttatccggtgattctggtccacggtacattcagcaaccagcgcttcaattggaatgccctggcgcctctgctgaagaacgcgggttactgcgtcttttcctttaactatggagcaacgcaaggtacggagcttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctacagtggcctgactgactttgctcgcttgctcggccctattggcaatgcagccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacgcgtcggagcatctggcgattccgtttgatcgtcgtgcgctggcttatgtcctgaacgcgttagatccggcccatccacgtccggtgccttgtgtcccggtagcgccgctgattggcggg

SEQ ID NO: 375 - protein, 319 aa, synthetic construct
MRMSRRAELRCALPLALVAVVAAFAPAANAQLAVDYSLPMALAAWAANPNGSPPGANDWSCRPSSLHPYPVILVHGTFSNQRFNWNALAPLLKNAGYCVFSFNYGATQGTELTGGFANGLAAVRDSARQLAAFVDRVRAATGAAEVDIVGHSQGGMMPRYYTNKLGGASKVRALVGLSPSNHGTTWSGLTDFARLLGPIGNNAVQLVTSFCPACGDQIRGSAFMQELNAGGDTVAGVAYTVIQTKYDEVVTPYDSAFLTPAASSHQVRNILVQKGCPIDASEHLAIPFDRRALAYVLNALDPAHPRPVCCVPVAPLIGG

SEQ ID NO: 376 - DNA, 957 bp, synthetic construct
  source 1..957: mol_type = other DNA
atgcgtatgtcccgtcgtgccgaactgcgctgcgccttaccccttgcgctggtagctgtggttgctgcctttgcacccgcggcaaatgcacaactggcggtggactatagcctgccgatggcgctcgctgcatgggcagccaatccgaatggcagcccaccgggtgcgaacgattggtcgtgtcgcccaagctccttgcacccttatccggtgattctggtccacggtacattcagcaaccagcgcttcaattggaatgccctggcgcctctgctgaagaacgcgggttactgcgtcttttcctttaactatggagcaacgcaaggtacggagcttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctggagtggcctgactgactttgctcgcttgctcggccctattggcaataacgccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacgcgtcggagcatctggcgattccgtttgatcgtcgtgcgctggcttatgtcctgaacgcgttagatccggcccatccacgtccggtgtgttgtgtcccggtagcgccgctgattggcggg

SEQ ID NO: 377 - protein, 319 aa, synthetic construct
MRMPRRAKLRCALPLALVAVVAAFAPAANAQLAVDYSLPMALAAWAANPNGSPPGANDWSCRPSSLHPYPVILVHGTFSNQRFNWNALAPLLKNAGYCVFSFNYGATQGTELTGGFANGLAAVRDSARQLAAFVDRVRAATGAAEVDIVGHSQGGMMPRYYTNKLGGASKVRALVGLSPSNHGTTYSGLTDFARLLGPIGNNAVQLVTSFCPACGDQIRGSAFMQELNAGGDTVAGVAYTVIQTKYDEVVTPYDSAFLTPAASSHQVRNILVQKGCPIDASEHLAIPFDRRALAYVLNALDPAHPRPVCCVPVAPLIGG

SEQ ID NO: 378 - DNA, 957 bp, synthetic construct
  source 1..957: mol_type = other DNA
atgcgtatgcctcgtcgtgccaaactgcgctgcgccttaccccttgcgctggtagctgtggttgctgcctttgcacccgcggcaaatgcacaactggcggtggactatagcctgccgatggcgctcgctgcatgggcagccaatccgaatggcagcccaccgggtgcgaacgattggtcgtgtcgcccaagctccttgcacccttatccggtgattctggtccacggtacattcagcaaccagcgcttcaattggaatgccctggcgcctctgctgaagaacgcgggttactgcgtcttttcctttaactatggagcaacgcaaggtacggagcttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctacagtggcctgactgactttgctcgcttgctcggccctattggcaataacgccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacgcgtcggagcatctggcgattccgtttgatcgtcgtgcgctggcttatgtcctgaacgcgttagatccggcccatccacgtccggtgtgttgtgtcccggtagcgccgctgattggcggg

SEQ ID NO: 379 - protein, 319 aa, synthetic construct
MRQSRRAKLRCALPLAPVAVVAAFAPAAWAWLAVDYSLPKALAAWAANPNGSPPGANDWSCRPSSLHPYPVILVHGTFENQRFNWNALAPLLKNAGYCVFSFNYGATQGTELTGGFANGLAAVRDSARQLAAFVDRVRAATGAAEVDIVGHSQGGMMPRYYTNKLGGASKVRALVGLSPSNHGTTYSGLTDFARLLGPIGNNAVQLVTSFCPACGDQIRGSAFMQELNAGGDTVAGVAYTVIQTKYDEVVTPYDSAFLTPAASSHQVRNILVQKGCPIDASEHLAIPFDRRALAYVLNALDPAHPRPVCCVPVAPLIGG

SEQ ID NO: 380 - DNA, 957 bp, synthetic construct
  source 1..957: mol_type = other DNA
atgcgtcagtctcgtcgtgccaaactgcgctgcgccttaccccttgcgcctgtagctgtggttgctgcctttgcacccgcggcatgggcatggctggcggtggactatagcctgccgaaagcgctcgctgcatgggcagccaatccgaatggcagcccaccgggtgcgaacgattggtcgtgtcgcccaagctccttgcacccttatccggtgattctggtccacggtacattcgagaaccagcgcttcaattggaatgccctggcgcctctgctgaagaacgcgggttactgcgtcttttcctttaactatggagcaacgcaaggtacggagcttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctacagtggcctgactgactttgctcgcttgctcggccctattggcaataacgccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacgcgtcggagcatctggcgattccgtttgatcgtcgtgcgctggcttatgtcctgaacgcgttagatccggcccatccacgtccggtgtgttgtgtcccggtagcgccgctgattggcggg

SEQ ID NO: 381 - protein, 319 aa, synthetic construct
MRMSRRAKLRCALPLAPVAVVAAFAPAANAQLAVDYSLPMALAAWAANPNGSPPGANDWSCRPSSLHPYPVILVHGTFSNQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTYSGLTDFARLLGPIGNNAVQLVTSFCPACGDQIRGSAFMQELNAGGDTVAGVAYTVIQTKYDEVVTPYDSAFLTPAASSHQVRNILVQKGCPIDASEHLAIPFDRRALAYVLNALAPAHPRPVPCVPVAPLIGG

SEQ ID NO: 382 - DNA, 957 bp, synthetic construct
  source 1..957: mol_type = other DNA
atgcgtatgtctcgtcgtgccaaactgcgctgcgccttaccccttgcgcctgtagctgtggttgctgcctttgcacccgcggcaaatgcacaactggcggtggactatagcctgccgatggcgctcgctgcatgggcagccaatccgaatggcagcccaccgggtgcgaacgattggtcgtgtcgcccaagctccttgcacccttatccggtgattctggtccacggtacattcagcaaccagcgcttcaattggaatgccctggcgcctctgctgaagaacgcgggttactgcgtcttttcctttaactatggagcaacgcaaggtacgcaacttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctacagtggcctgactgactttgctcgcttgctcggccctattggcaataacgccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacgcgtcggagcatctggcgattccgtttgatcgtcgtgcgctggcttatgtcctgaacgcgttagcgccggcccatccacgtccggtgccttgtgtcccggtagcgccgctgattggcggg

SEQ ID NO: 383 - protein, 319 aa, synthetic construct
MRQSRRAKLRCALPLAPVAVVAAFAPAANAQLAVDYSLPKALAAWAANPNGSPPGANDWSCRPSSLHPYPVILVHGTFSNQRFNWNALAPLLKNAGYCVFSFNYGATQGTELTGGFANGLAAVRDSARQLAAFVDRVRAATGAAEVDIVGHSQGGMMPRYYTNKLGGASKVRALVGLSPSNHGTTWSGLTDAARLLGPIGNNAVQLVTSFCPACGDQIRGSAFMQELNAGGDTVAGVAYTVIQTKYDEVVTPYDSAFLTPAASSHQVRNILVQKGCPIDASEHLAIPFDRRALAYVLNALDPAHPRPVPCVPVAPLIGG

SEQ ID NO: 384 - DNA, 957 bp, synthetic construct
  source 1..957: mol_type = other DNA
atgcgtcagtctcgtcgtgccaaactgcgctgcgccttaccccttgcgcctgtagctgtggttgctgcctttgcacccgcggcaaatgcacaactggcggtggactatagcctgccgaaagcgctcgctgcatgggcagccaatccgaatggcagcccaccgggtgcgaacgattggtcgtgtcgcccaagctccttgcacccttatccggtgattctggtccacggtacattcagcaaccagcgcttcaattggaatgccctggcgcctctgctgaagaacgcgggttactgcgtcttttcctttaactatggagcaacgcaaggtacggagcttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctggagtggcctgactgacgctgctcgcttgctcggccctattggcaataacgccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacgcgtcggagcatctggcgattccgtttgatcgtcgtgcgctggcttatgtcctgaacgcgttagatccggcccatccacgtccggtgccttgtgtcccggtagcgccgctgattggcggg

SEQ ID NO: 385 - protein, 319 aa, synthetic construct
MRQSRRAKLRCALPLAPAAVVAAFAPAANAQLAVDYSLPKALAAWAANPNGSPPGANDWSCRPSSLHPYPVILVHGTFSNQRFNWNALAPLLKNAGYCVFSFNYGATQGTELTGGFANGLAAVRDSARQLAAFVDRVRAATGAAEVDIVGHSQGGMMPRYYTNKLGGASKVRALVGLSPSNHGTTYSGLTDFARLLGPIGNAAVQLVTSFCPACGDQIRGSAFMQELNAGGDTVAGVAYTVIQTKYDEVVTPYDSAFLTPAASSHQVRNILVQKGCPIDASEHLAIPFDRRALAYVLNALAPAHPRPVPCVNVAPLIGG

SEQ ID NO: 386 - DNA, 957 bp, synthetic construct
  source 1..957: mol_type = other DNA
atgcgtcagtctcgtcgtgccaaactgcgctgcgccttaccccttgcgcctgccgctgtggttgctgcctttgcacccgcggcaaatgcacaactggcggtggactatagcctgccgaaagcgctcgctgcatgggcagccaatccgaatggcagcccaccgggtgcgaacgattggtcgtgtcgcccaagctccttgcacccttatccggtgattctggtccacggtacattcagcaaccagcgcttcaattggaatgccctggcgcctctgctgaagaacgcgggttactgcgtcttttcctttaactatggagcaacgcaaggtacggagcttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctacagtggcctgactgactttgctcgcttgctcggccctattggcaatgcagccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacgcgtcggagcatctggcgattccgtttgatcgtcgtgcgctggcttatgtcctgaacgcgttagcgccggcccatccacgtccggtgccttgtgtcaacgtagcgccgctgattggcggg

SEQ ID NO: 387 - protein, 319 aa, synthetic construct
MRQSRRAKLRCALPLAPAAVVAAFAPAANAQLAVDYSLPMALAAWAANPNGSPPGANDWSCRPSSLHPYPVILVHGTFSNQRFTWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTYSGLTDFARLLGPIGNNAVQLVTSFCPACGDQIRGSAFMQELNAGGDTVAGVAYTVIQTKYDEVVTPYDSAFLTPAASSHQVRNILVQKGCPIDASEHLAIPFDRRALAYVLNALDPAHPRPVPCVPVAPLIGG

SEQ ID NO: 388 - DNA, 957 bp, synthetic construct
  source 1..957: mol_type = other DNA
atgcgtcagtctcgtcgtgccaaactgcgctgcgccttaccccttgcgcctgccgctgtggttgctgcctttgcacccgcggcaaatgcacaactggcggtggactatagcctgccgatggcgctcgctgcatgggcagccaatccgaatggcagcccaccgggtgcgaacgattggtcgtgtcgcccaagctccttgcacccttatccggtgattctggtccacggtacattcagcaaccagcgcttcacttggaatgccctggcgcctctgctgaagaacgcgggttactgcgtcttttcctttaactatggagcaacgcaaggtacgcaacttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctacagtggcctgactgactttgctcgcttgctcggccctattggcaataacgccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacgcgtcggagcatctggcgattccgtttgatcgtcgtgcgctggcttatgtcctgaacgcgttagatccggcccatccacgtccggtgccttgtgtcccggtagcgccgctgattggcggg

SEQ ID NO: 389 - protein, 319 aa, synthetic construct
MRMSRRAKLRCALPLAPVAVVAAFAPAANAQLAVDYSLPMALAAWAANPNGSPPGANDWSCRPSSLHPYPVILVHGTFSNQRFTWNALAPLLKNAGYCVFSFNYGATQGTELTGGFANGLAAVRDSARQLAAFVDRVRAATGAAEVDIVGHSQGGMMPRYYTNKLGGASKVRALVGLSPSNHGTTYSGLTDFARLLGPIGNNAVQLVTSFCPACGDQIRGSAFMQELNAGGDTVAGVAYTVIQTKYDEVVTPYDSAFLTPAASSHQVRNILVQKGCPIDASEHLAIPFDRRALAYVLNALAPAHPRPVPCVPVAPLIGG

SEQ ID NO: 390 - DNA, 957 bp, synthetic construct
  source 1..957: mol_type = other DNA
atgcgtatgtctcgtcgtgccaaactgcgctgcgccttaccccttgcgcctgtagctgtggttgctgcctttgcacccgcggcaaatgcacaactggcggtggactatagcctgccgatggcgctcgctgcatgggcagccaatccgaatggcagcccaccgggtgcgaacgattggtcgtgtcgcccaagctccttgcacccttatccggtgattctggtccacggtacattcagcaaccagcgcttcacttggaatgccctggcgcctctgctgaagaacgcgggttactgcgtcttttcctttaactatggagcaacgcaaggtacggagcttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctacagtggcctgactgactttgctcgcttgctcggccctattggcaataacgccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacgcgtcggagcatctggcgattccgtttgatcgtcgtgcgctggcttatgtcctgaacgcgttagcgccggcccatccacgtccggtgccttgtgtcccggtagcgccgctgattggcggg

SEQ ID NO: 391 - protein, 319 aa, synthetic construct
MRQPRRAKLRCALPLAPVAVVAAFAPAANAQLAVDYSLPMALAAWAANPNGSPPGANDWSCRPSSLHPYPVILVHGTFSNQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTYSGLTDFARLLGPIGNNAVQLVTSFCPACGDQIRGSAFMQELNAGGDTVAGVAYTVIQTKYDEVVTPYDSAFLTPAASSHQVRNILVQKGCPIDASEHLAIPFDRRALAYVLNALAPAHPRPVPCVPVAPLIGG

SEQ ID NO: 392 - DNA, 957 bp, synthetic construct
  source 1..957: mol_type = other DNA
atgcgtcagcctcgtcgtgccaaactgcgctgcgccttaccccttgcgcctgtagctgtggttgctgcctttgcacccgcggcaaatgcacaactggcggtggactatagcctgccgatggcgctcgctgcatgggcagccaatccgaatggcagcccaccgggtgcgaacgattggtcgtgtcgcccaagctccttgcacccttatccggtgattctggtccacggtacattcagcaaccagcgcttcaattggaatgccctggcgcctctgctgaagaacgcgggttactgcgtcttttcctttaactatggagcaacgcaaggtacgcaacttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctacagtggcctgactgactttgctcgcttgctcggccctattggcaataacgccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacgcgtcggagcatctggcgattccgtttgatcgtcgtgcgctggcttatgtcctgaacgcgttagcgccggcccatccacgtccggtgccttgtgtcccggtagcgccgctgattggcggg

SEQ ID NO: 393 - protein, 319 aa, synthetic construct
MRMSRRAKLRCALPLALVAVVAAFAPAAWAWLAVDYSLPKALAAWAANPNGSPPGANDWSCRPSSLHPYPVILVHGTFENQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTWSGLTDFARLLGPIGNAAVQLVTSFCPACGDQIRGSAFMQELNAGGDTVAGVAYTVIQTKYDEVVTPYDSAFLTPAASSHQVRNILVQKGCPIDLSEHLAIPFDRRALAYVLNALDPAHPRPVPCVNVAPLIGG

SEQ ID NO: 394 - DNA, 957 bp, synthetic construct
  source 1..957: mol_type = other DNA
atgcgtatgtcccgtcgtgccaaactgcgctgcgccttaccccttgcgctggtagctgtggttgctgcctttgcacccgcggcatgggcatggctggcggtggactatagcctgccgaaagcgctcgctgcatgggcagccaatccgaatggcagcccaccgggtgcgaacgattggtcgtgtcgcccaagctccttgcacccttatccggtgattctggtccacggtacattcgagaaccagcgcttcaattggaatgccctggcgcctctgctgaagaacgcgggttactgcgtcttttcctttaactatggagcaacgcaaggtacgcaacttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctggagtggcctgactgactttgctcgcttgctcggccctattggcaatgcagccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacttatcggagcatctggcgattccgtttgatcgtcgtgcgctggcttatgtcctgaacgcgttagatccggcccatccacgtccggtgccttgtgtcaacgtagcgccgctgattggcggg

SEQ ID NO: 395 - protein, 319 aa, synthetic construct
MRMSRRAKLRCALPLALVAVVAAFAPAANAQLAVDYSLPKALAAWAANPNGSPPGANDWSCRPSSLHPYPVILVHGTFENQRFNWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTYSGLTDAARLLGPIGNAAVQLVTSFCPACGDQIRGSAFMQELNAGGDTVAGVAYTVIQTKYDEVVTPYDSAFLTPAASSHQVRNILVQKGCPIDLSEHLAIPFDRRALAYVLNALDPAHPRPVCCVNVAPLIGG

SEQ ID NO: 396 - DNA, 957 bp, synthetic construct
  source 1..957: mol_type = other DNA
atgcgtatgtctcgtcgtgccaaactgcgctgcgccttaccccttgcgctggtagctgtggttgctgcctttgcacccgcggcaaatgcacaactggcggtggactatagcctgccgaaagcgctcgctgcatgggcagccaatccgaatggcagcccaccgggtgcgaacgattggtcgtgtcgcccaagctccttgcacccttatccggtgattctggtccacggtacattcgagaaccagcgcttcaattggaatgccctggcgcctctgctgaagaacgcgggttactgcgtcttttcctttaactatggagcaacgcaaggtacgcaacttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctacagtggcctgactgacgctgctcgcttgctcggccctattggcaatgcagccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacttatcggagcatctggcgattccgtttgatcgtcgtgcgctggcttatgtcctgaacgcgttagatccggcccatccacgtccggtgtgttgtgtcaacgtagcgccgctgattggcggg

SEQ ID NO: 397 - protein, 319 aa, synthetic construct
MRMSRRAKLRCALPLALVAVVAAFAPAAWAWLAVDYSLPKALAAWAANPNGSPPGANDWSCRPSSLHPYPVILVHGTFENQRFNHNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTYSGLTDFARLLGPIGNAAVQLVTSFCPACGDQIRGSAFMQELNAGGDTVAGVAYTVIQTKYDEVVTPYDSAFLTPAASSHQVRNILVQKGCPIDLSEHLAIPFDRRALAYVLNALDPAHPRPVPCVNVAPLIGG

SEQ ID NO: 398 - DNA, 957 bp, synthetic construct
  source 1..957: mol_type = other DNA
atgcgtatgtctcgtcgtgccaaactgcgctgcgccttaccccttgcgctggtagctgtggttgctgcctttgcacccgcggcatgggcatggctggcggtggactatagcctgccgaaagcgctcgctgcatgggcagccaatccgaatggcagcccaccgggtgcgaacgattggtcgtgtcgcccaagctccttgcacccttatccggtgattctggtccacggtacattcgagaaccagcgcttcaatcataatgccctggcgcctctgctgaagaacgcgggttactgcgtcttttcctttaactatggagcaacgcaaggtacgcaacttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctacagtggcctgactgactttgctcgcttgctcggccctattggcaatgcagccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacttatcggagcatctggcgattccgtttgatcgtcgtgcgctggcttatgtcctgaacgcgttagatccggcccatccacgtccggtgccttgtgtcaacgtagcgccgctgattggcggg

SEQ ID NO: 399 - protein, 319 aa, synthetic construct
MRMSRRAKLRCALPLALVAVVAAFAPAANAQLAVDYSLPMADAAWAANPNGSPPGANDWSCRPSSLHPYPVILVHGTFSNQRFTWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTYSGLTDAARLLGPIGNAAVQLVTSFCPACGDQIRGSAFMQELNAGGDTVAGVAYTVIQTKYDEVVTPYDSAFLTPAASSHQVRNILVQKGCPIDLSEHLAIPFDRRALAYVLNALAPAHPRPVPCVPVAPLIGG

SEQ ID NO: 400 - DNA, 957 bp, synthetic construct
  source 1..957: mol_type = other DNA
atgcgtatgtctcgtcgtgccaaactgcgctgcgccttaccccttgcgctggtagctgtggttgctgcctttgcacccgcggcaaatgcacaactggcggtggactatagcctgccgatggcggacgctgcatgggcagccaatccgaatggcagcccaccgggtgcgaacgattggtcgtgtcgcccaagctccttgcacccttatccggtgattctggtccacggtacattcagcaaccagcgcttcacatggaatgccctggcgcctctgctgaagaacgcgggttactgcgtcttttcctttaactatggagcaacgcaaggtacgcaacttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctacagtggcctgactgacgcggctcgcttgctcggccctattggcaatgcagccgttcagcttgtcaccagcttctgcccggcatgtggggatcagatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacttatcggagcatctggcgattccgtttgatcgtcgtgcgctggcttatgtcctgaacgcgttagcgccggcccatccacgtccggtgccttgtgtcccggtagcgccgctgattggcggg

SEQ ID NO: 401 - protein, 319 aa, synthetic construct
MRMSRRAKLRCALPLALVAVVAAFAPAANAQLAVDYSLPKALAAWAANPNGSPPGANDWSCRPSSLHPYPVILVHGTFSNQRFTWNALAPLLKNAGYCVFSFNYGATQGTQLTGGFANGLAAVRDSARQLAAFVDRVRAATGAAEVDIVGHSQGGMMPRYYTNKLGGASKVRALVGLSPSNHGTTYSGLTDVARLLGPIGNAAVQLVTSFCPACGDMIRGSAFMQELNAGGDTVAGVAYTVIQTKYDEVVTPYDSAFLTPAASSHQVRNILVQKGCPIDLSEHLAIPFDRRALAYVLNALAPAHPRPVPCVPVAPLIGG

SEQ ID NO: 402 - DNA, 957 bp, synthetic construct
  source 1..957: mol_type = other DNA
atgcgtatgtctcgtcgtgccaaactgcgctgcgccttaccccttgcgctggtagctgtggttgctgcctttgcacccgcggcaaatgcacaactggcggtggactatagcctgccgaaagcgctcgctgcatgggcagccaatccgaatggcagcccaccgggtgcgaacgattggtcgtgtcgcccaagctccttgcacccttatccggtgattctggtccacggtacattcagcaaccagcgcttcacatggaatgccctggcgcctctgctgaagaacgcgggttactgcgtcttttcctttaactatggagcaacgcaaggtacgcaacttaccggcggttttgccaacgggttggcggccgttcgcgactctgctcggcaattggccgcgttcgttgatcgcgtgcgtgcagccactggcgcagccgaagttgacatcgtggggcattcccagggcggtatgatgccgcgctactacacgaacaaactgggcggtgcaagcaaagtgcgcgcgttagtcggactgtcaccctctaaccacggtacaacctacagtggcctgactgacgtggctcgcttgctcggccctattggcaatgcagccgttcagcttgtcaccagcttctgcccggcatgtggggatatgatccgcggtagtgctttcatgcaggaactgaatgcgggtggcgataccgtagcgggagttgcctatacggtgattcagaccaagtacgatgaagtggtgaccccgtatgattcagcgtttctcaccccagctgcgtcgagtcatcaggtacggaacatcctcgttcagaaaggctgcccgatcgacttatcggagcatctggcgattccgtttgatcgtcgtgcgctggcttatgtcctgaacgcgttagcgccggcccatccacgtccggtgccttgtgtcccggtagcgccgctgattggcggg